(19) United States
(12) Patent Application Publication
(10) Pub. No.: US 2006/0079554 A1
(43) Pub. Date: Apr. 13, 2006

(54) Inhibitors of monomine uptake

(76) Inventors: Peter Clark Barry, Hampshire (GB); Manuel Javier Cases-Thomas, Reading (GB); Peter Thaddeus Gallagher, Hampshire (GB); Jeremy Gilmore, Surrey (GB); John Joseph Masters, Fishers, IN (US); Graham Henry Timms, Hampshire (GB); Maria Ann Whatton, Bracknell (GB); Virginia Ann Wood, Surrey (GB)

Correspondence Address: ELI LILLY & COMPANY, PATENT DIVISION, P.O. BOX 6288, INDIANAPOLIS, IN, 46206-6288, US

(21) Appl. No.: 10/536,295

(22) PCT Filed: Nov. 25, 2003
(86) PCT No.: PCT/US03/35972

§ 371 Date: May 25, 2005

Related U.S. Application Data
(60) Provisional application No. 60/434,720, filed on Dec. 18, 2002.

(30) Foreign Application Priority Data
Dec. 6, 2002 (GB) 0228482.6

(51) Int. Cl. A61K 31/445; C07D 211/56

(52) U.S. Cl. 514/317; 546/223

(57) ABSTRACT

N,N-disubstituted 4-amino-piperidines of the general Formula (I) are inhibitors of the uptake of serotonin and/or norepinephrine and/or dopamine. As such, they may be useful for the treatment of disorders of the central and/or peripheral nervous system.

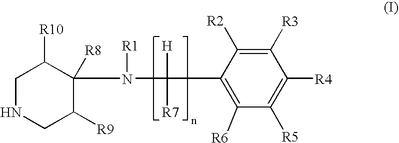

The present invention is directed to compounds which inhibit the uptake of one or more physiologically active monoamines selected from serotonin (also called 5-hydroxytryptamine or 5-HT), norepinephrine (also called noradrenaline) and dopamine. There is a large body of scientific evidence pointing to the physiological role of these monoamines as neurotransmitters. Consequently, compounds which are capable of inhibiting the uptake of one or more of these monoamines find utility in the treatment of disorders of the central and/or peripheral nervous system.

It is known that the 3-aryloxy-3-substituted-1-aminopropane class of compounds have demonstrated particular diversity in their ability to inhibit the uptake of monoamines. Fluoxetine (N-methyl 3-((4-trifluoromethylphenyl)oxy)-3-phenyl-1-aminopropane hydrochloride), for example, is a selective serotonin uptake inhibitor that has found great market acceptance in the treatment of depression and has also been approved for the treatment of a number of other disorders. Atomoxetine ((−)-N-methyl 3-((2-methylphenyl)oxy)-3-phenyl-1-aminopropane hydrochloride), is a selective norepinephrine uptake inhibitor that is approved for the treatment of attention deficit/hyperactivity disorder. Duloxetine ((+)-N-methyl 3-(1-naphthalenyloxy)-3-(2-thienyl)-1-aminopropane hydrochloride), is a dual serotonin and norepinephrine uptake inhibitor that is in clinical development for the treatment of depression.

EP-A2-0112776 discloses the compound N-ethyl-N-benzyl-4-piperidinamine as an intermediate in the synthesis of naphthalene- or azanaphthalene-carboxamides.

It would be advantageous to provide further compounds which are capable of inhibiting the uptake of one or more monoamines selected from serotonin, norepinephrine and dopamine. Preferably, such compounds would exhibit one or more of the following characteristics when compared with known monoamine uptake inhibitors—(i) improved potency in their inhibition of one or more of these monoamines, (ii) improved selectivity in their inhibition of one or more of these monoamines, (iii) improved bioavailability, (iv) minimal interaction with metabolic enzymes such as CYP2D6 and (v) improved acid stability.

Accordingly, the present invention provides a compound of formula I 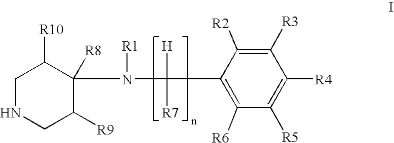
wherein
n is 1, 2 or 3;
R1 is C₂-C₁₀alkyl, C₂-C₁₀alkenyl, C₃-C₈cycloalkyl or C₄-C₁₀cycloalkylalkyl, wherein one C—C bond within any cycloalkyl moiety is optionally substituted by an O—C, S—C or C═C bond and wherein each group is optionally substituted with from 1 to 7 halogen substituents and/or with from 1 to 3 substituents each independently selected from hydroxy, cyano, C₁-C₄alkyl, C₁-C₄alkylthio (optionally substituted with from 1 to 3 halogen atoms) and C₁-C₄alkoxy (optionally substituted with from 1 to 3 halogen atoms);
R2 is H, C₁-C₄alkyl (optionally substituted with from 1 to 7 halogen atoms), C₁-C₄alkyl-S(O)ₓ— wherein x is 0, 1 or 2 (optionally substituted with from 1 to 7 halogen atoms), C₁-C₄alkoxy (optionally substituted with from 1 to 7 halogen atoms), cyano, halogen, phenyl (optionally substituted with from 1 to 3 substituents each independently selected from halogen, C₁-C₄alkyl and C₁-C₄alkoxy), phenoxy (optionally substituted with from 1 to 3 substituents each independently selected from halogen, C₁-C₄alkyl and C₁-C₄alkoxy) or —CO₂(C₁-C₄alkyl), or together with R3 forms a further benzene ring (optionally substituted with from 1 to 3 substituents each independently selected from halogen, C₁-C₄alkyl and C₁-C₄alkoxy); R3 is H, C₁-C₄alkyl (optionally substituted with from 1 to 7 halogen atoms), C₁-C₄alkyl-S(O)ₓ— wherein x is 0, 1 or 2 (optionally substituted with from 1 to 7 halogen atoms), C₁-C₄alkoxy (optionally substituted with from 1 to 7 halogen atoms), cyano, halogen, phenyl (optionally substituted with from 1 to 3 substituents each independently selected from halogen, C₁-C₄alkyl and C₁-C₄alkoxy), phenoxy (optionally substituted with from 1 to 3 substituents each independently selected from halogen, C₁-C₄alkyl and C₁-C₄alkoxy) or —CO₂(C₁-C₄alkyl), or together with R2 or R4 forms a further benzene ring (optionally substituted with from 1 to 3 substituents each independently selected from halogen, C₁-C₄alkyl and C₁-C₄alkoxy);
R4 is H, C₁-C₄alkyl (optionally substituted with from 1 to 7 halogen atoms), C₁-C₄alkyl-S(O)ₓ— wherein x is 0, 1 or 2 (optionally substituted with from 1 to 7 halogen atoms), C₁-C₄alkoxy (optionally substituted with from 1 to 7 halogen atoms), cyano, halogen, phenyl (optionally substituted with from 1 to 3 substituents each independently selected from halogen, C₁-C₄alkyl and C₁-C₄alkoxy), phenoxy (optionally substituted with from 1 to 3 substituents each independently selected from halogen, C₁-C₄alkyl and C₁-C₄alkoxy) or —CO₂(C₁-C₄alkyl), or together with R3 forms a further benzene ring (optionally substituted with from 1 to 3 substituents each independently selected from halogen, C₁-C₄alkyl and C₁-C₄alkoxy);
R5 is H, C₁-C₄alkyl (optionally substituted with from 1 to 7 halogen atoms), C₁-C₄alkoxy (optionally substituted with from 1 to 7 halogen atoms) or halogen;
R6 is H, C₁-C₄alkyl (optionally substituted with from 1 to 7 halogen atoms), C₁-C₄alkoxy (optionally substituted with from 1 to 7 halogen atoms) or halogen;
R7 is H or C₁-C₄alkyl;
R8 is H or C₁-C₄alkyl;
R9 is H, halogen, hydroxy, cyano, C₁-C₄alkyl or C₁-C₄alkoxy; and
R10 is H, halogen, hydroxy, cyano, C₁-C₄alkyl or C₁-C₄alkoxy;
   or a pharmaceutically acceptable salt thereof,
   with the proviso that the compound N-ethyl-N-benzyl-4-piperidinamine is excluded.

In a further embodiment, the present invention provides a compound of formula I above wherein

R1 is C₂-C₁₀alkyl, C₂-C₁₀alkenyl, C₃-C₈cycloalkyl or C₄-C₁₀cycloalkylalkyl, wherein one C—C bond within any cycloalkyl moiety is optionally substituted by an O—C or C═C bond and wherein each group is optionally substituted with from 1 to 7 halogen substituents and/or with from 1 to 3 substituents each independently selected from hydroxy, cyano, C₁-C₄alkyl and C₁-C₄alkoxy; and
n; R2; R3; R4; R5; R6; R7; R8; R9; and R10 are as defined above;
   or a pharmaceutically acceptable salt thereof,
   with the proviso that the compound N-ethyl-N-benzyl-4-piperidinamine is excluded.

In the present specification the term “C₂-C₁₀alkyl” means a monovalent unsubstituted saturated straight-chain or branched-chain hydrocarbon radical having from 2 to 10 carbon atoms.

In the present specification the term “C₂-C₁₀alkenyl” means a monovalent unsubstituted unsaturated straight-chain or branched-chain hydrocarbon radical having from 2 to 10 carbon atoms and containing at least one carbon-carbon double bond.

In the present specification the term “C₃-Cgcycloalkyl” means a monovalent unsubstituted saturated cyclic hydrocarbon radical having from 3 to 8 carbon atoms.

In the present specification the term “C₄-C₁₀cycloalkylalkyl” means a monovalent unsubstituted saturated cyclic hydrocarbon radical having from 3 to 9 carbon atoms linked to the point of substitution by a divalent unsubstituted saturated straight-chain or branched-chain hydrocarbon radical having at least 1 carbon atom.

In the present specification the phrase “wherein one C—C bond within any cycloalkyl moiety is optionally substituted by an O—C, S—C or C═C bond” means that either (i) any two adjacent carbon atoms within a cycloalkyl ring may be linked by a double bond rather than a single bond (with the number of substituents on each carbon atom being reduced accordingly), or that (ii) one of any two adjacent C atoms within a cycloalkyl ring (and any substituents thereon) may be replaced by an oxygen or sulphur atom. Examples of R1 groups encompassed by this phrase include but are not limited to:

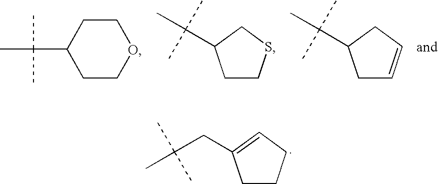

In the present specification the term “halo” or “halogen” means F, Cl, Br or I.

In the present specification the term “C₁-C₄alkylthio” means a monovalent unsubstituted saturated straight-chain or branched-chain hydrocarbon radical having from 1 to 4 carbon atoms linked to the point of substitution by a S atom.

In the present specification the term “C₁-C₄alkoxy” means a monovalent unsubstituted saturated straight-chain or branched-chain hydrocarbon radical having from 1 to 4 carbon atoms linked to the point of substitution by an O atom.

In the present specification the term “phenoxy” means a monovalent unsubstituted phenyl radical linked to the point of substitution by an O atom.

In the above definitions, similar terms specifying different numbers of C atoms take an analogous meaning.

In a preferred embodiment, n is 1 or 2. More preferably, n is 1.

In a preferred embodiment, R7 is H or methyl. More preferably R7 is H.

In a preferred embodiment, R8 is H.

In a preferred embodiment, R9 is H or fluoro. More preferably, R9 is H.

In a preferred embodiment, R10 is H or fluoro. More preferably, R10 is H.

In a preferred embodiment, R1 is C₂-C₆alkyl, C₂-C₆alkenyl, C₃-C₆cycloalkyl or C₄-C₇cycloalkylalkyl, wherein one C—C bond within any cycloalkyl moiety is optionally substituted by an O—C bond and wherein each group is optionally substituted with from 1 to 3 halogen atoms or a hydroxy, cyano, C₁-C₄alkylthio (optionally substituted with from 1 to 3 halogen atoms) or C₁-C₄alkoxy (optionally substituted with from 1 to 3 halogen atoms) radical. More preferably, R1 is C₂-C₆alkyl, C₂-C₆alkenyl, C₃-C₆cycloalkyl or C₄-C₇cycloalkylalkyl, wherein one C—C bond within any cycloalkyl moiety is optionally substituted by an O—C bond and wherein each group is optionally substituted with from 1 to 3 halogen atoms or a hydroxy, cyano, methylthio, methoxy, trifluoromethoxy, ethoxy, or isopropoxy radical. More preferably, R1 is C₂-C₆alkyl (optionally substituted with from 1 to 3 halogen atoms or a hydroxy, cyano, methylthio, methoxy, trifluoromethoxy, ethoxy, or isopropoxy radical), C₂-C₆alkenyl, C₃-C₆cycloalkyl or C₄-C₇cycloalkylalkyl (optionally substituted with a halogen atom or hydroxy radical), wherein one C—C bond within any cycloalkyl moiety is optionally substituted by an O—C bond. Suitable C₂-C₆alkyl groups (optionally substituted with from 1 to 3 halogen atoms or a hydroxy, cyano, methylthio, methoxy, trifluoromethoxy, ethoxy, or isopropoxy radical) include, for example, ethyl, 2-cyanoethyl, 2-hydroxyethyl, 2-methoxyethyl, 2-trifluoromethoxyethyl, 2-methylthioethyl, 2-ethoxyethyl, 2-isopropoxyethyl, 2,2,2-trifluoroethyl, n-propyl, isopropyl, 3-methoxypropyl, 3-hydroxypropyl, 3-cyanopropyl, 3,3,3-trifluoropropyl, n-butyl, isobutyl, 4-methoxybutyl, 4,4,4-trifluorobutyl, 2-methoxy-2-methylpropyl, 2-hydroxy-2-methylpropyl, 2-cyano-2-methylpropyl, n-pentyl, 3-methylbutyl, 3-cyano-3-methylbutyl, 3-hydroxy-3-methylbutyl, 1,2-dimethylpropyl, 2,2-dimethylpropyl, 2,2-dimethyl-3-hydroxypropyl, 1-ethylpropyl, 3,3-dimethylbutyl, 2-ethylbutyl and 2methylpentyl. Suitable C₂-C₆alkenyl groups include, for example, 2-methyl-2-propenyl. Suitable C₃-C₆cycloalkyl groups wherein one C—C bond within the cycloalkyl moiety is optionally substituted by an O—C bond include, for example, cyclopentyl and tetrahydro-2H-pyran-4-yl. Suitable C₄-C₇cycloalkylalkyl groups (optionally substituted with a halogen atom or hydroxy radical) wherein one C—C bond within the cycloalkyl moiety is optionally substituted by an O—C bond include, for example, cycloheptylmethyl, cyclohexylmethyl, tetrahydro-2H-pyran-4-ylmethyl, cyclopentylmethyl, hydroxycyclopentylmethyl, cyclobutylmethyl, cyclopropylmethyl and fluorocyclopropylmethyl.

In another preferred embodiment, R1 is C₂-C₆alkyl, C₂-C₆alkenyl, C₃-C₆cycloalkyl or C₄-C₇cycloalkylalkyl, each of which is optionally substituted with from 1 to 3 halogen atoms or a methoxy radical. More preferably, R1 is C₂-C₆alkyl (optionally substituted with from 1 to 3 halogen atoms or a methoxy radical), C₂-C₆alkenyl, C₃-C₆cycloalkyl or C₄-C₇cycloalkylalkyl. Suitable C₂-C₆alkyl groups (optionally substituted with from 1 to 3 halogen atoms or a methoxy radical) include, for example, ethyl, n-propyl, isopropyl, n-butyl, isobutyl, n-pentyl, 3-methylbutyl, 1,2-dimethylpropyl, 1-ethylpropyl, 3,3-dimethylbutyl, 2-ethylbutyl, 3,3,3-trifluoropropyl, 4,4,4-trifluorobutyl and 2-methoxyethyl. Suitable C₂-C₆alkenyl groups include, for example, 2-methyl-2-propenyl. Suitable C₃-C₆cycloalkyl groups include, for example, cyclopentyl. Suitable C₄-C₇cycloalkylalkyl groups include, for example, cyclohexylmethyl or cyclopropylmethyl.

In another preferred embodiment, R1 is a C₂-C₁₀alkyl group optionally substituted with from 1 to 7 halogen substituents and/or with from 1 to 3 substituents each independently selected from hydroxy, cyano, C₁-C₄alkyl, C₁-C₄alkylthio (optionally substituted with from 1 to 3 halogen atoms) and C₁-C₄alkoxy (optionally substituted with from 1 to 3 halogen atoms). More preferably, R1 is a C₂-C₁₀alkyl group optionally substituted with from 1 to 7 halogen substituents and/or with from 1 to 3 substituents each independently selected from hydroxy, cyano, C₁-C₄alkylthio (optionally substituted with from 1 to 3 halogen atoms) and C₁-C₄alkoxy (optionally substituted with from 1 to 3 halogen atoms). More preferably, R1 is a C₂-C₁₀alkyl group optionally substituted with from 1 to 3 substituents each independently selected from halogen, hydroxy, cyano, C₁-C₄alkylthio and C₁-C₄alkoxy (optionally substituted with from 1 to 3 fluorine atoms). More preferably R1 is C₂-C₆alkyl optionally substituted with from 1 to 3 halogen atoms or a hydroxy, cyano, methylthio, methoxy, trifluoromethoxy, ethoxy, or isopropoxy radical. Still more preferably R1 is C₂-C₆alkyl optionally substituted with from 1 to 3 fluorine atoms or a hydroxy, cyano, methylthio, methoxy, trifluoromethoxy, ethoxy, or isopropoxy radical. Still more preferably, R1 is selected from ethyl, 2-cyanoethyl, 2-hydroxyethyl, 2-methoxyethyl, 2-trifluoromethoxyethyl, 2-methylthioethyl, 2-ethoxyethyl, 2-isopropoxyethyl, 2,2,2-trifluoroethyl, n-propyl, isopropyl, 3-methoxypropyl, 3-hydroxypropyl, 3-cyanopropyl, 3,3,3-trifluoropropyl, n-butyl, isobutyl, 4-methoxybutyl, 4,4,4-trifluorobutyl, 2-methoxy-2-methylpropyl, 2-hydroxy-2-methylpropyl, 2-cyano-2-methylpropyl, n-pentyl, 3-methylbutyl, 3-cyano-3-methylbutyl, 3-hydroxy-3-methylbutyl, 1,2-dimethylpropyl, 2,2-dimethylpropyl, 2,2-dimethyl-3-hydroxypropyl, 1-ethylpropyl, 3,3-dimethylbutyl, 2-ethylbutyl and 2-methylpentyl. Most preferably R1 is selected from u-propyl, n-butyl, isobutyl, 3-methoxypropyl, 3-hydroxypropyl, 3-cyanopropyl, 4-methoxybutyl, 2-hydroxy-2-methylpropyl, 2-cyano-2-methylpropyl, 3-hydroxy-2,2-dimethylpropyl and 3-cyano-3-methylbutyl.

In another preferred embodiment, R1 is a C₂-C₁₀alkyl group optionally substituted with from 1 to 7 halogen substituents and/or with from 1 to 3 substituents each independently selected from hydroxy, cyano and C₁-C₄alkoxy. More preferably, R1 is a C₂-C₁₀alkyl group optionally substituted with from 1 to 3 substituents each independently selected from halogen, hydroxy and C₁-C₄alkoxy. More preferably R1 is C₂-C₆alkyl optionally substituted with from 1 to 3 halogen atoms or a methoxy radical. Still more preferably R1 is C₂-C₆alkyl. Still more preferably, R1 is selected from ethyl, n-propyl, isopropyl, n-butyl, isobutyl, n-pentyl, 3-methylbutyl, 1,2-dimethylpropyl, 1-ethylpropyl, 3,3-dimethylbutyl and 2-ethylbutyl. Most preferably R1 is selected from n-propyl, n-butyl and isobutyl.

In another preferred embodiment, R1 is a C₂-C₁₀alkenyl group optionally substituted with from 1 to 7 halogen substituents and/or with from 1 to 3 substituents each independently selected from hydroxy, cyano, C₁-C₄alkyl, C₁-C₄alkylthio (optionally substituted with from 1 to 3 halogen atoms) and C₁-C₄alkoxy (optionally substituted with 5 from 1 to 3 halogen atoms). More preferably, R1 is a C₂-C₁₀alkenyl group optionally substituted with from 1 to 7 halogen substituents and/or with from 1 to 3 substituents each independently selected from hydroxy, cyano, C₁-C₄alkylthio (optionally substituted with from 1 to 3 halogen atoms) and C₁-C₄alkoxy (optionally substituted with from 1 to 3 halogen atoms). More preferably, R1 is a C₂-C₁₀alkenyl group optionally substituted with from 1 to 3 substituents each independently selected from halogen, hydroxy, cyano, C₁-C₄alkylthio and C₁-C₄alkoxy (optionally substituted with from 1 to 3 fluorine atoms). More preferably R1 is C₂-C₆alkenyl optionally substituted with from 1 to 3 halogen atoms or a hydroxy, cyano, methylthio, methoxy, trifluoromethoxy, ethoxy, or isopropoxy radical. Still more preferably R1 is C₂-C₆alkenyl. Still more preferably, R1 is 2-methyl-2-propenyl.

In another preferred embodiment, R1 is a C₃-C₈cycloalkyl group, wherein one C—C bond within the cycloalkyl moiety is optionally substituted by an O—C, S—C or C═C bond and wherein the group is optionally substituted with from 1 to 7 halogen substituents and/or with from 1 to 3 substituents each independently selected from hydroxy, cyano, C₁-C₄alkyl, C₁-C₄alkylthio (optionally substituted with from 1 to 3 halogen atoms) and C₁-C₄alkoxy (optionally substituted with from 1 to 3 halogen atoms). More preferably, R1 is a C₃-C₈cycloalkyl group, wherein one C—C bond within the cycloalkyl moiety is optionally substituted by an O—C, S—C or C═C bond. More preferably, R1 is a C₄-C₆cycloalkyl group, wherein one C—C bond within the cycloalkyl moiety is optionally substituted by an O—C, S—C or C═C bond. Still more preferably, R1 is cyclopentyl or tetrahydro-2H-pyran-4-yl

In another preferred embodiment, R1 is a C₄-C₁₀cycloalkylalkyl group, wherein one C—C bond within the cycloalkyl moiety is optionally substituted by an O—C, S—C or C═C bond and wherein the group is optionally substituted with from 1 to 7 halogen substituents and/or with from 1 to 3 substituents each independently selected from hydroxy, cyano, C₁-C₄alkyl, C₁-C₄alkylthio (optionally substituted with from 1 to 3 halogen atoms) and C₁-C₄alkoxy (optionally substituted with from 1 to 3 halogen atoms). More preferably, R1 is a C₄-C₁₀cycloalkylalkyl group, wherein one C—C bond within the cycloalkyl moiety is optionally substituted by an O—C, S—C or C═C bond and wherein the group is optionally substituted with from 1 to 3 substituents each independently selected from halogen, hydroxy, cyano, C₁-C₂alkyl, C₁-C₂alkylthio (optionally substituted with from 1 to 3 halogen atoms) and C₁-C₂alkoxy (optionally substituted with from 1 to 3 halogen atoms). More preferably, R1 is a C₄-C₇cycloalkylalkyl group (optionally substituted with a halogen atom or hydroxy radical) wherein one C—C bond within the cycloalkyl moiety is optionally substituted by an O—C bond. Still more preferably, R1 is cycloheptylmethyl, cyclohexylmethyl, tetrahydro-2H-pyranylmethyl, cyclopentylmethyl, hydroxycyclopentylmethyl, cyclobutylmethyl, cyclopropylmethyl or fluorocyclopropylmethyl.

In a preferred embodiment, R2 is H, C₁-C₄alkyl (optionally substituted with from 1 to 7 halogen atoms), C₁-C₄alkyl-S(O)ₓ— wherein x is 0 or 2 (optionally substituted with from 1 to 7 halogen atoms), C₁-C₄alkoxy (optionally substituted with from 1 to 7 halogen atoms), cyano, halogen, phenyl (optionally substituted with from 1 to 3 substituents each independently selected from halogen, C₁-C₄alkyl and C₁-C₄alkoxy) or phenoxy (optionally substituted with from 1 to 3 substituents each independently selected from halogen, C₁-C₄alkyl and C₁-C₄alkoxy), or together with R3 forms a further benzene ring (optionally substituted with from 1 to 3 substituents each independently selected from halogen, C₁-C₄alkyl and C₁-C₄alkoxy). More preferably, R2 is H, C₁-C₂alkyl (optionally substituted with from 1 to 5 halogen atoms), C₁-C₄alkyl-S(O)ₓ— wherein x is 0 or 2 (optionally substituted with from 1 to 5 halogen atoms), C₁-C₂alkoxy (optionally substituted with from 1 to 5 halogen atoms), cyano, halogen, phenyl (optionally substituted with from 1 to 3 substituents each independently selected from halogen, C₁-C₂alkyl and C₁-C₂alkoxy) or phenoxy (optionally substituted with from 1 to 3 substituents each independently selected from halogen, C₁-C₂alkyl and C₁-C₂alkoxy), or together with R3 forms a further benzene ring (optionally substituted with from 1 to 3 substituents each independently selected from halogen, C₁-C₂alkyl and C₁-C₂alkoxy). Still more preferably, R2 is H, methyl, trifluoromethyl, methylthio, tert-butylthio, trifluoromethylthio, methylsulfonyl, methoxy, ethoxy, difluoromethoxy, trifluoromethoxy, cyano, fluoro, chloro, bromo, phenyl or phenoxy, or together with R3 forms a further benzene ring.

In another preferred embodiment, R2 is not H. More preferably, R2 is C₁-C₄alkyl (optionally substituted with from 1 to 7 halogen atoms), C₁-C₄alkyl-S(O)ₓ— wherein x is 0 or 2 (optionally substituted with from 1 to 7 halogen atoms), C₁-C₄alkoxy (optionally substituted with from 1 to 7 halogen atoms), cyano, halogen, phenyl (optionally substituted with from 1 to 3 substituents each independently selected from halogen, C₁-C₄alkyl and C₁-C₄alkoxy) or phenoxy (optionally substituted with from 1 to 3 substituents each independently selected from halogen, C₁-C₄alkyl and C₁-C₄alkoxy), or together with R3 forms a further benzene ring (optionally substituted with from 1 to 3 substituents each independently selected from halogen, C₁-C₄alkyl and C₁-C₄alkoxy). More preferably, R2 is C₁-C₂alkyl (optionally substituted with from 1 to 5 halogen atoms), C₁-C₂alkyl-S(O)ₓ— wherein x is 0 or 2 (optionally substituted with from 1 to 5 halogen atoms), C₁-C₂alkoxy (optionally substituted with from 1 to 5 halogen atoms), cyano, halogen, phenyl (optionally substituted with from 1 to 3 substituents each independently selected from halogen, C₁-C₂alkyl and C₁-C₂alkoxy) or phenoxy (optionally substituted with from 1 to 3 substituents each independently selected from halogen, C₁-C₂alkyl and C₁-C₂alkoxy), or together with R3 forms a further benzene ring (optionally substituted with from 1 to 3 substituents each independently selected from halogen, C₁-C₂alkyl and C₁-C₂alkoxy). Still more preferably, R2 is methyl, trifluoromethyl, methylthio, tert-butylthio, trifluoromethylthio, methylsulfonyl, methoxy, ethoxy, difluoromethoxy, trifluoromethoxy, cyano, fluoro, chloro, bromo, phenyl or phenoxy, or together with R3 forms a further benzene ring.

In a preferred embodiment, R3 is H, C₁-C₄alkyl (optionally substituted with from 1 to 7 halogen atoms), C₁-C₄alkyl-S-(optionally substituted with from 1 to 7 halogen atoms), C₁-C₄alkoxy (optionally substituted with from 1 to 7 halogen atoms), cyano, halogen, phenyl (optionally substituted with from 1 to 3 substituents each independently selected from halogen, C₁-C₄alkyl and C₁-C₄alkoxy), phenoxy (optionally substituted with from 1 to 3 substituents each independently selected from halogen, C₁-C₄alkyl and C₁-C₄alkoxy) or —CO₂(C₁-C₄alkyl), or together with R2 or R4 forms a further benzene ring (optionally substituted with from 1 to 3 substituents each independently selected from halogen, C₁-C₄alkyl and C₁-C₄alkoxy). More preferably, R3 is H, C₁-C₂alkyl (optionally substituted with from 1 to 5 halogen atoms), C₁-C₂alkyl-S-(optionally substituted with from 1 to 5 halogen atoms), C₁-C₂alkoxy (optionally substituted with from 1 to 5 halogen atoms), cyano, halogen, phenyl (optionally substituted with from 1 to 3 substituents each independently selected from halogen, C₁-C₂alkyl and C₁-C₂alkoxy), phenoxy (optionally substituted with from 1 to 3 substituents each independently selected from halogen, C₁-C₂alkyl and C₁-C₂alkoxy) or —CO₂(C₁-C₂alkyl), or together with R2 or R4 forms a further benzene ring (optionally substituted with from 1 to 3 substituents each independently selected from halogen, C₁-C₂alkyl and C₁-C₂alkoxy). Still more preferably, R3 is H, methyl, trifluoromethyl, trifluoromethylthio, methoxy, ethoxy, difluoromethoxy, trifluoromethoxy, cyano, fluoro, chloro, bromo, phenyl, phenoxy or CO₂CH₃, or together with R2 or R4 forms a further benzene ring.

In a preferred embodiment, R4 is H, C₁-C₄alkyl (optionally substituted with from 1 to 7 halogen atoms), C₁-C₄alkyl-S-(optionally substituted with from 1 to 7 halogen atoms), C₁-C₄alkoxy (optionally substituted with from 1 to 7 halogen atoms), cyano, halogen, phenyl (optionally substituted with from 1 to 3 substituents each independently selected from halogen, C₁-C₄alkyl and C₁-C₄alkoxy), or —CO₂(C₁-C₄alkyl), or together with R3 forms a further benzene ring (optionally substituted with from 1 to 3 substituents each independently selected from halogen, C₁-C₄alkyl and C₁-C₄alkoxy). More preferably, R4 is H, C₁-C₂alkyl (optionally substituted with from 1 to 5 halogen atoms), C₁-C₂alkyl-S-(optionally substituted with from 1 to 5 halogen atoms), C₁-C₂alkoxy (optionally substituted with from 1 to 5 halogen atoms), cyano, halogen, phenyl (optionally substituted with from 1 to 3 substituents each independently selected from halogen, C₁-C₂alkyl and C₁-C₂alkoxy), or —CO₂(C₁-C₂alkyl), or together with R3 forms a further benzene ring (optionally substituted with from 1 to 3 substituents each independently selected from halogen, C₁-C₂alkyl and C₁-C₂alkoxy). Still more preferably, R4 is H, methyl, trifluoromethyl, methylthio, methoxy, trifluoromethoxy, cyano, fluoro, chloro, phenyl or CO₂CH₃, or together with R3 forms a further benzene ring.

In a preferred embodiment, R5 is H, C₁-C₄alkyl (optionally substituted with from 1 to 5 halogen atoms), C₁-C₄alkoxy (optionally substituted with from 1 to 5 halogen atoms) or halogen. More preferably, R5 is H, C₁-C₄alkyl, C₁-C₄alkoxy or halogen. Still more preferably, R5 is H, methyl, methoxy, fluoro or chloro.

In a preferred embodiment, R6 is H, C₁-C₄alkyl (optionally substituted with from 1 to 5 halogen atoms) or halogen. More preferably, R6 is H, C₁-C₄alkyl or halogen. Still more preferably, R6 is H, methyl, fluoro or chloro.

In a further preferred embodiment of the present invention, the group 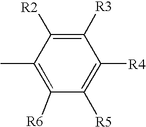
is phenyl, 2-methylphenyl, 2-(trifluoromethyl)phenyl, 2-(methylthio)phenyl, 2-(tertbutylthio)phenyl, 2-(trifluoromethylthio)phenyl, 2-(methylsulfonyl)phenyl, 2-methoxyphenyl, 2-ethoxyphenyl, 2-(difluoromethoxy)phenyl, 2-(trifluoromethoxy)phenyl, 2-cyanophenyl, 2-fluorophenyl, 2-chlorophenyl, 2-bromophenyl, 2-biphenyl, 2-phenoxyphenyl, 3-methylphenyl, 3-(trifluoromethyl)phenyl, 3-(trifluoromethylthio)phenyl, 3-methoxyphenyl, 3-ethoxyphenyl, 3-(difluoromethoxy)phenyl, 3-(trifluoromethoxy)phenyl, 3-cyanophenyl, 3-fluorophenyl, 3-chlorophenyl, 3-bromophenyl, 3-biphenyl, 3-phenoxyphenyl, 3-(methoxycarbonyl)phenyl, 4-methylphenyl, 4-(trifluoromethyl)phenyl, 4-(methylthio)phenyl, 4-methoxyphenyl, 4-(trifluoromethoxy)phenyl, 4-cyanophenyl, 4-fluorophenyl, 4-chlorophenyl, 4-biphenyl, 4-(methoxycarbonyl)phenyl, 2,3-dichlorophenyl, 2-chloro-3-methylphenyl, 2-chloro-3-(trifluoromethyl)phenyl, 2,4-dimethylphenyl, 2,4-bis(trifluoromethyl)phenyl, 2,4-dimethoxyphenyl, 2,4-difluorophenyl, 2,4-dichlorophenyl, 2-chloro-4-fluorophenyl, 2-fluoro-4 (trifluoromethyl)phenyl, 2-chloro-4-(methylsulfonyl)phenyl 2,5-dimethylphenyl, 2,5-dichlorophenyl, 2-chloro-5-(trifluoromethyl)phenyl, 2,6-dimethylphenyl, 2,6-dichlorophenyl, 2-chloro-6-fluorophenyl, 2-fluoro-6-(trifluoromethyl)phenyl, 3-chloro-2-methylphenyl, 3-chloro-2-fluorophenyl, 3-chloro-2-(trifluoromethyl)phenyl, 3,4-dichlorophenyl, 3-chloro-4-fluorophenyl, 3,5-dimethylphenyl, 3,5-dimethoxyphenyl, 3,5-difluorophenyl, 3,5-dichlorophenyl, 3-fluoro-5-(trifluoromethyl)phenyl, 5-fluoro-2-(trifluoromethylphenyl), 5-fluoro-2-methoxyphenyl, 4-fluoro-2-(trifluoromethyl)phenyl, 4-chloro-3-(trifluoromethyl)phenyl, 2,3,6-trichlorophenyl, 2,3,5-trichlorophenyl, 3-chloro-2-fluoro-6-(trifluoromethyl)phenyl, 3-chloro-2-fluoro-5-(trifluoromethyl)phenyl, 2-chloro-6-fluoro-3-methylphenyl, 2-chloro-6-fluoro-5-methylphenyl, 1-naphthyl or 2-naphthyl.

A further embodiment of the present invention provides a group (Group A) of compounds of formula I above, wherein R2, R3, R4, R5 and R6 are all H.

A further embodiment of the present invention provides a group (Group B) of compounds of formula I above, wherein one of R2, R3, R4, R5 and R6 is not H and the others are H.

Compounds of Group B include those (Group B2) wherein R3, R4, R5 and R6 are all H and R2 is C₁-C₄alkyl (optionally substituted with from 1 to 7 halogen atoms), C₁-C₄alkyl-S(O)ₓ— wherein x is 0, 1 or 2 (optionally substituted with from 1 to 7 halogen atoms), C₁-C₄alkoxy (optionally substituted with from 1 to 7 halogen atoms), cyano, halogen, phenyl (optionally substituted with from 1 to 3 substituents each independently selected from halogen, C₁-C₄alkyl and C₁-C₄alkoxy), phenoxy (optionally substituted with from 1 to 3 substituents each independently selected from halogen, C₁-C₄alkyl and C₁-C₄alkoxy) or —CO₂(C₁-C₄alkyl).

Compounds of Group B also include those (Group B3) wherein R2, R4, R5 and R6 are all H and R3 is C₁-C₄alkyl (optionally substituted with from 1 to 7 halogen atoms), C₁-C₄alkyl-S(O)ₓ— wherein x is 0, 1 or 2 (optionally substituted with from 1 to 7 halogen atoms), C₁-C₄alkoxy (optionally substituted with from 1 to 7 halogen atoms), cyano, halogen, phenyl (optionally substituted with from 1 to 3 substituents each independently selected from halogen, C₁-C₄alkyl and C₁-C₄alkoxy), phenoxy (optionally substituted with from 1 to 3 substituents each independently selected from halogen, C₁-C₄alkyl and C₁-C₄alkoxy) or —CO₂(C₁-C₄alkyl).

Compounds of Group B also include those (Group B4) wherein R2, R3, R5 and R6 are all H and R4 is C₁-C₄alkyl (optionally substituted with from 1 to 7 halogen atoms), C₁-C₄alkyl-S(O)ₓ— wherein x is 0, 1 or 2 (optionally substituted with from 1 to 7 halogen atoms), C₁-C₄alkoxy (optionally substituted with from 1 to 7 halogen atoms), cyano, halogen, phenyl (optionally substituted with from 1 to 3 substituents each independently selected from halogen, C₁-C₄alkyl and C₁-C₄alkoxy), phenoxy (optionally substituted with from 1 to 3 substituents each independently selected from halogen, C₁-C₄alkyl and C₁-C₄alkoxy) or —CO₂(C₁-C₄alkyl).

A further embodiment of the present invention provides a group (Group C) of compounds of formula I above, wherein two of R2, R3, R4, R5 and R6 are not H and the others are H.

Compounds of Group C include those (Group C_{2,3}) wherein R4, R5 and R6 are all H; R2 is C₁-C₄alkyl (optionally substituted with from 1 to 7 halogen atoms), C₁-C₄alkyl-S(O)ₓ— wherein x is 0, 1 or 2 (optionally substituted with from 1 to 7 halogen atoms), C₁-C₄alkoxy (optionally substituted with from 1 to 7 halogen atoms), cyano, halogen, phenyl (optionally substituted with from 1 to 3 substituents each independently selected from halogen, C₁-C₄alkyl and C₁-C₄alkoxy), phenoxy (optionally substituted with from 1 to 3 substituents each independently selected from halogen, C₁-C₄alkyl and C₁-C₄alkoxy) or —CO₂(C₁-C₄alkyl), or together with R3 forms a further benzene ring (optionally substituted with from 1 to 3 substituents each independently selected from halogen, C₁-C₄alkyl and C₁-C₄alkoxy); and
R3 is C₁-C₄alkyl (optionally substituted with from 1 to 7 halogen atoms), C₁-C₄alkyl-S(O)ₓ— wherein x is 0, 1 or 2 (optionally substituted with from 1 to 7 halogen atoms), C₁-C₄alkoxy (optionally substituted with from 1 to 7 halogen atoms), cyano, halogen, phenyl (optionally substituted with from 1 to 3 substituents each independently selected from halogen, C₁-C₄alkyl and C₁-C₄alkoxy), phenoxy (optionally substituted with from 1 to 3 substituents each independently selected from halogen, C₁-C₄alkyl and C₁-C₄alkoxy) or —CO₂(C₁-C₄alkyl), or together with R2 forms a further benzene ring (optionally substituted with from 1 to 3 substituents each independently selected from halogen, C₁-C₄alkyl and C₁-C₄alkoxy).

Compounds of Group C also include those (Group C_{2,4}) wherein R3, R5 and R6 are all H;
R2 is C₁-C₄alkyl (optionally substituted with from 1 to 7 halogen atoms), C₁-C₄alkyl-S(O)ₓ— wherein x is 0, 1 or 2 (optionally substituted with from 1 to 7 halogen atoms), C₁-C₄alkoxy (optionally substituted with from 1 to 7 halogen atoms), cyano, halogen, phenyl (optionally substituted with from 1 to 3 substituents each independently selected from halogen, C₁-C₄alkyl and C₁-C₄alkoxy), phenoxy (optionally substituted with from 1 to 3 substituents each independently selected from halogen, C₁-C₄alkyl and C₁-C₄alkoxy) or —CO₂(C₁-C₄alkyl); and
R4 is C₁-C₄alkyl (optionally substituted with from 1 to 7 halogen atoms), C₁-C₄alkyl-S(O)ₓ— wherein x is 0, 1 or 2 (optionally substituted with from 1 to 7 halogen atoms), C₁-C₄alkoxy (optionally substituted with from 1 to 7 halogen atoms), cyano, halogen, phenyl (optionally substituted with from 1 to 3 substituents each independently selected from halogen, C₁-C₄alkyl and C₁-C₄alkoxy), phenoxy (optionally substituted with from 1 to 3 substituents each independently selected from halogen, C₁-C₄alkyl and C₁-C₄alkoxy) or —CO₂(C₁-C₄alkyl).

Compounds of Group C also include those (Group C2,5) wherein R3, R4 and R6 are all H;
R2 is C₁-C₄alkyl (optionally substituted with from 1 to 7 halogen atoms), C₁-C₄alkyl-S(O)ₓ— wherein x is 0, 1 or 2 (optionally substituted with from 1 to 7 halogen atoms), C₁-C₄alkoxy (optionally substituted with from 1 to 7 halogen atoms), cyano, halogen, phenyl (optionally substituted with from 1 to 3 substituents each independently selected from halogen, C₁-C₄alkyl and C₁-C₄alkoxy), phenoxy (optionally substituted with from 1 to 3 substituents each independently selected from halogen, C₁-C₄alkyl and C₁-C₄alkoxy) or —CO₂(C₁-C₄alkyl); and
R5 is C₁-C₄alkyl (optionally substituted with from 1 to 7 halogen atoms), C₁-C₄alkoxy (optionally substituted with from 1 to 7 halogen atoms) or halogen.

Compounds of Group C also include those (Group C_{2,6}) wherein R3, R4 and RS are all H;
R2 is C₁-C₄alkyl (optionally substituted with from 1 to 7 halogen atoms), C₁-C₄alkyl-S(O)ₓ— wherein x is 0, 1 or 2 (optionally substituted with from 1 to 7 halogen atoms), C₁-C₄alkoxy (optionally substituted with from 1 to 7 halogen atoms), cyano, halogen, phenyl (optionally substituted with from 1 to 3 substituents each independently selected from halogen, C₁-C₄alkyl and C₁-C₄alkoxy), phenoxy (optionally substituted with from 1 to 3 substituents each independently selected from halogen, C₁-C₄alkyl and C₁-C₄alkoxy) or —CO₂(C₁-C₄alkyl); and
R6 is C₁-C₄alkyl (optionally substituted with from 1 to 7 halogen atoms), C₁-C₄alkoxy (optionally substituted with from 1 to 7 halogen atoms) or halogen.

Compounds of Group C also include those (Group C_{3,4}) wherein R2, R5 and R6 are all H;
R3 is C₁-C₄alkyl (optionally substituted with from 1 to 7 halogen atoms), C₁-C₄alkyl-S(O)ₓ— wherein x is 0, 1 or 2 (optionally substituted with from 1 to 7 halogen atoms), C₁-C₄alkoxy (optionally substituted with from 1 to 7 halogen atoms), cyano, halogen, phenyl (optionally substituted with from 1 to 3 substituents each independently selected from halogen, C₁-C₄alkyl and C₁-C₄alkoxy), phenoxy (optionally substituted with from 1 to 3 substituents each independently selected from halogen, C₁-C₄alkyl and C₁-C₄alkoxy) or —CO₂(C₁-C₄alkyl), or together with R4 forms a further benzene ring (optionally substituted with from 1 to 3 substituents each independently selected from halogen, C₁-C₄alkyl and C₁-C₄alkoxy); and
R4 is C₁-C₄alkyl (optionally substituted with from 1 to 7 halogen atoms), C₁-C₄alkyl-S(O)ₓ— wherein x is 0, 1 or 2 (optionally substituted with from 1 to 7 halogen atoms), C₁-C₄alkoxy (optionally substituted with from 1 to 7 halogen atoms), cyano, halogen, phenyl (optionally substituted with from 1 to 3 substituents each independently selected from halogen, C₁-C₄alkyl and C₁-C₄alkoxy), phenoxy (optionally substituted with from 1 to 3 substituents each independently selected from halogen, C₁-C₄alkyl and C₁-C₄alkoxy) or —CO₂(C₁-C₄alkyl), or together with R3 forms a further benzene ring (optionally substituted with from 1 to 3 substituents each independently selected from halogen, C₁-C₄alkyl and C₁-C₄alkoxy).

Compounds of Group C also include those (Group C3,5) wherein R2, R4 and R6 are all H;
R3 is C₁-C₄alkyl (optionally substituted with from 1 to 7 halogen atoms), C₁-C₄alkyl-S(O)ₓ— wherein x is 0, 1 or 2 (optionally substituted with from 1 to 7 halogen atoms), C₁-C₄alkoxy (optionally substituted with from 1 to 7 halogen atoms), cyano, halogen, phenyl (optionally substituted with from 1 to 3 substituents each independently selected from halogen, C₁-C₄alkyl and C₁-C₄alkoxy), phenoxy (optionally substituted with from 1 to 3 substituents each independently selected from halogen, C₁-C₄alkyl and C₁-C₄alkoxy) or —CO₂(C₁-C₄alkyl); and
R5 is C₁-C₄alkyl (optionally substituted with from 1 to 7 halogen atoms), C₁-C₄alkoxy (optionally substituted with from 1 to 7 halogen atoms) or halogen.

A further embodiment of the present invention provides a group (Group D) of compounds of formula I above, wherein three of R2, R3, R4, R5 and R6 are not H and the others are H.

Compounds of Group D include those (Group D2,3,5) wherein R4 and R6 are both H;
R2 is C₁-C₄alkyl (optionally substituted with from 1 to 7 halogen atoms), C₁-C₄alkyl-S(O)ₓ— wherein x is 0, 1 or 2 (optionally substituted with from 1 to 7 halogen atoms), C₁-C₄alkoxy (optionally substituted with from 1 to 7 halogen atoms), cyano, halogen, phenyl (optionally substituted with from 1 to 3 substituents each independently selected from halogen, C₁-C₄alkyl and C₁-C₄alkoxy), phenoxy (optionally substituted with from 1 to 3 substituents each independently selected from halogen, C₁-C₄alkyl and C₁-C₄alkoxy) or —CO₂(C₁-C₄alkyl), or together with R3 forms a further benzene ring (optionally substituted with from 1 to 3 substituents each independently selected from halogen, C₁-C₄alkyl and C₁-C₄alkoxy);
R3 is C₁-C₄alkyl (optionally substituted with from 1 to 7 halogen atoms), C₁-C₄alkyl-S(O)ₓ— wherein x is 0, 1 or 2 (optionally substituted with from 1 to 7 halogen atoms), C₁-C₄alkoxy (optionally substituted with from 1 to 7 halogen atoms), cyano, halogen, phenyl (optionally substituted with from 1 to 3 substituents each independently selected from halogen, C₁-C₄alkyl and C₁-C₄alkoxy), phenoxy (optionally substituted with from 1 to 3 substituents each independently selected from halogen, C₁-C₄alkyl and C₁-C₄alkoxy) or —CO₂(C₁-C₄alkyl), or together with R2 forms a further benzene ring (optionally substituted with from 1 to 3 substituents each independently selected from halogen, C₁-C₄alkyl and C₁-C₄alkoxy); and
R5 is C₁-C₄alkyl (optionally substituted with from 1 to 7 halogen atoms), C₁-C₄alkoxy (optionally substituted with from 1 to 7 halogen atoms) or halogen.

Compounds of Group D include those (Group D2,3,6) wherein R4 and R5 are both H;
R2 is C₁-C₄alkyl (optionally substituted with from 1 to 7 halogen atoms), C₁-C₄alkyl-S(O)ₓ— wherein x is 0, 1 or 2 (optionally substituted with from 1 to 7 halogen atoms), C₁-C₄alkoxy (optionally substituted with from 1 to 7 halogen atoms), cyano, halogen, phenyl (optionally substituted with from 1 to 3 substituents each independently selected from halogen, C₁-C₄alkyl and C₁-C₄alkoxy), phenoxy (optionally substituted with from 1 to 3 substituents each independently selected from halogen, C₁-C₄alkyl and C₁-C₄alkoxy) or —CO₂(C₁-C₄alkyl), or together with R3 forms a further benzene ring (optionally substituted with from 1 to 3 substituents each independently selected from halogen, C₁-C₄alkyl and C₁-C₄alkoxy);
R3 is C₁-C₄alkyl (optionally substituted with from 1 to 7 halogen atoms), C₁-C₄alkyl-S(O)ₓ— wherein x is 0, 1 or 2 (optionally substituted with from 1 to 7 halogen atoms), C₁C₄alkoxy (optionally substituted with from 1 to 7 halogen atoms), cyano, halogen, phenyl (optionally substituted with from 1 to 3 substituents each independently selected from halogen, C₁-C₄alkyl and C₁-C₄alkoxy), phenoxy (optionally substituted with from 1 to 3 substituents each independently selected from halogen, C₁-C₄alkyl and C₁-C₄alkoxy) or —CO₂(C₁-C₄alkyl), or together with R2 forms a further benzene ring (optionally substituted with from 1 to 3 substituents each independently selected from halogen, C₁-C₄alkyl and C₁-C₄alkoxy); and
R6 is C₁-C₄alkyl (optionally substituted with from 1 to 7 halogen atoms), C₁-C₄alkoxy (optionally substituted with from 1 to 7 halogen atoms) or halogen.

For compounds of Formula I falling within any one of groups A, B, B2, B3, B4, C, C_{2,3}, C_{2,4}, C2,5, C_{2,6}, C_{3,4}, C3,5, D, D2,3,5 and D2,3,6 described above, n is preferably 1 or 2, more preferably 1.

For compounds of Formula I falling within any one of groups A, B, B2, B3, B4, C, C_{2,3}, C_{2,4}, C2,5, C_{2,6}, C_{3,4}, C3,5, D, D2,3,5 and D2,3,6 described above, R7 is preferably H or methyl, more preferably H.

For compounds of Formula I falling within any one of groups A, B, B2, B3, B4, C, C_{2,3}, C_{2,4}, C2,5, C_{2,6}, C_{3,4}, C3,5, D, D2,3,5 and D2,3,6 described above, R8 is preferably H.

For compounds of Formula I falling within any one of groups A, B, B2, B3, B4, C, C_{2,3}, C_{2,4}, C2,5, C_{2,6}, C_{3,4}, C3,5, D, D2,3,5 and D2,3,6 described above, R9 is preferably H or fluoro, more preferably H.

For compounds of Formula I falling within any one of groups A, B, B2, B3, B4, C, C_{2,3}, C_{2,4}, C2,5, C_{2,6}, C_{3,4}, C3,5, D, D2,3,5 and D2,3,6 described above, R10 is preferably H or fluoro, more preferably H.

For compounds of Formula I falling within any one of groups A, B, B2, B3, B4, C, C_{2,3}, C_{2,4}, C2,5, C_{2,6}, C_{3,4}, C3,5, D, D2,3,5 and D2,3,6 described above, R1 is preferably a C₂-C₁₀alkyl group optionally substituted with from 1 to 7 halogen substituents and/or with from 1 to 3 substituents each independently selected from hydroxy, cyano, C₁-C₄alkyl, C₁-C₄alkylthio (optionally substituted with from 1 to 3 halogen atoms) and C₁-C₄alkoxy (optionally substituted with from 1 to 3 halogen atoms). More preferably, R1 is a C₂-C₁₀alkyl group optionally substituted with from 1 to 7 halogen substituents and/or with from 1 to 3 substituents each independently selected from hydroxy, cyano and C₁-C₄alkoxy.

For compounds of Formula I falling within any one of groups A, B, B2, B3, B4, C, C_{2,3}, C_{2,4}, C2,5, C_{2,6}, C_{3,4}, C3,5, D, D2,3,5 and D2,3,6 described above, n is preferably 1, R7, R8, R9 and R10 are preferably H and R1 is preferably a C₂-C₁₀alkyl group optionally substituted with from 1 to 7 halogen substituents and/or with from 1 to 3 substituents each independently selected from hydroxy, cyano, C₁-C₄alkyl, C₁-C₄alkylthio (optionally substituted with from 1 to 3 halogen atoms) and C₁-C₄alkoxy (optionally substituted with from 1 to 3 halogen atoms). More preferably, R1 is a C₂-C₁₀alkyl group optionally substituted with from 1 to 7 halogen substituents and/or with from 1 to 3 substituents each independently selected from hydroxy, cyano and C₁-C₄alkoxy.

Particularly preferred compounds of Formula I include:
N-(1-methylethyl)-N-{[2-(trifluoromethyl)phenyl]methyl}piperidin-4-amine,
N-(2-methylpropyl)-N-{[4-(methoxy)phenyl]methyl}piperidin-4-amine,
N-(2-methylpropyl)-N-{[4-(chloro)phenyl]methyl}piperidin-4-amine,
N-(2-methylpropyl)-N-(phenylmethyl)piperidin-4-amine,
N-(2-methylpropyl)-N-{[4-(methyl)phenyl]methyl}piperidin-4-amine,
N-(2-methylpropyl)-N-{[3,4-(dichloro)phenyl]methyl}piperidin-4-amine,
N-(2-methylpropyl)-N-{[2-(trifluoromethyl)phenyl]methyl}piperidin-4-amine,
N-(cyclohexylmethyl)-N-(phenylmethyl)piperidin-4-amine,
N-(cyclohexylmethyl)-N-{[4-(chloro)phenyl]methyl}piperidin-4-amine,
N-(cyclohexylmethyl)-N-{[4-(methoxy)phenyl]methyl}piperidin-4-amine,
N-(cyclohexylmethyl)-N-{[4-(methyl)phenyl]methyl}piperidin-4-amine,
N-(cyclohexylmethyl)-N-{[3,4-(dichloro)phenyl]methyl}piperidin-4-amine,
N-(cyclohexylmethyl)-N-{[2-(trifluoromethyl)phenyl]methyl}piperidin-4-amine,
N-(cyclopropylmethyl)-N-{[4-(chloro)phenyl]methyl}piperidin-4-amine,
N-(cyclopropylmethyl)-N-{[4-(methoxy)phenyl]methyl}piperidin-4-amine,
N-(cyclopropylmethyl)-N-{[4-(methyl)phenyl]methyl}piperidin-4-amine,
N-(cyclopropylmethyl)-N-{[3,4-(dichloro)phenyl]methyl}piperidin-4-amine,
N-(cyclopropylmethyl)-N-{[2-(trifluoromethyl)phenyl]methyl}piperidin-4-amine,
N-(butyl)-N-{[4-(chloro)phenyl]methyl}piperidin-4-amine,
N-(butyl)-N-{[4-(methoxy)phenyl]methyl}piperidin-4-amine,
N-(butyl)-N-{[4-(methyl)phenyl]methyl}piperidin-4-amine,
N-(butyl)-N-{[3,4-(dichloro)phenyl]methyl}piperidin-4-amine,
N-(butyl)-N-{[2-(trifluoromethyl)phenyl]methyl}piperidin-4-amine,
N-(2-methylpropyl)-N-{[2-(cyano)phenyl]methyl}piperidin-4-amine,
N-(2-methylpropyl)-N-{[4-(trifluoromethyl)phenyl]methyl}piperidin-4-amine,
N-(2-methylpropyl)-N-{[3-(methyl)phenyl]methyl}piperidin-amine,
N-(2-methylpropyl)-N-{[4-(phenyl)phenyl]methyl}piperidin-4-famine,
N-(2-methylpropyl)-N-{[5-fluoro-2-(trifluoromethyl)phenyl]methyl}piperidin-4-amine,
N-(2-methylpropyl)-N-{[2,4-di-(trifluoromethyl)phenyl]methyl}piperidin-4-amine,
N-(2-methylpropyl)-N-{[2-naphthyl]methyl}piperidin-4-amine,
N-(2-methylpropyl)-N-{[2-(methylthio)phenyl]methyl}piperidin-4-amine,
N-(2-methylpropyl)-N-{[1-naphthyl]methyl}piperidin-4-amine,
N-(2-methylpropyl)-N-[(3-cyanophenyl)methyl]piperidin-4-amine,
N-(2-methylpropyl)-N-[(3,5-chlorophenyl)methyl]piperidin-4-amine,
N-(2-methylpropyl)-N-[(2,4-dimethoxyphenyl)methyl]piperidin-4-amine,
N-(2-methylpropyl)-N-[(2,3-dichlorophenyl)methyl]piperidin-4-amine,
N-(2-methylpropyl)-N-{[4-(methoxycarbonyl)phenyl]methyl}piperidin-4-amine,
N-(2-methylpropyl)-N-[(2,4-difluorophenyl)methyl]piperidin-4-amine,
N-(2-methylpropyl)-N-{[2-(trifluoromethoxy)phenyl]methyl}piperidin-4-amine,
N-(2-methylpropyl)-N-[(2-fluorophenyl)methyl]piperidin-4-amine,
N-(2-methylpropyl)-N-[(2-chlorophenyl)methyl]piperidin-4-amine,
N-(2-methylpropyl)-N-[(2-methoxyphenyl)methyl]piperidin-4-amine,
N-(2-methylpropyl)-N-[(2-methylphenyl)methyl]piperidin-4-amine,
N-(2-methylpropyl)-N-[(2-bromophenyl)methyl]piperidin-4-amine,
N-(2-methylpropyl)-N-[(3-fluorophenyl)methyl]piperidin-4-amine,
N-(2-methylpropyl)-N-[(3-chlorophenyl)methyl]piperidinamine,
N-(2-methylpropyl)-N-[(3-methoxyphenyl)methyl]piperidinamine,
N-(2-methylpropyl)-N-{[3-(trifluoromethyl)phenyl]methyl}piperidin-4-amine,
N-(2-methylpropyl)-N-{[3-(trifluoromethoxy)phenyl]methyl}piperidin-4-amine,
N-(2-methylpropyl)-N-[(2,6-dichlorophenyl)methyl]piperidin-4-amine,
N-(2-methylpropyl)-N-[(4-methylthiophenyl)methyl]piperidin-4-amine,
N-(2-methylpropyl)-N-[(2,4-dimethylphenyl)methyl]piperidin-4-amine,
N-ethyl-N-{[2-(trifluoromethyl)phenyl]methyl}piperidin-4-amine,
N-propyl-N-{[2-(trifluoromethyl)phenyl]methyl}piperidin-4-amine,
N-pentyl-N-{[2-(trifluoromethyl)phenyl]methyl}piperidin-4-amine,
N-(3-methylbutyl)-N-{[2-(trifluoromethyl)phenyl]methyl}piperidin-4-amine,
N-(3,3-dimethylbutyl)-N-{[2-(trifluoromethyl)phenyl]methyl}piperidin-4-amine,
N-(2-ethylbutyl)-N-{[2-(trifluoromethyl)phenyl]methyl}piperidin-4-amine,
N-(2-methylprop-2-enyl)-N-{[2-(trifluoromethyl)phenyl]methyl}piperidin-4-amine,
N-(2-methylpropyl)-N-{[3-(trifluoromethylthio)phenyl]methyl}piperidin-4-amine,
N-(2-methylpropyl)-N-{[2-(trifluoromethylthio)phenyl]methyl}piperidin-4-amine,
N-(2-methylpropyl)-N-[(3-bromophenyl)methyl]piperidin-4-amine,
N-(2-methylpropyl)-N-[(3-phenoxyphenyl)methyl]piperidin-4-amine,
N-(2-methylpropyl)-N-{[3-(difluoromethoxy)phenyl]methyl}piperidin-4-amine,
N-(2-methylpropyl)-N-[(2,5-dimethylphenyl)methyl]piperidin-4-amine,
N-(2-methylpropyl)-N-[(4-cyanophenyl)methyl]piperidin-4-amine,
N-(2-methylpropyl)-N-[(2-ethoxyphenyl)methyl]piperidin-4-amine,
N-(2-methylpropyl)-N-[(4-fluorophenyl)methyl]piperidin-4-amine,
N-(2-methylpropyl)-N-[(3-ethoxyphenyl)methyl]piperidin-4-amine,
N-(2-methylpropyl)-N-[(2-chloro-6-fluorophenyl)methyl]piperidin-4-amine,
N-(2-methylpropyl)-N-[(2-biphenyl)methyl]piperidin-4-amine,
N-(2-methylpropyl)-N-[(3-biphenyl)methyl]piperidin-4-amine,
N-(2-methylpropyl)-N-{[2-fluoro-6-(trifluoromethyl)phenyl]methyl}piperidin-4-amine,
N-(2-methylpropyl)-N-[(3,5-difluorophenyl)methyl]piperidin-4-amine,
N-(2-methylpropyl)-N-[(3,5-dimethylphenyl)methyl]piperidin-4-amine,
N-(2-methylpropyl)-N-[(3,5-dimethoxyphenyl)methyl]piperidin-4-amine,
N-(2-methylpropyl)-N-{[3-fluoro-5-(trifluoromethyl)phenyl]methyl}piperidin-4-amine,
N-(2-methylpropyl)-N-[(5-fluoro-2-methoxyphenyl)methyl]piperidin-4-amine,
N-(2-methylpropyl)-N-{[4-(trifluoromethoxy)phenyl)methyl]piperidin-4-amine,
N-(2-methylpropyl)-N-{[3-(methoxycarbonyl)phenyl]methyl}piperidin-4-amine,
N-(2-methylpropyl)-N-[(2,6 diethylphenyl)methyl]piperidin-4-amine,
N-(2-methylpropyl)-N-{[2-(tert-butylthio)phenyl]methyl}piperidin-4-amine,
N-(2-methylpropyl)-N-{[4-fluoro-2-(trifluoromethyl)phenyl]methyl}piperidin-4-amine,
N-(3,3-dimethylbutyl)-N-[(2-bromophenyl)methyl]piperidin-4-amine,
N-(3,3-dimethylbutyl)-N-[(2-methylphenyl)methyl]piperidin-4-amine,
N-(2-methylpropyl)-N-[(2,4-dichlorophenyl)methyl]piperidin-4-amine,
N-(2-methylpropyl)-N-{[2-(difluoromethoxy)phenyl]methyl}piperidin-4-amine,
N-(3,3-dimethylbutyl)-N-{[5-fluoro-2-(trifluoromethyl)phenyl]methyl}piperidin-4-amine,
N-(3-ethylbutyl)-N-[(2-bromophenyl)methyl]piperidin-4-amine,
N-(3-ethylbutyl)-N-[(2-methylphenyl)methyl]piperidin-4-amine,
N-propyl-N-[(2-chlorophenyl)methyl]piperidin-4-amine,
N-(3,3-dimethylbutyl)-N-[(2-phenoxyphenyl)methyl]piperidin-4-amine,
N-(3-ethylbutyl)-N-[(2-chloro-6-fluorophenyl)methyl]piperidin-4-amine,
N-(3,3-dimethylbutyl)-N-[(2-biphenyl)methyl]piperidin-4-amine,
N-(2-methoxyethyl)-N-{[2-(trifluoromethyl)phenyl]methyl}piperidin-4-amine,
N-(2-ethylbutyl)-N-{[5-fluoro-2-(trifluoromethyl)phenyl]methyl}piperidin-4-amine,
N-cyclopentyl-N-{[2-(trifluoromethyl)phenyl]methyl}piperidin-4-amine,
N-(3,3,3-trifluoropropyl)-N-f{[2-(trifluoromethyl)phenyl]methyl}piperidin-4-amine,
N-(4,4,4-trifluorobutyl)-N-{[2-(trifluoromethyl)phenyl]methyl}piperidin-4-amine,
N-(2,2-dimethylpropyl)-N-{[2-(trifluoromethyl)phenyl]methyl}piperidin-4-amine,
N-(2-methylpropyl)-N-{[2-(trifluoromethyl)phenyl]ethyl}piperidin-4-amine,
N-(2-methylpropyl)-N²-{[2-(methylsulphonyl)phenyl]methyl}piperidin-4-amine,
N-(2-ethylbutyl)-N-[(2-biphenyl)methyl]piperidin-4-amine,
N-(cyclohexylmethyl)-N-[(2-biphenyl)methyl]piperidin-4-amine,
N-(2-ethylbutyl)-N-[(2-phenoxyphenyl)methyl]piperidin-4-amine,
N-(2-methylpropyl)-N-[(2-phenoxyphenyl)methyl]piperidin-4-amine,
N-(cyclohexylmethyl)-N-[(2-phenoxyphenyl)methyl]piperidin-amine,
N-(2-ethylpropyl)-N-{[2-(trifluoromethyl)phenyl]methyl}piperidin-4-amine,
N-butyl-N-{[2-(trifluoromethyl)phenyl]methyl}piperidinamine,
N-(cyclopropylmethyl)-N-[(2-biphenyl)methyl]piperidin-4-amine,
N-(cyclopropylmethyl)-N-[(2-phenoxyphenyl)methyl]piperidin-4-amine,
N-(2-methoxyethyl)-N-[(2-methylthio)methyl}piperidin-4-amine,
N-(2-methoxyethyl)-N-{[2-(difluoromethoxy)phenyl]methyl}piperidin-4-amine,
N-(2-methoxyethyl)-N-[(2-methyl)methyl}piperidin-4-amine,
N-(2-methoxyethyl)-N-[(2-chlorophenyl)methyl]piperidin-4-amine,
N-(2-methoxyethyl)-N-{[4-fluoro-2-(trifluoromethyl)phenyl]methyl}piperidin-4-amine,
N-(2-methoxyethyl)-N-[(2,4-dichlorophenyl)methyl]piperidin-4-amine,
N-(cyclopropylmethyl)-N-[(2-methylthiophenyl)methyl]piperidin-4-amine,
N-(cyclopropylmethyl)-N-[(2-methylphenyl)methyl]piperidin-4-amine,
N-(cyclopropylmethyl)-N-[(2-chlorophenyl)methyl]piperidinamine,
N-(cyclopropylmethyl)-N-{[4-fluoro-2-(trifluoromethyl)phenyl]methyl}piperidin-4-amine,
N-(cyclopropylmethyl)-N-[(2,4-dichlorophenyl)methyl]piperidin-4-amine,
N-(3-methylbutyl)-N-[(2-phenoxyphenyl)methyl]piperidin-4-amine,
N-(3-methylbutyl)-N-[(2-biphenyl)methyl]piperidin-4-amine,
N-(2,3-dimethylpropyl)-N-{[2-(trifluoromethyl)phenyl]methyl}piperidin-4-amine (racemate),
N-(2,3-dimethylpropyl)-N-{[2-(trifluoromethyl)phenyl]methyl}piperidin-4-amine (R isomer),
N-(2,3-dimethylpropyl)-N-{[2-(trifluoromethyl)phenyl]methyl}piperidin-4-amine (S isomer),
N-propyl-N-[(2-methylthiophenyl)methyl]piperidin-4-amine,
N-propyl-N-{[2-(difluoromethoxy)phenyl]methyl}piperidin-4-amine,
N-propyl-N-[(2-methylphenyl)methyl]piperidin-4-amine,
N-propyl-N-{[4-fluoro-2-(trifluoromethyl)phenyl]methyl}piperidin-4-amine,
N-propyl-N-[(2,4-dichlorophenyl)methyl]piperidin-4-amine,
N-butyl-N-[(2-methylthiophenyl)methyl]piperidin-4-amine,
N-butyl-N-{[2-(difluoromethoxy)phenyl]methyl}piperidin-4-amine,
N-butyl-N-[(2-methylphenyl)methyl]piperidin-4-amine,
N-butyl-N-[(2-chlorophenyl)methyl]piperidin-4-amine,
N-butyl-N-{[4-fluoro-2-(trifluoromethyl)phenyl]methyl}piperidin-4-amine,
N-butyl-N-[(2,4-dichlorophenyl)methyl]piperidin-4-amine,
N-(2-Methylpropyl)-N-{(2,3,6-(trichloro)phenyl]methyl}piperidin-4-amine,
N-(2-Methylpropyl)-N-{(2,3,5-(trichloro)phenyl]methyl}piperidin-4-amine,
N-(2-Methylpropyl)-N-{[(3-chloro-4-fluoro)phenyl]methyl}piperidin-4-amine,
N-(2-Methylpropyl)-N-{[2-chloro-3-(trifluoromethyl)phenyl]methyl}piperidin-4-amine,
N-(2-Methylpropyl)-N-{[(2,5-chloro)phenyl]methyl}piperidin-4-amine,
N-(2-Methylpropyl)-N-{[3-chloro-2-fluoro-6-(trifluoromethyl))phenyl]methyl}piperidin-4-amine,
N-(2-Methylpropyl)-N-{[(3-chloro-2-fluoro-5-(trifluoromethyl))phenyl]methyl}piperidin-4-amine,
N-(2-Methylpropyl)-N-{[(3-chloro-2-fluoro)phenyl]methyl}piperidin-4-amine,
N-(2-Methylpropyl)-N-{[(4-chloro-3-(trifluoromethyl))phenyl]methyl}piperidin-4-amine,
N-(2-Methylpropyl)-N-{[(2-chloro-5-(trifluoromethyl))phenyl]methyl}piperidin-4-amine,
N-(2-Methylpropyl)-N-{[(2-chloro-6-fluoro-3-methylphenyl]methyl}piperidin-4-amine,
N-(2-Methylpropyl)-N-{[(6-chloro-2-fluoro-3-methylphenyl]methyl}piperidin-4-amine,
N-(1-Propyl)-N-{[(2,3-dichloro)phenyl]methyl}piperidin-4-amine,
N-(1-Butyl)-N-{[(2,3-dichloro)phenyl]methyl}piperidin-4-amine,
N-(Cyclopropylmethyl)-N-{[(2,3-dichloro)phenyl]methyl}piperidin-4-amine,
N-(2,2-dimethylpropyl)-N-{[(2,3-dichloro)phenyl]methyl}piperidin-4-amine,
N-(2-Methylpropyl)-N-{[(3-chloro-2-methyl)phenyl]methyl}piperidin-4-amine,
N-(2-Methylpropyl)-N-{[(2-chloro-3-methyl)phenyl]methyl}piperidin-4-amine,
N-(2,2-Dimethylpropyl)-N-{[1,1-biphenyl]-2-yl-methyl}piperidin-4-amine,
N-(2,2-Dimethylpropyl)-N-{(2-phenoxyphenyl)methyl}piperidin-4-amine,
N-(2-Methylpropyl)-N-{[(2-chloro-4-fluoro)phenyl]methyl}piperidin-4-amine,
N-(1-Propyl)-N-{[(2-chloro-4-fluoro)phenyl]methyl}piperidin-4-amine,
N-(Cyclohexylmethyl)-N-{[(4-fluoro-2-(trifluoromethyl))phenyl]methyl}piperidin-4-amine,
N-(Cyclobutylmethyl)-N-{[(4-fluoro-2-(trifluoromethyl))phenyl]methyl}piperidin-4-amine,
N-(Cyclopentylmethyl)-N-{[(4-fluoro-2-(trifluoromethyl))phenyl]methyl}piperidin-4-amine,
N-(Cycloheptylmethyl)-N-{[(4-fluoro-2-(trifluoromethyl))phenyl]methyl}piperidin-4-amine,
N-(Cyclobutylmethyl)-N-{[(2,4-dichloro-phenyl)]methyl}piperidin-4-amine,
N-(2-Methylpropyl)-N-{[(2-fluoro-4-(trifluoromethyl))phenyl]methyl}piperidin-4-amine,
N-{[(2-Trifluoromethyl)phenyl]methyl}-N-tetrahydro-2H-pyran-4-yl-piperidin-4-amine,
N-(Cyclopentyl)-N-{[(2,3-dichloro)phenyl]methyl}piperidinamine,
N-(3,3,3-Trifluoropropyl)-N-{[(2,3-dichloro)phenyl]methyl}piperidin-4-amine,
N-{[(2,3-dichloro)phenyl]methyl}-N-tetrahydro-2H-pyranyl-piperidin-4-amine,
N-(2-Methylpentyl)-N-{[(2,3 dichloro)phenyl]methyl}piperidin-4-amine,
N-(2-Methylpropyl)-4-methyl-N-{[(2-(trifluoromethyl)phenyl]methyl}piperidin-4-amine,
N-(2-Methylpropyl)-N-{[(3-chloro-2-(trifluoromethyl))phenyl]methyl}piperidin-4-amine,
N-(2-Hydroxyethyl)-N-{[(4-fluoro-2-(trifluoromethyl))phenyl]methyl}piperidin-4-amine,
N-(2,2,2-Trifluoroethyl)-N-{[(2-(trifluoromethyl))phenyl]methyl}piperidinamine,
N-(2-Methylpropyl)-N {[2-chloro-4-(methylsulfonyl)phenyl]methyl}-piperidin-4-N-(3-Methoxypropyl)-N-{[(2-(trifluoromethyl))phenyl]methyl}piperidin-4-amine,
N-(3-Methoxypropyl)-N-{[(2,4-dichloro)phenyl]methyl}piperidin-4-amine,
N-(3-Methoxypropyl)-N-{[(4-fluoro-2-(trifluoromethyl))phenyl]methyl}piperidin-4-amine,
N-{2-[(1-Methylethyl)oxy]ethyl})-N-{[(2-(trifluoromethyl))phenyl]methyl}piperidin-4-amine,
N-{2-[(1-Methylethyl)oxy]ethyl})-N-{[(2,4-dichloro)phenyl]methyl}piperidin-4-amine,
N-{2-[(1-Methylethyl)oxy]ethyl})-N-{[(4-fluoro-2-(trifluoromethyl))phenyl]methyl}piperidin-4-amine,
N-[2-(Ethyloxy)ethyl]-N-{[(2-(trifluoromethyl))phenyl]methyl}piperidin-4-amine,
N-[2-(Ethyloxy)ethyl]-N-{[2,4-dichlorophenyl]methyl}piperidin-4-amine,
N-[2-(Ethyloxy)ethyl]-N-{[(4-fluoro-2-(trifluoromethyl))phenyl]methyl}piperidin-4-amine,
N-{[(4-Fluoro-2-trifluoromethyl)phenyl]methyl}-N-(tetrahydro-2H-pyran-4ylmethyl)-piperidin-4-amine,
N-[(2-(Methylthio)ethyl]-N-{[(4-fluoro-2-(trifluoromethyl))phenyl]methyl}piperidin-4-amine,
N-{[(2,3-Dichloro)phenyl]methyl}-N-tetrahydro-2H-pyran-4-yl-piperidin-4-amine,
N-{4-[(Methyl)oxy]butyl}-N-{[(2,4-dichloro)phenyl]methyl}piperidin-4-amine,
N-(3-hydroxy-3-methylbutyl)-N-{[(2,4-dichlorophenyl)methyl}piperidin-4-amine,
N-(2-hydroxy-2-methylpropyl)-N-{[(2,4-dichlorophenyl)methyl}piperidin-4-amine,
N-{2-[(Trifluoromethyl)oxy]ethyl}-N-{[(2,4-chloro)phenyl]methyl}piperidin-4-amine,
N-{2-[(Trifluoromethyl)oxy]ethyl}-N-{[(4-fluoro-2-(trifluoromethyl))phenyl]methyl}piperidin-amine,
N-{2-[(Trifluoromethyl)oxy]ethyl}-N-{[(2-(trifluoromethyl))phenyl]methyl}piperidin-4-amine,
N-(Cyclopropylmethyl)-N-{[(4-fluoro-2-(trifluoromethyl))phenyl]methyl}piperidin-4-amine,
2-Methylpropan-2-ol1-[[(4-fluoro-2-(trifluoromethyl)phenyl)methyl]piperidin-4-amine],
N-[1-(4-Fluoro-2-(trifluoromethyl)phenyl)ethyl]-N-(cyclopropylmethyl)piperidin-4-amine,
N-(3-Hydroxypropyl)-N-[[(2,4-dichlorophenyl)methyl](piperidin-4-amine,
N-(2-Hydroxyethyl)-N-[[(2,4-Dichlorophenyl)methyl](piperidin-4-amine],
3-[[(4-Fluoro-2-(trifluoromethyl)phenyl)methyl](piperidin-4-yl)amino]propanenitrile,
3-[[(4-Fluoro-2-(trifluoromethyl)phenyl)methyl)(piperidin-4-yl)amino]butanenitrile,
N-(Cyclopropylmethyl)-N-{[(2,3-Dichloro)phenyl]methyl}piperidin-4-amine,
3-[[(4-Fluoro-2-(trifluoromethyl)phenyl)methyl](piperidin-4-yl)amino]2,2-dimethylpropanenitrile,
4-[[(2,4-Dichlorophenyl)methyl](piperidin-4-yl)amino]-2,2-dimethylbutanenitrile,
4-[[(4-Fluoro-2-(trifluoromethyl))phenyl)methyl]piperidin-4-yl)amino]-2,2-dimethylbutanenitrile,
3-[[(2,4-Dichloro)phenyl]methyl}(piperidin-4-yl)amino]-butanenitrile,
3-[[(2(Trifluoromethyl)phenyl]methyl}piperidin-4-yl)amino]-butanenitrile,
N-(3-Methyl-3-hydroxybutyl)-N-{[4-fluoro-2-(trifluoromethyl)phenyl]methyl}piperidin-4-amine,
1-{[(2,4-Dichloro)phenyl]methyl}(piperidin-4-yl)amino] cyclopentanol, and
N-[1-Fluorocyclopropyl)methyl]-N-{[(2,4-dichloro)phenyl]methyl}piperidin-4-amine.

The present invention includes pharmaceutically acceptable salts of the compounds of formula I. Suitable salts include acid addition salts, including salts formed with inorganic acids (for example hydrochloric, hydrobromic, nitric, sulphuric or phosphoric acid) or with organic acids, such as organic carboxylic acids (for example fumaric, pyruvic, lactobionic, glycolic, oxalic, maleic, hydroxymaleic, malic, citric, salicylic, o-acetoxybenzoic or tartaric acid), or organic sulphonic acids (for example toluene-p-sulphonic, bisethanesulphonic or methanesulphonic acid).

It will be appreciated that certain compounds of formula I may possess one or more chiral centres. Where a structural formula does not specify the stereochemistry at one or more chiral centres, it encompasses all possible stereoisomers and all possible mixtures of stereoisomers (including, but not limited to, racemic mixtures) which may result from stereoisomerism at each of the one or more chiral centers.

As mentioned above, the compounds of the present invention and their pharmaceutically acceptable salts inhibit the uptake of one or more of the monoamine neurotransmitters serotonin, dopamine and norepinephrine.

In view of these properties, the compounds of the present invention and their pharmaceutically acceptable salts are indicated for use in treating disorders which are caused by or linked to decreased neurotransmission of one or more of these monoamines. Such disorders include disorders of the central and/or peripheral nervous system such as, for example, adjustment disorders (including depressed mood, anxiety, mixed anxiety and depressed mood, disturbance of conduct, and mixed disturbance of conduct and mood), age-associated learning and mental disorders (including Alzheimer's disease), alcohol addiction, antinociceptive pain, apathy, attention-deficit (or other cognitive) disorders due to general medical conditions, attention-deficit hyperactivity disorder (ADD)), autism, bipolar disorder, borderline personality disorder, brain trauma, cardiovascular disorders, chronic fatigue syndrome, chronic or acute stress, Chron's disease, cognitive disorders including mild cognitive impairment (MCI), conduct disorder, cyclothymic disorder, dementia of ageing, dementia of the Alzheimers type (DAT), depression (including adolescent depression and minor depression), dyspepsia, disruptive behavior disorders, drug addiction including cocaine abuse, dysthymic disorder, eating disorders (including bulimia and anorexia nervosa), emesis, emotional dysregulation, epilepsy, fibromyalgia and other somatoform disorders (including somatization disorder, conversion disorder, pain disorder, hypochondriasis, body dysmorphic disorder, undifferentiated somatoform disorder, and somatoform NOS), functional bowel disorders, gastric motility disorders, gastroesophageal reflux for functional bowel disorders, gastrointestinal disorders, generalized anxiety disorder (GAD), headache, hot flushes/flashes, hypertension, hypotensive states including orthostatic hypotension, iletis, impulsive control disorders, incontinence (i.e., stress incontinence, genuine stress incontinence, urge incontinence and mixed incontinence), inflammatory bowel disorders, inhalation disorders, insterstitial cystitis, intoxication disorders (alcohol addiction), irritable bowel syndrome, ischemic bowel disease, mania, memory loss, mutism, nicotine addiction, obesity (i.e., reducing the weight of obese or overweight patients), obsessive compulsive disorders and related spectrum disorders, oppositional defiant disorder, pain (including chronic pain, inflammatory pain, neuropathic pain, non-neuropathic non-inflammatory pain, persistent pain, persistent pain of inflammatory and/or neuropathic origin, headache and migraine), panic disorders, Parkinsonism, peripheral neuropathy, post-traumatic stress disorder, premenstrual dysphoric disorder (i.e., premenstrual syndrome and late luteal phase dysphoric disorder), psoriasis, psychoactive substance use disorders, psychotic disorders (including schizophrenia, schizoaffective and schizophreniform disorders), seasonal affective disorder, selective serotonin reuptake inhibition (SSRI) “poop out” syndrome (i.e., wherein a patient who fails to maintain a satisfactory response to SSRI therapy after an initial period of satisfactory response), senile dementia, sexual dysfunction (including premature ejaculation and erectile difficulty), sleep disorders (such as narcolepsy and enuresis), smoking cessation, social phobia (including social anxiety disorder), specific developmental disorders, substance abuse (including alcohol addiction, tobacco abuse, symptoms caused by withdrawal or partial withdrawal from the use of tobacco or nicotine and drug addiction including cocaine abuse), TIC disorders (e.g., Tourette's Disease), tobacco addiction, trichotilomania, ulcerative colitis, urethral syndrome, vascular dementia and cognitive impairment associated with schizophrenia (CIAS).

One preferred group of compounds of the present invention selectively inhibit the reuptake of serotonin and norepinephrine over doparine. Preferably said group of compounds of the present invention selectively inhibit the serotonin and norepinephrine transporters relative to the dopamine transporter by a factor of at least five, and even more preferably by a factor of at least ten. Compounds of the present invention with this pharmacological profile are particularly useful for the treatment of depression, eating disorders (including bulimia and anorexia nervosa), inflammatory bowel disorders, functional bowel disorders, dyspepsia, Chron's disease, iletis, ischemic bowel disease, ulcerative colitis, gastroesophageal reflux for functional bowel disorders, irritable bowel syndrome, obesity, insterstitial cystitis, urethral syndrome, gastric motility disorders, substance abuse (including alcoholism, tobacco abuse, symptoms caused by withdrawal or partial withdrawal from the use of tobacco or nicotine and drug addiction including cocaine abuse), pain (including inflammatory pain, neuropathic pain, non-neuropathic non-inflammatory pain, persistent pain, persistent pain of inflammatory and/or neuropathic origin, headache and migraine), incontinence (including stress urinary incontinence and urge incontinence), dementia of ageing, senile dementia, Alzheimer's, memory loss, Parkinsonism, attention-deficit disorder (including attention-deficit hyperactivity disorder), anxiety, social phobia, disruptive behavior disorders, impulsive control disorders, borderline personality disorder, chronic fatigue syndrome, panic disorders, obsessive compulsive disorder, post-traumatic stress disorder, schizophrenia, gastrointestinal disorders, cardiovascular disorders, hot flushes/flashes, emesis, sleep disorders, cognitive disorders, psychotic disorders, brain trauma, premenstrual syndrome or late luteal syndrome, sexual dysfunction (including premature ejaculation and erectile difficulty), autism, mutism and trichotilomania They are more particularly useful for the treatment of depression, incontinence (particularly stress urinary incontinence) and pain (particularly persistent pain). They are most particularly useful for the treatment of persistent pain.

For clinical purposes, pain may be divided into two categories: acute pain and persistent pain. Acute pain is provoked by noxious stimulation produced by injury and/or disease of skin, deep somatic structures or viscera, or abnormal function of muscle or viscera that does not produce actual tissue damage. On the other hand, persistent pain can be defined as pain that persists beyond the usual course of an acute disease or a reasonable time for an injury to heal or that is associated with a chronic pathologic process that causes continuous pain or the pain recurs at intervals for months or years. If pain is still present after a cure should have been achieved, it is considered persistent pain. For the purpose of the present invention, persistent pain can be chronic non-remitting or recurrent. The difference in definition between acute and persistent pain is not merely semantic but has an important clinical relevance. For example, a simple fracture of the wrist usually remains painful for a week to 10 days. If the pain is still present beyond the typical course of treatment, it is likely that the patient is developing reflex sympathetic dystrophy, a persistent pain syndrome that requires immediate effective therapy. Early and effective intervention potentially prevents the undue disability and suffering, and avoids the potential development of a condition that becomes refractory to therapy.

Acute and persitant pain differ in etiology, mechanisms, pathophysiology, symptomatology, diagnosis, therapy, and physiological responses. In contrast to the transitory nature of acute pain, persistent pain is caused by chronic pathologic processes in somatic structures or viscera, by prolonged and sometimes permanent dysfunction of the peripheral or central nervous system, or both. Also, persistent pain can sometimes be attributed to psychologic mechanisms and/or environmental factors.

More specifically, persistent pain can be segmented into neuropathic pain (e.g. diabetic neuropathy, infectious neuropathic pain associated with AIDS, non-surgical carpal tunnel syndromes, post-herpetic neuralgia, cervical, thoracic and lumbosacral radiculopathies, stroke-related central pains, trigeminal neuralgia and complex regional pain syndromes I and II), inflammatory pain (e.g. polymyalgia, rheumatoid arthritis and osteoarthritis), and non-neuropathic non-inflammatory pain, non-neuropathic non-inflammatory chronic pain (NNNICP) (e.g. chronic fatigue syndrome, chronic back pain without radiculopathy, fibromyalgia, chronic tension type headaches, inflammatory bowel disorders, irritable bowel syndrome, whiplash injuries, chronic pelvic pain, TMJD and failed back).

Current therapies for persistent pain include opiates, barbiturate-like drugs such as thiopental sodium and surgical procedures such as neurectomy, rhizotomy, cordotomy, and cordectomy.

Another preferred group of compounds of the present invention selectively inhibit the reuptake of norepinephrine over serotonin and dopamine. Preferably said group of compounds of the present invention selectively inhibit the norepinephrine transporter relative to the serotonin and dopamine transporters by a factor of at least five, and even more preferably by a factor of at least ten. Compounds of the present invention with this pharmacological profile are particularly useful for the treatment of adjustment disorders (including depressed mood, anxiety, mixed anxiety and depressed mood, disturbance of conduct, and mixed disturbance of conduct and mood), age-associated learning and mental disorders (including Alzheimer's disease), alcohol addiction, anorexia nervosa, antinociceptive pain, apathy, attention-deficit (or other cognitive) disorders due to general medical conditions, attention-deficit hyperactivity disorder (ADHD), bipolar disorder, bulimia nervosa, chronic fatigue syndrome, chronic or acute stress, cognitive disorders including mild cognitive impairment (MCI), conduct disorder, cyclothymic disorder, dementia of the Alzheimers type (DAT), depression (including adolescent depression and minor depression), dysthymic disorder, emotional dysregulation, fibromyalgia and other somatoform disorders (including somatization disorder, conversion disorder, pain disorder, hypochondriasis, body dysmorphic disorder, undifferentiated somatoform disorder, and somatoform NOS), generalized anxiety disorder (GAD), hypotensive states including orthostatic hypotension, incontinence (i.e., stress incontinence, genuine stress incontinence, and mixed incontinence), inhalation disorders, intoxication disorders (alcohol addiction), mania, migraine headaches, neuropathic pain, nicotine addiction, obesity (i.e., reducing the weight of obese or overweight patients), obsessive compulsive disorders and related spectrum disorders, oppositional defiant disorder, pain including chronic pain, panic disorder, peripheral neuropathy, post-traumatic stress disorder, premenstrual dysphoric disorder (i.e., premenstrual syndrome and late luteal phase dysphoric disorder), psoriasis, psychoactive substance use disorders, psychotic disorders (including schizophrenia, schizoaffective and schizophreniform disorders), seasonal affective disorder, selective serotonin reuptake inhibition (SSRI) “poop out” syndrome (i.e., wherein a patient who fails to maintain a satisfactory response to SSRI therapy after an initial period of satisfactory response), sleep disorders (such as narcolepsy and enuresis), social phobia (including social anxiety disorder), somatoform disorders, specific developmental disorders, TIC disorders (e.g., Tourette's Disease), tobacco addiction, vascular dementia and cognitive impairment associated with schizophrenia (CIAS). They are most particularly useful for the treatment of ADHD and schizophrenia.

Another preferred group of compounds of the present invention inhibit the reuptake of norepinephrine, serotonin and dopamine. Compounds of the present invention with this pharmacological profile are particularly useful for the treatment of a variety of conditions such as depression, obesity, compulsive disorders (including bulimia, obsessive compulsive disorder, drug addiction including cocaine abuse and alcohol addiction), hypertension, senile dementia, Alzheimer's, memory loss, attention-deficit hyperactivity disorder (ADHD), sexual dysfunction, Parkinsonism, anxiety, chronic fatigue syndrome, panic disorders, cognitive disorders, schizophrenia, gastrointestinal disorders, headache, cardiovascular disorders, epilepsy, smoking cessation, pain including chronic pain, urinary incontinence, emesis and sleep disorders. They are most particularly useful for the treatment of depression, chronic pain, smoking cessation and obesity.

Accordingly, the present invention provides a compound of Formula I or a pharmaceutically acceptable salt thereof for use in therapy. In particular, the present invention provides a compound of Formula I or a pharmaceutically acceptable salt thereof for use as an inhibitor of the uptake of one or more of the monoamine neurotransmitters serotonin, dopamine and norepinephrine.

In another embodiment, the present invention provides a method for inhibiting the uptake of one or more monoamines selected from serotonin, dopamine and norepinephrine in a mammal, comprising administering to a mammal in need of such inhibition an effective amount of a compound of Formula I or a pharmaceutically acceptable salt thereof. In particular, the present invention provides a method for treating a disorder which is caused by or linked to decreased neurotransmission of one or more monoamines selected from serotonin, dopamine and norepinephrine in a mammal, comprising administering to a mammal in need of such treatment an effective amount of a compound of Formula I or a pharmaceutically acceptable salt thereof. Such disorders include, for example, disorders of the central and/or peripheral nervous system.

In the context of the present specification the terms “treating” and “treatment” include prophylactic treatment as well as curative treatment.

In another alternative embodiment, the present invention provides for the use of a compound of Formula I or a pharmaceutically acceptable salt thereof for the manufacture of a medicament for inhibiting the uptake of one or more monoamines selected from serotonin, dopamine and norepinephrine. In particular, the present invention provides for the use of a compound of Formula I or a pharmaceutically acceptable salt thereof for the manufacture of a medicament for the treatment of a disorder which is caused by or linked to decreased neurotransmission of one or more monoamines selected from serotonin, dopamine and norepinephrine. Such disorders include, for example, disorders of the central and/or peripheral nervous system.

The compounds may be administered by various routes and are usually employed in the form of a pharmaceutical composition.

Accordingly, in a further embodiment, the present invention provides a pharmaceutical composition comprising a compound of Formula I or a pharmaceutically acceptable salt thereof together with a pharmaceutically acceptable diluent or carrier.

Such compositions may be prepared by methods well known in the pharmaceutical art and normally comprise at least one active compound in association with a pharmaceutically acceptable diluent or carrier. In making the compositions of the present invention, the active ingredient will usually be mixed with a carrier or diluted by a carrier, and/or enclosed within a carrier which may, for example, be in the form of a capsule, sachet, paper or other container.

The compositions indicated can be sterilized and/or can contain auxiliaries such as lubricants, preservatives, stabilizers and/or wetting agents, emulsifiers, salts for affecting the osmotic pressure, buffer substances, colourants, flavourings and/or one or more further active compounds. Compositions of the invention may be formulated so as to provide, quick, sustained or delayed release of the active ingredient after administration to the patient by employing procedures well known in the art.

The compositions are preferably formulated in a unit dosage form, each dosage containing from about 5 to about 500 mg of the active ingredient. In the context of the present specification, the term “unit dosage form” refers to physically discrete units suitable as unitary doses for human subjects and other mammals, each unit containing a predetermined quantity of one or more compounds of Formula I or pharmaceutically acceptable salts thereof, calculated to produce the desired therapeutic effect, together with a pharmaceutically acceptable diluent or carrier.

Compounds of formula I may be prepared by conventional organic chemistry techniques and also by solid phase synthesis.

In the present specification the abbreviation “boc” or “BOC” refers to the N-protecting group t-butyloxycarbonyl.

In the present specification the abbreviation “TFA” refers to trifluoroacetic acid.

In the present specification the abbreviation “DMF” refers to dimethylformamide.

In the present specification the abbreviation “SPE” refers to solid phase extraction.

In the present specification the abbreviation “ACE-Cl” refers to α-chloroethyl chloroformate.

When R8 is H, a suitable three-step conventional synthesis is outlined in the scheme shown below.

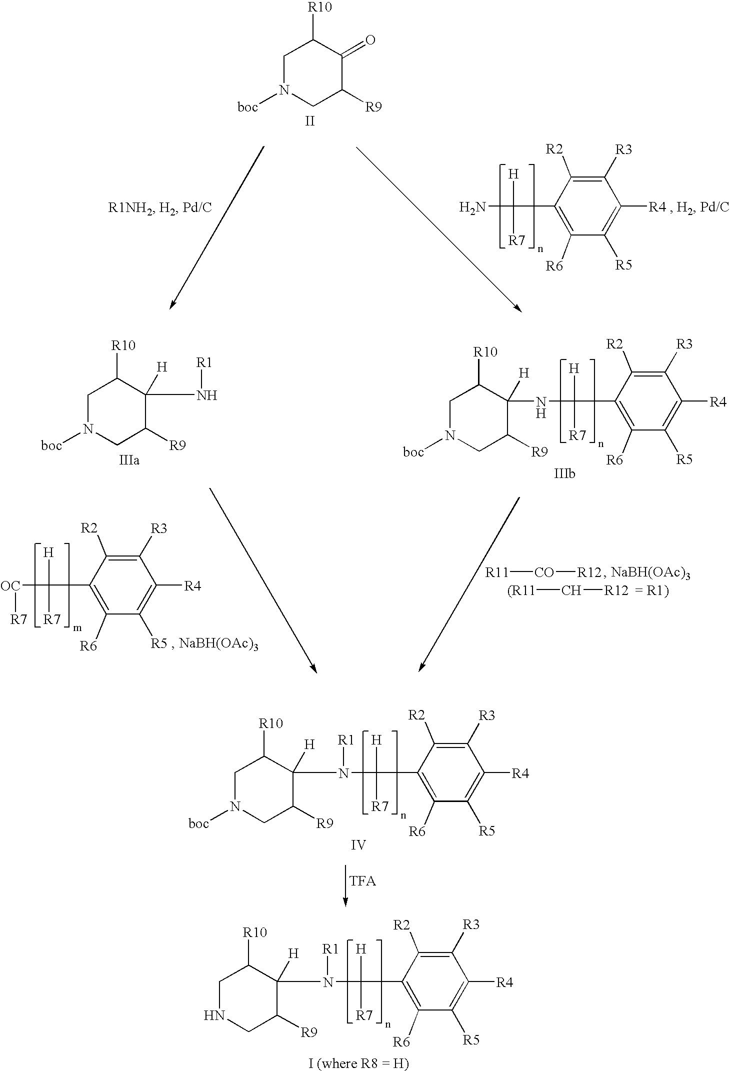

A boc-protected 4-piperidone (II) is reductively aminated with an amine to provide a 4-amino-piperidine (IIIa or IIIb). A second reductive amination with an aldehyde or ketone provides a boc-protected compound of formula I (IV). The boc group is removed under acidic conditions to provide a compound of formula I (where R8 is H). If desired, the compound of formula I (where R8 is H) may be converted to a suitable salt by addition of a suitable quantity of a suitable acid. In the schemes above (and below) R1 to R7, R9, R10 and n are as previously defined, m is 0, 1 or 2 and R11 and R12 are chosen such that R11-CH-R12=R1.

Although the boc N-protecting group is used in the above illustration, it will be appreciated that other N-protecting groups (for example acetyl, benzyl or benzoxycarbonyl) could also be used together with a deprotection step appropriate for the N-protecting group used. Similarly, other reducing agents (for example NaBH₄ or LiAlH₄) may be used in the reductive amination steps and other acids (for example HCl) may be used in the deprotection step′.

As an alternative to the second reductive amination step, compound IIIa or IIIb) may be subjected to an alkylation step as shown below (L represents a suitable leaving group—for example Br or tosyl).

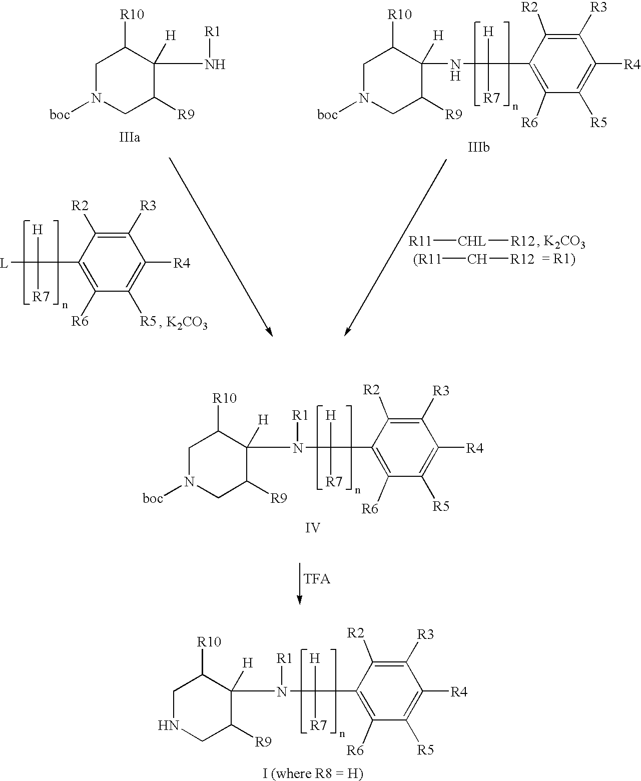

Once again, N-protection other than boc may also be used together with a suitable deprotection step. Similarly, bases other than potassium carbonate (e.g NaH) may be used for the alkylation step

Using essentially the same chemical reactions as in the first scheme above, the compounds of formula I (where R8 is H) may also be prepared by a solid phase parallel synthesis technique as outlined in the scheme shown below.

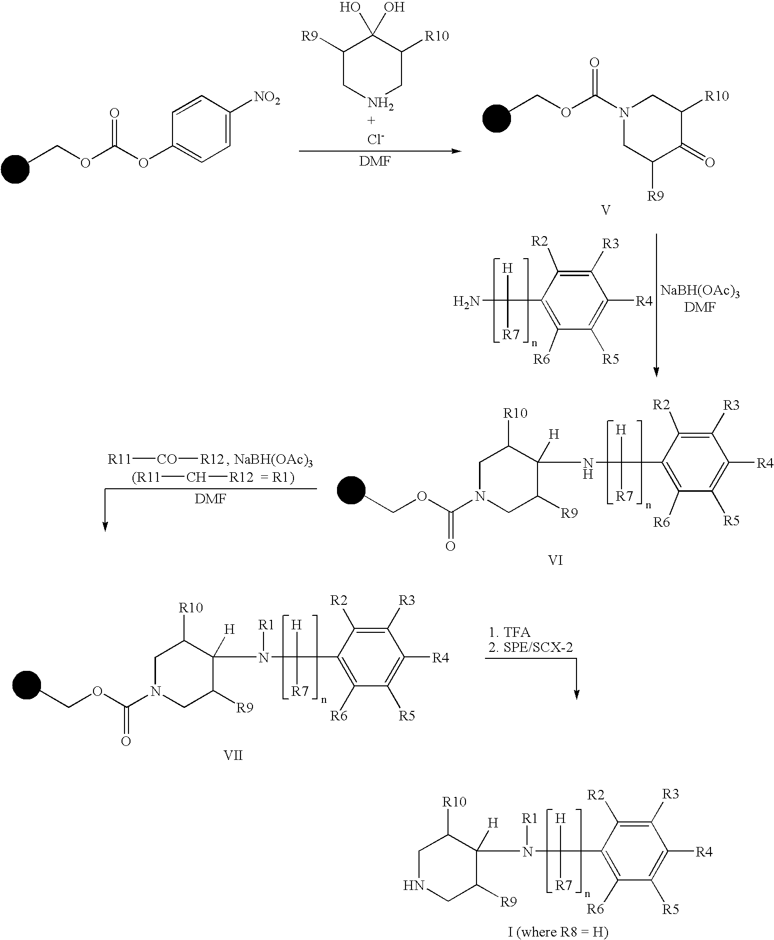

A piperidone hydrate is attached to a polystyrene resin to provide a resin bound piperidone (V). Aliquots are reductively aminated to provide a resin bound secondary amine (VI) that can undergo a further reductive amination with an aldehyde or ketone to give the tertiary amine (VII). Acidic cleavage from the resin and SPE provides compounds of formula I (where R8 is H) which may be purified using, for example, the SCX-2 derivatised silica.

Although NaBH(OAc)₃ is used in the above illustration, it will be appreciated that other reducing agents (for example NaBH₄ or LiAlH₄) may be used in the reductive amination steps and other acids (for example HCl) may be used in the deprotection step. Solid phase resins other than the p-nitrophenylcarbonate-polystyrene resin illustrated above may also be employed.

When R8 is C₁-C₄alkyl, a conventional synthetic route is outlined in the scheme shown below.

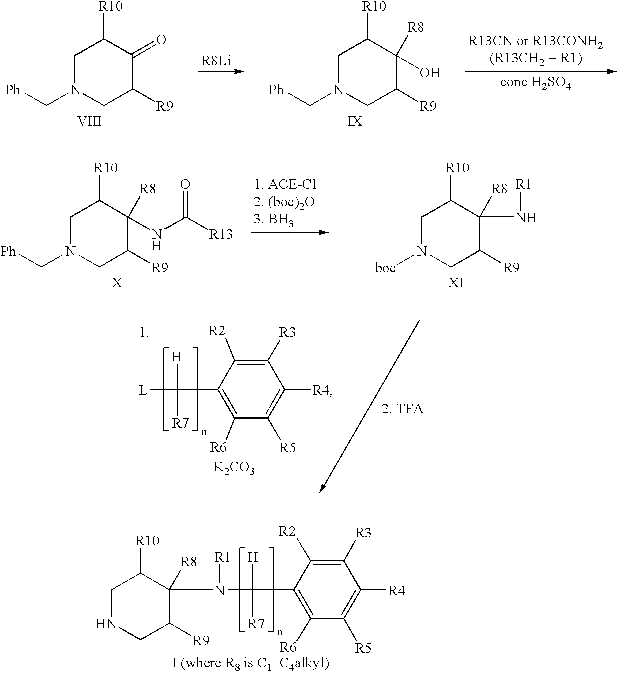

A benzyl-protected 4-piperidone (VIII) is alkylated with an alkyllithium reagent to provide a 4-amino-piperidinol (IX). Treatment with an alkylnitrile or alkylamide under strongly acidic conditions provides a secondary amide (X) which may be deprotected, boc-protected and reduced to provide a secondary amine (XI). Alkylation of the secondary amine (I followed by removal of the boc group provides a compound of formula I (where R8 is C₁-C₄alkyl). In the scheme above L is a leaving group as previously defined and R13 is chosen such that R13-CH₂=R1.

Although the benzyl and boc N-protecting groups are used in the above illustration, it will be appreciated that other N-protecting groups could also be used in their place together with deprotection steps appropriate for those N-protecting groups. Similarly, other reducing agents may be used in the amidecarbonyl reduction step and other organometallics or bases may be used in the respective alkylation steps.

The present invention also provides a process for producing a compound of formula I above, which comprises deprotecting a compound of the formula 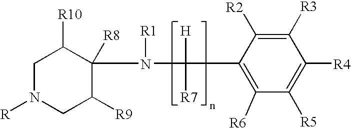
where R is an N-protecting group. Suitable N-protecting groups will be known to the person skilled in the art and include, for example, boc, benzyl, benzyloxycarbonyl and acetyl.

### EXAMPLE 1

### N-(2-methylpropyl)-N-{[2-(trifluoromethyl)phenyl]methyl}piperidin-4-amine fumarate

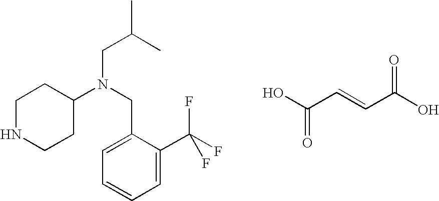 Method 1

(i) N-BOC-piperidone (1.25 g, 6.27 mmol) and 2-trifluoromethylbenzylamine (1.1 g, 6.28 mmol) were hydrogenated at 60 psi in ethanol (30 ml) in the presence of 5% palladium on charcoal (0.3 g) using a Parr hydrogenator. After 2.5 h, the catalyst was filtered off and the filtrate was evaporated to give an oil (2.5 g). The oil was purified by flash chromatography over silica, ramping the solvent mixture from 20% cyclohexane in ethyl acetate to ethyl acetate to give 1,1-dimethylethyl 4-({[2-(trifluoromethyl)phenyl]methyl}amino)piperidine-1-carboxylate as an oil (1.8 g, 80%). δ_{H} (300 MHz, CDCl₃) 7.62-7.66 (2H, dd, 2 ArH). 7.50-7.55 (1H, t, ArH), 7.32-7.37 (1H, t, ArH), 3.97 (2H, s, ArCH2), 3.97-4.19 (2H, m, NCH2), 2.78-2.86 (2H, brt, NCH2), 2.64-2.74 (1H, m, NCH), 1.85-1.90 (2H, dd, CCH2), 1.50-1.24 (2H, m, CCH2), 1.46 (9H, s, 3×CCH₃); LCMS 6 min gradient method, Rt=5.28 min (M⁺+H)=415.

(ii) Sodium triacetoxyborohydride (0.497 g, 2.35 mmol) was added in two lots over 15 min to a stirred mixture of 1,1-dimethylethyl 4-({[2-(trifluoromethyl)phenyl]methyl}amino)piperidine-1-carboxylate (0.6 g, 1.68 mmol), isobutyraldehyde (0.483 g, 6.70 mmol), acetic acid (1.01 g, 16.7 mmol) and 1,2-dichloroethane (10 ml). After stirring for 3 days under nitrogen, excess SM sodium hydroxide was added and the mixture was stirred for 30 min. The mixture was extracted 3 times with dichloromethane. The dichloromethane extracts were combined, washed (H₂O), dried (MgSO₄) and evaporated to give an oil (0.7 g). The oil was dissolved in ethanol (10 ml) and 2M HCl in ether (3.5 ml) was added and the mixture was stirred at room temperature under nitrogen for 1 day. The solution was evaporated in vacuo at 50° C. and the resulting oil was converted to the free base using a SCX-2 ion exchange column, eluting with methanol and then a 2.3M solution of ammonia in methanol to give an oil. The oil was converted to the fumarate salt (ethanol/ether) give the title product as a white solid (0.151 g, 21%). 5 (300 MHz, MeOD) 7.95-7.98 (1H, d), 7.58-7.67 (2H, m), 7.38-7.43 (1H, t), 6.69 (2H, s), 3.87 (2H, s), 3.43-3.47 (2H, d), 2.77-2.97 (3H, m), 3.32-3.35 (2H,d), 2.02-2.06 (2H, m), 2.02-2.06 (3H, m), 0.90-0.92 (6H, d); LCMS 12 min gradient method, Rt=5.6 min, (M⁺+H)=315.

Method 2

(i) To a mixture of N-BOC-piperidone (22 g, 110 mmol) and 2-trifluoromethylbenzylamine (19.3 g, 110 mmol) in a Parr bottle was placed ethanol (300 ml). Palladium on carbon (5%, 6 g) was then added and the mixture hydrogenated at 65 psi for 3 hr. Reaction filtered through Celite, concentrated in vacuo to give 1,1-dimethylethyl 4-({[2-(trifluoromethyl)phenyl]methyl}amino)piperidine-1-carboxylate as an oil (37.2 g, 94%). LCMS—6 mins gradient Rt=2.69 (MF+1) 359.2 1H NMR (CDCl₃) δ=7.65-7.60 (2H, m), 7.55-7.50 (1H, m), 7.46-7.33 (1H, m), 4.10-3.95 (4H, m), 2.90-2.75 (2H, m), 2.70-2.61 (1H, m), 1.91-1.85 (2H, m), 1.49 (9H, s), 1.38-1.22 (2H, m).

(ii) A solution of 1,1-dimethylethyl 4-({[2-(trifluoromethyl)phenyl]methyl}amino)piperidine-1-carboxylate (28.0 g, 80.7 mmol), iso-butylaldehyde (23.27 g, 29.4 ml, 322.8 mmol) and acetic acid (807 mmol, 48.46 g, 46.2 ml) in 1,2-dichloroethane (500 ml) at room temperature under nitrogen was stirred for 30 min. Then sodium triacetoxyborohydride (112.9 mmol, 23.9 g) was added portion wise over 15 min and the reaction left to stir at room temperature for 16 h. Excess 2M NaOH was added to the reaction mixture until pH>12. The mixture was extracted into dichloromethane (3×400 ml). The combined organics were dried MgSO4), filtered, and concentrated in vacuo to give the crude product as an oil. Purified using a silica column eluting with 0-5% ethyl acetate in cyclohexane: to yield an oil in two batches (12 g and 10 g each).

(iii) To a solution of the above oil (11.9 g, 28.7 mmol) in ethanol was placed HCl (conc. 28 ml) and stirred at room temperature for 5 days. An additional 50 ml of HCl added and stirred for an additional 16 h. Solvent removed in vacuo and then the crude was partitioned between dichloromethane/water and 2M NaOH added until pH>10. The phases were then separated, aqueous washed with dichloromethane and combined organics dried, (MgSO₄), filtered and concentrated in vacuo to give a crude oil (7.5 g, 83%). Compound was purified on silica column DCM:MeOH:NH3 (100:5:1) to give a N-(2-methylpropyl)-N-{[2-(trifluoromethyl)phenyl]methyl}piperidin-4-amine (4.5 g, 50%). Crude fractions were then combined with the column fractions of a second similar reaction to give a total yield of (11.3 g, 68%). The fumarate salt was made by taking the free base in ethanol (150 ml) and whilst heating adding fumaric acid (1 equivalent) as an ethanol solution. The salt was washed with diethyl ether to yield the title product as a white solid (12.6 g, 81% for salt formation).

### EXAMPLE 2

### N-(2-methylpropyl)-N-{[2-(trifluoromethyl)phenyl]methyl}piperidin-4-amine L-tartrate

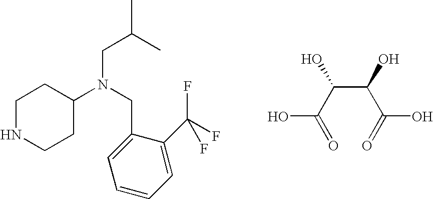

(i) To a solution of N-BOC-piperidin-4-one (500 g, 2.5 mol) in 2.3 L absolute ethanol, was added at room temperature isobutylamine (187.5 g, 2.56 mol). After 30 min stirring reaction mixture was transferred to a stainless steel hydrogenator and 40 g anhydrous 10% Pd/C was added. Reactor was carefully purged with nitrogen and pressurised with hydrogen to 15 psi over atmospheric pressure. Exothermic reaction immediately took place, temperature was not allowed to exceed 27° C. After 30 min hydrogen was no longer consumed. Pressure was then increased to 22 psi for a 15 min period in order to assure reaction completion. Residual hydrogen pressure was released and reactor purged with nitrogen. Reaction mixture was then filtered on celite and solvent stripped out under vacuum up to 50° C. The product 4-isobutylamino-piperidine-1-carboxylic acid tert-butyl ester was isolated as pale yellow oil, 631 g (98% yield). 1H NMR (250 MHz, CDCl₃) 8 ppm: 0.92 (d, J=6.57 Hz, 6H), 1.27 (m, 2H), 1.46 (s, 9H), 1.72 (m, 1H), 1.84 (d, J=12.13 Hz, 2H), 2.45 (d, J=6.82 Hz, 2H), 2.58 (t, J=10.11 Hz, 1H), 2.81 (t, J=11.87 Hz, 2H), 4.03 (m, 2H). ¹³C NMR (250 MHz, CDCl₃) 3 ppm: 21.04, 28.85, 28.98, 33.02, 42.97, 55.28, 55.34, 79.61, 155.09.

(ii) To a solution of 4-isobutylamino-piperidine-1-carboxylic acid tert-butyl ester (20 g, 0.078 mol) in 200 ml anhydrous THF was added 2-trifluoromethylbenzaldehyde (16.37 g, 0.094 mol) and then sodium triacetoxyborohydride (24.8 g, 0.117 mol) in one portion. Very slight exothermic reaction was observed (temperature increased from 23 to 27° C.). After 17 h reaction at room temperature an additional portion of 2-trifluoromethylbenzaldehyde (3.3 g, 0.019 mol) and then sodium triacetoxyborohydride (5 g, 0.023 mol) was added. After 7 h at room temperature no more N-BOC-4-isobutylaminopiperidine was detected.

Reaction mixture was cooled down to 0° C. and a 1M NaOH solution 200 ml was added. The mixture was extracted twice with 200 ml methyl tert-butyl ether, organic layers were isolated then dried over MgSO₄, filtered, and collected liquors were concentrated under vacuum. The crude compound was then chromatographed on silica gel (eluant: heptane/ethyl acetate 95/5) to give 26.3 g of pure compound 4-[isobutyl-(2-trifluoromethyl-benzyl)amino]piperidine-1-carboxylic acid tert-butyl ester (81% yield). As an alternative to chromatography the crude 4-[isobutyl-(2-trifluoromethyl-benzyl)amino]piperidine-1-carboxylic acid tert-butyl ester (10 g) was dissolved in MeOH (34 ml) at 40° C. and water (8.5 ml) was added dropwise under stirring. After cooloing to 20° C., the white solid was filtered, washed 3 times with 3 ml of 80:20 (v:v) MeOH/water and dried at 50° C. under vacuum overnight (88% yield). 1H NMR (250 MHz, CDCl₃) 5 ppm 0.72 (d, J=6.61 Hz, 6H), 1.28 (m, 11H), 1.46 (m, 1H), 1.57 (d, J=13.53 Hz, 2H), 2.10 (d, J=6.92 Hz, 2H), 2.37 (m, 3H), 3.64 (s, 2H), 3.99 (d, J=12.59 Hz, 2H), 7.13 (m, 1H), 7.35 (t, J=7.55 Hz, 1H), 7.43 (d, J=7.87 Hz, 1H), 7.78 (d, J=7.87 Hz, 1H). ¹³C NMR (250 MHz, CDCl₃) 8 ppm: 21.17, 27.3, 28.12, 28.84, 44.17, 51.36, 58.43, 19.07, 79.73, 122.81, 125.74-125.84, 126.66, 127.17-128.17-128.64 (CF3), 130.24, 132.04, 140.94, 155.11.

(iii) To a mixture of 36 ml ethanol and 37% HCl 16 ml heated up to 50° C. was added portion-wise 4-[isobutyl-(2-trifluoromethyl-benzyl)amino]piperidine-1-carboxylic acid tert-butyl ester (8 g, 0.01936 mol) gas evolution was observed. Reaction mixture was then heated to 60° C. After 30 min reaction was completed.

The crude hydrochloride salt (8.4 g material) was then isolated by concentration of the reaction mixture under vacuum up to 50° C. This was neutralised by 100 ml 1M NaOH solution, then extracted twice with 100 ml methyl tert-butyl ether. Upper organic layer was then isolated, washed twice with 10 ml 10% NaCl, dried over MgSO₄. N-(2-methylpropyl)-N-{[2-(trifluoromethyl)phenyl]methyl}piperidin-4-amine 6.3 g was then isolated as pale yellow oil by filtration of the salts and evaporation of the solvent under vacuum up to 50° C. 1H NMR (250 MHz, CDCl₃) 8 ppm 0.89 (d, J=6.61 Hz, 6H), 1.45 (m, 21), 1.65 (m, 2H), 1.78 (d, J=13.53 Hz, 2H), 2.3 (d, J=6.92 Hz, 2H), 2.49 (m, 3H), 3.11 (d, J=9.91 Hz, 21), 3.82 (s, 2H), 7.30 (t, J=7.55 Hz, 1H), 7.51 (t, J=7.55 Hz, 1H), 7.60 (d, J=7.87 Hz, 1H), 7.98 (d, J=7.87 Hz, 1H).

(iv). A solution of N-(2-methylpropyl)-N-{[2-(trifluoromethyl)phenyl]methyl}piperidin-4-amine (6.3 g, 0.02 mol) dissolved in isopropanol (70 ml), then L-tartaric acid (3 g, 0.02 mol) was added in one portion, then the mixture was heated up to 65° C. After 1 h at 65° C. the mixture was allowed to stir at room temperature for another 1 h. The title compound (8.4 g, 90% yield) was then isolated by filtration; washed twice with 10 ml isopropanol, dried under vacuum up to 40° C. 1H NMR (400 MHz, DMSO-D6) 8 ppm 0.81 (d, J=6.57 Hz, 6H), 1.58 (m, 1H), 1.73 (m, 2H), 1.83 (m, 2H), 2.24 (d, J=7.07, 2H), 2.71 (t, J=11.62 Hz, 1H), 2.79 (m, 2H), 3.29 (d, J=12.13 Hz, 211), 3.76 (s, 2H), 3.96 (s, 2H), 4.60 (s, 5H), 7.44 (t, J=7.45 Hz, 1H), 7.66 (m, 1H), 7.67 (d, J=7.83 Hz, 1H), 7.90 (d, J=8.08 Hz, 1H). 13C NMR (250 MHz, DMSO-D6) 3 ppm: 20.94, 24.72, 26.63, 43.53, 51.01, 55.34, 58.64,72.00, 125.78, 126.77, 127.12, 127.24, 127.38, 130.28, 132.78, 140.10, 174.90.

### EXAMPLE 3

### N-(1-methylethyl)-N-{[2-(trifluoromethyl)phenyl]methyl}piperidin-4-amine fumarate

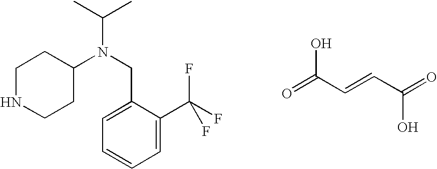

(i) Sodium triacetoxyborohydride (0.5 g, 2.36 mmol) was added to a mixture of 1,1-dimethylethyl 4-({[2-(trifluoromethyl)phenyl]methyl}amino)piperidine-1-carboxylate (0.6 g, 1.68 mmol), acetone (0.39 g, 6.70 mmol), acetic acid (1.01 g, 16.7 mmol) and 1,2-dichloroethane. After stirring under nitrogen at room temperature for 15 min, the mixture was heated at 55° C. for 3 days. More acetone (0.30 g, 6.7 mmol) and sodium triacetoxyborohydride (0.5 g, 2.36 mmol) were added and the mixture was reheated for 2 days. After cooling, water (10 ml) and excess 5M NaOH solution were added and, after stirring at room temperature for 30 min, the product was extracted into dichloromethane. The dichloromethane extract was washed (brine), dried (MgSO4) and evaporated in vacuo to give an oil (0.55 g). The oil was purified by MS guided preparative LC to give 1,1-dimethylethyl 4-((2-methylpropyl){[2-(trifluoromethyl)phenyl]methyl}amino)piperidine-1-carboxylate as the acetate salt (0.082 g, 12%); LCMS 6 min, Rt=3.96 min, (M⁺+H)=401.

(ii) 4M HCl in dioxane (1 ml) was added to 1,1-dimethylethyl 4-((2-methylpropyl){[2-(trifluoromethyl)phenyl]methyl}amino)piperidine-1-carboxylate acetate salt (82 mg, 0.16 mmol) in ethanol (5 ml). After stirring for 6 h at-room temperature, the solution was evaporated in vacuo at 50° C. to give an oil which was converted to the free base using a SCX-2 ion exchange column, eluting with methanol and then a 2.3M solution of ammonia in methanol. The free base was converted to the fumarate salt (ethanol/ether) to give the title product as a white solid (43.3 mg, 65%). δ_{H} (300, MeOD) 8.12-8.19 (1H, d), 7.70-7.78 (2H, dd), 7.48-7.53 (1H, t), 6.82 (2H, s), 4.06 (2H, s), 3.51-3.55 (2H, bd), 3.20-3.29 (1H, quintet), 3.06-3.15 (3H, m), 2.12-2.22 (2H, br d), 1.84-1.97 (2H, m), 1.22-1.24 (6H, d). LCMS 12 min, Rt=4.67 min, (M⁺+H)=301.

### EXAMPLE 4

### N-(2-methylpropyl)-N-{[4-(methoxy)phenyl]methyl}piperidin-4-amine

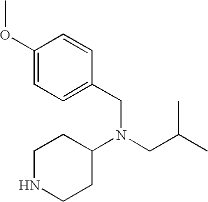

Compounds were prepared by solid phase synthesis by the route shown below. The sequence is preferably performed on a polystyrene resin. The process may be run in a combinatorial fashion such that all possible compounds from sets of precursors ArCH₂NH₂ and RCHO may be prepared. The sequence is performed without characterisation of the resin-bound intermediates.

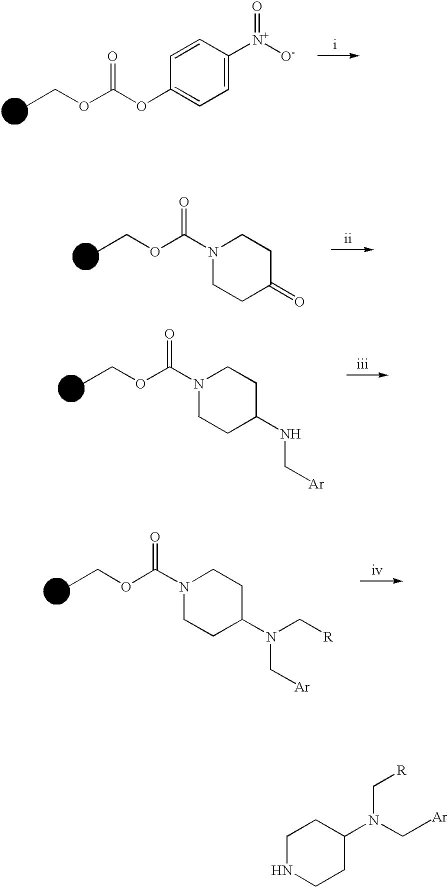

i) To suspension of p-nitrophenyl carbonate resin (Novabiochem, 1.56 g, 1.5 mmol) in DMF (20 ml) was added 4-piperidone hydrate hydrochloride (691 mg, 4.5 mmol) and N,N-diisopropylethylamine (1.56 ml, 9 mmol). The mixture was agitated gently for 69 h, then filtered and washed with DMF (3×50 ml). The resin was resuspended in DMF (20 ml), N,N-diisopropylethylamine (2 ml) added, and the mixture agitated gently for 5 min. The resin was filtered off, washed with DMF (2×50 ml) and MeOH (3×50 ml) and dried in a vacuum oven at 45° C.

(ii) Aliquots (50 mg, 0.05 mmol) of the resin prepared in step i) were dispensed into a Titan 24-well Filter Plate (Radleys) fitted with 5 mm PTFE frits. The bottom of the filter plate was closed with a PTFE seal retained by a Combi-Clamp (Radleys). To each well was added a 1M solution of a substituted benzylamine in DMF (0.5 ml, 0.5 mmol) and a 0.5M solution of sodium triacetoxyborohydride in DMF (0.5 ml, 0.25 mmol). The top of the plate was closed with a PTFE seal retained by the Combi-Clamp and the whole assembly agitated by orbital shaking for 22 h. After removal of the seals the reactions were filtered under a slight vacuum and washed with DMF (4×2.5 ml).

(iii) The bottom of the filter plate was closed with a PTFE seal retained by a Combi-Clamp. To each well was added a 1M solution of an aldehyde in DMF (0.5 ml, 0.5 mmol) and a 0.5M solution of sodium triacetoxyborohydride in DMF (0.5 ml, 0.25 mmol). The top of the plate was closed with a PTFE seal retained by the Combi-Clamp and the whole assembly agitated by orbital shaking for 43 h. After removal of the seals the reactions were filtered under a slight vacuum and washed with DMF (1×2.5 ml), EtOH (2×2.5 ml) and dichloromethane (4×2.5 ml), and partially dried under vacuum.

(iv) The bottom of the filter plate was closed with a PTFE seal retained by a Combi-Clamp. To each well was added a TFA/H₂O mixture (95:5 v/v, 1 ml). The top of the plate was closed with a PTFE seal retained by the Combi-Clamp and the whole assembly agitated by orbital shaking for 6 h. After removal of the seals the reactions were filtered under a slight vacuum and washed with dichloromethane (2×2 ml). Appropriate filtrates and washings were combined and volatile components removed by vacuum evaporation. Each residue was dissolved in MeOH (1 ml) and the solutions applied to MeOH-washed SCX-2 cartridges (0.5 g/2.5 ml) (Jones Chromatography). After draining under gravity the cartridges were washed with MeOH (2.5 ml) and the products then eluted using a 2M solution of ammonia in MeOH (2.5 ml). Removal of volatile components by vacuum evaporation gave the desired products in ca. 50% overall yield.

By this means using 4-methoxybenzylamine and isobutyraldehyde was prepared the title compound N-(2-methylpropyl)-N-{[4-(methoxy)phenyl]methyl}-piperidin-4-amine, m/e 277.2 [M+H], δH (300 MHz CDCl₃) 7.26-7.23 (2H d Ar), 6.84-6.81 (2H d Ar), 3.80 (3H s CH3OAr), 3.54 (2H s ArCH2N), 3.11-3.07 (2H d CH2NH), 2.52-2.45 (3H m CH2N), 2.22-2.19 (2H d iPrCH2N), 1.84-1.63 (3H m ring CH2, Me2CH), 1.60-1.36 (2H m ring CH2), 0.84-0.82 (6H d CH3CH).

### EXAMPLE 5

### N-(2-methylpropyl-N-{[4-(chloro)phenyl]methyl}piperidin-4-amine

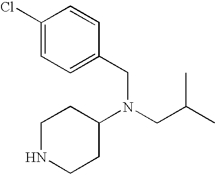

The title product m/e 281.1 (M+H) was prepared by the method described in example 4.

### EXAMPLE 6

### N-(2-methylpropyl)-N-(phenylmethyl)piperidin-4-amine

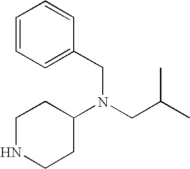

The title product m/e 247.2 (M+H) was prepared by the method described in example 4.

### EXAMPLE 7

### N-(2-methylpropyl)-N-{[4-(methyl)phenyl]methyl}piperidin-4-amine

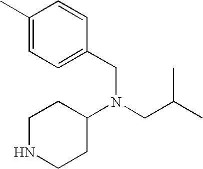

The title product m/e 261.2 (M+H) was prepared by the method described in example 4.

### EXAMPLE 8

### N-(2-methylpropyl)-N-{[3,4-(dichloro)phenyl]methyl}piperidin-4-amine

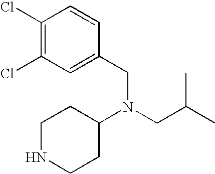

The title product m/e 315.1 (M+H) was prepared by the method described in example 4.

### EXAMPLE 9

### N-(2-methylpropyl)-N-{[2-(trifluoromethyl)phenyl]methyl}piperidin-4-amine

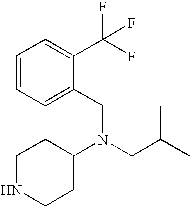

The title product m/e 315.2 (M+H) was prepared by the method described in example 4.

### EXAMPLE 10

### N-(cyclohexylmethyl)-N-(phenylmethyl)piperidin-4-amine

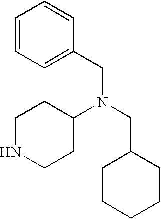

The title product m/e 287.2 (M+H) was prepared by the method described in example 4.

### EXAMPLE 11

### N-(cyclohexylmethyl)-N-{[4-(chloro)phenyl]methyl}piperidin-4-amine

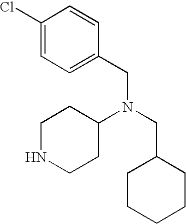

The title product m/e 321.2 (M+H) was prepared by the method described in example 4.

### EXAMPLE 12

### N-(cyclohexylmethyl)-N-{[4-(methoxy)phenyl]methyl}piperidin-4-amine

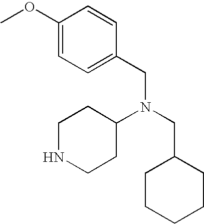

The title product m/e 317.2 (M+H) was prepared by the method described in example 4.

### EXAMPLE 13

### N-(cyclohexylmethyl)-N-{[4-(methyl)phenyl]methyl}piperidin-4-amine

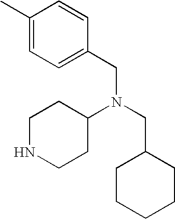

The title product m/e 301.2 (M+H) was prepared by the method described in example 4.

### EXAMPLE 14

### N-(cyclohexylmethyl)-N-{[3,4-(dichloro)phenyl]methyl}piperidin-4-amine

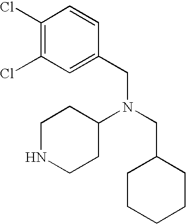

The title product m/e 355.1 (M+H) was prepared by the method described in example 4.

### EXAMPLE 15

### N-(cyclohexylmethyl)-N-{[2-(trifluoromethyl)phenyl]methyl}piperidin-4-amine

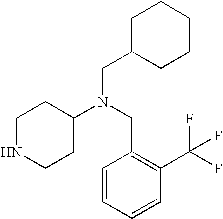

The title product m/e 355.2 (M+H) was prepared by the method described in example 4.

### EXAMPLE 16

### N-(cyclopropylmethyl)-N-{[4-(chloro)phenyl]methyl}piperidin-4-amine

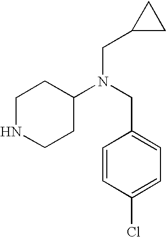

The title product m/e 279.1 (M+H) was prepared by the method described in example 4.

### EXAMPLE 17

### N-(cyclopropylmethyl)-N-{[4-(methoxy)phenyl]methyl}piperidin-4-amine

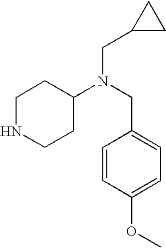

The title product m/e 275.2 (M+H) was prepared by the method described in example 4.

### EXAMPLE 18

### N-(cyclopropylmethyl)-N-1 [4-(methyl)phenyl]methyl piperidin-4-amine

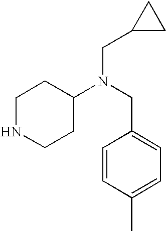

The title product m/e 259.1 (M+H) was prepared by the method described in example 4.

### EXAMPLE 19

### N-(cyclopropylmethyl)-N-{[3,4-(dichloro)phenyl]methyl}piperidin-4-amine

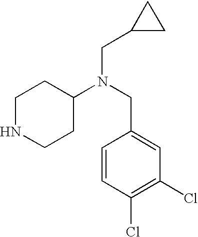

The title product m/e 313.1 (M+H) was prepared by the method described in example 4.

### EXAMPLE 20

### N-(cyclopropylmethyl)-N-{[2-(trifluoromethyl)phenyl]methyl}piperidin-4-amine

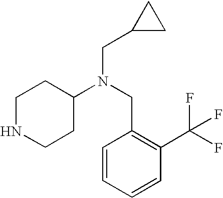

The title product m/e 313.2 (M+H) was prepared by the method described in example 4.

### EXAMPLE 21

### N-(butyl)-N-{[4-(chloro)phenyl]methyl}piperidin-4-amine

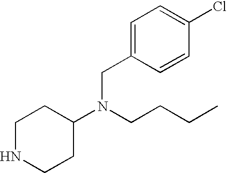

The title product m/e 281.1 (M+H) was prepared by the method described in example 4.

### EXAMPLE 22

### N-(butyl)-N-{[4-(methoxy)phenyl]methyl}piperidin-4-amine

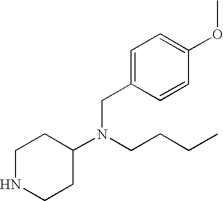

The title product m/e 277.2 (M+H) was prepared by the method described in example 4.

### EXAMPLE 23

### N-(butyl)-N-{[4-(methyl)phenyl]methyl}piperidin-4-amine

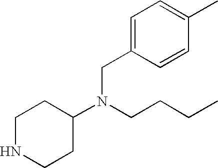

The title product m/e 261.2 (M+H) was prepared by the method described in example 4.

### EXAMPLE 24

### N-(butyl)-N-{[3,4-(dichloro)phenyl]methyl}piperidin-4-amine

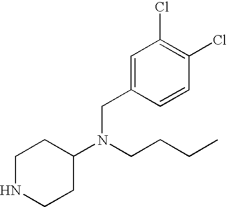

The title product m/e 315.1 (M+H) was prepared by the method described in example 4.

### EXAMPLE 25

### N-(butyl)-N-{[2-(trifluoromethyl)phenyl]methyl}piperidin-4-amine

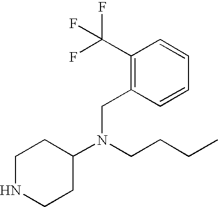

The title product m/e 315.2 (M+H) was prepared by the method described in example 4.

### EXAMPLE 26

### N-(2-methylpropyl)-N-{[2-(cyano)phenyl]methyl}piperidin-4-amine fumarate

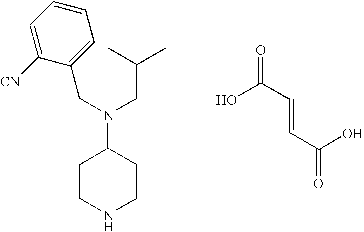

To a solution of 1,1-dimethylethyl 4-[(2-methylpropyl)amino]piperidine-1-carboxylate (0.38 g, 1.5 mmol, 1 eq) in 1,2-dichloroethane (10 ml) was added 2-cyanobenzaldehyde (295 mg, 2.25 mmol, 1.5 eq) in 1,2-dichloroethane (1 ml). After stirring for 15 minutes sodium triacetoxyborahydride (0.48 g, 2.25 mmol, 1.5 eq) was added and the mixture left to stir for a further for 48 h. A further portion of 2-cyanobenzaldehyde (295 mg, 2.25 mmol, 1.5 eq) in 1,2-dichloroethane (1 ml) and sodium triacetoxyborahydride (0.48 g, 2.25 mmol 1.5 eq) in dimethylformamide (2 ml) were added and the reaction stirred for a further 16 h. Methanol (10 ml) was added and the product purified using SCX-2 ion exchange cartridge (2×10 g) washing with methanol (100 ml) and eluting the product with 2M ammonia in methanol solution (100 ml). The solvent removed in vacuo to give 1,1-dimethylethyl 4-[[(2-cyanophenyl)methyl](2-methylpropyl)amino]-piperidine-1-carboxylate as a colourless oil. To this oil was added a 95% trifluoroacetic acid in water solution (10 ml) and the mixture stirred at room temperature for 16 h. The solvent removed in vacuo and the residue diluted with methanol (5 ml) and loaded onto SCX-2 ion exchange cartridge (5 g). The column was washed with methanol (15 ml) and eluting the product with 2M ammonia in methanol solution (15 ml) the solvent removed in vacuo to give (309 mg, 76%) as a colourless oil. The product was taken up in diethyl ether (15 ml) and a few drops of methanol to solubilise and a hot solution of fumaric acid (132 mg, 1.1 mmol, 1 eq) in methanol (1 ml) was added. The solution was heated and a few drops of methanol added until all solid was in solution, then the mixture was allowed to slowly cool to 0° C. The product was collected by filtration and dried in a vacuum oven (40° C. for 2 h) to give N-(2-methylpropyl)-N-{[2-(cyano)phenyl]methyl}piperidin-4-amine fumarate (279 mg, 48%) as a white solid. δ_{H} (300 MHz, MeOD) 7.71 (1H, d, J=7.5 Hz, ArH), 7.65-7.64 (2H, m, ArH), 7.47-7.41 (1H, m, ArH), 6.70 (2H, s, fumarate CH), 3.86 (2H, s, CH₂Ar), 3.50-3.46 (2H, m, NCH₂), 3.03-2.85 (3H, m, NCH, NCH₂), 2.30 (2H, d, J=7.2 Hz, NCH₂), 2.13-2.09 (2H, m, CCH₂), 1.90-1.75 (2H, m, CCH₂), 1.63-1.54 (1H, m, CH(CH₃)₂) and 0.81 (6H, d, J=6.6 Hz, CH₃); LCMS 12 min, Rt=4.4 min, (M⁴+1)=272.2.

### EXAMPLE 27

### N-(2-methylpropyl)-N-{[4-(trifluoromethyl)phenyl]methyl}piperidin-4-amine fumarate

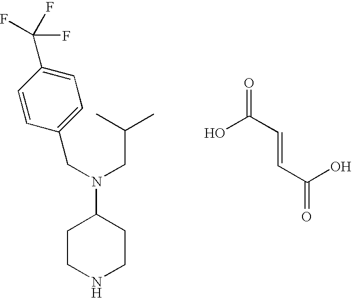

As example 26 with 1,1-dimethylethyl 4-[(2-methylpropyl)amino]piperidine-1-carboxylate and 4-(trifluorormethyl)benzaldehyde to give N-(2-methylpropyl)-N-{[4-(trifluoromethyl)phenyl]methyl}piperidin-4-amine fumarate (242 mg, 37%) as a white solid. δ_{H} (300 MHz, MeOD) 7.63-7.56 (4H, m, ArH), 6.70 (2H, s, fumarate CH), 3.76 (2H, s, CH₂Ar), 3.46-3.42 (2H, m, NCH₂), 2.98-2.77 (3H, m, NCH, NCH₂), 2.31 (2H, d, J=7.2 Hz, NCH₂), 2.05-2.00 (2H, m, CCH₂), 1.84-1.66 (3H, m, CCH₂ and CH(CH₃)₂) and 0.89 (6H, d, J=6.6 Hz, CH₃); LCMS 12 min, Rt=4.15 min, (M⁺+1)=315.2

### EXAMPLE 28

### N-(2-methylpropyl)-N-{[3-(methyl)phenyl]methyl}piperidin-4-amine fumarate

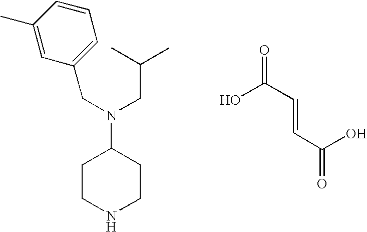

As example 26 with 1,1-dimethylethyl 4-[(2-methylpropyl)amino]piperidine-1-carboxylate and 3-methylbenzaldehyde, further purification by recrystalisation from diethyl ether and ethanol gave N-(2-methylpropyl)-N-{[3-(methyl)phenyl]methyl}piperidin-4-amine fumarate (156 mg, 28%) as a white solid. δ_{H} (300 MHz, MeOD) 7.21-7.14 (3H, m, ArH), 7.10-7.03 (1H, m, ArH), 6.70 (2H, s, fumarate CH), 3.64 (2H, s, CH₂Ar), 3.45-3.41 (2H, m, NCH₂), 2.95-2.77 (3H, m, NCH, NCH₂), 2.33 (3H, s, CH₃), 2.28 (2H, d, J=7.1 Hz, NCH₂), 2.02-1.98 (2H, m, CCH₂), 1.83-1.66 (3H, m, CCH₂ and CH(CH₃)₂) and 0.89 (6H, d, J=6.6 Hz, CH₃); LCMS 12 min, Rt=1.32 min, (M⁺+1)=261.3.

### EXAMPLE 29

### N-(2-methylpropyl)-N-{[4-(phenyl)phenyl]methyl}piperidin-4-amine fumarate

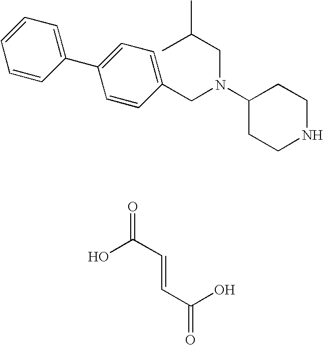

As example 26 with 1,1-dimethylethyl 4-[(2-methylpropyl)amino]piperidine-1-carboxylate and 4-biphenylcaroxaldehyde to give N-(2-methylpropyl)-N-{[4-(phenyl)phenyl]methyl}piperidin-4-amine fumarate (208 mg, 32%) as a white solid. δ_{H} (300 MHz, MeOD) 7.63-7.56 (4H, m, ArH), 7.46-7.41 (4H, m, ArH), 7.35-7.30 (1H, m, ArH), 6.70 (2H, s, fumarate CH), 3.72 (2H, s, CH₂Ar), 3.46-3.42 (2H, m, NCH₂), 2.97-2.81 (3H, m, NCH, NCH₂), 2.33 (2H, d, J=7.2 Hz, NCH₂), 2.05-2.01 (2H, m, CCH₂), 1.85-1.72 (3H, m, CCH₂ and CH(CH₃)₂) and 0.92 (6H, d, J=6.6 Hz, CH₃); LCMS 12 min, Rt=4.08 nin, (M⁺+1)=323.2.

### EXAMPLE 30

### N-(2-methylpropyl)-N-{[5-fluoro-2-(trifluoromethyl)phenyl]methyl}piperidin-4-amine fumarate

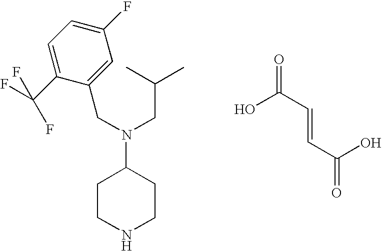

As example 26 with 1,1-dimethylethyl 4-[(2-methylpropyl)amino]piperidine-1 carboxylate and 5-fluoro-2-(trifluorormethyl)benzaldehyde, further purification by recrystalisation from diethyl ether and ethanol gave N-(2-methylpropyl)-N-{[5-fluoro-2-(trifluoromethyl)phenyl]methyl}piperidin-4-amine fumarate (160 mg, 24%) as a white solid. δ_{H} (300 z, MeOD) 7.51-7.48 (2H, m, ArH), 6.96-6.91 (1H, m, ArH), 6.48 (2H, s, fumarate CH), 3.65 (2H, s, CH₂Ar), 3.26-3.22 (2H, m, NCH₂), 2.79-2.59 (3H, m, NCH, NCH₂), 2.13 (2H, d, J=7.0 Hz, NCH₂), 1.86-1.82 (2H, m, CCH₂), 1.61-1.42 (3H, m, CCH₂ and CH(CH₃)₂) and 0.71 (6H, d, J=6.6 Hz, CH₃); LCMS 12 min, Rt=6.08 min (M⁺+1)=333.1.

### EXAMPLE 31

### N-(2-methylpropyl)-N-{[2,4-(trifluoromethyl)phenyl]methyl}piperidin-4-amine fumarate

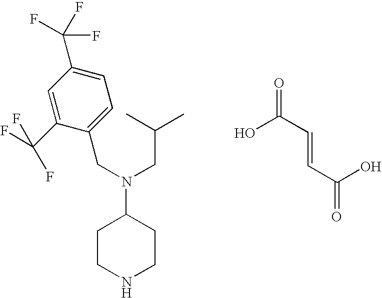

As example 26 with 1,1-dimethylethyl 4-[(2-methylpropyl)amino]piperidine-1-carboxylate and 2,4-bis(trifluoromethyl)benzaldehyde to give N-(2-methylpropyl)-N-{[2,4-di-(trifluoromethyl)phenyl]methyl}piperidin-4-amine fumarate (249 mg, 33%) as a white solid. δ_{H} (300 MHz, MeOD) 8.23 (1H, d, J=8.1 Hz, ArH), 7.97-7.93 (2H, m, ArH), 6.70 (2H, s, fumarate CH), 3.95 (2H, s, CH₂Ar), 3.48-3.44 (2H, m, NCH₂), 3.00-2.78 (3H, m, NCH, NCH₂), 2.36 (2H, d, J=7.2 Hz, NCH₂), 2.08-2.04 (2H, m, CCH₂), 1.86-1.63 (3H, m, CCH₂ and CH(CH₃)₂) and 0.92 (6H, d, J=6.6 Hz, CH₃); LCMS 12 min, Rt=6.74 min, (M⁺+1)=383.1.

### EXAMPLE 32

### N-(2-methylpropyl)-N-{[2-naphthyl]methyl}piperidin-4-amine fumarate

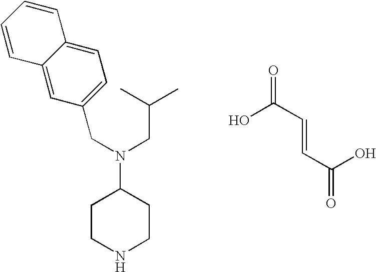

As example 26 with 1,1-dimethylethyl 4-[(2-methylpropyl)amino]piperidine-1-carboxylate and 2-naphthaldehyde to give N-(2-methylpropyl)-N-{[2-naphthyl]methyl}piperidin-4-amine fumarate (227 mg, 37%) as a white solid. δ_{H} (300 MHz, MeOD) 7.85-7.79 (4H, m, ArH), 7.56-7.53 (1H, m, ArH), 7.50-7.42 (2H, m, ArH), 6.70 (2H, s, fumarate CH), 3.84 (2H, s, CH₂Ar), 3.45-3.41 (2H, m, NCH₂), 2.94-2.82 (3H, m, NCH, NCH₂), 2.35 (2H, d, J=7.1 Hz, NCH₂), 2.08-2.03 (2H, m, CCH₂), 1.87-1.71 (3H, m, CCH₂ and CH(CH₃)₂) and 0.91 (6H, d, J=6.6 Hz, CH₃); LCMS 12 min, Rt=3.46 min, (M⁺+1)=297.2.

### EXAMPLE 33

### N-(2-methylpropyl)-N-{[2-(methylthio)phenyl]methyl}piperidin-4-amine fumarate

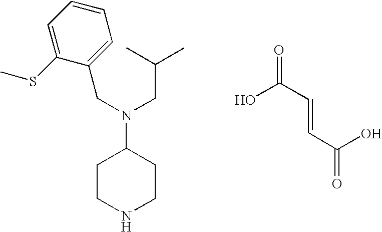

As example 26 with 1,1-dimethylethyl 4-[(2-methylpropyl)amino]piperidine-1-carboxylate and 2-methylthiobenzaldehyde, further purification by recrystalisation from diethyl ether and ethanol gave N-(2-methylpropyl)-N-{[2-(methylthio)phenyl]methyl}piperidin-4-amine fumarate (172 mg, 28%) as a white solid. δ_{H} (300 MHz, MeOD) 7.44 (1H, d, J=7.7 Hz, ArH), 7.29-7.22 (2H, m, ArH), 7.15-7.10 (1H, m, ArH), 6.70 (2H, s, fumarate CH), 3.75 (2H, s, CH₂Ar), 3.47-3.42 (2H, m, NCH₂), 2.96-2.77 (3H, m, NCH, NCH₂), 2.46 (3H, s, SCH₃), 2.29 (2H, d, J=7.0 Hz, NCH₂), 2.08-2.03 (2H, m, CCH₂), 1.87-1.60 (3H, m, CCH₂ and CH(CH₃)₂) and 0.86 (6H, d, J=6.6 Hz, CH₃); LCMS 12 min, Rt=2.92 min, (M⁺+1)=293.2.

### EXAMPLE 34

### N-(2-methylpropyl)-N-{[1-naphthyl]methyl}piperidin-4-amine hemifumarate

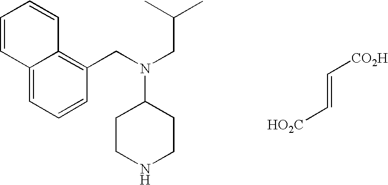

As example 26 with 1,1-dimethylethyl 4-[(2-methylpropyl)amino]piperidine-1-carboxylate and 1-naphthaldehyde, further purification by recrystalisation from diethyl ether and ethanol gave N-(2-methylpropyl)-N-{[1-naphthyl]methyl}piperidin-4-amine hemifumarate (170 mg, 27%) as a white solid. δ_{H} (300 MHz, MeOD) 8.448.41 (1H, m, ArH), 7.97-7.86 (2H, m, ArH), 7.66-7.49 (4H, m, ArH), 6.75 (1H, s, fumarate CH), 4.24 (2H, s, CH₂Ar), 3.51-3.47 (2H, m, NCH₂), 2.96-2.88 (3H, m, NCH, NCH₂), 2.46 (2H, d, J=7.2 Hz, NCH₂), 2.15-2.10 (2H, m, CCH₂), 1.98-1.71 (3H, m, CCH₂ and CH(CH₃)₂) and 0.92 (6H, d, J=6.6 Hz, CH₃); LCMS 12 min, Rt=3.96 min, (M⁺+1)=297.2.

### EXAMPLE 35

### N-(cyclopropylmethyl)-N-{[2-(trifluoromethyl)phenyl]methyl}piperidin-4-amine fumarate

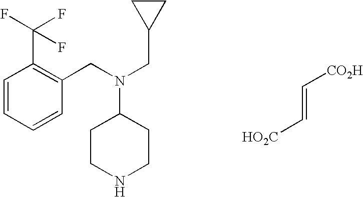

(i) To a solution of 1,1-dimethylethyl 4-({[2-(trifluoromethyl)phenyl]methyl}amino)piperidine-1-carboxylate (8 g, 22.3 mmol, 1 eq) in 1,2-dichloroethane (125 ml) was added cyclopropane carboxaldehyde (5.4 ml, 72.2 mmol, mmol, 3.2 eq). After stirring for 15 minutes sodium triacetoxyborahydride (6.62 g, 31.2 mmol, 1.4 eq) was added and the mixture in two portions and left to stir for 16 h. 2M aqueous sodium hydroxide was added (50 ml), the aqueous layer was separated and extracted with dichloromethane (3×100 ml). The combined organic extracts were dried (MgSO₄) and the solvent removed in vacuo to give a colourless oil which was purified by flash chromatography with 10% ethyl acetate in iso-hexane to give 1,1-dimethylethyl 4-[{[2-(trifluoromethyl)phenyl]methyl}(cyclopropylmethyl)amino]-piperidine-1-carboxylate (6.6 g, 72%) as a white crystalline solid. δ_{H} (300 MHz, MeOD) 8.01 (1H, d, J=7.8 Hz, ArH), 7.58 (1H, d, J=7.8 Hz, ArH), 7.53-7.48 (1H, m, ArH), 7.31-7.29 (1H, m, ArH), 4.21-4.04 (2H, m, NCH₂), 3.86 (2H, s, CH₂Ar), 2.83-2.50 (3H, m, NCH, NCH₂), 2.40 (2H, d, J=6.4 Hz, NCH₂), 1.80-1.69 (2H, m, CCH₂), 1.50-1.31 (1H, m, CCH₂ and C(CH₃)₃), 0.89-0.70 (1H, m, CH), 0.43-0.33 (2H, m, CH₂) and 0.09-0.00 (2H, m, CH₂); LCMS 6 min. Rt=3.54 min, (M⁺+1)=413.3.

(ii) To a solution of 1,1-dimethylethyl 4-[{[2-(trifluoromethyl)phenyl]methyl}(cyclopropylmethyl)amino]-piperidine-1-carboxylate (6.6 g, 16 mmol, 1 eq) in ethanol (20 ml) was added a solution of concentrated hydrochloric acid (41.1 ml, 480 mmol, 30 eq) in ethanol (80 ml) and the solution left to stir at room temperature for 120 h. The solvent removed in vacuo and the oil taken up in dichloromethane (50 ml) and washed with saturated potassium carbonate (100 ml). The aqueous layer was separated and extracted with dichloromethane (3×50 ml), the combined organic extracts were dried (MgSO₄) and the solvent removed in vacuo. The residue was purified by flash chromatography with a gradient of 50% ethanol in 5% ammonia in methanol to give (4.08 g, 82%) as a colourless oil. The product was taken up in diethyl ether (150 ml) and a few drops of methanol to solubilize and a hot solution of fumaric acid (1.5 g, 13.1 mmol, 1 eq) in methanol (10 ml) was added. The solution was heated and a few drops of methanol added until all solid was in solution, then the mixture was allowed to slowly cool to 0° C. The product was collected by filtration and recrystallised from ethyl acetate/ethanol mixture to give N-(cyclopropylmethyl)-N-{[2-(trifluoromethyl)phenyl]methyl}piperidin-4-amine fumarate (2.3 g, 34%) as a white solid. δ_{H} (300 MHz, MeOD) 7.95 (1H, d, J=7.7 Hz, ArH), 7.57-7.48 (2H, m, ArH), 7.33-7.28 (1H, m, ArH), 6.60 (2H, s, fumarate CH), 3.86 (2H, s, CH₂Ar), 3.38-3.34 (2H, m, NCH₂), 3.00-2.83 (3H, m, NCH, NCH₂), 2.38 (2H, d, J=6.4 Hz, NCH₂), 2.00-1.95 (2H, m, CCH₂), 1.77-1.62 (2H, m, CCH₂), 0.79-0.72 (1H, m, CH), 0.39-0.33 (2H, m, CH₂) and 0.02-0.00 (2H, m, CH₂); LCMS 12 min, Rt=3.56 min. (M⁺+1)=313.1.

### EXAMPLE 36

### N-(2-methylpropyl)-N-[(3-cyanophenyl)methyl]piperidin-4-amine fumarate

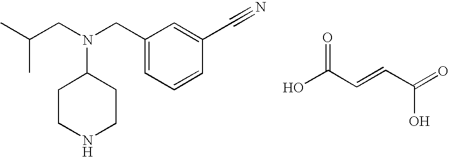

(i) To 10% Pd/C (3.0 g, 10% wt), under nitrogen, was added a solution of the 1-Boc-4-piperidone (30 g, 150.56 mmol, 1.0 eq.) and isobutylamine (11.23 g, 180.3 mmol, 1.2 eq.) in ethanol (300 ml). This was hydrogenated for 1.5 h at 65 psi using a Parr hydrogenator. The catalyst was removed by filtration through Celite. Solvent was removed under vacuum to give 4-isobutylamino-piperidine-1-carboxylic acid tert-butyl ester as a colourless oil (31.2 g) with >98% purity. LCMS-6 mins gradient Rt=2.79 (M⁺+1) 257.2. 1H NMR (CDCl₃) 3=4.01 (2H, brs), 2.75-2.82 (2H, m), 2.54-2.61 (1H, m), 2.43 (2H, d, J=12.4 Hz), 1.81-1.85 (2H, m), 1.67-1.76 (1H, m), 1.56 (1H, br s), 1.45 (9H, s), 1.18-1.31 (2H, m), 0.91 (6H, d, J=6.4 Hz).

(ii) General method: To a solution of secondary amine (0.5 g, 1.0 eq) in 1,2-dichloroethane (10 ml) was added the desired benzaldehyde (3.0 eq.). To this was added a solution of sodium triacetoxyborohydride (3.0 eq.) in dimethylformamide (2 ml). This mixture was left to stir under nitrogen, at room temperature, for 3 days. To the reaction mixture was then added water (10 ml) and the mixture stirred vigorously for several minutes. The chlorinated organic layer was then run through a hydrophobic frit to remove water. The resulting organic solution was diluted with methanol (10 ml) and loaded onto an SCX-2 (10 g) column. The column was washed with methanol (20 ml) then basic material eluted with 2M ammonia in methanol (20 ml). The ammonia/methanol solution was concentrated in vacuo to give the N-Boc-piperidine product.

To a solution of the oil (1.0 eq.) in dichloromethane (10 ml) was added trifluoroacetic acid (TFA) (15 eq). The solution was stirred at room temperature for 4 h. The solvent and TFA were removed in vacuo. The resulting oil was taken up in methanol and loaded onto an SCX-2 (10 g) column. The column was washed with methanol (20 ml). Basic material was then eluted using 2M ammonia in methanol (20 ml). Removal of solvent from the ammonia/methanol mixture under vacuum, gave the desired compound as an oil. The oil was taken up in diethylether and a solution of fumaric acid (1 eq) in hot ethanol was added. The mixture was left at room temperature for a few minutes before precipitation occurred or if necessary the solution was placed in the fridge for a few hours. The resulting precipitate was collected by filtration to give the fumarate salt as a white solid. The title product was prepared by the general method above using tert-butyl 4-isobutylamino-piperidine-1-carboxylate and 3-cyanobenzaldehyde. LCMS 12 mins gradient Rt=2.69 M⁺+1) 272.1, 1H NMR (d6-DMSO) δ=7.80-7.65 (3H, m), 7.60-7.51 (1H, m), 6.45 (2H, s), 3.61 (2H, s), 3.23 (2H, brd), 2.80-2.61 (3H, m), 2.20 (2H, d), 1.85-1.75 (2H, m), 1.70-1.52 (3H, m), 0.77 (6H, d, J=6.9 Hz).

### EXAMPLE 37

### N-(2-methylpropyl)-N-[(3,5-dichlorophenyl)methyl]piperidin-4-amine fumarate

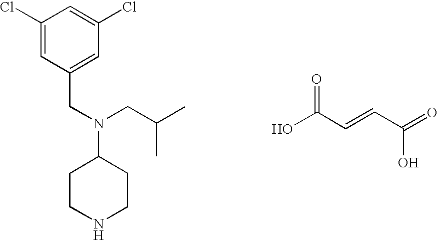

The title product was prepared from tert-butyl 4-isobutylamino-piperidine-1-carboxylate and 3,5-dichlorobenzaldehyde using the method described in example 36. LCMS 12 mins gradient Rt=5.38 (M⁺+1) 315.1, 1H NMR (d6-DMSO) δ=7.4 (1H, s), 7.35 (2H, s), 6.35 (1H, s), 3.58 (2H, s), 3.15 (2H, brd), 2.75-2.55 (3H, m), 2.21 (2H, d), 1.85-1.45 (5H, m), 0.85 (6H, d, J=6.4 Hz).

### EXAMPLE 38

### N-(2-methylpropyl)-N-[(2,4-dimethoxyphenyl)methyl]piperidin-4-amine fumarate

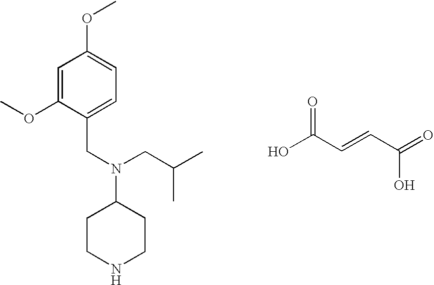

The title product was prepared from tert-butyl 4-isobutylamino-piperidine-1-carboxylate and 2,4-dimethoxybenzaldehyde using the method described in example 36. LCMS 12 mins gradient Rt=2.03 (M⁺+1) 307.3. 1H NMR (d6-DMSO) δ=7.23 (1H, d, J=7.91 Hz), 6.50 (2H, m), 6.45 (2H, s), 3.76 (3H, s, OMe), 3.75 (3H, s, OMe), 3.49 (2H, s), 3.25 (2H, brd), 2.80-2.61 (3H, m), 2.16 (2H, d, J=6.97 Hz), 1.77-1.58 (5H, m), 0.80 (6H, d, J=6.41 Hz).

### EXAMPLE 39

### N-(2-methylpropyl)-N-[(2,3-dichlorophenyl)methyl]piperidin-4-amine fumarate

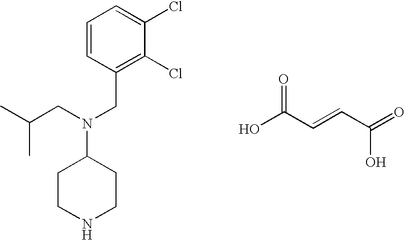

The title product was prepared from tert-butyl 4-isobutylamino-piperidine-1-carboxylate and 2,3-dichlorobenzaldehyde using the method described in example 36. LCMS 12 mins gradient Rt=5.34 (M⁺+1) 315.2. 1H NMR (d6-DMSO) δ=7.57-7.51 (2H, m), 7.40-7.33 (1H, m), 6.47 (2H, s), 3.71 (2H, s), 3.25 (2H, brd), 2.85-2.63 (4H, m), 2.30 (2H, d, J=6.97 Hz), 1.85-1.52 (5H, m), 0.80 (6H, d, J=6.59 Hz).

### EXAMPLE 40

### N-(2-methylpropyl)-N-{[4-(methoxycarbonyl)phenyl]methyl}piperidin-4-amide fumarate

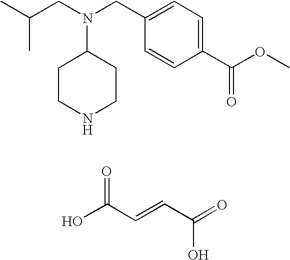

The title product was prepared from tert-butyl 4-isobutylamino-piperidine-1-carboxylate and methyl 4-formylbenzoate using the method described in example 36. LCMS 12 mins gradient Rt=2.99 (M⁺+1) 305.2. 1H NMR (d6-DMSO) δ=7.90 (2H, d, J=8.29 Hz), 7.40 (2H, d, J=8.29 Hz), 6.60 (2H, s), 4.08 (3H, s), 3.84 (2H, s), 3.30 (2H, brd), 2.842.65 (4H, m), 2.49 (1H, s), 2.20 (2H, brd), 1.70-1.59 (4H, m), 0.79 (6H, d, J=6.6 Hz).

### EXAMPLE 41

### N-(2-methylpropyl)-N-[(2,4-difluorophenyl)methyl]piperidin-4-amine fumarate

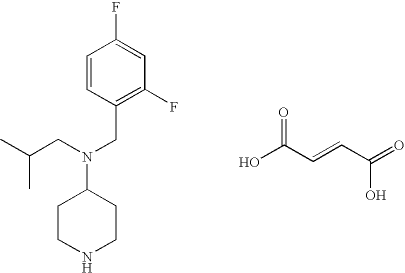

The title product was prepared from tert-butyl 4-isobutylamino-piperidine-1-carboxylate and 2,4-difluorobenzaldehyde using the method described in example 36. LCMS 12 mins gradient Rt=2.79 (M⁺+1) 283.2. 1H NMR (d6-DMSO) δ=7.52-7.44 (1H, m), 7.19-7.12 (1H, m), 7.08-7.01 (1H, m), 6.42 (2H, s), 3.57 (2H, s), 3.22 (2H, brd), 2.78-2.62 (4H, m), 2.18 (2H, d), 1.79-1.53 (5H, m), 0.75 (6H, d, J=6.9 Hz).

### EXAMPLE 42

### N-(2-methylpropyl)-N-{[2-(trifluoromethoxy-phenyl]methyl}piperidin-4-amine fumarate

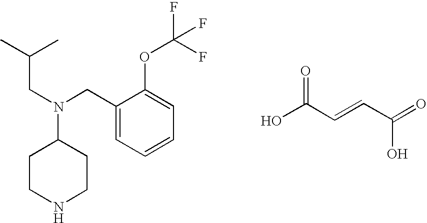

The title product was prepared from tert-butyl 4-isobutylamino-piperidine-1-carboxylate and 2-(trifluoromethoxy)benzaldehyde using the method described in example 36. LCMS 12 mins gradient Rt=4.45 (M⁺+1) 331.2. 1H NMR (d6-DMSO) δ=7.64-7.60 (1H, m), 7.40-7.37 (2H, m), 7.36-7.31 (1H, m), 6.42 (2H, s), 3.63 (2H, s), 3.23 (2H, brd), 2.76-2.62 (4H, m), 2.20 (2H, d, J=6.97 Hz), 1.80-1.61 (3H, m), 1.59-1.52 (2H, m), 0.61 (6H, d, J=6.6 Hz).

### EXAMPLE 43

### N-(2-methylpropyl)-N-[(2-fluorophenyl)methyl]piperidin-4-amine fumarate

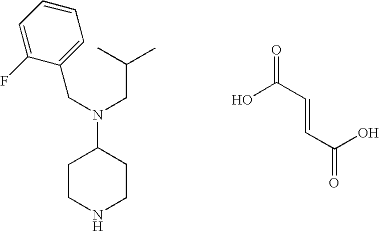

To a dry boiling tube (50 ml), under nitrogen, was added tert-butyl-4-(2-methylpropylamino)-piperidine-1-carboxylate (0.200 g, 0.780 mmol), 2-fluorobenzaldehyde (0.087 ml, 0.102 g, 0.819 mmol), and titanium isopropoxide (0.268 ml, 0.937 mmol) to give a yellow/orange solution. This was heated to 90° C. for 2 hours. Solution cooled, and ethanol (5 ml) added. Sodium borohydride (0.030 g, 0.780 mmol) was then added and allowed to stir for 2 days. Further sodium borohydride (0.300 g, 7.80 mmol) was added, and after 6 hours, this was diluted with methanol (10 ml) with stirring for 20 hours. This was concentrated in vacuo, dissolved in dichloromethane (5 ml), and acetic anhydride (0.371 ml, 39.00 mmol) added with stirring for 30 minutes. Solution was diluted with methanol (10 ml), and passed through an SCX-2 column to give an oil (0.150 g, 0.412 mmol).

The resultant oil was dissolved in dichloromethane (5 ml), and trifluoroacetic acid (2 ml) added. Reaction was monitored by thin layer chromatography (100% ethyl acetate; reactant. r.f. 0.4, product r.f 0.0). After 2 hours, reaction was concentrated in vacuo, azeotroped with dichloromethane (c.a. 25 ml), taken up in methanol (c.a. 5 ml), and passed through an SCX-2 column. The resultant colourless oil was purified using reverse phase chromatography, concentrated in vacuo, taken up in 5 M hydrochloric acid (10 ml), and heated to 90° C. for 3 hours. This solution was freeze dried to give an oil (0.049 g, 0.185 mmol). Resultant oil was passed through an SCX-2 column, dissolved in aqueous acetonitrile (c.a. 20 ml), and fumaric acid (0.0214 g, 0.1850 mmol) added. After 5 minutes, this was freeze dried to give a white solid (0.070 g, 0.185 mmol) as the title compound. δ_{H} (300 MHz, MeOD) 7.47 (1H, t, Ar), 7.25 (1H, m, Ar), 7.13 (1H, t, Ar), 7.02 (1H, t, Ar), 6.70 (2H, s, fumarate), 3.21 (2H, s, NCH₂Ar), 3.45 (2H, d, CH), 2.95 (2H, t, CH), 2.82 (1H, t, CH), 2.29 (2H, d, NCH2), 2.00 (2H, d, CH), 1.80 (2H, t, m), 1.68 (1H, t, CH), 0.85 (6H, d, CHMe2). LCMS 12 minute gradient, Rt=1.99 mins, (++1)=265.2

### EXAMPLE 44

### N-(2-methylpropyl)-N-[(2-chlorophenyl)methyl]piperidin-4-amine fumarate

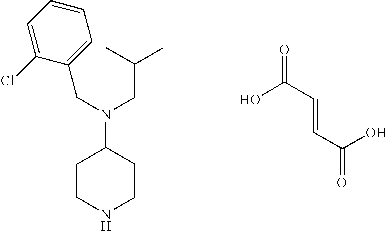

The title product was prepared similarly to Example 43 using 2-chlorobenzaldehyde. δ_{H} (300 MHz, MeOD/CDCl₃) 7.52 (1H, d, Ar), 7.30 (1H, d, Ar), 7.20 (2H, m, Ar), 6.75 (2H, s, fumarate), 3.75 (2H, s, NCH₂Ar), 3.45 (2H, d, CH), 2.79 (2H, t, CH), 2.65 (1H, t, CH), 2.29 (2H, d, NCH2), 2.00 (2H, d, CH), 1.82 (2H, t, CH), 1.68 (1H, t, CH), 0.85 (6H, d, CHMe2). LCMS 12 minute gradient, Rt=3.37 mins, (M⁺+1)=281.2/283.2

### EXAMPLE 45

### N-(2-methylpropyl)-N-[(2-methoxyphenyl)methyl]piperidin-4-amine fumarate

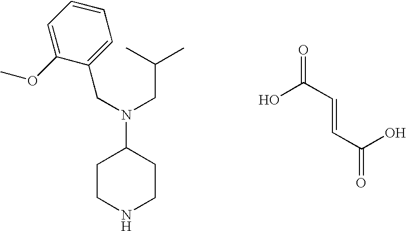

The title product was prepared similarly to Example 43 using 2-methoxybenzaldehyde. δ_{H} (300 MHz, MeOD) 7.30 (1H, d, Ar), 7.13 (1H, t, Ar), 6.83 (2H, m, Ar), 6.60 (2H, s, fumarate), 3.25 (3H, s, ArOMe), 3.25 (2H, s, NCH₂Ar), 3.35 (2H, d, CH), 2.90-2.72 (3H, m, CH), 2.29 (2H, d, NCH2), 1.95 (2H, d, CH), 1.75 (2H, t, CH), 1.65 (1H, m, CH), 0.75 (6H, d, CHMe2). LCMS 12 minute gradient, Rt=1.21 mins, (M⁺+1)=277.3

### EXAMPLE 46

### N-(2-methylpropyl)-N-[(2-methylphenyl)methyl]piperidin-4-amine fumarate

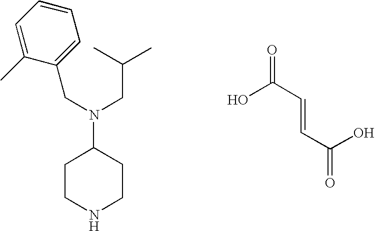

The title product was prepared similarly to Example 43 using 2-methylbenzaldehyde. δ_{H} (300 MHz, MeOD/CDCl₃) 7.18 (1H, m, Ar), 7.00 (3H, m, Ar), 6.58 (2H, s, fumarate), 3.48 (2H, s, NCH₂Ar), 3.28 (2H, d, CH), 2.70-2.45 (3H, m, CH), 2.20 (3H, s, ArMe), 2.10 (2H, d, NCH2), 1.80 (2H, d, CH), 1.68 (2H, t, CH), 1.59 (1H, m, CH), 0.70 (6H, d, CHMe2). LCMS 12 minute gradient, Rt=2.71 mins, (M⁺+1)=261.3

### EXAMPLE 47

### N-(2-methylpropyl)-N-[(2-bromophenyl)methyl]piperidin-4-amine fumarate

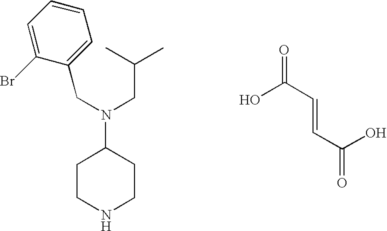

The title product was prepared similarly to Example 43 using 2-bromobenzaldehyde. δ_{H} (300 MHz, MeOD/CDCl₃) 7.55 (2H, t, Ar), 7.29 (1H, t, Ar), 7.10 (1H, t, Ar), 6.72 (2H, s, fumarate), 3.75 (2H, s, NCH₂Ar), 3.42 (2H, d, CH), 2.80 (2H, t, CH), 2.65 (1H, t, CH), 2.27 (2H, d, NCH2), 2.00 (2H, d, CH), 1.82 (2H, t, CH), 1.68 (1H, m, CH), 0.89 (6H, d, CHMe2). LCMS 12 minute gradient, Rt=3.85 mins, (M⁺+1)=323.1/325.1

### EXAMPLE 48

### N-(2-methylpropyl)-N-[(3-fluorophenyl)methyl]piperidin-4-amine fumarate

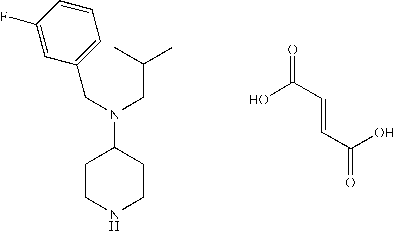

The title product was prepared similarly to Example 43 using 3-fluorobenzaldehyde. δ_{H} (300 MHz, MeOD) 7.19 (1H, q, Ar), 7.05 (2H, t, Ar), 6.85 (1H, t, Ar), 6.60 (2H, s, fumarate), 3.58 (2H, s, NCH₂Ar), 3.32 (2H, d, CH), 2.82 (2H, t, CH), 2.70 (1H, t, CH), 2.19 (2H, d, NCH2), 1.90 (2H, d, CH), 1.75-1.50 (3H, m, CH), 0.78 (6H, d, CHMe2). LCMS 12 minute gradient, Rt=2.29 mins, (M⁺+1)=265.2

### EXAMPLE 49

### N-(2-methylpropyl)-N-[(3-chlorophenyl)methyl]piperidin-4-amine fumarate

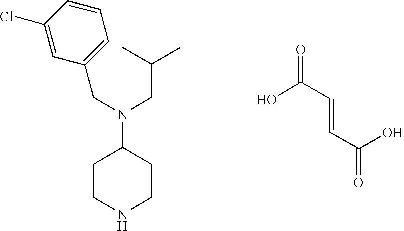

The title product was prepared similarly to Example 43 using 3-chlorobenzaldehyde. δ_{H} (300 MHz, MeOD) 7.40 (1H, s, Ar), 7.30 (2H, m, Ar), 7.25 (1H, m, Ar), 6.72 (2H, s, fumarate), 3.68 (2H, s, NCH₂Ar), 3.45 (2H, d, CH), 2.95 (2H, t, CH), 2.83 (1H, t, CH), 2.39 (2H, d, NCH2), 2.02 (2H, d, CH), 1.88-1.62 (3H, m, CH), 0.90 (6H, d, CHMe2). LCMS 12 minute gradient, Rt=3.24 mins, (M⁺+1)=281.2/283.2

### EXAMPLE 50

### N-(2-methylpropyl-N-[(3-methoxyphenyl)methyl]piperidin-4-amine fumarate

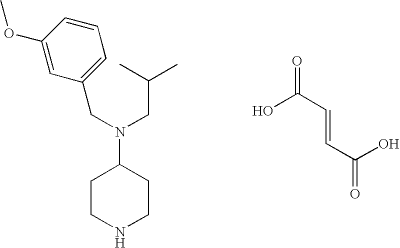

The title product was prepared similarly to Example 43 using 3-methoxybenzaldehyde. δ_{H} (300 MHz, MeOD) 7.20 (1H, t, Ar), 6.95 (2H, m, Ar), 6.79 (1H, dd, Ar), 6.70 (2H, s, fumarate), 3.78 (3H, s, ArOMe), 3.65 (2H, s, NCH₂Ar), 3.42 (2H, d, CH), 2.92 (2H, t, CH), 2.82 (1H, t, CH), 2.30 (2H, d, NCH2), 2.00 (2H, d, CH), 1.87-1.64 (3H, m, CH), 0.90 (6H, d, CHMe2). LCMS 12 minute gradient, Rt=1.56 mins, (M⁺+1)=277.3

### EXAMPLE 51

### N-(2-methylpropyl)-N-{[3-(trifluoromethyl)phenyl]methyl}piperidin-4-amine fumarate

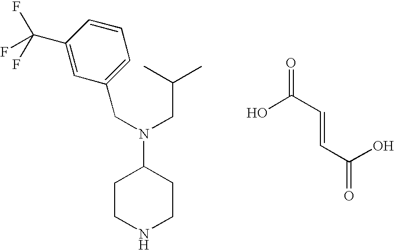

The title product was prepared similarly to Example 43 using 3-trifluoromethylbenzaldehyde. δ_{H} (300 MHz, MeOD/CDCl₃) 7.59 (1H, s, Ar), 7.51 (2H, m, Ar), 7.42 (1H, q, Ar), 6.68 (2H, s, fumarate), 3.65 (2H, s, NCH₂Ar), 3.42 (2H, d, CH), 2.82-2.60 (31, m, CH), 2.22 (2H, d, NCH2), 1.95 (2H, d, CH), 1.80 (2H, t, CH), 1.65 (1H, m, CH), 0.89 (6H, d, CHMe2). LCMS 12 minute gradient, Rt=4.15 mins, (M⁺+1)=315.2

### EXAMPLE 52

### N-(2-methylpropyl)-N-{[3-(trifluoromethoxy)phenyl]methyl}piperidin-4-amine fumarate

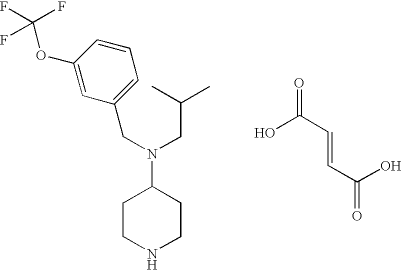

The title product was prepared similarly to Example 43 using 3-trifluoromethoxybenzaldehyde. δ_{H} (300 z, MeOD/CDCl₃) 7.32 (1H, m, Ar), 7.22 (2H, m, Ar), 7.08 (1H, d, Ar), 6.80 (2H, s, fumarate), 3.68 (2H, s, NCH₂Ar), 3.40 (2H, br, CH), 2.79 (2H, t, CH), 2.70 (1H, m, CH), 2.24 (2H, d, NCH2), 2.00-1.75 (4H, m, CH), 1.62 (1H, m, CH), 0.85 (6H, d, CHMe2). LCMS 12 minute gradient, Rt=4.33 mins, (M⁺+1)=331.2

### EXAMPLE 53

### N-(2-methylpropyl)-N-[(2,6-dichlorophenyl)methyl]piperidin-4-amine fumarate

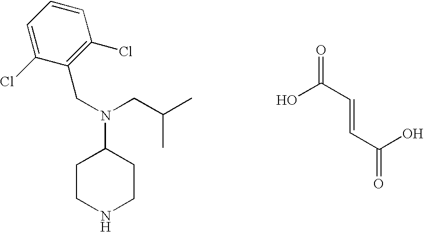

The title product was prepared similarly to Example 43 using 2,6-dichlorobenzaldehyde. δ_{H} (300 M, MeOD) 7.28 (2H, m, Ar), 7.15 (1H, t, Ar), 6.59 (2H, s, fumarate), 3.85 (2H, s, NCH₂Ar), 3.38 (2H, d, CH), 2.85 (2H, t, CH), 2.75 (1H, t, CH), 2.29 (2H, d, NCH2), 1.98 (2H, d, CH), 1.78 (2H, m, CH), 1.48 (1H, m, CH), 0.63 (6H, d, CHMe2). LCMS 12 minute gradient, Rt=4.80 mins, (M⁺+1)=315.1/317.2

### EXAMPLE 54

### N-(2-methylpropyl)-N-[(4-methylthiophenyl)methyl]piperidin-4-amine fumarate

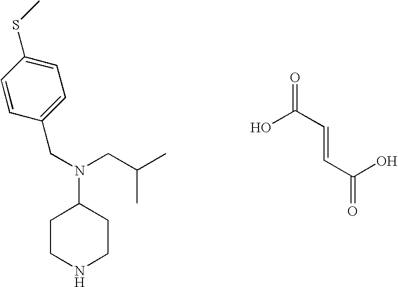

The title product was prepared from tert-butyl 4-isobutylamino-piperidine-1-carboxylate and 4-(methylthio)benzaldehyde using the method described in example 36. LCMS 12 mins gradient Rt=2.83 (M⁺+1) 293.2. 1H NMR (d6-DMSO) δ=7.25 (2H, d, J=8.29 7.20 (2H, d, J=8.48 Hz), 6.42 (2H, s), 3.52 (2H, s), 3.25 (2H, brd), 2.81-2.59 (3H, m), 2.45 (3H, s, SMe), 2.16 (2H, d, J=7.16 Hz), 1.78-1.57 (5H, m), 0.80 (6H, d, J=6.6 Hz).

### EXAMPLE 55

### N-(2-methylpropyl)-N-[(2,4-dimethylphenyl)methyl]piperidin-4-amine fumarate

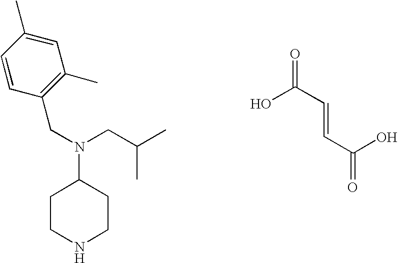

The title product was prepared from tert-butyl 4-isobutylamino-piperidine-1-carboxylate and 2,4-dimethylbenzaldehyde using the method described in example 36. LCMS 12 mins gradient Rt=3.408 (M⁺+1) 275.3. 1H NMR (d6-DMSO) δ=7.17 (1H, d, J=8.10 Hz), 6.93-6.91 (2H, m), 6.42 (2H, s), 3.51 (2H, s), 3.38 (2H, brd), 2.74-2.63 (4H, m), 2.51 (3H, s), 2.50 (3H, s), 2.24 (2H, d, J=8.48 Hz), 1.79-1.66 (4H, m), 1.58-1.49 (1H, m), 0.75 (6H, d, 3=6.4 Hz).

### EXAMPLE 56

### N-ethyl-N-{[2-(trifluoromethyl)phenyl]methyl}piperidin-4-amine fumarate

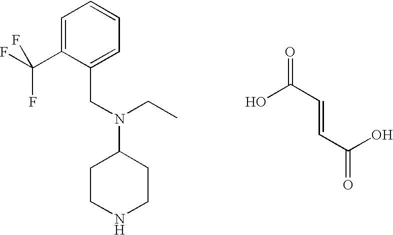

To a solution of 1,1-dimethylethyl 4-({[2-(trifluoromethyl)phenyl]methyl}amino)piperidine-1-carboxylate (0.54 g, 1.5 mmol, 1 eq) in 1,2-dichloroethane (10 ml) was added a solution of sodium triacetoxyborahydride (0.95 g, 4.5 mmol, 3 eq) in dimethylformamide (2 ml) followed by a solution of acetaldehyde (132 mg, 4.5 mmol, 3 eq) in 1,2-dichloroethane (1 ml) and the mixture left to stir for 16 h. The reaction was quenched with water (10 ml) and the organic layer separated by passing through a hydrophobic frit. This was diluted with methanol (10 ml) and loaded onto a SCX-2 ion exchange cartridge (5 g) washed with methanol (15 ml) and the product eluted with 2M ammonia in methanol solution (15 ml). The solvent removed in vacuo to give 1,1-dimethylethyl 4-[{[2-(trifluoromethyl)phenyl]methyl}(ethyl)amino]-piperidine-1-carboxylate as a colourless oil. To this oil was added a solution of anisole (1.4 ml) and trifluoroacetic acid (1.4 ml, 18.3 mmol, 12 eq), in dichloromethane (7 ml) and the mixture stirred at room temperature for 16 h. The solvent removed in vacuo and the residue diluted with methanol (5 ml) and loaded onto SCX-2 ion exchange cartridge (5 g). The column was washed with methanol (15 ml) and eluting the product with 2M ammonia in methanol solution (15 ml) the solvent removed in vacuo to give (306 mg, 71%) as a colourless oil. The product was taken up in diethyl ether (15 ml) and a few drops of methanol to solubilize and a hot solution of fumaric acid (122 mg, 1.1 mol, 1 eq) in methanol (1 ml) was added. The solution was heated and a few drops of methanol added until all solid was in solution, then the mixture was allowed to slowly cool to 0° C. The product was collected by filtration and dried in a vacuum oven (40° C. for 2 h) to give N-ethyl-N-{[2-(trifluoromethyl)phenyl]methyl}piperidin-4-amine fumarate (247 mg, 41%) as a white solid. δ_{H} (300 MHz, MeOD) 7.98 (1H, d, J=7.7 Hz, ArH), 7.67-7.58 (2H, m, ArH), 7.42-7.37 (1H, m, ArH), 6.70 (2H, s, fumarate CH), 3.88 (2H, s, CH₂Ar), 3.47-3.43 (2H, m, NCH₂), 3.02-2.87 (3H, m, NCH, NCH₂), 2.65 (2H, q, J=7.1 Hz, CH₂), 2.08-2.02 (2H, m, CCH₂), 1.86-1.72 (2H, m, CCH₂) and 1.05 (3H, t, J=7.1 Hz, CH₃); LCMS 12 min, Rt=2.16 min, (M⁺+1)=287.2.

### EXAMPLE 57

### N-propyl-N-{[2-(trifluoromethyl)phenyl]methyl}piperidin-4-amine fumarate

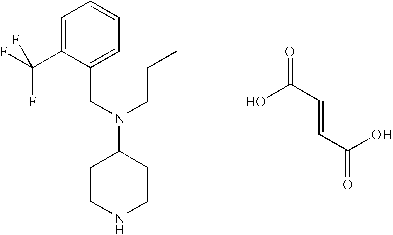

As example 56 with 1,1-dimethylethyl 4-({[2-(trifluoromethyl)phenyl]methyl}amino)piperidine-1-carboxylate and propionaldehyde to give N-propyl-N-{[2-(trifluoromethyl)phenyl]methyl}piperidin-4-amine fumarate (223 mg, 36%) as a white solid. ⁶R (300 MHz, MeOD) 7.96 (1H, d, J=7.7 Hz, ArH), 7.67-7.58 (2H, m, ArH), 7.42-7.38 (1H, m, ArH), 6.70 (2H, s, fumarate CH), 3.88 (2H, s, CH₂Ar), 3.47-3.43 (2H, m, NCH₂), 3.01-2.82 (3H, m, NCH, NCH₂), 2.54 (2H, t, J=7.3 Hz, CH₂), 2.07-2.02 (2H, m, CCH₂), 1.86-1.72 (2H, m, CCH₂), 1.46 (2H, septet, J=7.3 Hz, CH₂) and 0.89 (3H, t, J=7.3 Hz, CH₃); LCMS 12 min, Rt=3.62 min, (M⁺+1)=301.2.

### EXAMPLE 58

### N-pentyl-N-{[2-(trifluoromethyl)phenyl]methyl}piperidin-4-amine fumarate

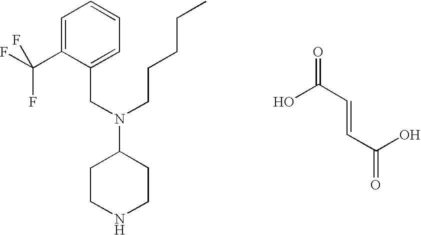

As example 56 with 1,1-dimethylethyl 4-({[2-(trifluoromethyl)phenyl]methyl}amino)piperidine-1-carboxylate and valeraldehyde to give N-pentyl-N-{[2-(trifluoromethyl)phenyl]methyl}piperidin-4-amine fumarate (236 mg, 35%) as a white solid. δ_{H} (300 MHz, MeOD) 7.95 (1H, d, J=7.9 Hz, ArH), 7.67-7.58 (2H, m, ArH), 7.43-7.39 (1H, m, ArH), 6.70 (2H, s, fumarate CH), 3.87 (2H, s, CH₂Ar), 3.47-3.43 (2H, m, NCH₂), 3.01-2.84 (3H, m, NCH, NCH₂), 2.57 (2H, t, J=7.2 Hz, CH₂), 2.07-2.03 (2H, m, CCH₂), 1.86-1.71 (2H, m, CCH₂), 1.44-1.42 (2H, m, CH₂), 1.29-1.17 (4H, m, CH₂) and 0.90-0.86 (3H, m, CH₃); LCMS 12 min, Rt=5.01 min, (M⁺+1)=329.2.

### EXAMPLE 59

### N-(3-methylbutyl)-N-{[2-(trifluoromethyl)phenyl]methyl}piperidin-4-amine fumarate

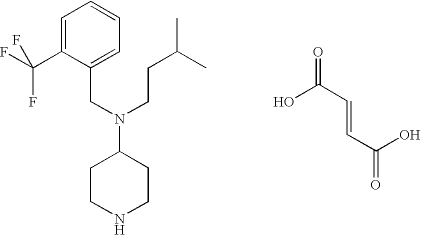

As example 56 with 1,1-dimethylethyl 4-({[2-(trifluoromethyl)phenyl]methyl}amino)piperidine-1-carboxylate and 3-methylbutyraldehyde to give N-(3-methylbutyl)-N-{[2-(trifluoromethyl)phenyl]methyl}piperidin-4-amine fumarate (348 mg, 52%) as a white solid. δ_{H} (300 MHz, MeOD) 7.94 (1H, d, J=7.9, ArH), 7.67-7.58 (2H, m, ArH), 7.43-7.38 (1H, m, ArH), 6.70 (2H, s, fumarate CH), 3.87 (2H, s, CH₂Ar), 3.48-3.44 (2H, m, NCH₂), 3.01-2.85 (3H, m, NCH, NCH₂), 2.59 (2H, t, J=7.5 Hz, NCH₂), 2.07-2.03 (2H, m, CCH₂), 1.87-1.73 (2H, m, CCH₂), 1.65-1.53 (1H, m, CH(CH₃)₂), 1.37-1.30 (2H, m, CH₂) and 0.84 (6H, d, J=6.6 Hz, CH₃); LCMS 12 min, Rt=4.87 min, (M⁺+1)=329.2.

### EXAMPLE 60

### N-(3,3-dimethylbutyl)-N-{[2-(trifluoromethyl)phenyl]methyl}piperidin-4-amine fumarate

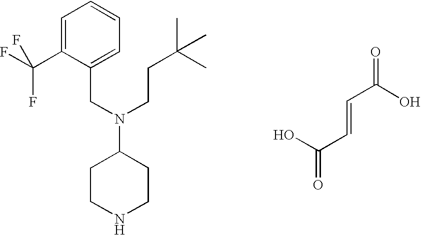

As example 56 with 1,1-dimethylethyl 4-({[2-(trifluoromethyl)phenyl]methyl}amino)piperidine-1-carboxylate and 3,3-dimethylbutyraldehyde to give N-(3,3-dimethylbutyl)-N-{[2-(trifluoromethyl)phenyl]methyl}piperidin-4-amine fumarate (102 mg, 15%) as a white solid. δ_{H} (300 MHz, MeOD) 7.95 (1H, d, J=7.7, ArH), 7.67-7.58 (2H, m, ArH), 7.42-7.38 (1H, m, ArH), 6.70 (2H, s, fumarate CM), 3.87 (2H, s, CH₂Ar), 3.47-3.44 (2H, m, NCH₂), 3.02-2.86 (3H, m, NCH, NCH₂), 2.63-2.57 (2H, m, NCH₂), 2.08-2.03 (2H, m, CCH₂), 1.87-1.73 (2H, m, CCH₂), 1.41-1.36 (2H, m, CH₂), and 0.86 (9H, s, CH₃); LCMS 12 min, Rt=5.35 min, (M⁺+1)=343.2.

### EXAMPLE 61

### N-(2-ethylbutyl)-N-{[2-(trifluoromethyl)phenyl]methyl}piperidin-4-amine fumarate

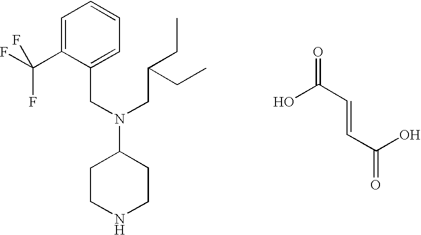

As example 56 with 1,1-dimethylethyl 4-({[2-(trifluoromethyl)phenyl]methyl}amino)piperidine-1-carboxylate and 2-ethylbutyraldehyde to give N-(2-ethylbutyl)-N-{[2-(trifluoromethyl)phenyl]methyl}piperidin-4-amine fumarate (302 mg, 44%) as a white solid. δ_{H} (300 MHz, MeOD) 7.93 (1H, d, J=7.9, ArH), 7.67-7.58 (2H, m, ArH), 7.43-7.38 (1H, m, ArH), 6.70 (2H, s, fumarate CH), 3.86 (2H, s, CH₂Ar), 3.47-3.43 (2H, m, NCH₂), 2.98-2.78 (3H, m, NCH, NCH₂), 2.42 (2H, d, J=6.2 Hz, NCH₂), 2.07-2.03 (2H, m, CCH₂), 1.86-1.72 (2H, m, CCH₂), 1.46-1.27 (5H, m, CH₂, CH), and 0.82 (6H, t, J=7.2, CH₃); LCMS 12 min, Rt=6.57 min, (M⁺+1)=343.3.

### EXAMPLE 62

### N-(2-methylprop-2-enyl)-N-{[2-(trifluoromethyl)phenyl]methyl}piperidin-4-amine fumarate

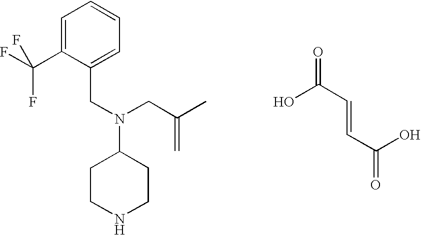

As example 56 with 1,1-dimethylethyl 4-({[2-(trifluoromethyl)phenyl]methyl}amino)piperidine-1-carboxylate and methacrolein to give N-(2-methylprop-2-enyl)-N-{[2-(trifluoromethyl)phenyl]methyl}piperidin-4-amine fumarate (224 mg, 35%) as a white solid. δ_{H} (300 MHz, MeOD) 7.95 (1H, d, J=7.7, ArH), 7.67-7.59 (2H, m, ArH), 7.44-7.39 (1H, m, ArH), 6.70 (2H, s, fumarate CH), 4.99 (2H, s, CCH₂), 3.84 (2H, s, CH₂Ar), 3.48-3.44 (2H, m, NCH₂), 3.10 (2H, s, CH₂), 2.98-2.81 (3H, m, NCH, NCH₂), 2.09-2.04 (2H, m, CCH₂), 1.90-1.80 (2H, m, CCH₂), 1.77 (3H, s, CH₃); LCMS 12 min, Rt=5.71 min, (M⁺+1)=313.2.

### EXAMPLE 63

### N-(2-methylpropyl)-N-{[3-(trifluoromethylthio)phenyl]methyl}piperidin-4-amine fumarate

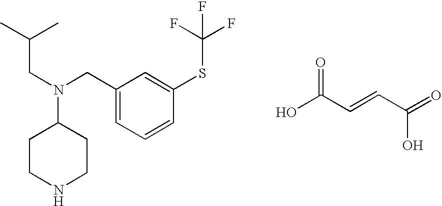

The procedure for reductive amination in example 36 applies for this compound using 3-(trifluoromethylthio)benzaldehyde. The N-Boc deprotection procedure was as follows: The boc-amine (0.65 mg, 1.46 mmol) was dissolved in dichloromethane (5 ml), and trifluoroacetic acid (2 ml) and anisole (2 ml) were added in one portion, under an atmosphere of nitrogen. The reaction was monitored by thin layer chromatography (100% ethyl acetate; reactant. r.f. 0.4, product r.f. 0.0). After 2 hours, the reaction was concentrated in vacuo, azeotroped with dichloromethane (c.a. 25 ml), taken up in methanol (c.a 5 ml) and passed through an SCX-2 column. The resultant colourless oil was purified by preparative HPLC using the UV-Flex system. The resulting colourless oil was dissolved in aqueous acetonitrile (c.a. 20 ml), and fumaric acid (1 eq) added. After 5 minutes, this was freeze dried to give a white solid (0.448 g, 0.96 mmol) as the title compound. δ_{H} (300 MHz, MeOD) 7.65 (1H, m), 7.52 (2H, m), 7.35 (1H, m), 6.55 (2H, s, fumarate), 3.65 (2H, s), 3.40 (2H, m), 2.81 (3H, m), 2.25 (2H, d), 1.95 (2H, m), 1.72 (3H, m), 0.79 (6H, d). LCMS 12 minute gradient, Rt=4.74 mins, (M⁺+1)=347.2

### EXAMPLE 64

### N-(2-methylpropyl)-N-{[2-(trifluoromethylthio)phenyl]methyl}piperidin-4-amine fumarate

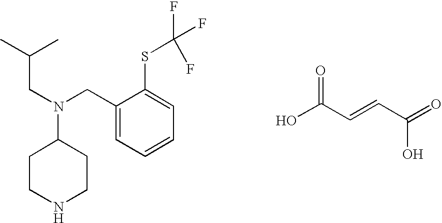

The compound was prepared similarly to example 63 using 4-isobutylamino-piperidine-1-carboxylic acid tert-butyl ester (0.40 g, 1.56 mmol) and 2-(trifluoromethylthio)benzaldehyde (0.90, 4.67 mmol) to give the title compound as a white solid (0.58 g, 1.25 mmol). 81, (300 MHz, MeOD) 7.67 (2H, m), 7.52 (1H, t), 7.35 (1H, m), 6.65 (2H, s, fumarate), 3.95 (2H, s), 3.50 (2H, m), 2.90 (3H, m), 2.27 (2H, d), 2.10 (2H, m), 1.87 (2H, m), 1.57 (1H, m), 0.79 (6H, d). LCMS 12 minute gradient, Rt=5.81 mins, (M⁺+1)=347.2

### EXAMPLE 65

### N-(2-methylpropyl)-N-[(3-bromophenyl)methyl]piperidin-4-amine fumarate

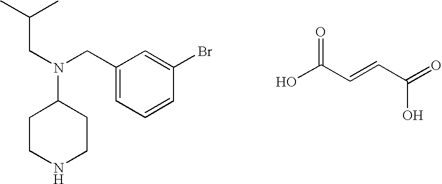

The compound was prepared similarly to example 63 using 4-isobutylamino-piperidine-1-carboxylic acid tert-butyl ester (0.40 g, 1.56 mmol) and 3-bromobenzaldehyde (0.86, 4.67 mmol) to give the title compound as an off-white solid (0.71 g, 1.59 mmol). δ_{H} (300 MHz, MeOD) 7.57 (1H, s), 7.35 (2H, m), 7.24 (1H, m), 6.58 (2H, s, fumarate), 3.67 (2H, s), 3.45 (2H, m), 2.96 (2H, m), 2.83 (1H, m), 2.30 (2H, d), 2.00 (2H, m), 1.75 (3H, m), 0.87 (6H, d). LCMS 12 minute gradient, Rt=3.62 mins, (M⁺+1)=326.1

### EXAMPLE 66

### N-(2-methylpropyl)-N-[(3-phenoxyphenyl)methyl]piperidin-4-amine fumarate

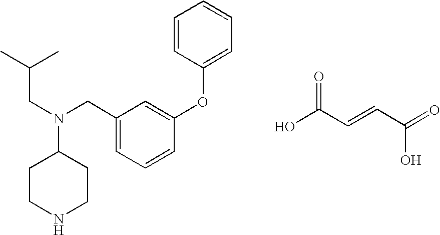

The compound was prepared similarly to example 63 using 4-isobutylamino-piperidine-1-carboxylic acid tert-butyl ester (0.40 g, 1.56 mmol) and 3-phenoxybenzaldehyde (0.93 g, 4.67 mmol) to give the title compound as a white solid (0.53 g, 1.16 mmol). δ_{H} (300 MHz, MeOD) 7.32 (3H, m), 7.12 (2H, m), 6.97 (3H, m), 6.88 (1H, m), 6.70 (2H, s, fumarate), 3.66 (2H, s), 3.40 (2H, m), 2.85 (3H, m), 2.26 (2H, d), 1.92 (2H, m), 1.69 (3H, m), 0.82 (6H, d). LCMS 12 minute gradient, Rt=4.21 mins, (Me+1)=339.3

### EXAMPLE 67

### N-(2-methylpropyl)-N-{[3-(difluoromethoxy)phenyl]methyl}piperidin-4-amine fumarate

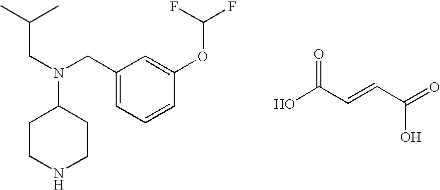

The compound was prepared similarly to example 63 using 4 isobutylamino-piperidine-1-carboxylic acid tert-butyl ester (0.40 g, 1.56 mmol) and 3-(difluoromethoxy)benzaldehyde (0.80 g, 4.67 mmol) to give the title compound as a white solid (0.53 g, 1.23 mmol). δ_{H} (300 MHz, MeOD) 7.32 (1H, m), 7.23 (2H, m), 7.05 (1H, m), 6.78-6.53 (1H, br-t, CHF₂), 6.68 (2H, s, fumarate), 3.69 (2H, s), 3.42 (2H, m), 2.86 (3H, m), 2.30 (2H, d), 2.00 (2H, m), 1.75 (3H, m), 0.89 (6H, d). LCMS 12 minute gradient, Rt=3.23 mins, (M⁺+1)=313.2

### EXAMPLE 68

### N-(2-methylpropyl)-N-[(2,5-dimethylphenyl)methyl]piperidin-4-amine fumarate

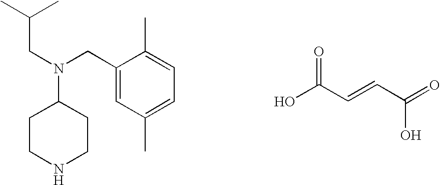

The compound was prepared similarly to example 63 using 4-isobutylamino-piperidine-1-carboxylic acid tert-butyl ester (0.40 g, 1.56 mmol) and 2,5-dimethylbenzaldehyde (0.76 g, 4.67 mmol) to give the title compound as a white solid (0.50 g, 1.28 mmol). δ_{H} (300 MHz, MeOD) 7.14 (1H, s), 6.97 (2H, m), 6.69 (2H, s, fumarate), 3.63 (2H, s), 3.42 (2H, m), 2.85 (3H, m), 2.32 (3H, s), 2.29 (3H, s), 2.28 (2H, d), 2.03 (2H, m), 1.85 (2H, m), 1.60 (1H, m), 0.83 (6H, d). LCMS 12 minute gradient, Rt=3.20 mins, M⁺+1)=275.3

### EXAMPLE 69

### N-(2-methylpropyl)-N-[(4-cyanophenyl)methyl]piperidin-4-amine fumarate

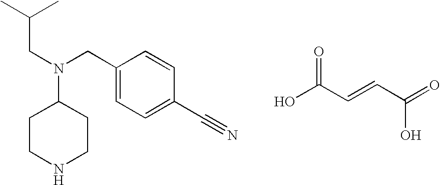

The compound was prepared similarly to example 63 using 4-isobutylamino-piperidine-1-carboxylic acid ten-butyl ester (0.50 g, 1.95 mmol) and 4-cyanobenzaldehyde (0.76 g, 5.85 mmol) to give the title compound as a white solid (0.45 g, 1.16 mmol). δ_{H} (300 z, MeOD) 7.92 (2H, d), 7.78 (2H, d), 6.91 (2H, s, fumarate), 3.97 (2H, s), 3.65 (2H, m), 3.08 (3H, m), 2.52 (2H, d), 2.24 (2H, m), 1.95 (3H, m), 1.08 (6H, d). LCMS 12 minute gradient, Rt=2.93 mins, (M⁺+1)=272.2

### EXAMPLE 70

### N-(2-methylpropyl)-N-[(2-ethoxyphenyl)methyl]piperidin-4-amine fumarate

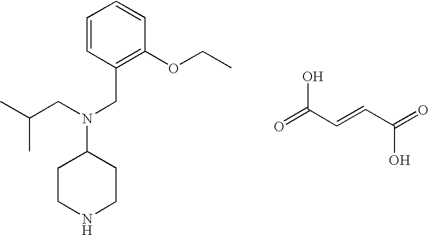

The compound was prepared similarly to example 63 using 4-isobutylamino-piperidine-1-carboxylic acid tert-butyl ester (0.40 g, 1.56 mmol) and 2-ethoxybenzaldehyde (0.70 g, 4.68 mmol) to give the title compound as a white solid (0.52 g, 1.28 mmol). δ_{H} (300 MHz, MeOD) 7.38 (1H, m), 7.17 (1H, m), 6.89 (2H, m), 6.66 (2H, s, fumarate), 4.05 (2H, q), 3.72 (2H, s), 3.43 (2H, m), 2.87 (3H, m), 2.32 (2H, d), 2.02 (2H, m), 1.76 (3H, m), 1.40 (3H, t), 0.85 (6H, d). LCMS 12 minute gradient, Rt=2.03 mins, (M⁺+1)=291.3

### EXAMPLE 71

### N-(2-methylpropyl)-N-[(4-fluorophenyl)methyl]piperidin-4-amine fumarate

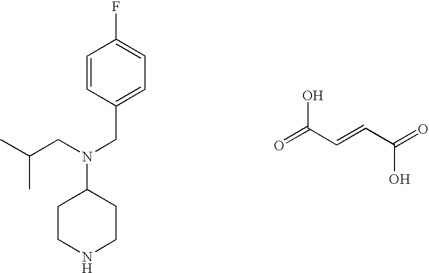

The compound was prepared similarly to example 63 using 4-isobutylamino-piperidine-1-carboxylic acid tert-butyl ester (0.40 g, 1.56 mmol) and 4-fluorobenzaldehyde (0.69 g, 4.68 mmol) to give the title compound as a white solid (0.41 g, 1.05 mmol). δ_{H} (300 z, MeOD) 7.35 (2H, m), 7.05 (2H, m), 6.67 (2H, s, fumarate), 3.62 (2H, s), 3.41 (2H, m), 2.82 (3H, m), 2.23 (2H, d), 1.96 (2H, m), 1.70 (3H, m), 0.83 (6H, d). LCMS 12 minute gradient, Rt=1.79 mins, (M⁺+1)=265.2

### EXAMPLE 72

### N-(2-methylpropyl)-N-[(3-ethoxyphenyl)methyl]piperidin-4-amine fumarate

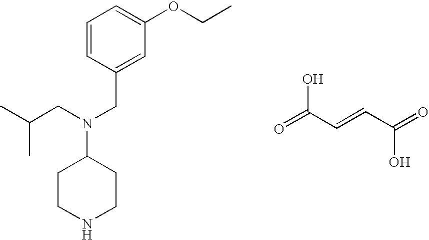

The compound was prepared similarly to example 63 using 4-isobutylamino-piperidine-1-carboxylic acid tert-butyl ester (0.40 g, 1.56 mmol) and 3-ethoxybenzaldehyde (0.69 g, 4.68 mmol) to give the title compound as a white solid (0.31 g, 0.74 mmol). δ_{H} (300 MHz, MeOD) 7.16 (1H, m), 6.91 (2H, m), 6.74 (1H, m), 6.71 (2H, s, fumarate), 4.05 (2H, q), 3.64 (2H, s), 3.41 (2H, m), 2.86 (3H, m), 2.28 (2H, d), 1.97 (2H, m), 1.72 (3H, m), 1.36 (3H, t), 0.86 (6H, d). LCMS 12 minute gradient, Rt=2.77 mins, (M⁺+1)=291.3

### EXAMPLE 73

### N-(2-methylpropyl)-N-[(2-chloro-6-fluorophenyl)methyl]piperidin-4-amine fumarate

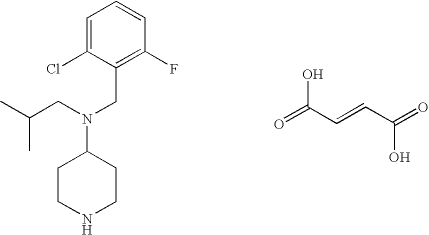

The compound was prepared similarly to example 63 using 4-isobutylamino-piperidine-1-carboxylic acid tert-butyl ester (0.40 g, 1.56 mmol) and 2-chloro-6-fluorobenzaldehyde (0.74 g, 4.67 mmol) to give the title compound as a white solid (0.42 g, 1.0 mmol). δ_{H} (300 MHz, MeOD) 7.26 (2H, m), 7.06 (1H, m), 6.65 (2H, s, fumarate), 3.79 (2H, s), 3.44 (2H, m), 2.82 (2H, dt), 2.78 (1H, m), 2.25 (2H, d), 2.04 (2H, m), 1.81 (2H, m), 1.62 (1H, m), 0.74 (6H, d). LCMS 12 minute gradient, Rt=3.96 mins, (M++I)=299.2

### EXAMPLE 74

### N-(2-methylpropyl)-N-[(2-biphenyl)methyl]piperidin-4-amine fumarate

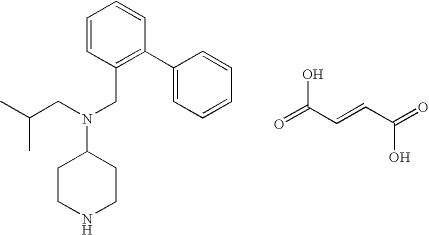

(i) The N-(2-methylpropyl)-N-[(2-bromophenyl)methyl]4-piperidin-1-amine carboxylic acid tert-butyl ester was prepared similarly to example 63 using 4-isobutylamino-piperidine-1-carboxylic acid tert-butyl ester (0.40 g, 1.56 mmol) and 2-bromobenzaldehyde (0.86 g, 4.67 mmol) to give a yellow oil (0.54 g, 1.27 mmol). LCMS 12 minute gradient, Rt=3.58 mins, (M⁺+1)=426.2

(ii) N-(2-methylpropyl)-N-[(2-bromophenyl)methyl]4-piperidin-1-amine carboxylic acid tert-butyl ester (0.85 g, 2.00 mmol) in tetrahydrofuran (50 ml) and water (50 ml) was added phenyl boronic acid, bis(triphenylphosphine)palladium (II) chloride (0.14 g, 0.20 mmol) and sodium carbonate (0.42 g, 4.00 mmol), under an atmosphere of nitrogen. The mixture was heated to 90° C. and stirred for 3 h. The mixture was cooled, poured into diethyl ether (200 ml) and washed with 2M NaOH (50 ml). The organic was further washed with brine (50 ml), dried (MgSO₄) and concentrated in vacuo to a yellow oil. The oil was taken up in methanol (c.a. 5 ml), and passed through an SCX-2 column to give a colourless oil (0.71 g, 1.68 mmol). The colourless oil (0.71 g, 1.68 mmol) was dissolved in dichloromethane (5 ml), and trifluoroacetic acid (2 ml) and anisole (2 ml) were added in portion, under an atmosphere of nitrogen. The reaction was monitored by thin layer chromatography (100% ethyl acetate; reactant r.f. 0.45, product r.f. 0.0). After 2 hours, the reaction was concentrated in vacuo, azeotroped with dichloromethane (c.a 25 ml), taken up in methanol (c.a. 5 ml), and passed through an SCX-2 column. The resultant colourless oil was purified by preparative HPLC using the WV-Flex system. The colourless oil was dissolved in aqueous acetonitrile (c.a. 20 ml), and fumaric acid (1 eq) added. After 5 minutes, this was freeze dried to give a white solid (0.73 g, 0.66 mmol) as the title compound. δ_{H} (300 MHz, MeOD) 7.48 (1H, m), 7.34-7.17 (7H, m), 7.05 (1H, m), 6.58 (2H, s, fumarate), 3.50 (2H, s), 3.21 (2H, m), 2.67 (2H, dt), 2.48 (1H, m), 2.02 (2H, d), 1.46 (5H, m), 0.58 (6H, d). LCMS 12 minute gradient, Rt=4.09 mins, (M⁺+1)=323.3

### EXAMPLE 75

### N-(2-methylpropyl)-N-[(3-biphenyl)methyl]piperidin-4-amine fumarate

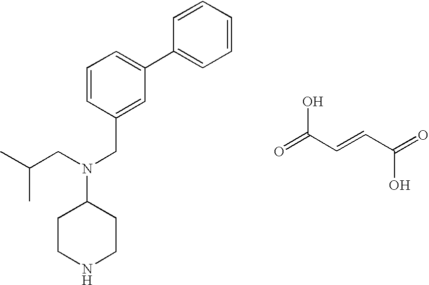

(i) N-(2-methylpropyl)-N-[(3-bromophenyl)methyl]4-piperidine-1-carboxylic acid tert-butyl ester was prepared similarly to example 63 using 4-isobutylamino-piperidine-1-carboxylic acid tert-butyl ester (0.40 g, 1.56 mmol) and 3-bromobenzaldehyde (0.86 g, 4.67 mmol) to give the title compound as a pale yellow oil (0.55 g, 1.27 mmol). LCMS 12 minute gradient, Rt=3.29 mins, (M⁺+1)=426.2.

(ii) The compound was prepared similarly to example 74 using N-(2-methylpropyl)-N-[(3-bromophenyl)methyl]4-piperidine-1-carboxylic acid tert-butyl ester (0.66, 1.56 mmol) and phenyl boronic acid (0.38 g, 3.14 mmol) to give the title compound as a white solid (0.44 g, 1.00 mmol). δ_{H} (300 MHz, MeOD) 7.62 (3H, m), 7.51-7.30 (6H, m), 7.05 (1H, m), 6.71 (2H, s, fumarate), 3.75 (2H, s), 3.42 (2H, m), 2.89 (3H, m), 2.33 (2H, d), 2.06 (2H, m), 1.78 (3H, m), 0.91 (6H, d). LCMS 12 minute gradient, Rt=4.05 mins, (M⁺+1)=323.3

### EXAMPLE 76

### N-(2-methylpropyl)-N-{[2-fluoro-6-(trifluoromethyl)phenyl]methyl}piperidin-4-amine fumarate

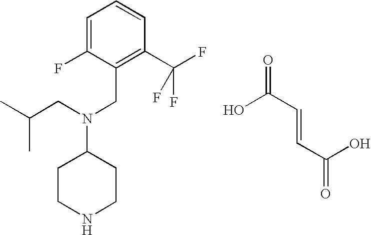

As example 26 with 1,1-dimethylethyl N-[(2-methylpropyl)amino]piperidine-1-carboxylate and 2-fluoro-6-(trifluoromethyl)benzaldehyde, further purified by MS guided preparative LC and loaded onto SCX-2 ion exchange cartridge (5 g). The column was washed with methanol (10 ml) and eluting the product with 2M ammonia in methanol solution (10 ml) the solvent removed in vacuo to give (117 mg, 23%) as a colourless oil. The product was taken up in diethyl ether (10 ml) and a few drops of methanol to solubilize and a hot solution of fumaric acid (41 mg, 0.3 mmol, 1 eq) in methanol (0.5 ml) was added. The solution was heated and a few drops of methanol added until all solid was in solution, then the mixture was allowed to slowly cool to 0° C. The product was collected by filtration and dried in a vacuum oven (40° C. for 2 h) to give N-(2-methylpropyl)-N-{[2-fluoro-6-(trifluoromethyl)phenyl]methyl}piperidin-4-amine fumarate (121 mg, 18%) as a white solid. δ_{H} (300 MHz, MeOD) 7.57-7.48 (2H, n, ArH), 7.42-7.36 (1H, m, ArH), 6.70 (2H, s, fumarate CH), 3.89 (2H, s, CH₂Ar), 3.50-3.45 (2H, m, NCH₂), 3.00-2.90 (2H, m, NCH₂), 2.87-2.77 (1H, m, NCH), 2.26 (2H, d, J=7.2 Hz, NCH₂), 2.04-2.00 (2H, m, CCH₂), 1.91-1.77 (2H, m, CCH₂) 1.59-1.48 (1H, m, CH(CH₃)₂) and 0.74 (6H, d, J=6.6 Hz, CH₃); LCMS 12 min. Rt=5.55 min, (M⁺+1)=333.2.

### EXAMPLE 77

### N-(2-methylpropyl)-N-[(3,5-difluorophenyl)methyl]piperidin-4-amine fumarate

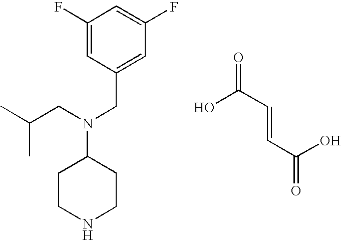

As example 56 with 1,1-dimethylethyl 4-[(2-methylpropyl)amino]piperidine-1-carboxylate and 3,5-difluorobenzaldehyde to give N-(2-methylpropyl)-N-[(3,5-difluorophenyl)methyl]piperidin-4-amine fumarate (264 mg, 44%) as a white solid. δ_{H} (300 MHz, MeOD) 7.01-6.98 (2H, m, ArH), 6.83-6.76 (1H, m, ArH), 6.70 (2H, s, fumarate CH), 3.69 (2H, s, CH₂Ar), 3.47-3.43 (2H, m, NCH₂), 3.00-2.90 (2H, m, NCH₂), 2.87-3.76 (1H, m, NCH), 2.30 (2H, d, J=7.2 Hz, NCH₂), 2.04-2.00 (2H, m, CCH₂), 1.84-1.66 (3H, m, CH(CH₃)₂ and CCH₂) and 0.91 (6H, d, J=6.6 Hz, CH₃); LCMS 12 min, Rt=4.15 min, (M⁺+1)=283.2.

### EXAMPLE 78

### N-(2-methylpropyl)-N-[(3,5-dimethylihenylmethyl]piperidin-4-amine fumarate

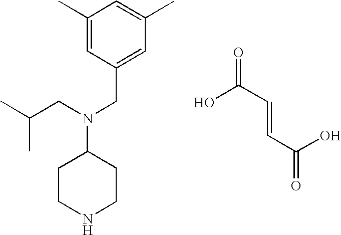

As example 56 with 1,1-dimethylethyl 4-[(2-methylpropyl)amino]piperidine-1-carboxylate and 3,5-dimethylbenzaldehyde to give N-(2-methylpropyl)-N-[(3,5-dimethylphenyl)methyl]piperidin-4-amine fumarate (356 mg, 61%) as a white solid. δ_{H} (300 MHz, MeOD) 6.97 (2H, s, ArH), 6.88 (1H, s, ArH), 6.70 (2H, s, fumarate CH), 3.61 (2H, s, CH₂Ar), 3.45-3.41 (2H, m, NCH₂), 2.96-2.79 (3H, m, NCH₂ and NCH), 2.30-2.29 (8H, m, Hz, NCH₂ and CH₃), 2.01-1.97 (2H, m, CCH₂), 1.84-1.67 (3H, m, CH(CH₃)₂ and CCH₂) and 0.89 (6H, d, J=6.6 Hz, CH₃); LCMS 12 min, Rt=3.08 min, (M⁺+1)=275.3.

### EXAMPLE 79

### N-(2-methylpropyl)-N-[(3,5-dimethoxyphenyl)methyl]piperidin-4-amine fumarate

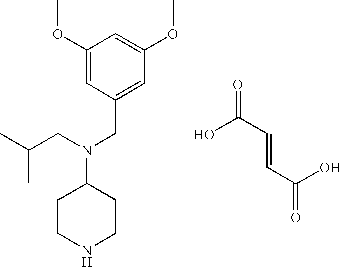

As example 56 with 1,1-dimethylethyl 4-[(2-methylpropyl)amino]piperidine-1-carboxylate and 3,5-dimethoxybenzaldehyde to give N-(2-methylpropyl)-N-[(3,5-dimethoxyphenyl)methyl]piperidin-4-amine fumarate (378 mg, 53%) as a white solid. δ_{H} (300 MHz, MeOD) 6.70 (2H, s, fumarate CH), 6.57-6.56 (2H, m, ArH), 6.36-6.35 (1H, m, ArH), 3.77 (6H, s, OCH₃), 3.62 (2H, s, CH₂Ar) 3.46-3.41(2H, m, NCH₂), 2.97-2.78 (3H, m, NCH₂ and NCH), 2.30 (2H, d, J=7.2 Hz, NCH₂), 2.02-1.98 (2H, m, CCH₂), 1.84-1.68 (3H, m, CH(CH₃)₂ and CCH₂) and 0.92 (6H, d, J=6.6 Hz, CH₃); LCMS 12 min, Rt=2.66 min, (M⁺+1)=307.2.

### EXAMPLE 80

### N-(2-methylpropyl)-N-{[3-fluoro-5-(trifluoromegaiphenyl]methyl}piperidin-4-amine fumarate

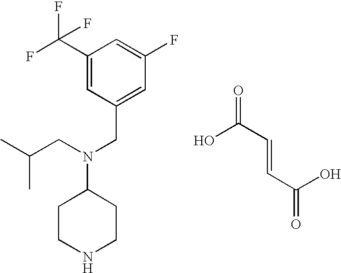

As example 56 with 1,1-dimethylethyl 4-[(2-methylpropyl)amino]piperidine-1-carboxylate and 3-fluoro-5-(trifluoromethyl)benzaldehyde to give N-(2-methylpropyl)-N-{[3-fluoro-5-(trifluoromethyl)phenyl]methyl}piperidin-4-amine fumarate (397 mg, 59%) as a white solid. δ_{H} (300 MHz, MeOD) 7.56 (1H, s, ArH), 7.43 (1H, d, J=9.4 Hz, ArH), 7.31 (1H, d, J=8.5 Hz, ArH), 6.70 (2H, s, fumarate CH), 3.77 (2H, s, CH₂Ar), 3.48-3.44 (2H, m, NCH₂), 3.01-2.79 (3H, m, NCH₂ and NCH), 2.32 (2H, d, J=7.2 Hz, NCH₂), 2.06-2.02 (2H, m, CCH₂), 1.86-1.63 (3H, m, CH(CH₃)₂ and CCH₂) and 0.89 (6H, d, J=6.6 Hz, CH₃); LCMS 12 min, Rt=5.61 min, (M⁺+1)=333.2.

### EXAMPLE 81

### N-(2-methylpropyl)-N-[(5-fluoro-2-methoxyphenyl)methyl]piperidin-4-amine fumarate

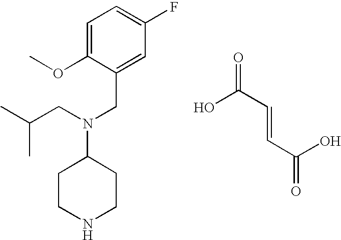

As example 56 with 1,1-dimethylethyl 4-[(2-methylpropyl)amino]piperidine-1-carboxylate and 5-fluoro-2-methoxybenzaldehyde to give N-(2-methylpropyl)-N-[(5-fluoro-2-methoxyphenyl)methyl]piperidin-4-amine fumarate (317 mg, 52%) as a white solid. δ_{H} (300 MHz, MeOD) 7.247.21 (1H, m, ArH), 6.96-6.90 (2H, m, ArH), 6.70 (2H, s, fumarate CH), 3.82 (3H, s, OCH₃), 3.68 (2H, s, CH₂Ar) 3.46-3.43 (2H, m, NCH₂), 2.98-2.77 (3H, m, NCH₂ and NCH), 2.32 (2H, d, J=7.2 Hz, NCH₂), 2.05-2.01 (2H, m, CCH₂), 1.85-1.67 (3H, m, CH(CH₃)₂ and CCH₂) and 0.90 (6H, d, J=6.6 Hz, CH₃); LCMS 12 min, Rt=2.03 min, (M⁺+1)=295.2.

### EXAMPLE 82

### N-(2-methylpropyl)-N-{[4-(trifluoromethoxy)phenyl]methyl}piperidin-4-amine fumarate

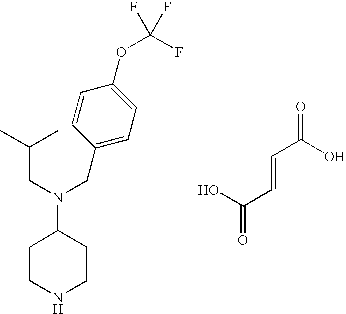

The title product was prepared from tert-butyl 4-isobutylamino-piperidine-1-carboxylate and 4-(trifluoromethoxy)benzaldehyde using the method described in example 36. LCMS 12 mins gradient Rt=4.19 (M⁺+1) 331.2. 1H NMR (d6-DMSO) δ=7.44 (2H, d, J=8.28 Hz), 7.30 (2H, d, J=8.28 Hz), 6.45 (2H, s), 3.60 (2H, s), 3.19 (2H, brd), 2.79-2.67 (4H, m), 2.18 (2H, d, J=6.97 Hz), 1.81-1.61 (5H, m), 0.79 (6H, d, J=6.40 Hz).

### EXAMPLE 83

### N-(2-methylpropyl)-N-{[3-(methoxycarbonyl)phenyl]methyl}piperidin-4-amine fumarate

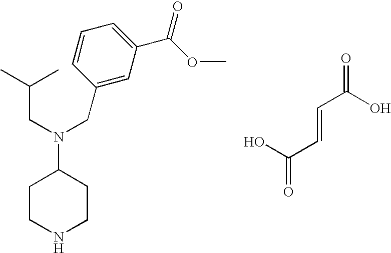

The title product was prepared from tert-butyl 4-isobutylamino-piperidine-1-carboxylate and methyl 3-formylbenzoate using the method described in example 36. LCMS 12 mins gradient Rt=2.94 (M⁺+1) 305.2. 1H NMR (d6-DMSO) δ=7.95 (1H, s), 7.81 (1H, s), 7.60 (1H, d), 7.48-7.46 (1H, m), 6.42 (2H, s), 3.85 (3H, s, OMe), 3.64 (2H, s), 3.38 (2H, brd), 2.77-2.50 (4H, m), 2.19 (2H, d, J=6.97 Hz), 1.80-1.59 (5H, m), 0.79 (6H, d, J=6.60 Hz).

### EXAMPLE 84

### N-(2-methylpropyl)-N-[(2,6-dimethylphenyl)methyl]piperidin-4-amine fumarate

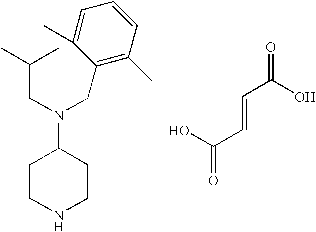

The title product was prepared from tert-butyl 4-isobutylamino-piperidine-1-carboxylate and 2,6-dimethylbenzaldehyde using the method described in example 36. LCMS 12 mins gradient Rt=4.04 (M⁺+1) 275.3. 1H NMR (d6-DMSO) δ=7.04-7.01 (3H, m), 6.41 (2H, s), 3.59 (2H, s), 3.28-3.16 (2H, m), 2.76-2.72 (4H, m), 2.34 (6H, s), 2.10 (2H, d, J=6.97 Hz), 1.86-1.65 (5H, m), 0.63 (6H, d, J=6.59 Hz).

### EXAMPLE 85

### N-(2-methylpropyl)-N-{[2-(tert-butylthio)phenyl]methyl}piperidin-4-amine fumarate

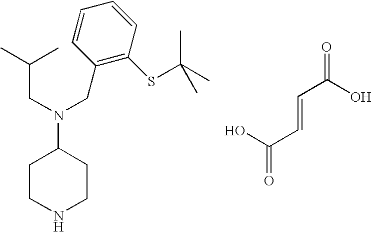

The title product was prepared from tert-butyl 4-isobutylamino-piperidine-1-carboxylate and 2-(tert-butylthio)benzaldehyde using the method described in example 36. LCMS 12 mins gradient Rt=4.33 (M⁺+1) 335.2. 1H NMR (d6-DMSO) δ=7.65 (1H, d, J=207.54 Hz), 7.48-7.40 (2H, m), 7.26-7.2 (1H, m), 6.42 (2H, s), 3.86 (2H, s), 3.24 (2H, brd), 2.75-2.62 (4H, m), 2.20 (2H, d, J=6.97 Hz), 1.78-1.54 (5H, m), 1.23 (9H, s, 3×Me), 0.78 (6H, d, J=6.60 Hz).

### EXAMPLE 86

### N-(2-methylpropyl)-N-{[4-fluoro-2-(trifluoromethyl)phenyl]methyl}piperidin-4-amine fumarate

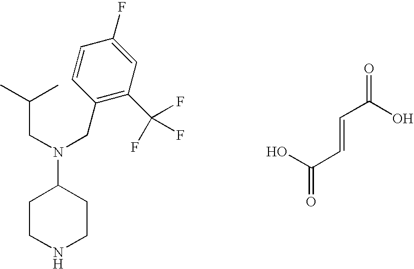 Method 1

The title product was prepared from tert-butyl 4-isobutylamino-piperidine-1-carboxylate and 2-(trifluoromethyl)-4-fluorobenzaldehyde using the method described in example 36. LCMS 12 mins gradient Rt=6.09 (M⁺+1) 333.5. 1H NMR (d6-DMSO) δ=7.95-7.85 (1H, m), 7.59-7.49 (2H, m), 6.49 (2H, s), 3.63 (2H, s), 3.25 (2H, brd), 2.86-2.62 (3H, m), 2.20 (2H, d, J=6.95 Hz), 1.85-1.5 (5H, m), 0.80 (6H, d, J=6.60 Hz)

Method 2

(i) To a solution of 4-isobutylamino-piperidine-1-carboxylic acid tert-butyl ester (10 g, 39 mmol, 1.0 eq) in 1,2-dichloroethane (100 ml) was added 2-(trifluoromethyl)-4-fluorobenzaldehyde (22.5 g, 117 mmol. 3.0 eq). To this was added a solution of sodium triacetoxyborohydride (24.8 g, 117 mmol, 3.0 eq) in dimethylformamide (20 ml). This mixture was left to stir under nitrogen, at room temperature, for 16 h. After this time the reaction mixture was quenched with water (50 ml) and subsequently stirred vigorously for several minutes. The reaction mixture was then separated with DCM, washing the organic layer with water (×3). The combined organics were dried over sodium sulfate and evaporated in vacuo to give an oil. Purification of this crude oil by chromatography on silica was then performed using an Isco system, eluting with 0-50% ethyl acetate:Hexane gradient conditions over 40 mins gave product which was taken directly onto the next step.

(ii) To this oil (39 mmol, 1.0 eq.) in dichloromethane (25 ml) was added a solution of 95% trifluoroacetic acid:water (20 ml). The solution was stirred at room temperature for 2 h. Solvent and TFA were removed in vacuo. The resulting oil was taken up in DCM and washed with saturated sodium carbonate. The organics were collected, dried over sodium sulfate, and evaporated in vacuo. The resulting oil was taken up in methanol and loaded onto an SCX-2 pad 120 g. The column was washed with methanol (250 ml). Basic material was then eluted using 2M ammonia in methanol (250 ml). Removal of solvent from the ammonia/methanol mixture under vacuum, gave the desired compound 5.17 g as a free base.

The oil was taken up in diethylether. To this solution was added a solution of fumaric acid (1.8 g, 1 eq) in hot ethanol. The mixture was left at room temperature for a few minutes before precipitation occurred. The resulting precipitate was collected by filtration to give the title compound as a white solid (3.29 g).

### EXAMPLE 87

### N-(3,3-dimethylbutyl)-N-[(2-bromophenyl)methyl]piperidin-4-amine fumarate

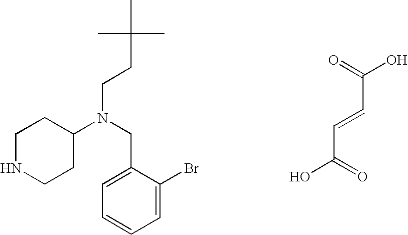

(i) To 10% Pd/C (1.0 g, 10% wt), under nitrogen, was added a solution of the 1-Boc-4-piperidone (10.77 g, 54.06 mmole, 1.0 eq.) and 3,3-dimethylbutylamine (5.58 g, 55.14 mmole, 1.02 eq.) in ethanol (65 ml). This was hydrogenated for 1.5 h, at 65 psi hydrogen, using a Parr hydrogenator. The catalyst was removed by filtration through Celite. Solvent was removed under vacuum to give a pale yellow oil (15.7 g). This was purified by automated flash chromatography using an ISCO Combiflash system (SiO₂ (110 g); 0-10% methanol in ethyl acetate gradient elution over 40 minutes) to give 1,1-dimethylethyl 4-[(3,3-dimethylbutyl)amino]piperidine-1-carboxylate as a colourless oil (3.53 g, 23%). δ_{H} (300 MHz, CDCl₃) 4.05-4.01 (2H, m, NCH₂), 2.83-2.75 (2H, m, NCH₂), 2.65-2.56 (3H, m, NCH, NCH₂), 1.86-1.82 (2H, m, CCH₂), 1.45 (9H, s, OC(CH₃)₃), 1.41-1.36 (2H, m, CCH₂), 1.31-1.18 (2H, m, CCH₂), 0.91 (9H, s, C(CH₃)₃); LCMS 6 min, Rt=2.7 min, (M⁺+1)=285. A second batch of 1,1-dimethylethyl 4-[(3,3dimethylbutyl)amino]piperidine-1-carboxylate, contaminated with a small amount of 1-Boc-4-piperidone, was isolated as a colourless oil (11.85 g).

(ii) To a solution of 1,1-dimethylethyl 4-[(3,3-dimethylbutyl)amino]piperidine-1-carboxylate (0.427 g, 1.50 mmole, 1.0 eq.) in 1,2-dichloroethane (10 ml) was added 2-bromobenzaldehyde (0.53 ml, 4.50 mmole, 3.0 eq.). To this was added a solution of sodium triacetoxyborohydride (0.954 g, 4.50 mmole, 3.0 eq.) in dimethylformamide (2 ml). This mixture was left to stir for 3 days under nitrogen, at room temperature. To the reaction mixture was added water (10 ml) and the mixture stirred vigorously for several minutes. The chlorinated organic layer was run through a hydrophobic frit to remove water, diluted with methanol (10 ml) and loaded onto an SCX-2 (10 g) column. The column was washed with methanol (50 ml) then basic material eluted with 2N ammonia in methanol. The ammonia/methanol solution was concentrated in vacuo to give 1,1-dimethylethyl 4-[(2-bromophenylmethyl)(3,3-dimethylbutyl)amino]piperidine-1-carboxylate as a colourless oil (0.681 g, 100%). To a solution of this oil (0.681 g, 1.50 mmole, 1.0 eq.) in dichloromethane (10 ml) was added trifluoroacetic acid (TFA) (1.67 ml, 22.5 mmole, 15 eq). The solution was stirred overnight at room temperature. Solvent and TFA were removed in vacuo. The resulting oil was taken up in methanol and loaded onto an SCX-2 (10 g) column. The column was washed with methanol (50 ml). Basic material was then eluted using 2M ammonia in methanol (50 ml). Removal of solvent from the ammonia/methanol mixture under vacuum, gave a colourless oil (0.530 g, 100%). The oil was taken up in methanol. To this solution was added a solution of fumaric acid (0.174 g, 1.50 mmole, 1 eq) in methanol. The mixture was left to stir for a couple of minutes, then ethyl acetate and cyclohexane were added. The resulting precipitate was collected by filtration to give the title compound as a white solid (0.642 g, 91%). δ_{H} (300 MHz, MeOD) 7.61 (1H, dd, ArH), 7.55 (1H, dd, ArH), 7.37-7.32 (1H, m, ArH), 7.19-7.13 (1H, m, ArH), 6.71 (2H, s, fumarate CH), 3.79 (2H, s, CH₂Ar), 3.49-3.44 (2H, m, NCH₂), 3.04-2.88 (3H, m, NCH, NCH₂), 2.65-2.59 (2H, m, NCH₂), 2.10-2.05 (2H, m, CCH₂), 1.90-1.75 (2H, m, CCH₂), 1.40-1.35 (2H, m, CH₂tBu), 0.87 (9H, s, CH₃); LCMS 12 min, Rt=3.9 min, (M⁺+1)=353, 355.

### EXAMPLE 88

### N-(3,3-dimethylbutyl)-N-[(2-methylphenyl)methyl]piperidin-4-amine fumarate

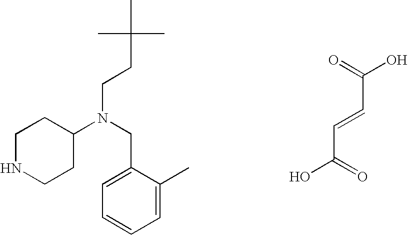

As method previously described for Example 87, using 1,1-dimethylethyl 4-[(3,3-dimethylbutyl)amino]piperidine-1-carboxylate and 2-methylbenzaldehyde. Isolation of the fumarate salt from methanol, ethyl acetate, cyclohexane yielded the title compound as a white solid (0.480 g, 79%). δ_{H} (300 z, MeOD) 7.35 (1H, dd, ArH), 7.16-7.14 (3H, m, ArH), 6.70 (2H, s, fumarate CH), 3.73 (2H, s, CH₂Ar), 3.49-3.45 (2H, m, NCH₂), 2.99-2.93 (3H, m, NCH₂, NCH), 2.62-2.56 (2H, m, NCH₂), 2.39 (31, s, ArCH₃), 2.09-2.04 (2H, m, CCH₂), 1.92-1.81 (2H, m, CCH₂), 1.37-1.32 (2H, m, CH₂tBu), 0.84 (9H, s, CH₃); LCMS 12 min, Rt=3.4 min, ++1)=289.

### EXAMPLE 89

### N-(2-methylpropyl)-N-[(2,4-dichlorophenyl)methyl]piperidin-4-amine fumarate

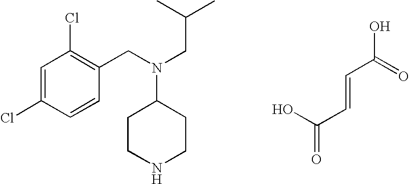 Method 1

The compound was prepared similarly to example 63 using 4-isobutylamino-piperidine-1-carboxylic acid tert-butyl ester (0.40 g, 1.56 mmol) and 2,4-dichlorobenzaldehyde (0.82 g, 4.67 mmol) to give the title compound as a white solid (0.30 g). δ_{H} (300 MHz, MeOD) 7.60 (1H, d), 7.43 (1H, s), 7.31 (1H, m), 6.60 (2H, s, fumarate), 3.78 (2H, s), 3.48 (2H, m), 2.92 (2H, m), 2.81 (1H, m), 2.30 (2H, d), 2.03 (2H, m), 1.75 (3H, m), 0.86 (6H, d). LCMS 12 minute gradient, Rt=5.45 mins, (M⁺+1)=316.1

Method 2

(i) To a solution of 4-isobutylamino-piperidine-1-carboxylic acid tert-butyl ester (9.0 g, 35.1 mmol) in 1,2-dichloroethane (100 ml) was added 2,4-dichlorobenzaldehyde (7.68 g, 43.9 mmol, 1.25 eq). To this was added a solution of sodium triacetoxyborohydride (11.2 g, 52.7 mmol, 1.5 eq) in 1,2-dichloroethane (100 ml). This mixture was left to stir under nitrogen, at room temperature, for 24 h. After this time the reaction mixture was quenched with water (100 ml). The organic phase was then separated and the aqueous phase extracted with dichloromethane. The combined organics were washed with brine, dried over magnesium sulfate, filtered and evaporated in vacuo to give an oil. Purification of this crude oil by chromatography on silica was then performed using an Isco system, eluting with 0-10% ethyl acetate in isohexane gradient conditions over 40 mins to give product that was taken directly onto the next step.

(ii) To this oil (11.2 g, 27.1 mmol) in dichloromethane (200 ml) was added trifluoroacetic acid (TFA) (33.8 ml). The solution was stirred at room temperature overnight. Solvent and TFA were removed in vacuo. The residue was diluted with dichloromethane and made basic by addition of aqueous sodium hydroxide (2M). The organic phase was washed with water (2×) and brine, dried over magnesium sulphate, filtered through celite and evaporated. The oil was taken up in isohexane and ethanol. To this solution was added a solution of fumaric acid (1 eq) in hot ethanol. The mixture was left at room temperature for a few minutes before precipitation occurred. After standing overnight, the resulting precipitate was collected by filtration to give the title compound as a white solid (8.6 g).

### EXAMPLE 90

### N-(2-methylpropyl)-N-{[2-(difluoromethoxy)phenyl]methyl}piperidin-4-amine fumarate

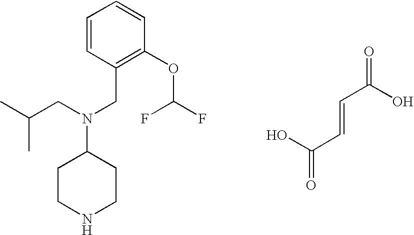

As example 26 with 1,1-dimethylethyl N-[(2-methylpropyl)amino]piperidine-1-carboxylate and 2-difluoromethoxybenzaldehyde, further purified by mass guided preparative LCMS and loaded onto SCX-2 ion exchange cartridge (5 g). The column was washed with methanol (10 ml) and the product eluted with 2M ammonia in methanol solution (10 ml), the solvent was removed in vacuo to give a colourless oil. This oil was taken up in dichloromethane (10 ml) and washed with aqueous saturated potassium carbonate (10 ml). The aqueous layer was extracted with dichloromethane (2×10 ml) and the combined organic layers dried (MgSO₄) and the solvent removed in vacuo to give a colourless oil (159 mg, 34%). The product was taken up in diethyl ether (10 ml) and a few drops of methanol to solubilize and a hot solution of fumaric acid (59 mg, 0.5 mmol, 1 eq) in methanol (0.5 ml) was added. The solution was heated and a few drops of methanol added until all solid was in solution, then the mixture was allowed to slowly cool to 0° C. The product was collected by filtration and dried in a vacuum oven (40° C. for 2 h) to give N-(2-methylpropyl)-N-{[2-(difluoromethoxy)phenyl]methyl}piperidin-4-amine fumarate (122 mg, 19%) as a white solid. OH (300 MHz, MeOD) 7.46-7.43 (1H, m, ArH), 7.21-7.07 (2H, m, ArH), 7.03-7.00 (1H, m, ArH), 6.76 (1H, t, J=74.5 Hz, OCHF₂), 6.58 (2H, s, fumarate CH), 3.62 (2H, s, CH₂Ar) 3.36-3.31 (2H, m, NCH₂), 2.86-2.64 (3H, m, NCH₂ and NCH), 2.19 (2H, d, J=7.2 Hz, NCH₂), 1.93-1.89 (2H, m, CCH₂), 1.75-1.52 (3H, m, CH(CH₃)₂ and CCH₂) and 0.75 (6H, d, J=6.6 Hz, CH₃); LCMS 12 min. Rt=3.20 min, (M⁺+1)=313.2.

### EXAMPLE 91

### N-(3,3-dimethylbutyl)-N-{[5-fluoro-2-(trifluoromethyl)phenyl]methyl}piperidin-4-amine fumarate

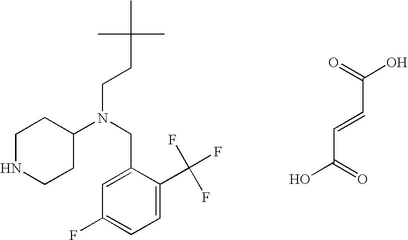

As method previously described for Example 87, using 1,1-dimethylethyl 4-[(3,3-dimethylbutyl)amino]piperidine-1-carboxylate and 5-fluoro-2-trifluoromethylbenzaldehyde. Isolation of the fumarate salt from methanol, ethyl acetate, cyclohexane yielded the title compound as a white solid (0.404 g, 57%). δ_{H} (300 MHz, MeOD) 7.63-7.58 (2H, m, ArH), 7.05-7.01 (1H, m, ArH), 6.58 (2H, s, fumarate CH), 3.76 (2H, s, CH₂Ar), 3.36-3.32 (2H, m, NCH₂), 2.93-2.77 (3H, m, NCH₂, NCH), 2.54-2.48 (2H, m, NCH₂), 2.00-1.93 (2H, m, CCH₂), 1.73-1.60 (2H, m, CCH₂), 1.30-1.25 (2H, m, CH₂tBu), 0.77 (9H, s, CH₃); LCMS 12 mill, Rt=6.1 min, (M⁺+1)=361.

### EXAMPLE 92

### N-(2-ethylbutyl) N-[(2-bromophenyl)methyl]piperidin-4-amine fumarate

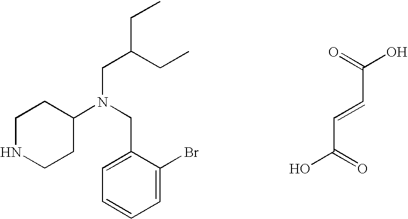

(i) To 10% Pd/C (0.97 g, 10% wt), under nitrogen, was added a solution of the 1-Boc-4-piperidone (9.65 g, 48.44 mmole, 1.0 eq.) and 2-ethyl-n-butylamine (5.00 g, 49.41 mmole, 1.02 eq.) in ethanol (65 ml). This was hydrogenated for 1.5 h, at 65 psi hydrogen, using a Parr hydrogenator. The catalyst was removed by filtration through Celite. Solvent was removed under vacuum to give a pale yellow oil (13.9 g). This was purified by automated flash chromatography using an ISCO Combiflash system (SiO₂ (110 g); 0-10% methanol in ethyl acetate gradient elution over 40 minutes) to give 1,1-dimethylethyl 4-[(2-ethylbutyl)amino]piperidine-1-carboxylate as a colourless oil (8.82 g, 64%), δ_{H} (300 MHz, CDCl₃) 4.03-3.99 (2H, m, NCH₂), 2.85-2.77 (2H, m, NCH₂), 2.61-2.51 (3H, m, NCH, NCH₂), 1.85-1.80 (2H, m, CCH₂), 1.45 (9H, s, OC(CH₃)₃), 1.41-1.186 (7H, m, CCH₂), 0.87 (6H, t, CH₃); LCMS 6 min, Rt=2.6 min, (M⁺+1)=285

(ii) As method previously described for Example 87 using 1,1-dimethylethyl 4-[(2-ethylbutyl)amino]piperidine-1-carboxylate and 2-bromobenzaldehyde. Isolation of the fumarate salt from methanol, diethyl ether, cyclohexane yielded the title compound as a white solid (0.611 g, 87%). δ_{H} (300 MHz, MeOD) 7.47-7.41 (2H, m, Ar), 7.24-7.19 (1H, m, ArH), 7.07-7.01 (1H, m, ArH), 6.58 (2H, s, fumarate CH), 3.66 (2H, s, CH₂Ar), 3.36-3.32 (2H, m, NCH₂), 2.88-2.66 (3H, m, NCH, NCH₂), 2.30 (2H, d, NCH₂), 1.98-1.93 (2H, m, CCH₂), 1.78-1.63 (2H, m, CCH₂), 1.34-1.05 (5H, m, CH(CH₂Me)₂), 0.67 (6H, t, CH₃); LCMS 12 min, Rt=5.3 min, (M⁺+1)=353, 355.

### EXAMPLE 93

### N-(2-ethylbutyl)-N-[(2-methylphenyl)methyl]piperidin-4-amine fumarate

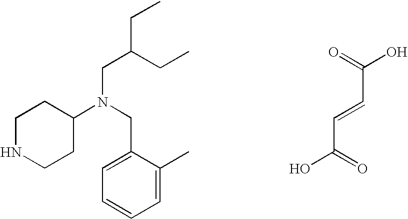

As method previously described for Example 87, using 1,1-dimethylethyl 4-[(2-ethylbutyl)amino]piperidine-1-carboxylate and 2-methylbenzaldehyde. Isolation of the fumarate salt from methanol, diethyl ether, cyclohexane yielded the title compound as a white solid (0.499 g, 82%). δ_{H} (300 MHz, MeOD) 7.19-7.18 (1H, m, ArH), 7.02-7.01 (3H, m, ArH), 6.58 (2H, s, fumarate CH), 3.56 (2H, s, CH₂Ar), 3.36-3.21 (2H, m, NCH₂), 2.85-2.66 (3H, m, NCH₂, NCH), 2.27-2.24 (5H, m, NCH₂, ArCH₃), 1.94-1.90 (2H, m, CCH₂), 1.78-1.65 (2H, m, CCH₂), 1.35-1. (5H, m, CH(CH₂Me)₂), 0.67 (6H, s, CH₃); LCMS 12 min, Rt=4.2 min, (M⁺+1)=289.

### EXAMPLE 94

### N-propyl-N-[(2-chlorophenyl)methyl]piperidin-4-amine fumarate

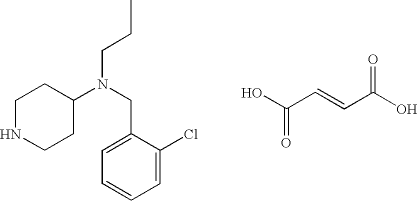

(i) 4-(propylamino)piperidine-1-carboxylic acid tert-butyl ester was prepared with n-propylamine using the method in example 36(i). LCMS-6 mins gradient Rt=1.83 (M⁺+1) 243.3. 1H NMR (CDCl₃) δ=4.10 (2H, brs), 3.75-3.65 (2H, m), 2.81-2.73 (2H, m), 2.61-2.55 (3H, m), 1.91-1.79 (2H, m), 1.46 (9H, s), 1.29-1.21 (4H, m), 0.95-0.92 (2H, m).

(ii) The title product was prepared with 2-chlorobenzaldehye using the method described in example 36(ii). LCMS 12 mins gradient Rt=1.35 (M⁺+1) 262.3. 1H NMR (d6-DMSO) δ=7.64 (1H, m), 7.29 (1H, d, J=7.54 Hz), 7.07 (1H, d, J=7.72 Hz), 6.42 (2H, s), 3.66 (2H, s), 3.25 (2H, brd), 2.78-2.70 (3H, m), 2.41 (3H, s, Me), 2.23 (2H, d, J=7.16 Hz), 1.83-1.57 (5H, m), 0.80 (6H, d, J=6.6 Hz).

### EXAMPLE 95

### N-(3,3-dimethylbutyl)-N-[(2-phenoxyphenyl)methyl]piperidin-4-amine fumarate

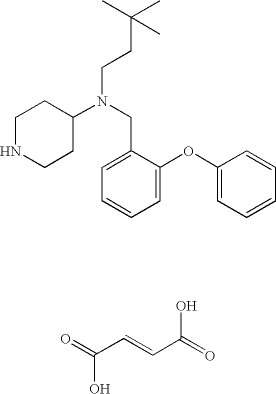

(i) To a 250 ml round bottomed flask was added 2-fluorobenzaldehyde (12.4 g, 100 mmole, 1.0 eq.), phenol (11.3 g, 120 mmole, 1.2 eq.), potassium carbonate (16.6 g, 120 mmole, 1.2 eq.) and dimethylacetamide (100 ml). The reaction mixture was heated at reflux for 16 hours. The mixture was then diluted with water and extracted with diethyl ether. The combined organic extracts were washed with water (3×) and brine. The washed extracts were dried (Na₂SO₄) and concentrated in vacuo to give a yellow oil (15.7 g). This was purified by automated flash chromatography using an ISCO Combiflash system (SiO₂ (120 g×2); 0-10% ethyl acetate in cyclohexane gradient elution over 40 minutes) to give 2-phenoxybenzaldehyde as a colourless oil (14.8 g, 75%). δ_{H} (300 MHz, CDCl₃) 10.52 (1H, s, CHO), 7.94 (1H, dd, ArH), 7.54-7.45 (1H, m, ArH), 7.42-7.36 (2H, m, ArH), 7.21-7.15 (2H, m, ArH), 7.09-7.04 (2H, m, ArH), 6.90 (1H, d, ArH); LCMS 6 min. Rt=4.0 min, (M⁺+1)=199.

(ii) As method previously described for Example 87, using 1,1-dimethylethyl 4-[(3,3-dimethylbutyl)amino]piperidine-1-carboxylate and 2-phenoxybenzaldehyde. Purification of N-(3,3-dimethylbutyl)-N-[(2-phenoxyphenyl)methyl]piperidin-4-amine using the MS-guided preparative LC purification system followed by SCX-2 treatment (to obtain the free base) yielded a colourless oil (0.29 g). Isolation of the fumarate salt (from ethyl acetate) using method described in Example 87 yielded the title compound as a white solid (0.276 g, 38%). δ_{H} (300 MHz, MeOD) 7.47 (1H, dd, ArH), 7.25-7.14 (3H, m, ArH), 7.09-7.04 (1H, m, ArH), 6.99-6.94 (1H, m, ArH), 6.81-6.78 (3H, m, ArH), 6.58 (2H, s, fumarate CH), 3.63 (2H, s, CH₂Ar), 3.31-3.27 (2H, m, NCH₂), 2.83-2.74 (3H, m, NCH₂, NCH), 2.51-2.46 (2H, m, NCH₂), 1.88-1.84 (2H, m, CCH₂), 1.73-1.61 (2H, m, CCH₂), 1.30-1.25 (2H, m, CH₂tBu), 0.75 (9H, s, CH₃); LCMS 12 min, Rt=4.2 min, (M⁺+1) 367.

### EXAMPLE 96

### N-(2-ethylbutyl)-N-[(2-chloro-6-fluorophenyl)methyl]piperidin-4-amine fumarate

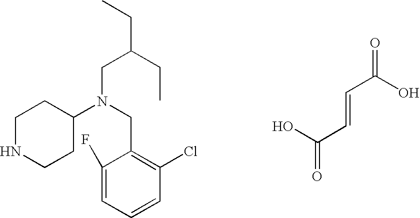

As method previously described for Example 87, using 1,1-dimethylethyl 4-[(2-ethylbutyl)amino]piperidine-1-carboxylate and 2-chloro-6-fluorobenzaldehyde. Isolation of the fumarate salt from methanol, ethyl acetate, cyclohexane yielded the title compound as a white solid (0.232 g, 38%). δ_{H} (300 MHz, MeOD) 7.23-7.12 (2H, m, ArH), 6.99-6.93 (1H, m, ArH), 6.58 (2H, s, fumarate CH), 3.70 (2H, s, CH₂Ar), 3.38-3.34 (2H, m, NCH₂), 2.85 (2H, dt, NCH₂), 2.77-2.69 (1H, m, NCH), 2.25 (2H, d, NCH₂), 1.96-1.92 (2H, m, CCH₂), 1.82-1.68 (2H, m, CCH₂), 1.24-1.13 (5H, m, CH(CH₂Me)₂), 0.63 (6H, t, CH₃); LCMS 12 min, Rt=5.3 min, U+1)=327.

### EXAMPLE 97

### N-(3,3-dimethylbutyl)-N-[(2-biphenyl)methyl]piperidin-4-amine fumarate

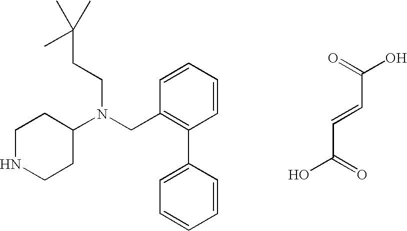

To a 100 ml round bottomed flask, under nitrogen, was added the 1,1-dimethylethyl 4-[(2-bromophenylmethyl)(3,3-dimethylbutyl)amino]piperidine-1-carboxylate (0.675 g, 1.49 mmole, 1.0 eq.), phenylboronic acid (0.363 g, 2.98 mmole, 2.0 eq.), dichlorobis(triphenylphosphine)palladium(II) (0.104 g, 0.15 mmole, 0.1 eq.), sodium carbonate (0.158 g, 2.98 mmole, 2.0 eq.) and a 1:1 mixture of tetrahydrofuran:water (50 ml). The mixture was heated at 90° C. for two hours. The reaction mixture was allowed to cool then poured into diethyl ether (100 ml). This organic mixture was washed with a solution of sodium hydroxide (2M, aqueous, 80 ml) then concentrated in vacuo to give a dark yellow oil (1.18 g). This oil was purified by automated flash chromatography using an ISCO Combiflash system (SiO₂ (120 g); 0-10% methanol (+5% 7M NH₃/MeOH) in dichloromethane gradient elution over 40 minutes) to give a yellow oil (0.683 g). This oil was further purified by automated flash chromatography using an ISCO Combiflash system (SiO₂ (120 g); ethyl acetate gradient elution over 40 minutes) to give 1,1-dimethylethyl 4-[({2-biphenyl}methyl)(3,3-dimethylbutyl)amino]piperidine-1-carboxylate as a yellow oil (0.549 g, 82%). To a solution of this oil (0.549 g, 1.22 mmole, 1.0 eq.) in dichloromethane (10 ml) was added trifluoroacetic acid (TFA) (1.36 ml, 18.27 mmole, 15 eq). The solution was stirred for one hour at room temperature. Solvent and TFA were removed in vacuo. The resulting oil was taken up in methanol and loaded onto an SCX-2 (10 g) column. The column was washed with methanol (50 ml). Basic material was then eluted using 2N ammonia in methanol (50 ml). Removal of solvent from the ammonia/methanol mixture under vacuum, gave a colourless oil (0.27 g). This oil was purified on the Biotage Parallel Flex Purification System (UV-guided HPLC) followed by SCX-2 treatment (to obtain the free base) to give a colourless oil (0.132 g). To a solution of this oil in methanol was added a solution of fumaric acid (0.044 g g, 0.38 mmole, 1 eq) in methanol. The mixture was left to stir for a couple of minutes, ethyl acetate and cyclohexane were then added. The resulting precipitate was collected by filtration to give the title compound as a white solid (0.121 g, 17%). δ_{H} (300 M, MeOD) 7.50-7.47 (1H, m, ArH), 7.35-7.18 (7H, m, ArH), 7.10-7.07 (1H, m, ArH), 6.61 (3H, s, fumarate CH), 3.58 (2H, s, CH₂Ar), 3.25-3.24 (2H, m, NCH₂), 2.74 (2H, dt, NCH₂), 2.67-2.57 (1H, m, NCH), 2.34-2.29 (2H, m, NCH₂), 1.65-1.45 (4H, m, CCH₂), 1.13-1.08 (2H, m, CH₂tBu), 0.70 (9H, s, CH₃); LCMS 12 min, Rt=4.3 min, (M⁺+1)=351.

### EXAMPLE 98

### N-(2-methoxyethyl)-N-4-{[2-(trifluoromethyl)phenyl]methyl}piperidin-4-amine fumarate

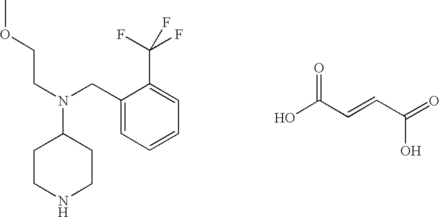

The title product was prepared with 4-(2-methoxyethylamino)piperidine-1-arboxylic acid tert-butyl ester and 2-(trifluoromethyl)benzaldehyde using the method described in example 36(ii). LCMS 12 mins gradient Rt=4.24 (M⁺+1) 287.1. 1H NMR (d6-DMSO) δ=7.92 (1H, d, J=7.54 Hz), 7.68-7.63 (2H, m), 7.43-7.41 (1H, m), 6.44 (2H, s), 3.83 (2H, s), 3.30-3.16 (4H, m), 3.14 (3H, s, OMe), 2.77-2.65 (5H, m), 1.83-1.63 (4H, m).

### EXAMPLE 99

### N-(2-ethylbutyl)-N-{[5-fluoro-2-(trifluoromethyl)phenyl]methyl}piperidin-4-amine fumarate

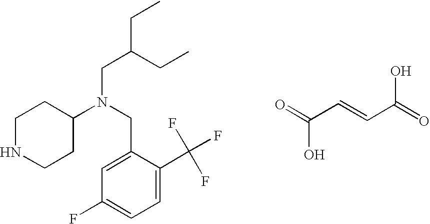

As method previously described for Example 87, using 1,1-dimethylethyl 4-[(2-ethylbutyl)amino]piperidine-1-carboxylate and 5-fluoro-2-trifluoromethylbenzaldehyde. Isolation of the fumarate salt from methanol, ethyl acetate, cyclohexane yielded the title compound as a white solid (0.419 g). Further purified using the Biotage Parallel Flex Purification System (UV-guided HPLC) followed by SCX-2 treatment (to obtain the free base) yielded a colourless oil (0.211 g). This was converted to the fumarate salt as previously to give the title compound as a white solid (0.51 g, 21%). δ_{H} (300 MHz, MeOD) 7.64-7.56 (2H, m, ArH), 7.06-7.02 (1H, m, ArH), 6.59 (2H, s, fumarate CH), 3.76 (2H, s, CH₂Ar), 3.37-3.33 (2H, m, NCH₂), 2.86 (2H, dt, NCH₂), 2.79-2.69 (1H, m, NCH), 2.33 (2H, d, NCH₂), 1.98-1.93 (2H, m, CCH₂), 1.75-1.62 (2H, m, CCH₂), 1.38-1.11 (5H, m, CH(CH₂Me)₂), 0.73 (6H, t, CH₃); LCMS 12 min, Rt=6.4 min, (Oe+1)=361.

### EXAMPLE 100

### N-cyclopentyl-N-{[2-(trifluoromethyl)phenyl]methyl}piperidin-4-amine fumarate

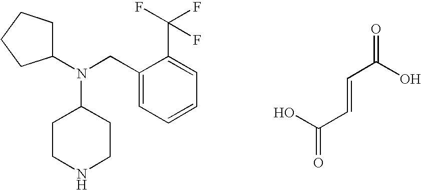

To a solution of 1,1-dimethylethyl 4-({[2-(trifluoromethyl)phenyl]methyl}amino)piperidine-1-carboxylate (1.08 g, 3.0 mmol, 1 eq) and acetic acid (0.17 ml, 3.0 mmol, 1 eq) in 1,2-dichloroethane (15 ml) was added a solution of cyclopentanone (0.8 ml, 9.0 mmol, 3 eq). This was stirred for 30 mins before addition of sodium triacetoxyborahydride (1.90 g, 9.0 mmol, 3 eq), the mixture left to stir for 48 h. The reaction was quenched with water (25 ml), the aqueous layer was separated and extracted with dichloromethane (3×25 ml), the combined organic layers were dried (MgSO₄) and the solvent removed in vacuo to give a colourless oil. This was purified by automated flash chromatography using an ISCO Combiflash system (35 g SiO₂) with a gradient of 040% ethyl acetate in heptane over 30 minutes to give the 1,1-dimethylethyl 4-[{[2-(trifluoromethyl)phenyl]methyl}(cyclopentyl)amino]-piperidine-1-carboxylate (1.032 g, 2.4 mmol, 80%) as a colourless oil. To this was added a solution of anisole (2.8 ml) and trifluoroacetic acid (2.8 ml, 36.6 mmol, 15.2 eq), in dichloromethane (14 ml) and the mixture stirred at room temperature for 16 h. The solvent removed in vacuo and the residue diluted with methanol (5 ml) and loaded onto SCX-2 ion exchange cartridge (10 g). The column was washed with methanol (50 ml) and eluting the product with 2M ammonia in methanol solution (50 ml) the solvent removed in vacuo to give (662 mg, 84%) as a colourless oil. The product was taken up in diethyl ether (30 ml) and a few drops of methanol to solubilize and a hot solution of fumaric acid (235 mg, 2.0 mmol, 1 eq) in methanol (2 ml) was added. The solution was heated and a few drops of methanol added until all solid was in solution, then the mixture was allowed to slowly cool to 0° C. The product was collected by filtration and dried in a vacuum oven (40° C. for 2 h) to give N-cyclopentyl-N-{[2-(trifluoromethyl)phenyl]methyl}piperidin-4-amine fumarate (570 mg, 54%) as a white solid. δ_{H} (300 M, MeOD) 8.06 (1H, d, J=7.7, ArH), 7.66-7.58 (2H, m, ArH), 7.41-7.36 (1H, m, ArH), 6.71 (2H, s, fumarate CH), 3.94 (2H, s, CH₂Ar) 3.45-3.39 (3H, m, NCH₂ and cyclopropyl-NCH), 3.04-2.93 (3H, m, NCH₂ and piperidine-NCH), 2.07-2.02 (2H, m, CCH₂), 1.81-1.45 (10H, m, CH₂); LCMS 12 min, Rt=5.07 min, (M⁺+1)=327.1.

### EXAMPLE 101

### N-(3,3,3-trifluoropropyl)-N-{[2-(trifluoromethyl)phenyl]methyl}piperidin-4-amine fumarate

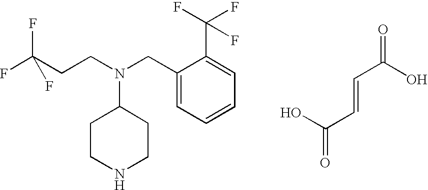

To a solution of 1,1-dimethylethyl 4-({[2-(trifluoromethyl)phenyl]methyl}amino)piperidine-1-carboxylate (1.13 g, 3.1 mmol, 1 eq) and acetic acid (0.17 ml, 3.1 mmol, 1 eq) in 1,2-dichloroethane (10 ml) was added a solution of 3,3,3-trifluoropropanal (1.04 g, 9.3 mmol, 3 eq). This was stirred for 30 mins before addition of sodium triacetoxyborahydride (1.97 g, 9.3 mmol, 3 eq), the mixture left to stir for 72 h. The reaction was quenched with water (10 ml), the aqueous layer was separated and extracted with dichloromethane (2×10 ml), the combined organic layers were washed with potassium carbonate (50 ml), dried (MgSO₄) and the solvent removed in vacuo to give a colourless oil. This was purified by automated flash chromatography using an ISCO Combiflash system (40 g SiO₂) with a gradient of 040% ethyl acetate in heptane over 30 minutes to give the 1,1-dimethylethyl 4-[{[2-(trifluoromethyl)phenyl]methyl}(3,3,3-trifluoropropyl)amino]-piperidine-1-carboxylate (1.109 g, 2.4 mmol, 78%) as a colourless oil. To this was added a solution of anisole (2.8 ml) and trifluoroacetic acid (2.8 ml, 36.6 mmol, 15.2 eq), in dichloromethane (14 ml) and the mixture stirred at room temperature for 16 h. The solvent removed in vacuo and the residue diluted with methanol (5 ml) and loaded onto SCX-2 ion exchange cartridge (10 g). The column was washed with methanol (50 ml) and eluting the product with 2M ammonia in methanol solution (50 ml) the solvent removed in vacuo to give (773 mg, 89%) as a colourless oil. The product was taken up in diethyl ether (30 ml) and a few drops of methanol to solubilize and a hot solution of fumaric acid (327 mg, 2.8 mmol, 1 eq) in methanol (2 ml) was added. The solution was heated and a few drops of methanol added until all solid was in solution, then the mixture was allowed to slowly cool to 0° C. The product was collected by filtration and dried in a vacuum oven (40° C. for 2 h) to give give N-(3,3,3-trifluoropropyl)-N-{[2-(trifluoromethyl)phenyl]methyl}piperidin-4-amine fumarate (885 mg, 78%) as a white solid. δ_{H} (300 MHz, MeOD) 7.92 (1H, d, J=7.7, ArH), 7.71-7.61 (2H, m, ArH), 7.48-7.43 (1H, m, ArH), 6.71 (2H, s, fumarate CH), 3.94 (2H, s, CH₂Ar), 3.50-3.46 (2H, m, NCH₂), 3.35-2.84 (5H, m, NCH₂, NCH and NCH₂), 2.41-2.29 (2H, m, CH₂CF₃), 2.10-2.06 (2H, m, CCH₂) and 1.87-1.75 (2H, m, CCH₂); LCMS 12 min, Rt=5.38 min, (M⁺+1)=355.1.

### EXAMPLE 102

### N-(4,4,4-trifluorobutyl)-N-{[2-(trifluoromethyl)phenyl]methyl}piperidin-4-amine fumarate

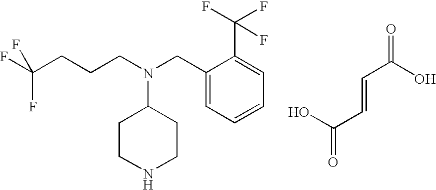

As example 101 with 1,1-dimethylethyl 4-({[2-(trifluoromethyl)phenyl]methyl}amino)piperidine-1-carboxylate and 4,4,4-trifluorobutyraldehyde to give N-(4,4,4-trifluorobutyl)-N-{[2-(trifluoromethyl)phenyl]methyl}piperidin-4-amine fumarate (728 mg, 75%) as a white solid. δ_{H} (300 z, MeOD) 7.77 (1H, d, J=7.9, ArH), 7.58-7.49 (2H, m, ArH), 7.34-7.29 (1H, m, ArH), 6.59 (2H, s, fumarate CI), 3.78 (2H, s, CH₂Ar), 3.36-3.33 (2H, m, NCH₂), 2.89-2.72 (3H, m, NCH₂ and NCH), 2.54 (2H, t, J=6.8 Hz, NCH₂), 2.08-1.92 (4H, m, CH₂) and 1.74-1.51 (4H, m, CH₂); LCMS 12 min, Rt=5.53 min, (M⁺+1)=369.1.

### EXAMPLE 103

### N-(2,2-dimethylpropyl)-N-{[2-(trifluoromethyl)phenyl]methyl}piperidin-4-amine fumarate

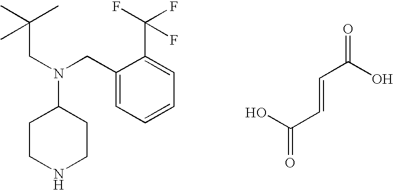

As example 101 with 1,1-dimethylethyl 4-({[2-(trifluoromethyl)phenyl]methyl}amino)piperidine-1-carboxylate and trimethylacetaldehyde to give N-(2,2-dimethylpropyl)-N-{[2-(trifluoromethyl)phenyl]methyl}piperidin-4-amine fumarate (493 mg, 56%) as a white solid. δ_{H} (300 MHz, MeOD) 8.00 (1H, d, J=7.7 Hz, ArH), 7.56-7.51 (2H, m, ArH), 7.33-7.18 (1H, m, ArH), 6.59 (2H, s, fumarate CH), 3.90 (2H, s, CH₂Ar), 3.35-3.30 (2H, m, NCH₂), 2.81-2.93 (2H, m, NCH₂), 2.59-2.51 (1H, m, NCH), 2.32 (2H, s, NCH₂), 2.00-1.94 (2H, m, CCH₂), 1.71-1.60 (2H, m, CCH₂) and 0.80 (9H, d, CH₃); LCMS 12 min, Rt=5.91 min, (M⁺+1)=329.2.

### EXAMPLE 104

### N-(2-methylpropyl-N-{[2-(trifluoromethyl)phenyl]ethyl}piperidin-4-amine fumarate

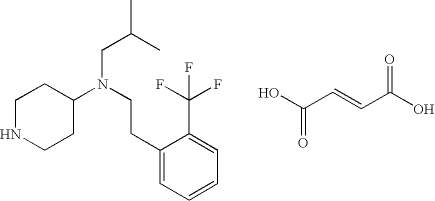

(i) 2-(Trifluoromethyl)phenethyl alcohol (3.00 g, 15.77 mmol) in dry dichloromethane (100 ml) was added pyridinium chlorochromate (4.08 g, 18.93 mmol) in one portion at room temperature, under an atmosphere of nitrogen. The orange mixture turns black after 20 mins. The reaction was monitored by thin layer chromatography (100% diethyl ether; reactant r.f. 0.5, product r.f. 0.9). After 1 hr the solvent was evaporated in vacuo to give a black oil and this was taken up in diethyl ether (100 ml) and filtered through a pad of silica and eluted with diethyl ether (100 ml). The filtrate was taken and concentrated in vacuo to a yellow oil (1.91 g, 10.09 mmol). The (2-trifluoromethylphenyl)acetaldehyde was taken to the next step without any purification. δ_{H} (300 MHz, CDCl₃) 9.75 (1H, S), 7.87-7.23 (4H, m), 3.94 (2H, s).

(ii) The compound was prepared similarly to example 63 using 4-isobutylamino-piperidine-1-carboxylic acid tert-butyl ester (0.50 g, 1.95 mmol) and (2-trifluoromethyl-phenyl)-acetaldehyde (1.09 g, 5.85 mmol) to give the title compound as an off-white solid (0.53 g, 1.19 mmol). δ_{H} (300 M, MeOD) 7.61-7.36 (4H, m), 6.69 (2H, s, fumarate), 3.48 (2H, m), 3.00 (5H, m), 2.72 (2H, m), 2.32 (2H, d), 1.98 (2H, m), 1.73 (3H, m), 0.92 (6H, d). LCMS 12 minute gradient, Rt=3.44 mins, (M⁺+1)=329.2

### EXAMPLE 105

### N-(2-methylpropyl)-N-{[2-(methylsulphonyl)phenyl]methyl}piperidin-4-amine D-tartrate

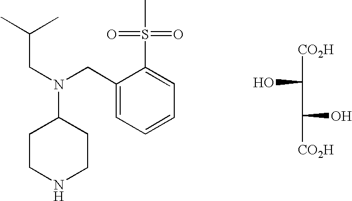

(i) 2-fluorobenzaldehyde (12.4 g, 0.1 mol, 1 eq) and methanesulphinic acid (11.2 g, 0.11 mol, 1.1 eq) were dissolved in DMSO (75 ml) and heated to 100° C. for 16 h. The reaction mixture was cooled to room temperature and poured onto crushed ice (100 g). The product was collected by filtration and dried in a vacuum oven at 45° C. for 16 h to give 2-(methylsulphonyl)benzaldehyde (9.2 g, 50%) as a yellow solid. δ_{H} (300 MHz, CDCl₃) 10.77 (1H, s, CHO), 8.18-8.16 (1H, m, ArH), 8.10-8.08 (1H, m, Arm), 7.83-7.80 (2H, m, ArH) and 3.28 (3H, s, SO₂CH₃); LCMS 6 min, Rt=1.93 min, (M⁺+1)=185.1.

(ii) To a solution of 1,1-dimethylethyl 4-[(2-methylpropyl)amino]piperidine-1-carboxylate (0.38 g, 1.5 mmol, 1 eq) in 1,2-dichloroethane (10 ml) was added 2-(methylsulphonyl)benzaldehyde (829 mg, 4.5 mmol, 3 eq), after 15 minutes sodium triacetoxyborahydride (0.95 g, 4.5 mmol, 3 eq) was added and the mixture left to stir for 16 h. The reaction was quenched with water (10 ml), the aqueous layer was separated and extracted with dichloromethane (2×20 ml). The combined organic layers were washed with brine (20 ml), dried (MgSO₄) and the solvent removed in vacuo. This was purified by automated flash chromatography using an ISCO Combiflash system (40 g SiO₂) with a gradient of 0-40% ethyl acetate in heptane over 30 minutes to give 1,1-dimethylethyl 4-[{[2-(methylsulphonyl)phenyl]methyl}(2-methylpropyl)amino]piperidine-1-carboxylate (0.41 g, 1.0 mmol, 65%) as a colourless oil. To this was added a solution of anisole (1.4 ml) and trifluoroacetic acid (1.4 ml, 18.3 mmol, 18.3 eq), in dichloromethane (7 ml) and the mixture stirred at room temperature for 16 h. The solvent removed in vacuo and the residue diluted with methanol (5 ml) and loaded onto SCX-2 ion exchange cartridge (5 g). The column was washed with methanol (15 ml) and eluting the product with 2M ammonia in methanol solution (15 ml) the solvent removed in vacuo to give (199 mg, 0.6 mmol, 60%) as a colourless oil. The product was taken up in cyclohexane/isopropanol (30 ml) and a hot solution of D-tartaric acid (92 mg, 0.6 mmol, 1 eq) in isopropanol (2 ml) was added. The solvent was removed in vacuo and the gum triturated with diethyl ether (3×20 ml) to give the title compound (278 mg, 60%) as a yellow solid. δ_{H} (300 MHz, MeOD) 7.93-7.83 (2H, m, ArH), 7.61-7.58 (1H, m, ArH), 7.42-7.37 (1H, m, ArH), 4.31 (2H, s, tartrate CH), 4.06 (2H, s, CH₂Ar), 3.36-3.32 (2H, m, NCH₂), 3.13 (3H, s, SO₂CH₃), 2.84-2.67 (3H, m, NCH and NCH₂), 2.27 (2H, d, J=7.0 Hz, NCH₂), 1.97-1.92 (2H, m, CCH₂), 1.76-1.56 (3H, m, CCH₂ and CH(CH₃)₂) and 0.80 (6H, d, J=6.6 Hz, CH₃); LCMS 12 min, Rt=3.03 min, (M⁺+1)=325.1.

### EXAMPLE 106

### N-(2-ethylbutyl)-N-[(2-biphenyl)methyl]piperidin-4-amine fumarate

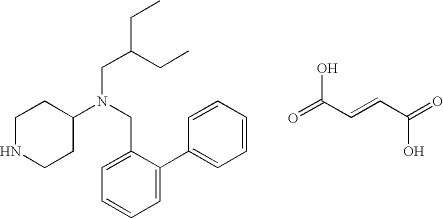

As method previously described for Example 97, using 1,1-dimethylethyl 4-[(2-bromophenylmethyl)(2-ethylbutyl)amino]piperidine-1-carboxylate. Isolation of the fumarate salt from methanol, diethyl ether, cyclohexane yielded the title compound as a white solid (0.238 g, 34%). δ_{H} (300 M, MeOD) 7.59-7.57 (1H, m, ArH), 7.45-7.27 (7H, m, ArH), 7.19-7.16 (1H, m, ArH), 6.69 (1.5H, s, fumarate CH), 3.62 (2H, s, CH₂Ar), 3.34-3.32 (2H, m, NCH₂), 2.79 (2H, dt, NCH₂), 2.66-2.57 (1H, m, NCH), 2.21 (2H, d, NCH₂), 1.64-1.50 (4H, m, CCH₂), 1.38-1.17 (5H, m, CH(CH₂Me)₂), 0.78 (6H, t, CH₃); LCMS 12 min, Rt=5.1 min, (M⁺=1)=351.

### EXAMPLE 107

### N-(cyclohexylmethyl)-N-[(2-biphenyl)methyl]piperidin-4-amine fumarate

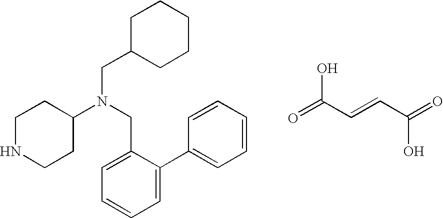

(i) To a solution of cyclohexylmethylamine (0.461 g, 4.08 mmole, 1.02 eq.) in 1,2-dichloroethane (10 ml) was added 1-Boc-4-piperidone (0.797 g ml, 4.00 mmole, 1.0 eq.). To this was added a solution of sodium triacetoxyborohydride (0.865 g, 4.08 mmole, 1.02 eq.) in dimethylformamide (2 ml). This mixture was left to stir under nitrogen, at room temperature, over the weekend. To the reaction mixture was then added water (10 ml) and the mixture stirred vigorously for several minutes. The chlorinated organic layer was then run through a hydrophobic frit then diluted with methanol (10 ml) and loaded onto an SCX-2 (10 g) column. The column was washed with methanol (50 ml) then basic material eluted with 2N ammonia in methanol. The ammonia/methanol solution was concentrated in vacuo to give a pale yellow oil (1.2 g). This was purified by automated flash chromatography using an ISCO Combiflash system (SiO₂ (40 g); 0-10% methanol in ethyl acetate gradient elution over 40 minutes) to give 1,1-dimethylethyl 4-[(cyclohexylmethyl)amino]piperidine-1-carboxylate as a colourless oil (0.98 g, 83%). δ_{H} (300 MHz, CDCl₃) 4.03-4.00 (2H, m, NCH₂), 2.83-2.75 (21, m, NCH₂), 2.60-2.49 (1H, m, NCH), 2.45 (2H, d, NCH₂), 1.18-0.83 (15H, m, CCH₂), 1.45 (9H, s, OC(CH₃)₃); LCMS 6 min, Rt=2.7 min, (M⁺+1)=297.

(ii) To a solution of 1,1-dimethylethyl 4-[(cyclohexylmethyl)amino]piperidine-1-carboxylate (0.245 g, 0.840 mmole, 1.0 eq.), 2-phenylbenzyl bromide (0.185 ml, 1.01 mmole, 1.2 eq.) in dry acetonitrile (5 ml) was added anhydrous potassium carbonate (0.19 g, 1.35 mmole, 1.6 eq.). The mixture was stirred overnight at room temperature. The reaction mixture was concentrated under vacuum to give a white solid. The white solid was taken up in dichloromethane (10 ml) and this washed with water (10 ml). The dichloromethane layer was passed through a hydrophobic frit then diluted with methanol (10 ml). This solution was loaded onto an SCX-2 (10 g) column. The column was washed with methanol (50 ml) then basic material was eluted using 2N ammonia in methanol (50 ml). Concentration of the ammonia/methanol solution under vacuum yielded a colourless oil (0.344 g, 90%). To a solution of this oil (0.344 g, 0.74 mmole, 1.0 eq.) in dichloromethane (10 ml) was added trifluoroacetic acid (TFA) (0.83 ml, 11.2 mmole, 15 eq). The solution was stirred overnight at room temperature. Solvent and TFA were removed in vacuo. The resulting oil was taken up in methanol and loaded onto an SCX-2 (10 g) column. The column was washed with methanol (50 ml). Basic material was then eluted using 2N ammonia in methanol (50 ml). Removal of solvent from the ammonia/methanol mixture under vacuum, gave a colourless oil (0.298 g, 99%). The oil was taken up in methanol. To this solution was added a solution of fumaric acid (0.095 g, 0.08 mmole, 1 eq) in methanol followed by diethyl ether and cyclohexane. The resulting precipitate was collected by filtration to give the title compound as a white solid (0.302 g, 76%). δ_{H} (300 z, MeOD) 7.58 (1H, d, ArH), 7.45-7.29 (7H, m, ArH), 7.18 (1H, d, ArH), 6.70 (2H, s, fumarate CH), 3.64 (2H, s, CH₂Ar), 3.33-3.32 (2H, m, NCH₂), 2.79 (2H, dt, NCH₂), 2.65-2.54 (1H, m, NCH), 2.17 (2H, d, NCH₂), 1.74-1.47 (9H, m, CCH₂), 1.28-1.11 (4H, m, CH, CCH₂), 0.78-0.67 (2H, m, CH₂); LCMS 12 min, Rt=5.0 min, (M⁺+1)=363.

### EXAMPLE 108

### N-(2-ethylbutyl)-N-[(2-phenoxyphenyl)methyl]piperidin-4-amine fumarate

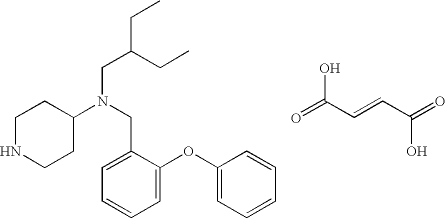

As method previously described for Example 87, using 2-phenoxybenzaldehyde and 1,1-dimethylethyl 4-[(2-bromophenylmethyl)(2-ethylbutyl)amino]piperidine-1-carboxylate. Isolation of the fumarate salt from methanol, diethyl ether, cyclohexane yielded the title compound as a white solid (0.562 g, 78%). δ_{H} (300 MHz, MeOD) 7.56 (1H, dd, ArH), 7.36-7.24 (3H, m, ArH), 7.19-7.15 (1H, m, ArH), 7.09-7.04 (1H, m, ArH), 6.92-6.89 (3H, m, ArH), 6.70 (2H, s, fumarate CH), 3.68 (2H, s, CH₂Ar), 3.41-3.37 (2H, m, NCH₂), 2.89-2.77 (3H, m, NCH, NCH₂), 2.35 (2H, d, NCH₂) 1.93-1.89 (2H, m, CCH₂), 1.81-1.67 (2H, m, CCH₂), 1.47-1.24 (5H, m, CH(CH₂Me)₂), 0.80 (6H, t, CH₃); LCMS 12 min, Rt=4.8 min, (M⁺+1)=367.

### EXAMPLE 109

### N-(2-methylpropyl)-N-[(2-phenoxyphenyl)methyl]piperidin-4-amine fumarate

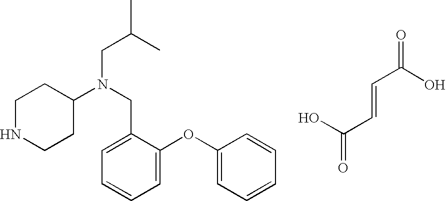

As method previously described for Example 87, using 2-phenoxybenzaldehyde and 1,1-dimethylethyl 4-[(2-methylpropyl)amino]piperidine-1-carboxylate. Isolation of the fumarate salt from methanol, diethyl ether, cyclohexane yielded the title compound as a white solid (0.521 g, 79%). δ_{H} (300 MHz, MeOD) 7.58 (1H, dd, ArH), 7.36-7.24 (3H, m, ArH), 7.20-7.14 (1H, m, ArH), 7.09-7.04 (1H, m, ArH), 6.91-6.88 (3H, m, ArH), 6.70 (2H, s, fumarate CH), 3.68 (2H, s, CH₂Ar), 3.40-3.31 (2H, m, NCH₂), 2.89-2.75 (3H, m, NCH, NCH₂), 2.27 (2H, d, NCH₂), 1.93-1.89 (2H, m, CCH₂), 1.78-1.63 (3H, m, CCH₂, CHMe₂), 1.47-1.24 (5H, m, CH(CH₂Me)₂), 0.86 (6H, d, CH₃); LCMS 12 min, Rt=4.0 min, (M⁺+1)=339.

### EXAMPLE 110

### N-(cyclohexylmethyl)-N-[(2-phenoxyphenyl)methyl]piperidin-4-amine fumarate

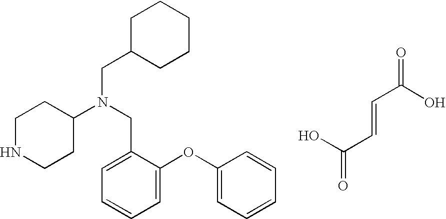

As method previously described for Example 87, using 2-phenoxybenzaldehyde prepared as for Example 95 and 1,1-dimethylethyl 4-[(cyclohexylmethyl)amino]piperidine-1-carboxylate as prepared for Example 107. Isolation of the fumarate salt from methanol, ethyl acetate, cyclohexane yielded the title compound as a white solid (0.155 g, 37%). δ_{H} (300 MHz, MeOD) 7.59 (1H, dd, ArH), 7.42-27 (3H, m, ArH), 7.23-7.18 (1H, m, ArH), 7.12-7.07 (1H, m, ArH), 6.95-6.92 (2H, m, ArH), 6.72 (3H, s, fumarate CH), 3.71 (2H, s, CH₂Ar), 3.43-3.37 (2H, m, NCH₂), 2.92-2.77 (3×, m, NCH, NCH₂), 2.33 (2H, d, NCH₂), 1.95-1.68 (9H, m, CCH₂), 1.50-1.38 (1H, m, CH), 1.28-1.18 (3H, m, CCH₂), 0.86-0.78 (2H, m, CH₂); LCMS 12 min, Rt=4.7 min, (M⁺+1)=379.

### EXAMPLE 111

### N-(1-ethylpropyl)-N-{[2-(trifluoromethyl)phenyl]methyl}piperidin-4-amine fumarate

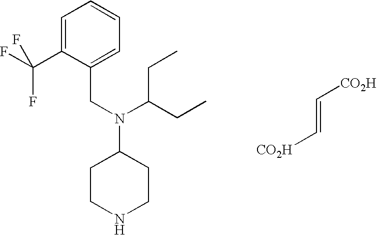

(i) To a solution of 1-Boc-4-piperidone (4.98 g, 25 mmol, 1 eq) in 1,2-dichloroethane (45 ml) was added 1-ethylpropylamine (2.91 ml, 25 mmol, 1 eq) and the solution stirred for 15 minutes. Sodium triacetoxyborahydride (5.29 g, 25 mmol, 1 eq) was added and the reaction mixture stirred for a further 16 hours. The mixture was diluted with water (25 ml) and saturated potassium carbonate (25 ml), then extracted with dichloromethane (3×50 ml). The combined organic extracts were washed with brine (100 ml), dried (MgSO₄) and the solvent removed in vacuo to give a yellow oil which was purified by automated flash chromatography using an ISCO Combiflash system (120 g SiO₂) with a gradient of 0-30% methanol in ethyl acetate over 30 minutes to give the 1,1-dimethylethyl 4-[(1-ethylpropyl)amino]piperidine-1-carboxylate (2.84 g, 42%) as a colourless oil. δ_{H} (300 MHz, CDCl₃) 4.03-3.92 (2H, m, NCH₂), 2.84-2.77 (2H, m, NCH₂), 2.69-2.60 (1H, m, NCH) 2.51-2.43 (1H, m, NCH), 1.84-1.79 (2H, m, CCH₂), 1.52-1.14 (15H, m, CH₂ and C(CH₃)₃) and 0.87 (6H, t, J=7.4 Hz, CH₃); LCMS 12 min, Rt=2.45 min, (M⁺+1) 271.3.

(ii) To a solution of 1,1-dimethylethyl 4-[(1-ethylpropyl)amino]piperidine-1-carboxylate (300 mg, 1.1 mmol, 1 eq) and 2-(trifluoromethyl)benzyl bromide (0.2 ml, 1.3 mmol, 1.2 eq) in acetonitrile (5 ml) was added potassium carbonate (243 mg, 1.7 mmol, 1.6 eq). The mixture was refluxed for 4 days then cooled to room temperature, diluted with water (10 ml) and extracted with dichloromethane (3×10 ml). The combined organic extracts were dried (MgSO₄) and the solvent removed in vacuo to give an oil which was diluted with methanol (5 ml) and loaded onto SCX-2 ion exchange cartridge (5 g). The column was washed with methanol (15 ml) and eluting the product with 2M ammonia in methanol solution (15 ml) the solvent removed in vacuo to give 1,1-dimethylethyl 4-[{[1-(trifluoromethyl)phenyl]methyl}(2-ethylpropyl)amino]-piperidine-1-carboxylate (275 mg, 0.6 mmol, 58%) as a colourless oil. To this was added a solution of anisole (1 ml) and trifluoroacetic acid (1 ml, 13.1 mmol, 21.2 eq), in dichloromethane (5 ml) and the mixture stirred at room temperature for 16 h. The solvent removed in vacuo and the residue diluted with methanol (5 ml) and loaded onto SCX-2 ion exchange cartridge (5 g). The column was washed with methanol (15 ml) and eluting the product with 2M ammonia in methanol solution (15 ml) the solvent removed in vacuo. This was further purified by MS guided preparative LC then loaded onto SCX-2 ion exchange cartridge (5 g). The column was washed with methanol (15 ml) and eluting the product with 2M ammonia in methanol solution (15 ml) the solvent removed in vacuo to give (134 mg, 68%) as a colourless oil. The product was taken up in diethyl ether (10 ml) and a few drops of methanol to solubilize and a hot solution of fumaric acid (47 mg, 2.0 mmol, 1 eq) in methanol (1 ml) was added. The solution was heated and a few drops of methanol added until all solid was in solution, then the mixture was allowed to slowly cool to 0° C. The product was collected by filtration and dried in a vacuum oven (40° C. for 2 h) to give N-(1-ethylpropyl)-N-{[2-(trifluoromethyl)phenyl]methyl}piperidin-4-amine fumarate (90 mg, 34%) as a white solid. δ_{H} (300 z, MeOD) 7.95 (1H, d, J=7.9 Hz, ArH), 7.66-7.58 (2H, m, ArH), 7.42-7.37 (1H, m, ArH), 6.70 (2H, s, fumarate CH), 3.96 (2H, s, CH₂Ar), 3.45-3.41 (2H, m, NCH₂), 3.00-2.86 (3H, m, NCH and NCH₂), 2.35-2.30 (1H, m, NCH), 2.07-2.02 (2H, m, CCH₂), 1.87-1.76 (2H, m, CCH₂), 1.60-1.39 (4H, m, CH₂) and 0.96 (6H, t, J=7.46 Hz, CH₃); LCMS 12 min, Rt=3.52 min, (M⁺+1)=329.2.

### EXAMPLE 112

### N-butyl-N-{[2-(trifluoromethyl)phenyl]methyl}piperidin-4-amine fumarate

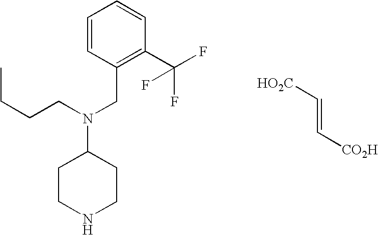

(i) A mixture of 1-tert-butoxycarbonyl-4-piperidone (25 g, 126 mmol), n-butylamine (11 g, 151 mmol) and 10% palladium on carbon (2.5 g) in ethanol (250 ml) was hydrogenated on a Parr apparatus for 1.5 h at 65 psi. The reaction mixture was filtered through a pad of celite and the filtrate evaporated to give 4-butylamino-piperidine-1-carboxylic acid tert-butyl ester as a colourless liquid (32 g). LCMS 6 min Rt=2.48 min m/e 257 (M⁺+H).

(ii) Sodium triacetoxyborohydride (11.4 g, 54 mmol) was added to a stirred solution of 4-butylamino-piperidine-1-carboxylic acid tert-butyl ester (9.2 g, 36 mmol) and 2-trifluoromethylbenzaldehyde (8.0 g 46 mmol) in 1,2-dichloroethane (100 ml) at room temperature under nitrogen atmosphere. After 16 h, water (100 ml) was added and the mixture stirred vigorously. The organic layer was separated and the aqueous solution was extracted with dichloromethane (50 ml). The combined organic solutions were washed with water, dried, filtered and evaporated to a semi-solid (20.5 g). Suspended in ethyl acetate (5 ml) iso-hexane (50 ml) and the solid filtered, washed iso-hexane. The filtrate was evaporated give the crude product as a colourless liquid (15.2 g). Purifed by flash chromatography eluting with 5-10% ethyl acetate in iso-hexane gave the product as an oil (10.0 g, 68%). LCMS 6 min, Rt=4.0 min, m/e 415 (M⁺+H).

(iii) Trifluoroacetic acid (18.3 ml, 237 mmol) was added to stirred solution of the above oil (9.8 g, 23.7 mmol) in dichloromethane (40 ml) at room temperature. After 1 h, diluted with dichloromethane (100 ml) and basified with aqueous sodium hydroxide (2M, 120 ml). The dichloromethane layer was separated, washed with water (100 ml), dried, filtered and evaporated to a colourless oil of N-butyl-N-{[2-(trifluoromethyl)phenyl]methyl}piperidin-4-amine (8.0 g). The free base in ethanol (40 ml) iso-hexane (40 ml) was converted to the fumarate salt by addition of a hot solution of fumaric acid (2.55 g) in ethanol (40 ml). Crystallised on standing as white flakes of the title product (8.60, 84%). LCMS 12 min, Rt=4.3 min, m/e 315 (M⁺+H). δ_{H} (300 MHz, d6-DMSO) 7.74 (1H, d), 7.46-7.55 (2H, m), 7.28 (1H, t), 6.28 (2H, s), 3.60 (2H, s), 3.10 (2H, d), 2.61 (3H, t), 2.26-2.33 (2H, m), 1.43-1.71 (4H, m), 1.00-1.21 (4H, m), 0.64 (3H, t).

### EXAMPLE 113

### N-(cyclopropylmethyl)-N-[(2-biphenyl)methyl]piperidin-4-amine fumarate

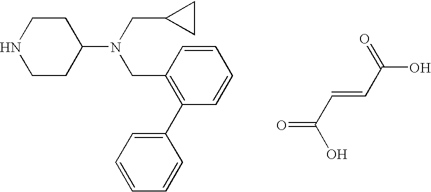

As method previously described for Example 107, using 1,1-dimethylethyl 4-[(cyclopropylmethyl)amino]piperidine-1-carboxylate and 2-phenylbenzyl bromide. Isolation of the fumarate salt from methanol and diethyl ether yielded the title compound as a white solid (0.485 g, 74%). δ_{H} (300 MHz, MeOD) 7.68 (1H, dd, ArH), 7.47-7.29 (7H, m, ArH), 7.21 (1H, d, ArH), 6.72 (2H, s, fumarate CH), 3.76 (2H, s, CH₂Ar), 3.38-3.34 (2H, m, NCH₂), 2.92-2.82 (3H, m, NCH, NCH₂), 2.32 (2H, d, NCH₂), 1.79-1.57 (4H, m, CCH₂), 0.77-0.66 (1H, m, CH), 0.46-0.40 (2H, m, CH₂), 0.03-⁻0.02 (2H, m, CH₂); LCMS 12 min, Rt=3.5 min, (M⁺+1)=321.

### EXAMPLE 114

### N-(cyclopropylmethyl)-N-[(2-phenoxyphenyl)methyl]piperidin-4-amine fumarate

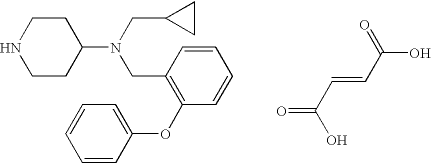

As method previously described for Example 87 using 1,1-dimethylethyl 4-[(cyclopropylmethyl)amino]piperidine-1-carboxylate and 2-phenoxybenzaldehyde. Isolation of the fumarate salt from methanol and diethyl ether yielded the title compound as a white solid (0.586 g, 86%). δ_{H} (300 M, MeOD) 7.54 (1H, dd, ArH), 7.28-7.16 (3H, m, ArH), 7.12-7.07 (1H, m, ArH), 7.02-6.96 (1H, m, ArH), 6.85-6.80 (3H, m, ArH), 6.60 (2H, s, fumarate CH), 3.75 (2H, s, CH₂Ar), 3.34-3.30 (2H, m, NCH₂), 3.04-2.96 (1H, m, NCH), 2.86-2.77 (2H, m, NCH₂), 2.41 (2H, d, NCH₂), 1.92-1.86 (2H, m, CCH₂), 1.77-1.63 (2H, m, CCH₂), 0.84-0.74 (1H, m, CH), 0.43-0.37 (2H, m, CH₂), 0.03-⁻0.02 (2H, m, CH₂); LCMS 12 min, Rt=3.6 min, ++1)=337.

### EXAMPLE 115

### N-(2-methoxyethyl-N-[(2-methylthio)methyl]piperidin-4-amine fumarate

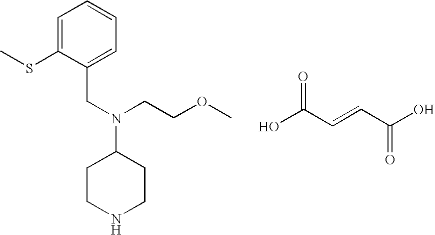

(i) 4-(2-methoxyethylamino)piperidine-1-carboxylic acid tert-butyl ester was prepared with 2-methoxyethylamine by the method in example 36(i). LCMS-6 mins gradient Rt=2.14 (M⁺+1) 259.3. 1H NMR (CDCl₃) δ=4.10 (2H, brs), 3.56-3.50 (2H, m), 3.45 (3H, s, OMe), 2.90-2.69 (3H, m), 2.68-2.61 (1H, m), 1.85-1.80 (4H, m), 1.46 (9H, s), 1.38-1.22 (2H, m).

(ii) The title product was prepared with 4-(2-methoxyethylamino)piperidine-1-carboxylic acid tert-butyl ester and 2-(methylthio)benzaldehyde using the method described in example 36(ii). LCMS 12 mins gradient Rt=2.63 M++1) 295.1. 1H NMR (d6-DMSO) δ=7.45 (1H, d), 7.26-7.25 (2H, m), 7.18-7.10 (1H, m), 6.42 (2H, s), 3.63 (2H, s), 3.31-3.15 (4H, m), 3.15 (3H, s, Ar—SMe), 2.81-2.68 (3H, m), 2.65-2.60 (2H, m), 2.40 (3H, s, CH₂—OMe), 1.90-1.75 (2H, m), 1.74-1.60 (2H, m).

### EXAMPLE 116

### N-(2-methoxyethyl)-N-{[2-(difluoromethoxy)phenyl]methyl}piperidin-4-amine fumarate

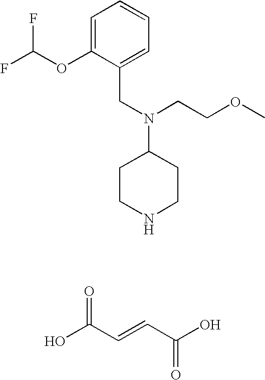

The title product was prepared with 4-(2-methoxyethylamino)piperidine-1-carboxylic acid tert-butyl ester and 2-(difluoromethoxy)benzaldehyde using the method described in example 36(ii). LCMS 12 mins gradient Rt=2.50 (M⁺+1) 315.1. 1H NMR (d6-DMSO) δ=7.54 (1H, d, J=7.34 Hz), 7.42-7.31 (2H, m), 7.29-7.13 (1H, m), 6.42 (2H, s), 3.68 (2H, s), 3.27-3.23 (4H, m), 3.13 (3H, s, OMe), 2.90-2.65 (3H, m), 2.63-2.61 (2H, m), 1.82-1.72 (2H, m), 1.68-1.65 (2H, m).

### EXAMPLE 117

### N-(2-methoxyethyl)-N-[(2-methyl)methyl]piperidin-4-amine fumarate

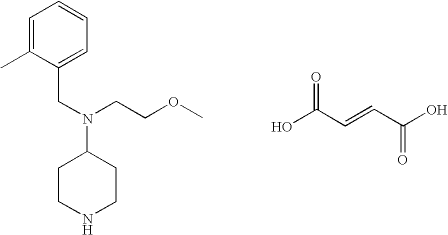

The title product was prepared with 4-(2-methoxyethylamino)piperidine-1-carboxylic acid tert-butyl ester and 2-methylbenzaldehyde using the method described in example 36(ii). LCMS 12 mins gradient Rt=2.49 (M⁺+1) 263.1. 1H NMR (d6-DMSO) δ=7.33-7.32 (1H, m), 7.15-7.12 (3H, m), 6.43 (2H, s), 3.63 (2H, s), 3.26-3.17 (4H, m), 3.13 (3H, s, Ar-Me), 2.76-2.71 (3H, m), 2.69-2.58 (2H, m), 2.29 (3H, s, OMe), 1.83-1.79 (2H, m), 1.71-1.68 (2H, m).

### EXAMPLE 118

### N-(2-methoxyethyl)-N-[(2-chlorophenyl)methyl]piperidin-4-amine fumarate

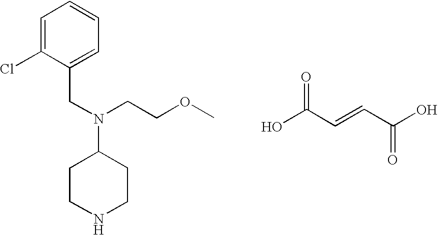

The title product was prepared with 4-(2-methoxyethylamino)piperidine-1-carboxylic acid tert-butyl ester and 2-chlorobenzaldehyde using the method described in example 36(ii). LCMS 12 mins gradient Rt=2.75 (M⁺+1) 283.1. 1H NMR (d6-DMSO) 3=7.60 (1H, d, J=6.03 Hz), 7.40-7.23 (3H, m), 6.42 (2H, s), 3.74 (2H, s), 3.38-3.22 (4H, m), 3.15 (3H, s, OMe), 2.78-2.65 (5H, m), 1.84-1.80 (2H, m), 1.72-1.65 (2H, m).

### EXAMPLE 119

### N-(2-methoxyethyl)-N-{[4-fluoro-2-(trifluoromethyl)phenyl]methyl}piperidin-4-amine fumarate

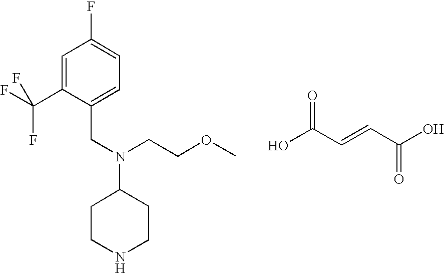

The title product was prepared with 4-(2-methoxyethylamino)piperidine-1-carboxylic acid tert-butyl ester and 4-fluoro-2-(trifluoromethyl)benzaldehyde using the method described in example 36(ii). LCMS 12 mins gradient Rt=4.65 (M⁺+1) 335.1. 1H NMR (d6-DMSO) δ=7.97-7.92 (1H, m), 7.56-7.50 (2H, m), 6.42 (2H, s), 3.80 (2H, s), 3.39-3.22 (4H, m), 3.14 (3H, s, OMe), 2.74-2.64 (5H, m), 1.82-1.60 (4H, m).

### EXAMPLE 120

### N-(2-methoxyethyl)-N-[(2,4-dichlorophenyl)methyl]piperidin-4-amine fumarate

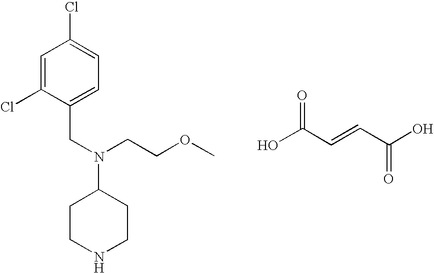

The title product was prepared with 4-(2-methoxyethylamino)piperidine-1-carboxylic acid tert-butyl ester and 2,4-dichlorobenzaldehyde using the method described in example 36(ii). LCMS 12 mins gradient Rt=4.05++1) 317.1. 1H NMR (d6-DMSO) δ=7.62 (1H, d, J=8.29 Hz), 7.54 (1H, d, J=2.07 Hz), 7.41 (1H, dd, J=2.07 Hz, J=2.07 Hz) 6.42 (2H, s), 3.72 (2H, s), 3.38-3.22 (4H, m), 3.15 (3H, s, OMe), 2.91-2.65 (5H, m), 1.83-1.64 (4H, m).

### EXAMPLE 121

### N-(cyclopropylmethyl)-N-[(2-methylthiophenyl)methyl]piperidin-4-amine fumarate

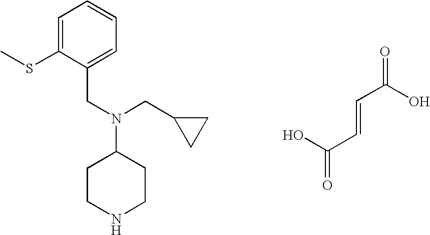

(i) 4-(cyclopropylmethylamino)piperidine-1-carboxylic acid tert-butyl ester was prepared with cyclopropylmethylamine by the method in example 36(i). 1H NMR (CDCl₃) δ=4.10 (2H, brs), 2.81-2.61 (3H, m), 2.51 (2H, d), 1.80-1.71 (4H, m), 1.49 (9H, s), 1.38-1.21 (2H, m), 1.01-0.95 (1H, m), 0.55-0.45 (2H, m), 0.15-0.10 (2H, m).

(ii) The title product was prepared with 2(methylthio)benzaldehyde using the method described in example 36(ii). LCMS 12 mins gradient Rt=2.67 (M⁺+1) 291.1. 1H NMR (d6-DMSO) δ=7.47 (1H, d, J=7.35 Hz), 7.24-7.23 (2H, m), 7.15-7.10 (1H, m), 6.42 (2H, s), 3.66 (2H, s), 3.25 (2H, brd), 2.90-2.86 (1H, m), 2.79-2.71 (2H, m), 2.43 (3H, s, SMe), 1.86-1.82 (2H, m), 1.74-1.67 (4H, m), 0.78-0.76 (1H, m), 0.36 (2H, d, J=5.47 Hz), 0.1 (4H, m).

### EXAMPLE 122

### N-(cyclopropylmethyl)-N-[(2-methylphenyl)methyl]piperidin-4-amine fumarate

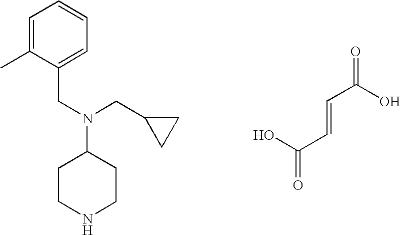

The title product was prepared with 4-(cyclopropylmethylamino)piperidine-1-carboxylic acid tert-butyl ester and 2-methylbenzaldehyde using the method described in example 36(ii). LCMS 12 mins gradient Rt=2.41 (M⁺+1) 259.2. 1H NMR (d6-DMSO) δ=7.37 (1H, m), 7.14-7.10 (3H, m), 6.42 (2H, s), 3.65 (2H, s), 3.25 (2H, brd), 2.90-2.55 (3H, m), 2.32 (3H, s), 2.31 (2H, s), 1.86-1.67 (4H, m), 0.78-0.76 (1H, m), 0.39-0.36 (2H, m), 0.1 (4H, m)

### EXAMPLE 123

### N-(cyclopropylmethyl)-N-[(2-chlorophenyl)methyl]piperidin-4-amine fumarate

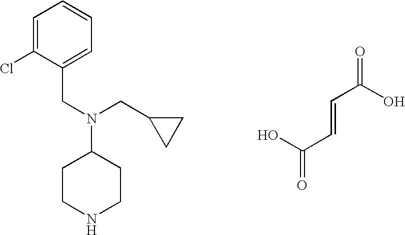

The title product was prepared with 4-(cyclopropylmethylamino)piperidine-1-carboxylic acid tert-butyl ester and 2-chlorobenzaldehyde using the method described in example 36(ii). LCMS 12 mins gradient Rt=2.45 (M⁺+1) 279.1. 1H NMR (d6-DMSO) δ=7.65 (1H, d), 7.40-7.18 (3H, m), 6.42 (2H, s), 3.71 (2H, s), 3.25 (2H, brd), 2.95-2.88 (1H, m), 2.79-2.71 (2H, m), 2.39 (2H, d), 1.86-1.75 (2H, m), 1.72-1.58 (2H, m), 0.78-0.76 (1H, m), 0.39-0.36 (2H, m), 0.10 (4H, m).

### EXAMPLE 124

### N-(cyclopropylmethyl)-N-{[4-fluoro-2-(trifluoromethyl)phenyl]methyl}piperidin-4-amine fumarate

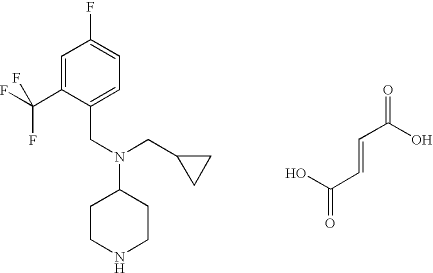

The title product was prepared with 4-(cyclopropylmethylamino)piperidine-1-carboxylic acid tert-butyl ester and 2-fluoro-4-(trifluoromethyl)benzaldehyde using the method described in example 36(ii). LCMS 12 mins gradient Rt=4.57 (M⁺+1) 331.1. 1H NMR (d6-DMSO) δ=7.37 (1H, m), 7.14-7.10 (2H, m), 6.42 (2H, s), 3.65 (2H, s), 3.25 (2H, brd), 2.90-2.55 (3H, m), 2.32 (3H, s), 2.31 (2H, d), 1.86-1.67 (4H, m), 0.78-0.76 (1H, m), 0.39-0.36 (2H, m), 0.1 (3H, m).

### EXAMPLE 125

### N-(cyclopropylmethyl)-N-[(2,4-dichlorophenyl)methyl]piperidin-4-amine fumarate

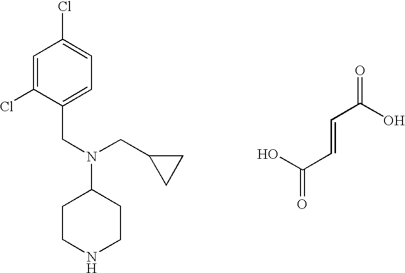

The title product was prepared with 4-(cyclopropylmethylamino)piperidine-1-carboxylic acid tert-butyl ester and 2,4-dichlorobenzaldehyde using the method described in example 36(ii). LCMS 12 mins gradient Rt=3.61 (M⁺+1) 313.0. 1H NMR (d6-DMSO) δ=7.37 (1H, m), 7.14-7.10 (1H, m), 7.41 (1H, m), 6.42 (2H, s), 3.65 (2H, s), 3.24 (2H, brd), 2.90-2.55 (3H, m), 2.30 (2H, d), 1.89-1.61 (4H, m), 0.78-0.76 (1H, m), 0.39-0.36 (2H, m), 0.1 (4H, m).

### EXAMPLE 126

### N-(3-methylbutyl)-N-[(2-phenoxyphenyl)methyl]piperidin-4-amine difumarate

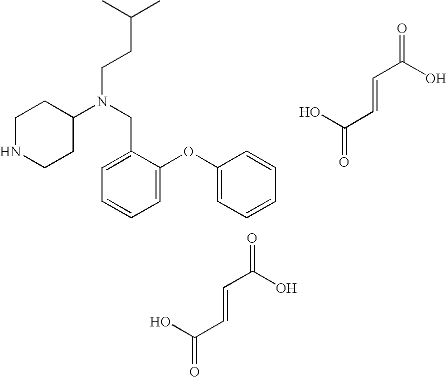

(i) To 10% Pd/C (1.0 g, 10% wt), under nitrogen, was added a solution of the 1-Boc-4-piperidone (10.0 g, 50.1 mmole, 1.0 eq.) and isoamylamine (4.46 g, 51.2 mmole, 1.02 eq.) in ethanol (60 ml). This was hydrogenated overnight, at 60 psi using a Parr hydrogenator. The catalyst was removed by filtration through Celite. Solvent was removed under vacuum to give 1,1-dimethylethyl 4-[(3-methylbutyl)amino]piperidine-1-carboxylate as a colourless, slightly cloudy, oil (13.59 g, 100%). δ_{H} (300 MHz, CDCl₃) 4.05-4.02 (2H, m, NCH₂), 2.82-2.75 (2H, m, NCH₂), 2.66-2.54 (3H, m, NCH, NCH₂), 1.86-1.82 (2H, m, CCH₂), 1.62 (1H, septet, CHMe₂), 1.45 (9H, s, OC(CH₃)₃), 1.41-1.17 (4H, m, CCH₂), 0.90 (6H, d, C(CH₃)₂); LCMS 6 min, Rt=2.7 min, (M⁺+1)=271.

(ii) As method previously described for Example 87, using 2-phenoxybenzaldehyde and 1,1-dimethylethyl 4-[(3-methylbutyl)amino]piperidine-1-carboxylate. Isolation of the fumarate salt from methanol and diethyl ether yielded the title compound as a white solid (0.264 g, 30%). δ_{H} (300 MHz, MeOD) 7.46 (1H, dd, ArH), 7.26-7.16 (3H, m, ArH), 7.10-7.04 (1H, m, ArH), 7.00-6.95 (1H, m, ArH), 6.86-6.79 (3H, m, ArH), 6.61 (4H, s, fumarate CH), 3.68 (2H, s, CH₂Ar), 3.33-3.28 (2H, m, NCH₂), 3.04-2.96 (3H, m, NCH, NCH₂), 2.56-2.51 (2H, m, NCH₂), 1.91-1.87 (2H, m, CCH₂), 1.76-1.62 (2H, m, CCH₂), 1.52-1.41 (1H, m, CH), 1.30-1.23 (2H, m, CH₂), 0.74 (6H, d, CH₃); LCMS 12 min, Rt=4.2 min, (Mf+1)=353.

### EXAMPLE 127

### N-(3-methylbutyl)-N-[(2-biphenyl)methyl]piperidin-4-amine difumarate

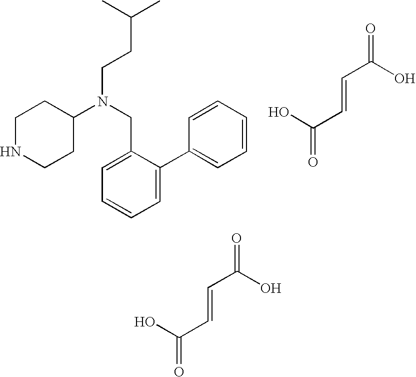

As method previously described for Example 107, using 1,1-dimethylethyl 4-[(3-methylbutyl)amino]piperidine-1-carboxylate and 2-phenylbenzyl bromide. Isolation of the fumarate salt from methanol and diethyl ether yielded the title compound as a white solid (0.239 g, 24%). δ_{H} (300 MHz, MeOD) 7.49 (1H, dd, ArH), 7.35-7.18 (7H, m, Ar), 7.10 (1H, dd, ArH), 6.61 (4H, s, fumarate CH), 3.62 (2H, s, CH₂Ar), 3.25 (2H, m, NCH₂), 2.78-2.59 (3H, m, NCH, NCH₂), 2.36-2.31 (2H, m, NCH₂), 1.64-1.45 (4H, m, CCH₂), 1.42-1.31 (1H, m, CH), 1.13-1.05 (2H, m, CH₂), 0.69 (6H, d, CH₃); LCMS 12 min, Rt=4.1 min, (M⁺+1)=337.

### EXAMPLE 128

### N-(1,2-dimethylpropyl)-N-{[2-(trifluoromethyl)phenyl]methyl}piperidin-4-amine fumarate isomer 1

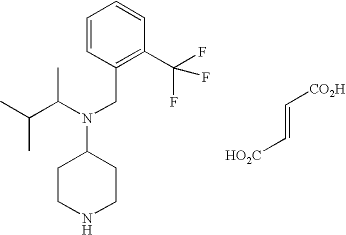

(i) As example 111(i) with 1,2-dimethylpropylamine to give 1,1-dimethylethyl 4-[(1,2-dimethylpropyl)amino]piperidine-1-carboxylate (3.15 g, 46%) as a colourless oil. δ_{H} (300 MHz, CDCl₃) 3.92-3.76 (2H, m, NCH₂), 2.71-2.46 (4H, m, NCH₂, NCH and NCH), 1.80-1.73 (2H, m, CCH₂), 1.62-1.51 (1H, m, CH), 1.38 (9H, m, C(CH₃)₃), 1.18-1.04 (2H, m, CH₂) 0.87 (3H, d, J=6.4 Hz, CH₃), 0.81 (3H, d, J=6.8 Hz, CH₃) and 0.77 (3H, d, J=6.8 Hz, CH₃); LCMS 12 min, Rt=2.48 min, (M⁺+1)=271.3.

(ii) To a solution of 1,1-dimethylethyl 4-[(1,2-dimethylpropyl)amino]piperidine-1-carboxylate (1.6 g, 6.0 mmol, 1 eq) and 2-(trifluoromethyl)benzyl bromide (1.1 ml, 7.2 mmol, 1.2 eq) in acetonitrile (30 ml) was added potassium carbonate (1.33 g, 9.6 mmol, 1.6 eq). The mixture was refluxed for 4 days then cooled to room temperature, diluted with water (50 ml) and extracted with ethyl acetate (3×50 ml). The combined organic extracts were washed with brine (100 ml), dried (MgSO₄) and the solvent removed in vacuo to give an oil which was purified by automated flash chromatography using an ISCO Combiflash system (120 g SiO₂) with a gradient of 0-40% methanol in ethyl acetate over 80 minutes to give 1,1-dimethylethyl 4-[{[2-(trifluoromethyl)phenyl]methyl}(1,2-dimethylpropyl)amino]-piperidine-1-carboxylate (1.77 g, 4.1 mmol, 70%) as a colourless oil. To This was added a solution of anisole (4 ml) and trifluoroacetic acid (5 ml, 52.3 mmol, 12.5 eq), in dichloromethane (20 ml) and the mixture stirred at room temperature for 16 h. The solvent removed in vacuo and the residue diluted with methanol (10 ml) and loaded onto SCX-2 ion exchange cartridge (2×10 g). Each column was washed with methanol (50 ml) and eluting the product with 2M ammonia in methanol solution (50 ml) the solvent removed in vacuo to give a colourless oil (723 mg, 53%). The racemic mixture was separated into their two enantiomers by chiral chromatography on a Chiralcel OD(3641) using 70% Heptane/30% ethanol and 0.2% diethylamine as the mobile phase at a rate of 0.5 ml/min. To a solution of the free base of the first eluting enantiomer (164 mg, 0.5 mmol, 1 eq) in diethyl ether (10 ml) and a few drops of methanol to solubilize was added a hot solution of fumaric acid (58 mg, 0.5 mmol, 1 eq) in methanol (1 ml) was added. The solution was heated and a few drops of methanol added until all solid was in solution, then the mixture was allowed to slowly cool to 0° C. The product was collected by filtration and dried in a vacuum oven (40° C. for 2 h) to give N-(1,2-dimethylpropyl)-N-{[2-(trifluoromethyl)phenyl]methyl}piperidin-4-amine fumarate isomer 1 (150 mg, 31%) as a white solid. δ_{H} (300 MHz, MeOD) 7.84 (1H, d, J=7.7 Hz, ArH), 7.55-7.47 (2H, m, ArH), 7.31-7.26 (1H, m, ArH), 6.58 (2H, s, fumarate CH), 3.90 (1H, d, J=16.0 Hz, CHHAr), 3.74 (1H, d, J=16.0 Hz, CHHAr), 3.34-3.27 (2H, m, NCH₂), 2.90-2.75 (3H, m, NCH and NCH₂), 2.35-2.30 (1H, m, NCH), 2.07-2.03 (1H, m, CH), 1.81-1.53 (4H, m, CCH₂ and CCH₂), 1.02 (3H, d, J=6.6 Hz, CH₃), 0.90 (3H, d, J=6.6 Hz, CH₃) and 0.77 (3H, d, J=6.6 Hz, CH₃); LCMS 12 min, Rt=6.00 min (M⁺+1) 329.2.

### EXAMPLE 129

### N-(1,2-dimethylpropyl)-N-{[2-(trifluoromethyl)phenyl]methyl}piperidin-4-amine fumarate isomer 2

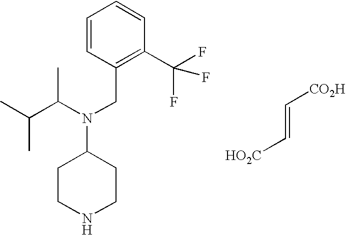

As example 128(ii) with the second eluting enantiomer to give N-(1,2-dimethylpropyl)-N-{[2-(trifluoromethyl)phenyl]methyl}piperidin-4-amine fumarate isomer 2 (139 mg, 28%) as a white solid. δ_{H} (300 z, MeOD) 7.96 (1H, d, J=7.7 Hz, ArH), 7.67-7.59 (2H, m, ArH), 7.42-7.40 (1H, m, ArH), 6.70 (2H, s, fumarate CH), 4.02 (1H, d, J=16.0 Hz, CHHAr), 3.86 (1H, d, J=16.0 Hz, CHHAr), 3.52-3.42 (2H, m, NCH₂), 3.01-2.88 (3H, m, NCH and NCH₂), 2.46-2.41 (1H, m, NCH), 2.19-2.15 (1H, m, CH), 1.94-1.70 (4H, m, CCH₂ and CCH₂), 1.13 (3H, d, J=6.6 Hz, CH₃), 1.01 (3H, d, J=6.6 Hz, CH₃) and 0.89 (3H, d, J=6.6 Hz, CH₃); LCMS 12 min, Rt=5.96 min, (M⁺+1)=329.2.

### EXAMPLE 130

### N-propyl-N-[(2-methylthiophenyl)methyl]piperidin-4-amine fumarate

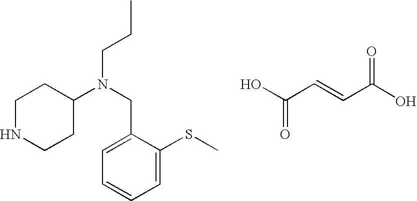

The title product was prepared with 4-(propylamino)piperidine-1-carboxylic acid tert-butyl ester and 2-(methylthio)benzaldehyde using the method described in example 36(ii). LCMS 12 mins gradient Rt=2.61 (+1) 279.1. 1H NMR (d6-DMSO) δ=7.43(1H, d, J=7.34 Hz), 7.23 (2H, m), 7.13 (1H, m), 6.42 (2H, s), 3.61(2H, s), 3.40(2H, m), 3.25 (2H, brd), 2.78-2.70 (3H, m), 2.49 (3H, s, SMe), 2.43-2.37 (2H, m), 1.84-1.71 (2H, m), 1.67-1.63 (2H, m), 1.35-1.28 (2H, m), 0.79-0.73 (3H, m).

### EXAMPLE 131

### N-propyl-N-{[2-(difluoromethoxy)phenyl]methyl}piperidin-4-amine fumarate

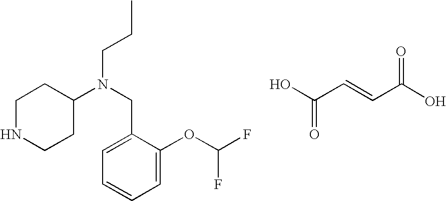

The title product was prepared with 4-(propylamino)piperidine-1-carboxylic acid tert-butyl ester and 2-(difluoromethoxy)benzaldehyde using the method described in example 36(ii). LCMS 12 mins gradient Rt=2.33 Q&+I) 299.1. 1H NMR (d6-DMSO) δ=7.55-7.52 (1H, m), 7.42-7.30 (2H, m), 7.27-7.20 (1H, m), 6.43 (2H, s), 3.62(2H, s), 3.25 (2H, brd), 2.78-2.71 (3H, m), 2.42-2.40 (2H, m), 1.82-1.64 (4H, m), 1.46-1.30 (2H, m), 0.83-0.74 (3H, m).

### EXAMPLE 132

### N-propyl-N-[(2-methylphenyl)methyl]piperidin-4-amine fumarate

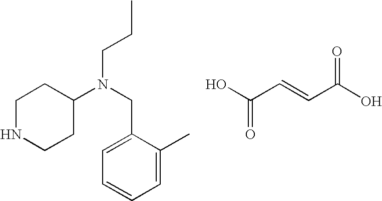

The title product was prepared with 4-(propylamino)piperidine-1-carboxylic acid tert-butyl ester and 2-methylbenzaldehyde using the method described in example 36(ii). LCMS 12 mins gradient Rt=2.40 (M⁺+1) 247.2. 1H NMR (d6-DMSO) δ=7.33-7.31 (1H, m), 7.14-7.11 (3H, m), 6.42 (2H, s), 3.57(2H, s), 3.25 (2H, brd), 2.76-2.69 (3H, m), 2.49-2.40 (2H, m), 2.38 (3H, s, Me), 1.82-1.70 (4H, m), 1.34-1.27 (2H, m), 0.77-0.72 (3H, m).

### EXAMPLE 133

### N-propyl-N-{[4-fluoro-2-(trifluoromethyl)phenyl]methyl}piperidin-4-amine fumarate

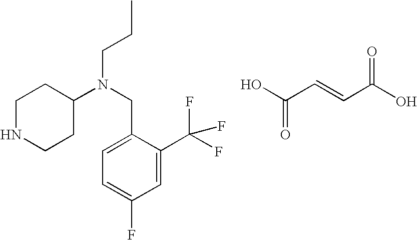

The title product was prepared with 4-(propylamino)piperidine-1-carboxylic acid tert-butyl ester and 4-fluoro-2-(trifluoromethyl)benzaldehyde using the method described in example 36(ii). LCMS 12 mins gradient Rt=4.69 (M⁺+1) 319.1. 1H NMR (d6-DMSO) δ=7.97-7.85 (1H, m), 7.59-7.49 (2H, m), 6.42 (2H, s), 3.66(2H, s), 3.25 (2H, brd), 2.78-2.67 (3H, m), 2.48-2.39 (2H, m), 1.84-1.61 (4H, m), 1.38-1.24 (2H, m), 0.79-0.72 (3H,

### EXAMPLE 134

### N-propyl-N-[(2,4-dichlorophenyl)methyl]piperidin-4-amine fumarate

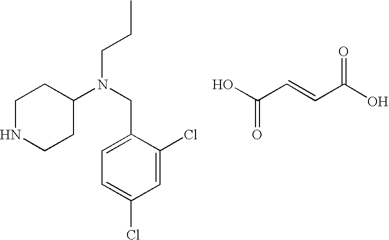

The title product was prepared with 4-(propylamino)piperidine-1-carboxylic acid tert-butyl ester and 2,4-dichlorobenzaldehyde using the method described in example 36(ii). LCMS 12 mins gradient Rt=3.59 (M⁺+1) 301.1. 1H NMR (d6-DMSO) δ=7.60-7.54 (2H, m), 7.43-7.40 (1H, m), 6.42 (2H, s), 3.65(2H, s), 3.25 (2H, brd), 2.79-2.71 (3H, m), 2.45-2.40 (2H, m), 1.82-1.78 (2H, m), 1.64-1.60 (2H, m), 1.37-1.32 (2H, m), 0.80-0.76 (3H, m).

### EXAMPLE 135

### N-butyl-N-[(2-methylthiophenyl)methyl]piperidin-4-amine fumarate

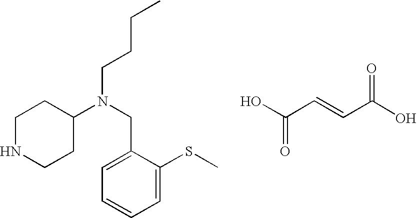

The title product was prepared with $(butylamino)piperidine-1-carboxylic acid tert-butyl ester and 2-(methythio)benzaldehyde using the method described in example 36(ii). LCMS 12 mins gradient Rt=3.04 (M⁺+1) 293.1. 1H NMR (d6-DMSO) δ=7.42 (1H, d, J=7.35 Hz), 7.23 (2H, d, J=3.96 Hz) 7.12 (1H, m), 6.42 (2H, s), 3.63 (2H, s), 3.25 (2H, brd), 2.78-2.70 (3H, m), 2.43 (3H, s, SMe) 2.40 (2H, m), 1.84-1.65 (4H, m), 1.30-1.21 (4H, m), 0.80-0.75 (3H, m).

### EXAMPLE 136

### N-butyl-N-{[2-(difluoromethoxy)phenyl]methyl}piperidin-4-amine fumarate

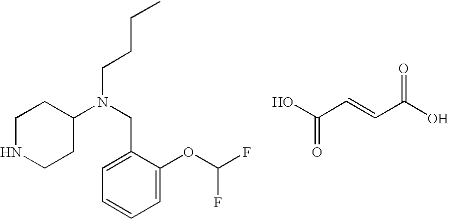

The title product was prepared with 4-(butylamino)piperidine-1-carboxylic acid tert-butyl ester and 2-(difluoromethoxy)benzaldehyde using the method described in example 36(ii). LCMS 12 mins gradient Rt=2.96 (M⁺+1) 313.1. 1H NMR (d6-DMSO) δ=7.55 (1H, d), 7.35-7.20 (2H, m) 7.19-7.11 (1H, m), 6.44 (2H, s), 3.62 (2H, s), 3.25 (2H, brd), 2.78-2.65 (3H, m), 2.43-2.40 (2H, m), 1.85-1.61 (4H, m), 1.41-1.15 (4H, m), 0.81-0.70 (3H, m).

### EXAMPLE 137

### N-butyl-N-[(2-methylphenyl)methyl]piperidin-4-amine fumarate

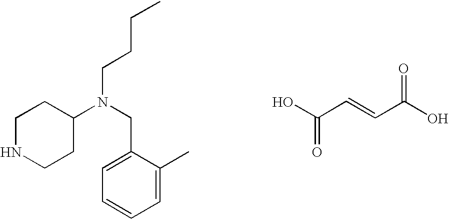

The title product was prepared with 4-(butylamino)piperidine-1-carboxylic acid tert-butyl ester and 2-methylbenzaldehyde using the method described in example 36(ii). LCMS 12 mins gradient Rt=2.98 (M⁺+1) 261.2. 1H NMR (d6-DMSO) δ=7.39-7.32 (1H, m), 7.15-7.10 (4H, m), 6.49 (2H, s), 3.55 (2H, s), 3.22 (2H, brd), 2.77-2.65 (3H, m), 2.45-2.39 (2H, m), 2.28 (3H, s, Me), 1.85-1.61 (4H, m), 1.35-1.10 (4H, m), 0.80-0.71 (3H, m)

### EXAMPLE 138

### N-butyl-N-[(2-chlorophenyl)methyl]piperidin-4-amine fumarate

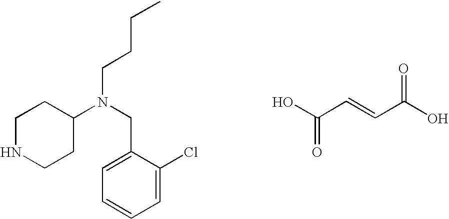

The title product was prepared with 4-(butylamino)piperidine-1-carboxylic acid tert-butyl ester and 2-chlorobenzaldehyde using the method described in example 36(ii). LCMS 12 mins gradient Rt=3.02 (M⁺+1) 281.2. 1H NMR (d6-DMSO) δ=7.56 (1H, d, J=6.03 Hz), 7.40-7.23 (3H, m), 6.42 (2H, s), 3.67 (2H, s), 3.25 (2H, brd), 2.79-2.71 (3H, m), 2.49-2.48 (2H, m), 1.84-1.64 (4H, m), 1.31-1.20 (4H, m), 0.81-0.76 (3H, m).

### EXAMPLE 139

### N-butyl-N-{[4-fluoro-2-(trifluoromethyl)phenyl]methyl}piperidin-4-amine fumarate

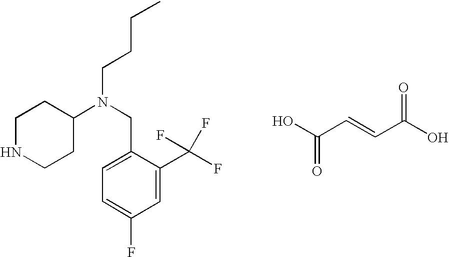

The title product was prepared with 4-(butylamino)piperidine-1-carboxylic acid tert-butyl ester and 4-fluoro-2-(trifluoromethyl)benzaldehyde using the method described in example 36(ii). LCMS 12 mins gradient Rt=5.30 (M⁺+1) 333.2. 1H NMR (d6-DMSO) δ=7.92-7.88 (1H, m), 7.52-7.49 (2H, m), 6.42 (2H, s), 3.71 (2H, s), 3.25 (2H, brd), 2.79-2.66 (3H, m), 2.49-2.47 (2H, m), 1.85-1.54 (4H, m), 1.41-1.15 (4H, m), 0.81-0.71 (3H, m).

### EXAMPLE 140

### N-butyl-N-[(2,4-dichlorophenyl)methyl]piperidin-4-amine fumarate

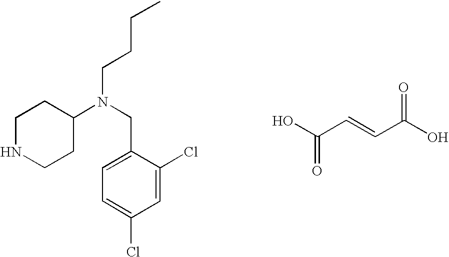

The title product was prepared with 4-(butylamino)piperidine-1-carboxylic acid tert-butyl ester and 2,4-dichlorobenzaldehyde using the method described in example 36(ii). LCMS 12 mins gradient Rt=4.17 (M⁺+1) 315.1. 1H NMR (d6-DMSO) δ=7.61-7.50 (2H, m), 7.45-7.39 (1H, m), 6.42 (2H, s), 3.67 (2H, s), 3.25 (2H, brd), 2.81-2.59 (3H, m), 2.49-2.48 (2H, m), 1.85-1.55 (4H, m), 1.48-1.15 (4H, m), 0.85-0.75 (3H, m).

Examples 141-152 and 157-158 shown in Table 1 below were prepared using a method similar to that described for example 56 using 1,1-dimethylethyl 4-[(2-methylpropyl)amino]piperidine-1-carboxylate and the appropriately substituted benzaldehyde. Examples 153-156 shown in Table 1 below were prepared similarly to method described for example 56 from 1,1-dimethylethyl 4-{[(2,3dichlorophenyl)methyl]amino}piperidine-1-carboxylate and the appropriately substituted aldehyde.

### 1,1-Dimethylethyl 4-{[(2,3-dichlorophenyl)methyl]amino}piperidine-1-carboxylate

To a solution of N-(tert-butoxycarbonyl)-4-piperidone (11.6 g, 60 mmol, 1 eq) and 2,3-dichlorobenzylamine (10.56 g, 60 mmol, 1 eq) in 1,2-dichloroethane (200 ml) was added sodium triacetoxyborohydride (17.8 g, 84 mmol, 1.4 eq) and the reaction mixture stirred for 5 hours. The mixture was diluted with water (100 ml) and 2N sodium hydroxide (140 ml), and then extracted with DCM (3×50 ml). The combined organic extracts were washed with brine (100 ml), dried (MgSO₄) and the solvent removed in vacuo to give a yellow oil 1,1-dimethylethyl 4-{[(2,3-dichlorophenyl)methyl]amino}piperidine-1-carboxylate (20.2 g, 94%) as a colourless oil; LCMS (6 min): Rt=3.00 min, (M⁺+1) 359.1.

### 1,1-Dimethylethyl 4-{[(4-fluorophenyl)methyl]amino}piperidine-1-carboxylate

The title compound was prepared using a method similar to that described for 1,1-Dimethylethyl 4-{[(2,3-dichlorophenyl)methyl]amino}piperidine-1-carboxylate with 4-fluorobenzylamine to give 1,1 dimethylethyl 4-{[(4-fluorophenyl)methyl]amino}piperidine-1-carboxylate (11.18 g, 98%) as a colourless solid; LCMS (12 min): Rt=2.40 min, (M⁺+1)=309.4.

### 2-Chloro-3-methylbenzaldehye

(i) To a solution of 2-chloro-3-methylbenzoic acid (1 g, 5.88 mmol, 1 eq) in dry THF (10 ml) under nitrogen atmosphere at 0° C. was added dropwise borane-dimethyl sulphide complex (0.523 ml, 6.47 mmol, 1.1 eq). After the addition was complete the mixture was heated to reflux for 3 hours, cooled to room temperature and poured slowly into water (100 ml). The aqueous layer was extracted with DCM (5×100 ml) and the combined organic layers were dried (MgSO₄) the solvent was removed in vacuo to yield the 2-chloro-3-methylbenzylalcohol (1 g, 100%) as a white solid; ¹H NMR (300 MHz, CDCl₃): δ=7.38-7.11 (3H, m), 4.75 (2H, s) and 2.39 (3H, s).

(ii) A solution of DCM (30 ml) was cooled to −78° C. and oxalyl chloride (1.34 ml, 15.2 mmol, 1.2 eq) was added under nitrogen followed by dropwise addition of DMSO (2.2 ml, 31.7 mmol 2.5 eq) in DCM (10 ml). After stirring for 15 min a solution of 2-chloro-3-methylbenzylalcohol (2 g, 12.7 mmol, 1 eq) in DCM (12 ml) was added dropwise. After a further 30 min triethylamine (9.03 ml, 63.5 mmol, 5 eq) was added in one portion and the mixture warmed to room temperature over 1 hour. The reaction mixture was quenched with saturated aqueous sodium hydrogen carbonate (50 ml) and the aqueous layer extracted with DCM (2×50 ml). The combined organic extracts were dried (MgSO₄) and the solvent removed in vacuo to give 2-chloro-3-methylbenzaldehyde (1.8 g, 91.4%) as a yellow oil; ¹H NMR (300 MHz, CDCl₃) δ=10.53 (1H, s, CHO), 7.81-7.74 (1H, m, ArH), 7.53-7.46 (1H, m, ArH), 7.34-7.23 (1H, m, ArH) and 2.46 (3H, s).

### 3-chloro-2-methylbenzaldehye

(i) The title compound was prepared using a method similar to that described when synthesising 2-chloro-3-methylbenzaldehye. Starting with 3-chloro-2-methylbenzoic acid, gave 3-chloro-2-methylbenzylalcohol (10 g, 100%) as a white solid; ¹H NMR (300 MHz, CDCl₃): δ=7.40-7.05 (3H, m), 4.75-4.62 (2H, m) and 2.35 (3H, s).

(ii) The title compound was prepared using a method similar to that described when synthesisng 2-chloro-3-methylbenzaldehye. Starting with starting with 3-chloro-2-methylbenzylalcohol, gave 3-chloro-2-methylbenzaldehyde (1.6 g, 81.2%) as a yellow oil; ¹H NMR (300 MHz, CDCl₃): δ=10.28 (1H, s, CHO), 7.78-7.69 (1H, m, ArH), 7.61-7.55 (1H, m, ArH), 7.35-7.25 (1H, m, ArH) and 2.70 (3H, s).

TABLE 1

| | | | LCMS |
|---|---|---|---|
| | | | RT (12 |
| Example | | | minute), |
| No. | Structure | Name | M⁺ + 1 |
| 141 | 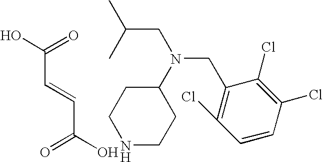 | N-(2-Methylpropyl)-N-{(2,3,6- (trichloro)phenyl]methyl}piperidin-4- amine fumarate | 5.98 min, 349.0 |
| 142 | 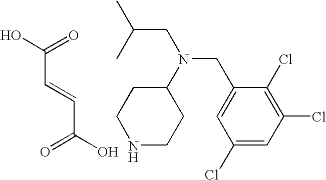 | N-(2-Methylpropyl)-N-{(2,3,5- (trichloro)phenyl]methyl}piperidin-4- amine fumarate | 6.35 min, 349.0 |
| 143 | 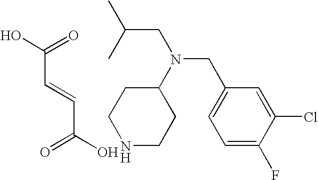 | N-(2-Methylpropyl)-N-{[(3-chloro-4- fluoro)phenyl]methyl}piperidin-4-amine fumarate | 4.26 min, 299.1 |
| 144 | 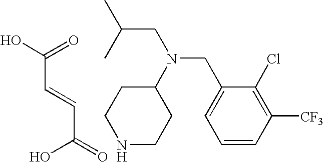 | N-(2-Methylpropyl)-N-{[2-chloro-3- (trifluoromethyl)phenyl]methyl}piperidin- 4-amine fumarate | 5.89 min, 349.1 |
| 145 | 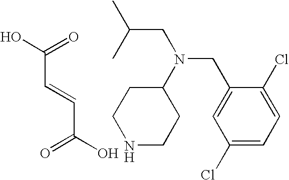 | N-(2-Methylpropyl)-N-{[(2,5- dichloro)phenyl]methyl}piperidin-4-amine fumarate | 5.64 min, 315.1 |
| 146 | 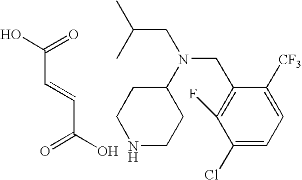 | N-(2-Methylpropyl)-N-{[3-chloro-2-fluoro- 6-(trifluoromethyl))phenyl]methyl}piperidin- 4-amine fumarate | 6.01 min, 367.1 |
| 147 | 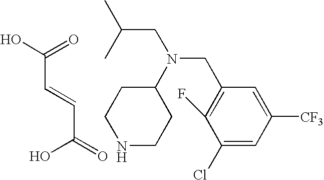 | N-(2-Methylpropyl)-N-{[(3-chloro-2-fluoro- 5-(trifluoromethyl))phenyl]methyl}piperidin- 4-amine fumarate | 6.22 min, 367.1. |
| 148 | 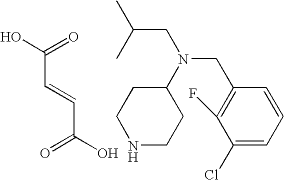 | N-(2-Methylpropyl)-N-{[(3-chloro-2- fluoro)phenyl]methyl}piperidin-4-amine fumarate | 4.52 min, 299.1 |
| 149 | 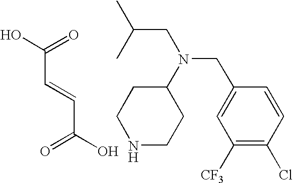 | N-(2-Methylpropyl)-N-{[(4-chloro-3- (trifluoromethyl))phenyl]methyl}piperidin- 4-amine fumarate | 5.65 min, 349.1 |
| 150 | 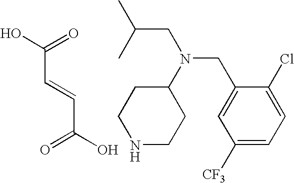 | N-(2-Methylpropyl)-N-{[(2-chloro-5- (trifluoromethyl))phenyl]methyl}piperidin- 4-amine fumarate | 6.06 min, 349.1 |
| 151 | 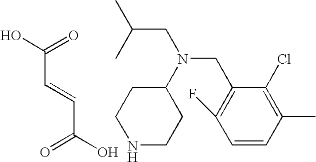 | N-(2-Methylpropyl)-N-{[(2-chloro-6- fluoro-3-methylphenyl]methyl}piperidin- 4-amine fumarate | 4.75 min, 313.1 |
| 152 | 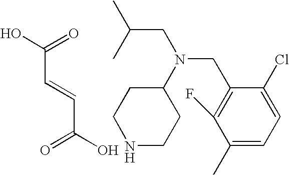 | N-(2-Methylpropyl)-N-{[(6-chloro-2- fluoro-3-methylphenyl]methyl}piperidin- 4-amine fumarate | 4.71 min, 313.1 |
| 153 | 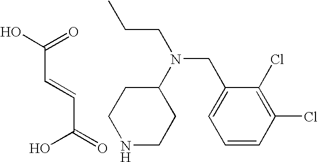 | N-(1-Propyl)-N-{[(2,3- dichloro)phenyl]methyl}piperidin-4-amine fumarate | 3.32 min, 301.1 |
| 154 | 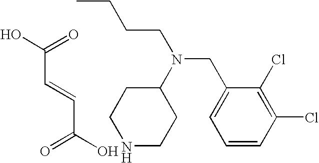 | N-(1-Butyl)-N-{[(2,3- dichloro)phenyl]methyl}piperidin-4-amine fumarate | 3.84 min, 315.1 |
| 155 | 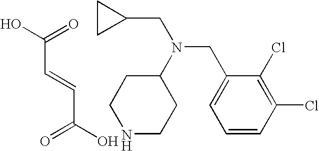 | N-(Cyclopropylmethyl)-N-{[(2,3- dichloro)phenyl]methyl}piperidin-4-amine fumarate | 3.34 min, 313.1 |
| 156 | 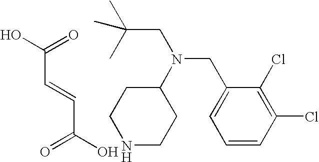 | N-(2,2-dimethylpropyl)-N-{[(2,3- dichloro)phenyl]methyl}piperidin-4-amine fumarate | 6.10 min, 329.1. |
| 157 | 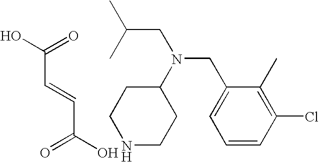 | N-(2-Methylpropyl)-N-{[(3-chloro-2- methyl)phenyl]methyl}piperidin-4-amine fumarate | 2.99 min, 295.2 |
| 158 | 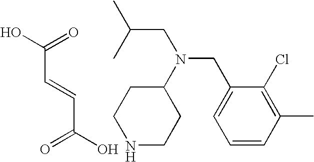 | N-(2-Methylpropyl)-N-{[(2-chloro-3- methyl)phenyl]methyl}piperidin-4-amine fumarate | 3.86 min, 295.2 |

### EXAMPLE 159

### N-(2,2-Dimethylpropyl)-N-{[1,1-biphenyl]-2-yl-methyl}piperidin-4-amine fumarate

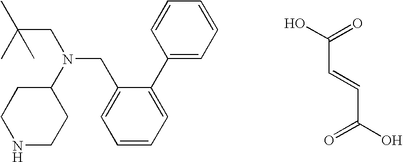

(I) To 10% Pd/C (1.0 g, 10% wt), under nitrogen, was added a solution of the N-(tert-butoxycarbonyl)-4-piperidone (10 g, 550.09 mmol, 1.0 eq) and neopentylamine (4.46 g, 51.19 mmol, 1.02 eq) in ethanol (60 ml). This was hydrogenated for 3 hrs, at 60 psi hydrogen, using a Parr Hydrogenator. The catalyst was removed by filtration through Celite. Solvent was removed under vacuum to give 1,1-dimethylethyl 4-[(2,2-dimethylpropyl)amino]piperidine-1-carboxylate as a colourless, slightly cloudy oil (13.60 g, 100%). LCMS: (6 min): Rt=2.6 mm, (M⁺+1)=271; ¹H NMR (300 MHz, CDCl₃): δ=4.00-3.97 (2H, m, NCH₂), 2.86-2.78 (2H, m, NCH₂), 2.58-2.49 (1H, m, NCH,), 2.35 (2H, s, NCH₂tBu), 1.84-1.78 (2H, m, CCH₂), 1.45 (9H, s, OC(CH₃)₃), 1.31-1.19 (2H, m, CCH₂), 0.91 (9H, s, C(CH₃)₃).

(ii) To a solution of 1,1-dimethylethyl 4-[(2,2-dimethylpropyl)amino]piperidine-1-carboxylate (0.41 g, 1.50 mmol, 1.0 eq), 2-phenylbenzyl bromide (0.133 ml, 1.80 mmol, 1.2 eq) in dry acetonitrile (5 ml) was added anhydrous potassium carbonate (0.33 g, 2.40 mmol, 1.6 eq). The mixture was stirred overnight at room temperature. The reaction mixture was concentrated under vacuum to give a white solid. The white solid was taken up in dichloromethane (10 ml) and this washed with water (10 ml). The dichloromethane layer was passed through a hydrophobic frit then diluted with methanol (10 ml). This solution was loaded onto an SCX-2 (10 g) column. The column was washed with methanol (50 ml) then basic material was eluted using 2N ammonia in methanol (50 ml). Concentration of the ammonia/methanol solution under vacuum yielded a white solid (0.60 g, 93%). To a solution of this oil (0.60 g, 1.37 mmol, 1.0 eq) in dichloromethane (10 ml) was added trifluoroacetic acid (TFA) (1.67 ml, 22.5 mmol, 16.4 eq). The solution was stirred overnight at room temperature. Solvent and TFA were removed in vacuo. The resulting oil was taken up in methanol and loaded onto an SCX-2 (log) column. The column was washed with methanol (50 ml). Basic material was then eluted using 2N ammonia in methanol (50 ml). Removal of solvent from the ammonia/methanol mixture under vacuum, gave a colourless oil (0.47 g, 100%). The oil was taken up in methanol. To this solution was added a solution of fumaric acid (0.16 g, 1.40 mmol, 1.01 eq) in methanol followed by diethyl ether. The resulting precipitate was collected by filtration to give the title compound as a white solid (0.59 g, 94%). LCMS: (12 min): Rt=5.9 min, (M⁺+1)=337; ¹H NMP (300 MHz, MeOD): δ=7.81-7.77 (1H, m, ArH), 7.47-7.28 (7H, m, ArH), 7.20-7.15 (1H, m, ArH), 6.70 (3H, s, fumarate CH), 3.71 (2H, s, CH₂Ar), 3.38-3.27 (2H, m, NCH₂), 2.79 (2H, dt, NCH₂), 2.61-2.51 (1H, m, NCH), 2.23 (2H, s, NCH₂tBu), 1.73-1.49 (4H, m, CCH₂), 0.85 (9H, s, CH₃).

### EXAMPLE 160

### N-(2,2-Dimethylpropyl)-N-{(2-phenoxyphenyl)methyl}piperidin-4-amine hemifumarate

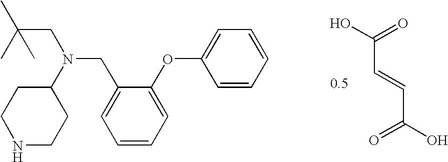

(i) To a 250 ml round bottomed flask was added 3-fluorobenzaldehyde (12.4 g, 100 mmol, 1.0 eq), phenol (11.3 g, 120 mmol, 1.2 eq), potassium carbonate (16.6 g, 120 mmol, 1.2 eq) and dimethylacetamide (100 ml). The reaction mixture was heated at reflux for 16 hours. The mixture was then diluted with water and extracted with diethyl ether. The combined organic extracts were washed with water (3×) and brine. The washed extracts were dried (Na₂SO₄) and concentrated in vacuo to give a yellow oil (15.7 g). This was purified by automated flash chromatography using an ISCO Combiflash system (SiO₂ (120 g×2); 0-10% ethyl acetate in cyclohexane gradient elution over 40 minutes) to give 3-phenoxybenzaldehyde as a colourless oil (14.8 g, 75%). LCMS: (6 min), Rt=4.0 min, (M⁺+1)=199; ¹H NMR (300 MHz, CDCl₃): δ=10.52 (1H, s, CHO), 7.94 (1H, dd, ArH), 7.54-7.45 (1H, m, ArH), 7.42-7.36 (2H, m, ArH), 7.21-7.15 (2H, m, ArH), 7.09-7.04 (2H, m, ArH), 6.90 (1H, d, ArH).

(ii) To a solution of 1,1-dimethylethyl 4-[(2,2-dimethylpropyl)amino]piperidine-1-carboxylate (0.41 g, 1.50 mmol, 1.0 eq), as prepared in Example (159), in 1,2-dichloroethane (10 ml) was added 3-phenoxybenzaldehyde (0.89 g, 4.50 mmol, 3.0 eq). To this was added a solution of sodium triacetoxyborohydride (0.95 g, 4.50 mmol, 3.0 eq) in dimethylformamide (2 ml). This mixture was left to stir under nitrogen, at room temperature, overnight. To the reaction mixture was added water (10 ml) and the mixture stirred vigorously for several minutes. The chlorinated organic layer was run through a hydrophobic frit to remove water, diluted with methanol (10 ml) and loaded onto an SCX-2 (10 g) column. The column was washed with methanol (50 ml) then basic material eluted with 2N ammonia in methanol. The ammonia/methanol solution was concentrated in vacuo to give 1,1-dimethylethyl 4-[(3-phenoxyphenylmethyl)(2,2-dimethylpropyll)amino]piperidine-1-carboxylate as a colourless oil (0.58 g, 74%). To a solution of this oil (0.58 g, 1.28 mmol, 1.0 eq) in dichloromethane (10 ml) was added trifluoroacetic acid (TFA) (1.67 ml, 22.5 mmol, 17.6 eq). The solution was stirred overnight at room temperature. Solvent and TFA were removed in vacuo. The resulting oil was taken up in methanol and loaded onto an SCX-2 (10 g) column. The column was washed with methanol (50 ml). Basic material was then eluted using 2N ammonia in methanol (50 ml). Removal of solvent from the ammonia/methanol mixture under vacuum, gave a colourless oil (0.45 g, 100%). The oil was taken up in methanol. To this solution was added a solution of fumaric acid (0.16 g, 1.33 mmol, 1.04 eq) in methanol. The mixture was left to stir for a couple of minutes, then, diethyl ether was added. The resulting precipitate was collected by filtration to give the title compound as a white solid (0.396 g, 76%). This was recrystallised a second time using the same solvent system to give the hemifumarate salt as a white solid (0.195 g, 37%). LCMS: (12 min), Rt=5.4 min, (M⁺+1)=353; ¹H NMR (300 MHz, MeOD): δ=7.76-7.72 (1H, m, ArH), 7.37-7.19 (4H, m, ArH), 7.11-7.03 (1H, m, ArH), 6.94-6.89 (3H, m, ArH), 6.66 (3H, s, fumarate CH), 3.81 (2H, s, CH₂Ar), 3.39 (2H, brd s, NCH₂), 2.84-2.63 (3H, m, NCH₂, NCH), 2.43 (2H, s, NCH₂tBu), 1.97-1.93 (2H, m, CCH₂), 1.79-1.64 (2H, m, CCH₂), 0.86 (9H, s, CH₃).

Table 2 examples 161-169 were similarly prepared.

TABLE 2

| | | | LCMS (12 |
|---|---|---|---|
| Example | | | min or 6 min*), |
| No. | Structure | Name | M⁺ + 1 |
| 161 | 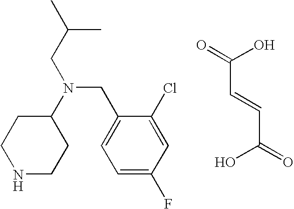 | N-(2-Methylpropyl)-N-{[(2-chloro- 4-fluoro)phenyl]methyl}piperidin-4- amine fumarate | 4.32 min, 299.1/301.1 |
| 162 | 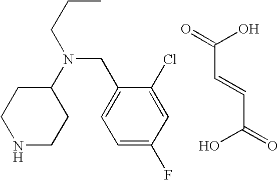 | N-(1-Propyl)-N-{[(2-cbloro-4- fluoro)phenyl]methyl}piperidin-4- amine fumarate | 2.63 min, 285.1/287.1 |
| 163 | 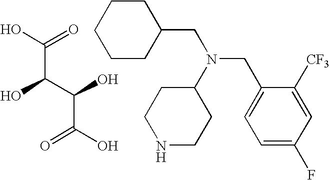 | N-(Cyclohexylmethyl)-N-{[(4- fluoro-2-(trifluoromethyl))phenyl]methyl}piperidin-4-amine L-Tartrate | 2.69 min*, 373.3 |
| 164 | 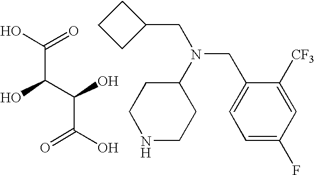 | N-(Cyclobutylmethyl)-N-{[(4- fluoro-2-(trifluoromethyl))phenyl]methyl}piperidin-4-amine L-Tartrate | 1.94 min*, 345.2 |
| 165 | 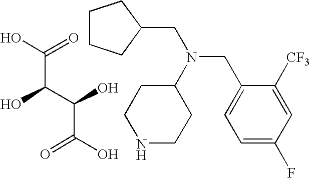 | N-(Cyclopentylmethyl)-N-{[(4- fluoro-2-(trifluoromethyl))phenyl]methyl}piperidin-4-amine L-Tartrate | 2.43 min*, 359.2 |
| 166 | 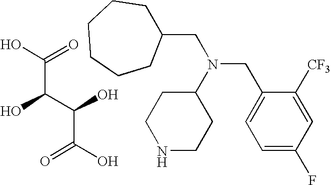 | N-(Cycloheptylmethyl)-N-{[(4- fluoro-2-(trifluoromethyl))phenyl]methyl}piperidin-4-amine L-Tartrate | 3.01 min*, 387.3 |
| 167 | 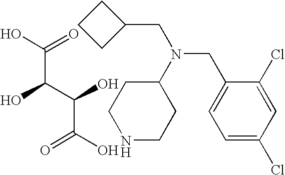 | N-(Cyclobutylmethyl)-N-{[(2,4- dichloro-phenyl)]methyl}piperidin- 4-amine L-Tartrate | 1.46 min*, 327.1 |
| 168 | 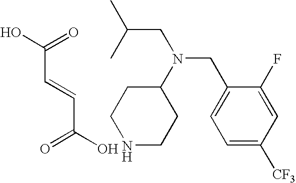 | N-(2-Methylpropyl)-N-{[(2-fluoro-4- (trifluoromethyl))phenyl]methyl}piperidin- 4-amine fumarate | 5.89 min, 333.1/334.1 |

### EXAMPLE 169

### N-{[(2-Trifluoromethyl)phenyl]methyl}-N-tetrahydro-2H-pyran-4yl-piperidin-4-amine fumarate

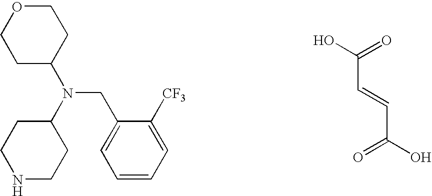

To a solution of 4-(2H-tetrahydropyran-4-yl)amino-piperidine-1-carboxylic acid tert-butyl ester (0.5 g, 1.39 mmol, 1.0 eq) in dichloromethane (10 ml) was added the 4-pyranone (0.41 g, 4.18 mmol, 3.0 eq). To this was added sodium triacetoxyborohydride (0.88 g, 4.18 mmol, 3.0 eq) and acetic acid (0.08 g, 1.39 mmol). This mixture was left to stir under nitrogen, at room temperature for 36 h. After this time, starting material was still evident therefore an additional 3 equivalents of pyranone were added. The reaction mixture was left for a further 16 h at room temperature. Starting material was still evident but the reaction was worked up by addition of water (10 ml). The mixture was stirred vigorously for several minutes. The chlorinated organic layer was then run through a hydrophobic frit to remove water. The resulting organic solution was diluted with methanol (10 ml) and loaded onto an SCX-2 (10 g) column. The column was washed with methanol (50 ml) then basic material eluted with 2N ammonia in methanol. The ammonia/methanol solution was concentrated in vacuo to give the product. To a solution of this oil (1.0 eq) in dichloromethane (10 ml) was added trifluoroacetic acid (TFA) (15 eq). The solution was stirred at room temperature for 4 h. Solvent and TFA were removed int vacuo. The resulting oil was taken up in methanol and loaded onto an SCX-2 (10 g) column. The column was washed with methanol (50 ml). Basic material was then eluted using 2N ammonia in methanol (50 ml). Removal of solvent from the ammonia/methanol mixture under vacuum, gave the desired compounds as an oil.

The oil was taken up in diethyl ether. To this solution was added a solution of fumaric acid (1 eq) in hot methanol and then cooled. The resulting precipitate was collected by filtration to give the title compound as a white solid (0.065 g). LCMS—(12 mins gradient): Rt=4.69 (M⁺+1) 343.1/344.2; ¹H NMR (MeOD) δ=7.90 (1H, d), 7.58-7.45 (2H, m), 7.32-7.25 (1H, m), 6.49 (4H, s), 3.90-3.80 (4H, m), 3.35-3.19 (5H, m), 3.15-2.81 (3H, m), 2.80-2.71 (1H, m), 1.95-1.85 (2H, brd), 1.79-1.49 (6H, m).

### EXAMPLE 170

### N-(Cyclopentyl)-N-{[(2,3-dichloro)phenyl]methy}piperidin-4-amine fumarate

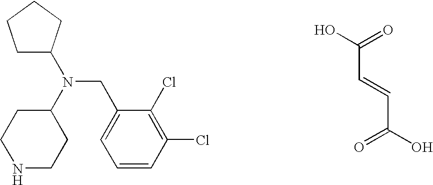

To a solution of 1,1-dimethylethyl 4-{[(2,3-dichlorophenyl)methyl]amino}piperidine-1-carboxylate (0.54 g, 1.5 mmol, 1 eq) and cyclopentanone (0.38 g, 4.5 mmol, 3 eq) in 1,2-dichloroethane (10 ml) was added acetic acid (0.09 ml, 1.5 mmol, 1 eq) and sodium triacetoxyborohydride (0.95 g, 4.5 mmol, 3 eq) in dimethylformamide (2 ml). After 16 hours the reaction was incomplete so a further portion of cyclopentanone (0.38 g, 4.5 mmol, 3 eq) was added and the mixture stirred for 16 hours. No further reaction was observed so additional portions of cyclopentanone (0.38 g, 4.5 mmol, 3 eq), acetic acid (0.09 ml, 1.5 mmol, 1 eq) and sodium triacetoxyborohydride (0.95 g, 4.5 mmol, 3 eq) were added and left to stir for 48 hours. The reaction was quenched with water (5 ml) and 2N NaOH (5 ml), and the organic layer separated by passing through a hydrophobic frit. This was diluted with methanol (10 ml) and loaded onto a SCX-2 ion exchange cartridge (5 g) washed with methanol (15 ml) and the product eluted with 2M ammonia in methanol solution (15 ml). The solvent removed in vacuo to give an oil which was purified by automated flash chromatography using an ISCO Combiflash system (40 g SiO₂) with a gradient of 0-30% ethyl acetate in iso-hexane over 40 minutes to give 1,1-dimethylethyl 4-{[(2,3-dichlorophenyl)methyl](cyclopentyl)amino}-piperidine-1-carboxylate as a colourless oil. To this oil was added a solution of anisole (1.4 ml) and trifluoroacetic acid (1.4 ml, 18.3 mmol, 12 eq), in DCM (7 ml) and the mixture stirred at room temperature for 16 hrs. The solvent removed in vacuo and the residue diluted with methanol (5 ml) and loaded onto SCX-2 ion exchange cartridge (5 g). The column was washed with methanol (15 ml) and the product eluted with 2M ammonia in methanol solution (15 ml) the solvent removed in vacuo to give (282 mg, 86%) as a colourless oil. The product was taken up in diethyl ether (15 ml) and a few drops of methanol were added to solubilize. A hot solution of fumaric acid (99.9 mg, 11.0 mmol, 1 eq) in methanol (1 ml) was added and the solution was heated adding a few drops of methanol added until all solid was in solution, then the mixture was allowed to slowly cool to 0° C. The product was collected by filtration and dried in a vacuum oven (40° C. for 2 hrs) to give N-(Cyclopentyl)-N-{[(2,3-dichloro)phenyl]methyl}piperidin-4-amine fumarate (282 mg, 64%) as a white solid. LCMS (12 min): Rt=4.38 min, (Mf++1)=327.1; ¹H NMR (300 MHz, MeOD): δ=7.62-7.59 (1H, m, ArH), 7.30-7.27 (1H, m, ArH), 7.17 (1H, t, J=7.8, ArH), 6.58 (2H, s, fumarate CH), 3.74 (2H, s, CH₂Ar) 3.32-3.20 (3H, m, NCH), 2.95-2.80 (3H, m, NCE), 1.91 (2H, br d, J=13.8, CH₂), 1.71-1.28 (10H, m, CH₂).

Examples 171-172 shown in table 3 were prepared using a method similar to that described for example 170 starting from 1,1-dimethylethyl 4-{[(2,3-dichlorophenyl)methyl]amino}piperidine-1-carboxylate and the appropriately substituted aldehyde or ketone. For example (172) the fumarate salt was recrystallised from methanol and ether to purify.

Table 3 examples 171-172 were similarly prepared.

TABLE 3

| | | | LCMS |
|---|---|---|---|
| Example | | | (12 minute) |
| No. | Structure | Name | M⁺ + 1 |
| 171 | 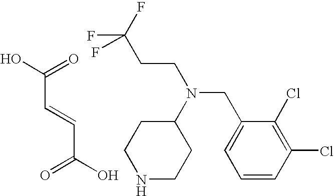 | N-(3,3,3-Trifluoropropyl)-N- {[(2,3-dichloro)phenyl]methyl}piperidin- 4-amine fumarate | 3.53 min, 355.1 |
| 172 | 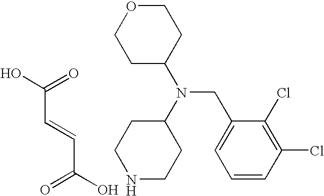 | N-{[(2,3-dichloro)phenyl]methyl}-N-tetrahydro-2H-pyran- 4-yl-piperidin-4-amine fumarate | 4.53 min, 343 |

### EXAMPLE 173

### N-(2-Methylpentyl)-N-{[(2,3 dichloro)phenyl]methyl}piperidin-4-amine fumarate-isomer 1

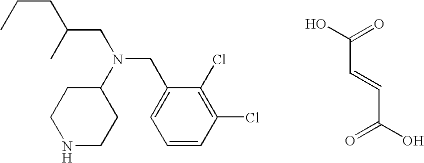

Prepared using a method similar to that described for example 141 starting with 1,1-dimethylethyl 4-{[(2,3-dichlorophenyl)methyl]amino}piperidine-1-carboxylate and 2-methylvaleraldehyde. Before formation of the fumarate salt the racemic mixture was 10 separated into their two enantiomers by chiral chromatography on a Chiralcel OD(3641) using 50% Heptane/50% ethanol and 0.2% DMEA as the mobile phase at a rate of 0.5 ml/min. To a solution of the free base of the first eluting enantiomer (201 mg, 0.5 mmol, 1 eq) in diethyl ether (10 ml) and a few drops of methanol were added to solubilize. A hot solution of fumaric acid (60 mg, 0.5 mmol, 1 eq) in methanol (1 ml) was added and the solution was heated adding a few drops of methanol added until all solid was in solution, then the mixture was allowed to slowly cool to 0° C. The product was collected by filtration and dried in a vacuum oven (40° C. for 2 hrs) to give N-(2-Methylpentyl)-N-[(2,3-dichlorophenyl)methyl]piperidin-4-amine fumarate isomer 1 (173 mg, 25.1%). LCMS (12 min): Rt=6.14 min, M⁺+1)=343.1; ¹H NMR (300 MHz, MeOD): δ=7.45-7.42 (1H, m, ArH), 7.32 (1H, dd, J=1.6 and 8.0, ArH), 7.17 (1H, t, J=8.0, ArH), 6.58 (2H, s, fumarate CH), 3.71 (2H, s, CH₂Ar) 3.40-3.22 (2H, m, NCH), 2.88-2.80 (2H, m, NCH), 2.75-2.67 (1H, m, NCH), 2.33 (1H, dd, J=12.8 and 6.6, CH), 2.19-2.16 (1H, dd, J=12.8 and 7.3, CH), 2.02-1.91 (2H, m, CH₂), 1.77-1.62 (2H, m, CH₂), 1.34-1.05 (4H, m, CH₂), 0.91-0.79 (1H, m, CH) and 0.76-0.72 (6H, m, 2×CH₃).

### EXAMPLE 174

### N-(2-Methylpentyl)-N-{[(2,3 dichloro)phenyl]methyl}piperidin-4-amine fumarate-isomer 2

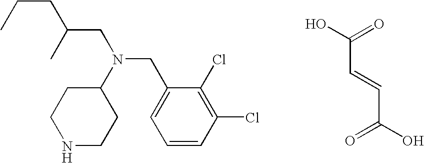

Prepared using a method similar to that described for example (173) taking the free base of the second eluting enantiomer (211 mg, 0.6 mmol, 1 eq) and forming the fumarate salt to give N-(2-Methylpentyl)-N-[(2,3-dichlorophenyl)methyl]piperidin-4-amine fumarate isomer 2 (215 mg, 31.2%). LCMS (12 min): Rt=6.14 min, (M⁺+1)=343.1; ¹H NMR (300 MHz, MeOD): δ=7.45-7.42 (1H, m, ArH), 7.32 (1H, dd, J=1.6 and 8.0, ArH), 7.17 (1H, t, J=8.0, ArH), 6.58 (2H, s, fumarate CH), 3.71 (2H, s, CH₂Ar) 3.40-3.22 (2H, m, NCH), 2.88-2.80 (2H, m, NCH), 2.75-2.67 (1H, m, NCH), 2.33 (1H, dd, J=12.8 and 6.6, CH), 2.19-2.16 (1H, dd, J=12.8 and 7.3, CH), 2.02-1.91 (2H, m, CH₂), 1.77-1.62 (2H, m, CH₂), 1.34-1.05 (4H, m, CH₂), 0.91-0.79(1H, m, CH) and 0.76-0.72 (6H, m, 2×CH₃).

### EXAMPLE 175

### N-(2-Methylpropyl)-4-methyl-N-{[(2-(trifluoromethyl)phenyl]methyl}piperidin-4-amine fumarate

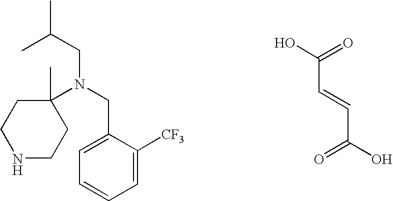

(i) To a stirred solution of 1-benzyl-4-piperidone (10.0 g, 52.84 mmol) in dry diethyl ether (100 ml) cooled to −78° C. under a nitrogen atmosphere, was added a solution of methyl lithium in diethyl ether (1.6M, 46.2 ml) dropwise. After addition, stirred at −78° C. for 1.5 h then quenched the reaction by addition of water. After warming to room temperature, the organic phase was separated and the aqueous phase washed with diethyl ether (3×). The combined organic phases was washed with brine, dried over magnesium sulphate, filtered and evaporated to an oil (11.1 g). The crude oil was purified using an ISCO combiflash on a 120 g cartridge eluting with ethyl acetate to remove starting material and then gradient elution with ethyl acetate-methanol 0 to 12% over 35 min. 1-benzyl-4-hydroxy-4-methylpiperidine was obtained as a pale yellow oil (5.64 g).

(ii) To a stirred solution of 1-benzyl-4-hydroxy-4-methylpiperidine (5.63 g, 27.42 mmol) in isobutyronitrile (30 ml) cooled to 5° C. was added conc. sulphuric acid (25 ml) dropwise. After addition, the suspension was stirred at room temperature overnight. The clear solution was cooled to 5° C. and adjusted to a pH of 9.2 by dropwise addition of 50% aqueous sodium hydroxide (50 ml) followed by aqueous sodium carbonate. The mixture was extracted with dichloromethane (3×) and the extracts washed with water (3×) and brine. The organic phase was dried over magnesium sulphate, filtered and evaporated. The brown oil was dried under vacuum to give the l-benzyl-4-isopropylcarboxamido-4-methylpiperidine (0.6 g).

(iii) To a stirred suspension of 1-benzyl-4 isopropylcarboxamido-4-methylpiperidine (2.03 g, 7.41 g), and polymer supported Hunig's base (7.97 g, 29.64 mmol) in dichloromethane (30 ml) at room temperature was added α-chloroethyl chloroformate (3.18 g, 22.23 mmol). After stirring at room temperature for 4 h, filtered and evaporated. The oil was stirred in methanol (20 ml) overnight at room temperature. Diluted with methanol (40 ml) and added powdered SCX-2, after stirring for 0.5 h, filtered and washed powder with 2 volumes of methanol and then eluted with 3 volumes of methanolic ammonia. Evaporation gave an oil (0.52 g).

The oil was dissolved in dichloromethane (10 ml) and triethylamine (418 mg, 4.14 mmol), a catalytic amount of 4-dimethylaminopyridine and a solution of di-t-butyl dicarbonate in tetrahydrofuran (1.0M, 3.86 ml) added. The solution was stirred at room temperature for 1.5 h then diluted with dichloromethane, washed with water (2×), aqueous 2M HCl, water and brine. Dried over magnesium sulphate, filtered and evaporated to give 1-butoxycarbonyl-4-(2-methylpropyl)carboxamido-4-methylpiperidine as an oil (0.68 g).

(iv) To a stirred solution of 1-butoxycarbonyl-4-isopropylcarboxamido-4-methylpiperidine (665 mg, 2.35 mmol) in dry tetrahydrofuran (10 ml) at room temperature under a nitrogen atmosphere was added a solution of borane in tetrahydrofuran (1.0M, 4.7 ml). The reaction mixture was heated at reflux for 1.5 h then cooled to 5° C. and aqueous SM HCl (0.6 ml) added. After stirring at 5° C. for 10 min, aqueous NaOH was added until reaction mixture was basic and then extracted with diethyl ether (2×). Extracts were washed with water (2×) and brine, dried over magnesium sulphate, filtered and evaporated to an oil. Purified on a CBA column (10 g) eluting with methanol and the methanolic ammonia to give 1-butoxycarbonyl-isobutylamino-4-methylpiperidine as a colourless oil (0.19 g).

(v) To a stirred suspension of 1-butoxycarbonyl-4-isobutylamino-4-methylpiperidine (188 mg, 0.70 mmol) and anhydrous potassium carbonate (125 mg, 0.90 mmol) in dry acetonitrile at room temperature under an atmosphere of nitrogen was added 2-trifluoromethylbenzyl bromide (200 mg, 0.84 mmol). The suspension was heated at reflux for 3 days, cooled to room temperature and filtered. Washed solids with methanol and the filtrate added to an SCX-2 column (10 g). After elution with methanol, elution with methanolic ammonia gave a mixture of required product 1-butoxycarbonyl-4-methyl-4-{N-(2-methylpropyl)-N-[(2-trifluoromethylphenyl)methyl]}-piperidin-4-amine and starting material in a 11 to 4 ratio as a colourless oil (0.23 g). The mixture was taken on to the next step.

(vi) To a stirred solution of 1-butoxycarbonylmethyl-4{N-(2-methylpropyl)-N-[(2-trifluoromethylphenyl)methyl]}-piperidin-4-amine and 1-butoxycarbonyl-4-isobutylamino-4-methylpiperidine (0.23 g) in dichloromethane (10 ml) at room temperature was added trifluoroacetic acid (0.41 ml, 5.30 mmol) Stirred at room temperature overnight and then evaporated. The resulting oil was dissolved in methanol and purified on SCX-2 column (5 g) eluting with methanol and methanolic ammonia to give a colourless oil (0.16 g). This mixture was then separated by preparative 1 cms to give the title product as its acetate salt. This was converted to its free base by passing down an SCX-2 column to give a colourless oil (96 mg). The oil was dissolved in diethyl ether and a hot solution of fumaric acid (34 mg) added. The resulting colourless crystals were filtered, washed with diethyl ether and dried in vacuo at 50° C. to give the title product (112 mg). ¹HNMR (CD₃OD): δ=8.15 (1H, d), 7.67-7.59 (2H, m), 7.40 (1H, t), 6.70 (2H, s), 3.98 (2H, s), 3.32-3.24 (2H, m), 3.09-2.97 (2H, m), 2.48 (2H, d), 2.00-1.76 (4H, m), 1.43-1.32 (1H, m), 1.22 (3H, s), 0.92 (6H, d); LCMS 5.83 min [M⁺+H]: 329.

### EXAMPLE 176

### N-(2-Methylpropyl)-N-{[(3-chloro-2-(trifluoromethyl))phenyl]methyl}piperidin-4-amine fumarate

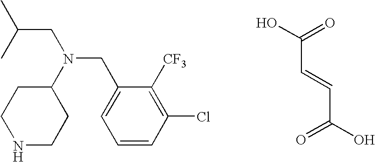

(i) To a solution of 1-chloro-3-methyl-2-(trifluoromethyl)benzene (2 g, 13.5 mmol, 1 eq) and N-bromosuccinimide (2.40 g, 13.5 mmol, 1 eq) in carbon tetrachloride (10 ml) was added a catalytic amount of dibenzoyl peroxide (16 mg, 0.7 mmol, 0.05 eq) and this was heated to reflux for 6 hours. The reaction mixture was cooled, water (10 ml) and DCM (10 ml) was added. The aqueous layer was separated and extracted with DCM (2×10 ml). The combined organic extracts were dried MgSO₄) and the solvent removed ill vacuo. The oil was purified by automated flash chromatography using an ISCO Combiflash system (120 g SiO₂) with a gradient of 0-40% ether in iso-hexane over 40 minutes to give 2-(trifluoromethyl)-3-chlorobenzylbromide (1.55 g, 50.6%); ¹H NMR (300 MHz, CDCl₃): δ=7.60 (3H, m, ArH) and 4.55 (2H, s, CH₂Br).

(ii) To a solution of 2-(trifluoromethyl)-3-chlorobenzylbromide (1.35 g, 4.9 mmol, 2.4 eq) and 1,1-dimethylethyl 4-[(2-methylpropyl)amino]piperidine-1-carboxylate (0.527 g, 2.0 mmol, 1 eq) in acetonitrile (25 ml) was added potassium carbonate (0.44 g, 3.2 mmol, 1.6 eq). The mixture was heated to reflux for 16 hours, the solution was cooled, filtered and the solvent removed in vacuo. The oil was purified by automated flash chromatography using an ISCO Combiflash system (40 g SiO₂) with a gradient of 0-20% ethyl acetate in iso-hexane over 40 minutes to give 1,1-dimethylethyl 4-[{[2-(trifluoromethyl)-3-chlorophenyl]methyl}(2-methylpropyl)amino]-piperidine-1-carboxylate (0.89 g, 40.7%) as an oil. ¹H NMR (300 MHz, CDCl₃): δ=7.92-7.90 (1H, m, ArH), 7.41-7.35 (2H, m, ArH), 4.16-4.09 (2H, m), 3.83-3.83 (2H, m, CH₂Ar), 2.61-2.42 (3H, m), 2.24 (2H, d, J=7.2, CH₂), 1.75-1.66 (2H, m), 1.63-1.51 (1H, m), 1.45-1.36 (11H, m) and 0.85 (6H, d, J=6.8,2×CH₃).

(iii) To a solution of 1,1-dimethylethyl 4-[{[2-(trifluoromethyl)-3-chlorophenyl]methyl}(2-methylpropyl)amino]-piperidine-1-carboxylate (0.90 g, 2.0 mmol, 1 eq) in DCM (3 ml) was added trifluoroacetic acid (1. ml, 20.0 mmol, 10 eq) and the mixture stirred at room temperature for 16 hr. The solvent removed in vacuo and the residue diluted with methanol (10 ml) and loaded onto SCX-2 ion exchange cartridge (10 g). The column was washed with methanol (50 ml) and the product eluted with 2M ammonia in methanol solution (50 ml) the solvent removed in vacuo to give a colourless oil. The product (0.41 mg, 59%) was taken up in diethyl ether (15 ml) and a few drops of methanol were added to solubilize. A hot solution of fumaric acid (137 mg, 1.2 mmol, 1 eq) in methanol (1 ml) was added and the solution was heated adding a few drops of methanol added until all solid was in solution, then the mixture was allowed to slowly cool to 0° C. The product was collected by filtration and dried in a vacuum oven (40° C. for 2 hr) to give N-(2-Methylpropyl)-N-{[(3-chloro-2-(trifluoromethyl)phenyl]methyl}piperidin-4-amine fumarate (405 mg, 43%) as a white solid. LCMS (12 min): Rt=5.99 min, (Oe+1)=349.1. ¹H NMR (300 MHz, CDCl₃): δ=7.79 (1H, d, J=6.9, ArH) 7.44-7.37 (2H, m, ArH), 6.58 (2H, s, fumarate CH), 3.81-3.80 (2H, m, CH₂Ar), 3.40-3.31 (2H, m), 2.88-2.78 (2H, m), 2.73-2.63 (1H, m), 2.20 (2H, d, J=7.2, CH₂), 1.95-1.90 (2H, m), 1.73-1.58 (2H, m,), 1.50-1.37 (1H, m) and 0.75 (6H, d, J=6.6, 2×CH₃).

### EXAMPLE 177

### N-(2-Hydroxyethyl)-N-{[(4-fluoro-2-(trifluoromethyl))phenyl]methyl}piperidin-4-amine fumarate

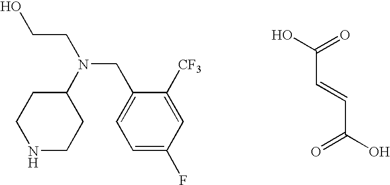

(i) To 10% Pd/C (0.5 g, 10% wt), under nitrogen, was added a solution of the N-(tert-butoxycarbonyl)-4-piperidone (5 g, 25 mmol, 1.0 eq) and ethanolamine (1.83 g, 30 mmol, 1.2 eq) in ethanol (50 ml). This was hydrogenated for 1.5 hrs, at 65 psi hydrogen, using a PARR hydrogenator. The catalyst was removed by filtration through Celite. Solvent was removed under vacuum to give the secondary amine as a colourless oil (6.4 g, 100%) with >98% purity. LCMS (6 mins gradient): Rt=1.87 (M⁺+1) 267.4.

(ii) To a solution of amine prepared in intial step (2.0 g, 8.19 mmol, 1.0 eq) in dichloroethane (20 ml) was added the 4-fluoro, 2-(trifluoromethyl)benzaldehyde (2.34 g, 12.2 mmol, 1.5 eq). To this was added sodium triacetoxyborohydride (2.58 g, 12.2 mmol, 1.5 eq.) in DMF (1 ml). This mixture was left to stir under nitrogen, at room temperature for 16 h. The reaction was worked up by addition of water (10 ml). The mixture was stirred vigorously for several minutes. The chlorinated organic layer was then run through a hydrophobic frit to remove water. The resulting organic solution was diluted with methanol (10 ml) and loaded onto an SCX-2 (10 g) column. The column was washed with methanol (50 ml) then basic material eluted with 2N ammonia in methanol. The ammonia/methanol solution was concentrated in vacuo to give an oil. This was further purified using ISCO chromatography, eluting with 0-40% ethyl acetate: iso-hexane ramp over 40 min to give the desired compound (0.173 g), which was taken onto the next step. To a solution of this oil (1.0 eq) in dichloromethane (10 ml) was added trifluoroacetic acid (TFA) (15 eq). The solution was stirred at room temperature for 4 h. Solvent and TFA were removed in vacuo. The resulting oil was taken up in methanol and loaded onto an SCX-2 (10 g) column. The column was washed with methanol (50 ml). Basic material was then eluted using 2N ammonia in methanol (50 ml). Removal of solvent from the ammonia/methanol mixture under vacuum, gave the desired compound as an oil. The oil was taken up in diethyl ether. To this solution was added a solution of fumaric acid (1 eq) in hot methanol and then cooled. The resulting precipitate was collected by filtration to give the title compound as a white solid (0.43 g); ¹HNMR (MeOD) δ=8.10-8.0 (1H, m), 7.45-7.33 (2H, m), 6.69 (2H, s), 3.91 (2H, s), 3.59-3.41 (4H, m), 3.30-3.21 (1H, s), 3.10-2.81 (3H, m), 2.75-2.68 (2H, m), 2.15 (2H, brd), 1.87-1.70 (2H, m).

### EXAMPLE 178

### N-(2,2,2-Trifluoroethyl)-N-{[(2-(trifluoromethyl))phenyl]methyl}piperidin-4-amine L-Tartrate

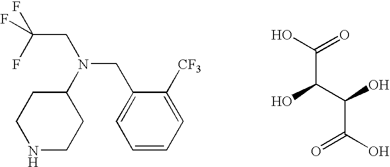

(i) To a solution of 1,1-dimethylethyl 4-({[2-(trifluoromethyl)phenyl]methyl}amino)piperidine-1-carboxylate (0.5 g, 1.4 mmol, 1 eq) in DCM (10 ml) was added triethylamine (0.40 ml, 2.8 mmol, 2 eq), trifluoroacetic anhydride (0.24 ml, 1.7 mmol, 1.2 eq) and 4-(dimethylamino)pyridine (0.086 g, 0.7 mmol, 0.5 eq). This was stirred at room temperature for 16 hours then quenched with saturated aqueous sodium hydrogen carbonate. The separated aqueous layer was extracted with DCM (3×20 ml), the combined organic layers were dried (MgSO₄) and the solvent removed in vacuo to give a colourless oil. This was purified by automated flash chromatography using an ISCO Combiflash system (40 g SiO₂) with a gradient of 20-60% ethyl acetate in iso-hexane over 40 minutes to give 1,1-dimethylethyl 4-[{[2-(trifluoromethyl)phenyl]methyl}(2,2,2-trifluoroacetyl)amino]-piperidine-1-carboxylate (518 mg, 81%). LCMS (6 min): Rt=4.95 min, (M⁺+23)=477.42.

(ii) To a solution of 1,1-dimethylethyl 4-[{[2-(trifluoromethyl)phenyl]methyl}(2,2,2-tri-fluoroacetyl)amino)-piperidine-1-carboxylate (518 mg, 1.1 mmol, 1 eq) in THF (2.5 ml) at 0° C. was added dropwise neat borane-tetrahydrofuran complex (0.32 ml, 3.3 mmol, 3 eq). The reaction mixture was heated to 55° C. for 1.5 hr then cooled and quenched with saturated sodium hydrogen carbonate (exothermic). The aqueous layer was separated and extracted with DCM (3×20 ml), the combined organic layers were dried (MgSO₄) and the solvent removed in vacuo to give 1,1-dimethylethyli[{[2-(trifluoromethyl)phenyl]methyl}(2,2,2-trifluoroethyl)amino]-piperidine-1-carboxylate (0.353 g, 70%) as a white solid. LCMS (6 min): Rt=3.42 min, (M⁺+1)=441.4.

(iii) To a solution of 1,1-dimethylethyl 4-[{[2-(trifluoromethyl)phenyl]methyl}(2,2,2-trifluoroethyl)amino]-piperidine-1-carboxylate in DCM (2 ml) was added trifluoroacetic acid (0.62 ml, 8 mmol, 10 eq) and the mixture stirred at room temperature for 16 h. The solvent removed in vacuo and the residue diluted with methanol (5 ml) and loaded onto SCX-2 ion exchange cartridge (5 g). The column was washed with methanol (15 ml) and the product eluted with 2M ammonia in methanol solution (15 ml) the solvent removed in vacuo to give an oil which was purified by mass guided preparative LCMS followed by repeat SCX-2 treatment to give a colourless oil. This was taken up in hot methanol (1.5 ml) and added to L-tartaric acid (105 mg, 1 eq), diethyl ether was added slowly until crystallization occurred. After 16 hours the crystals were collected by filtration and dried in a vacuum oven at 40° C. for 8 h to give N-(2,2,2-trifluoroethyl)-N-{[(2-(trifluoromethyl))phenyl]methyl}piperidin-4-amine-L-Tartrate (154 mg, 39%) as a colourless solid. LCMS (12 min): Rt=5.05 min, (1@+1)=341.1; ¹H NMR (300 MHz, MeOD): δ=7.81 (1H, d, J=7.9, ArH), 7.58-7.49 (2H, m, ArH), 7.34-7.30 (1H, t, J=7.5, ArH), 4.30 (2H, s, tartrate CH), 4.01 (2H, s, CH₂Ar), 3.36-3.25 (4H, m, NCH2), 2.83-2.69 (3H, m, NCH and NCH2), 1.94 (2H, d, J=13.0 Hz), 1.72-1.64 (2H, m, CCH₂).

### EXAMPLE 179

### N-(2-Methylpropyl)-N{[2-chloro-4-(methylsulfonyl)phenyl]methyl}-piperidin-4-amine L-Tartrate

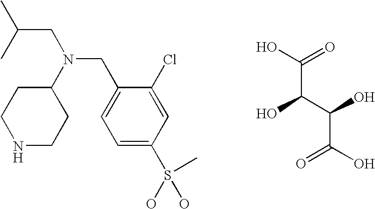

(i) 2-Chloro-4-fluorobenzaldehyde (5 g, 31.5 mmol, 1 eq) and methanesulphinic acid sodium salt (3.5 g, 34.7 mmol, 1.1 eq) were dissolved in dry DMSO (30 ml) and heated to 100° C. for 16 hrs. The mixture was cooled and poured onto crushed ice (50 g). After ice had melted the solid was filtered and dried in a vacuum oven at 50° C. for 2 hours to give 2-chloro 4-(methylsulphonyl)benzaldehyde (4.85 g, 70%) as a colourless solid. ¹H NMR (300 MHz, CDCl₃): δ=10.52 (1H, s, CHO), 8.16-7.91 (3H, m, ArH) and 3.10 (3H, s, CH₃). GCMS: Rt=7.63 min, (M⁺)=218.

(ii) To a solution of 1,1-dimethylethyl 4-[(2-methylpropyl)amino]piperidine-1-carboxylate (0.38 g, 1.5 mmol, 1 eq) and 2-chloro 4-(methylsulphonyl)benzaldehyde (984 mg, 4.5 mmol, 3 eq) in THF (10 ml) was added sodium triacetoxyborohydride (0.95 g, 4.5 mmol, 3 eq) and the mixture left to stir for 16 h. The reaction was quenched with water (10 ml), then 2N aqueous sodium hydroxide (10 ml), the aqueous layer was separated and extracted with ethyl acetate (2×20 ml). The combined organic layers were washed with brine (20 ml), dried (MgSO₄) and the solvent removed in vacuo. This was diluted with methanol (10 ml) and loaded onto a SCX-2 ion exchange cartridge (5 g) washed with methanol (15 ml) and the product eluted with 2M ammonia in methanol solution (15 ml). The solvent removed in vacuo to give an oil which was purified by automated flash chromatography using an ISCO Combiflash system (40 g SiO₂) with a gradient of 0-40% ethyl acetate in iso-hexane over 40 minutes to give 1,1-dimethylethyl 4-[{[2-chloro-4-(methylsulphonyl)phenyl]methyl}(2-methylpropyl)amino]-piperidine-1-carboxylate (0.45 g, 75%) as a colourless oil. This was taken up in DCM (2 ml) and trifluoroacetic acid (1.0 ml, 13.1 mmol, 15 eq) was added, the mixture stirred at room temperature for 16 h. The solvent removed in vacuo and the residue diluted with methanol (5 ml) and loaded onto SCX-2 ion exchange cartridge (5 g). The column was washed with methanol (15 ml) and the product eluted with 2M ammonia in methanol solution (15 ml) the solvent removed in vacuo to give a colourless oil (320 mg, 100%). This was taken up in hot methanol (1.5 ml) and added to L-tartaric acid (105 mg, 1 eq), diethyl ether was added slowly until crystallization occurred. After 16 hours the crystals were collected by filtration and dried in a vacuum oven at 40° C. for 8 hours to give N-(2-Methylpropyl)-N{[2-chloro-4-(methylsulfonyl)phenyl]methyl}-piperidin-4-amine L-Tartrate (302 mg, 66%) as a colourless solid. LCMS (12 min): Rt=4.25 min, (M⁺+1)=359.1; ¹H NMR (300 MHz, MeOD): δ=7.95-7.87 (3H, m, ArH), 4.41 (2H, s, tartrate CH), 3.89 (2H, s, CH₂Ar), 3.54-3.39 (2H, m, NCH₂), 3.32 (3H, s, CH₃), 2.99-2.78 (3H, m, NCH, NCH₂), 2.38 (2H, d, J=7.1, NCH₂), 2.06 (2H, br d, J=13.4, CCH₂), 1.89-1.61 (3H, m, CCH₂ and CH(CH₃)₂) and 0.89 (6H, d, J=6.6 Hz, 2×CH₃).

### EXAMPLE 180

### N-(3-Methoxypropyl)-N-{[(2-(trifluoromethyl))phenyl]methyl}piperidin-4-amine-L-Tartrate

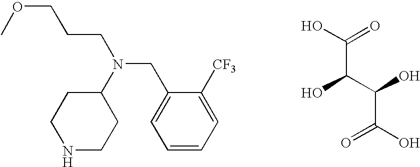

(i) A solution of N-butoxycarbonyl-4-piperidone (20.0 g, 0.1 mol) and 3-methoxypropylamine (13.4 g, 0.15 mol) in ethanol (120 ml) was hydrogenated at 60 psi over 10% palladium-carbon (2 g) for 3 h. The catalyst was removed by filtration through celite and the filtrate evaporated to 1-butoxycarbonyl-N-(3-methoxypropyl)piperidin-4-amine as a colourless oil.

(ii) To a stirred solution of 1-butoxycarbonyl-N-(3-methoxypropyl)piperidin-4-amine (0.50 g, 1.84 mmol) and 2-trifluoromethylbenzaldehyde (0.64 g, 3.67 mmol) in dry tetrahydrofuran (10 ml) at room temperature was added sodium triacetoxyborohydride (0.97 g, 4.60 mmol). Aq. saturated sodium bicarbonate was added followed by dichloromethane (15 ml). After stirring for 5 min, the organic phase was isolated using a phase separator and evaporated to give a crude oil. Purified on a 40 g cartridge of silica using an ISCO combiflash by gradient elution with iso-hexane-ethyl acetate (10 to 40%) to give the required product 1-butoxycarbonyl-N-(3-methoxypropyl)-N-{[(2-(trifluoromethyl))phenyl]methyl}piperidin-4-amine contaminated with 2-trifluoromethylbenzyl alcohol as a colourless oil. Taken on to next step without further purification.

(iii) A solution of 1-Butoxycarbonyl-N-(3-methoxypropyl)-N-{[(2-(trifluoromethyl))phenyl]methyl}piperidinamine (0.72 g) in dichloromethane (10 ml) was stirred at room temperature with trifluoroacetic acid (1.29 ml) overnight. The reaction mixture was evaporated and the resulting oil was dissolved in methanol and purified on SCX-2 column (5 g) eluting with methanol and methanolic ammonia to give a colourless oil (290 mg). The oil was dissolved in methanol and L-tartaric acid (132 mg) added, warmed to give a clear solution then allowed to stand with vapour of diethyl ether. The crystals were filtered, washed with diethyl ether and dried in vacuo at 60° C. to give the title product as a colourless solid (347 mg). ¹NMR (d⁶-DMSO): δ=7.90 (1H, d), 7.70-7.64 (2H, m), 7.47 (1H, t), 3.88 (2H, s), 3.79 (2H, s), 3.34-3.23 (4H, m), 3.17 (3H, s), 2.88-2.70 (3H, m), 2.57-2.50 (2H, m), 1.88-1.77 (2H, m), 1.76-1.50 (4H, m). LCMS 3.69 min. [M+H]: 331.

### EXAMPLE 181

### N-(3-Methoxypropyl)-N-{[(2,4-dichloro)phenyl]methyl}piperidin-4-amine L-Tartrate

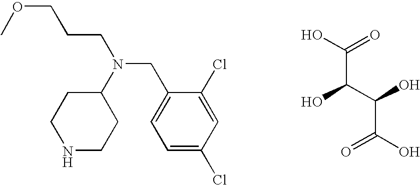 Method 1

(i) To a stirred solution of 1-butoxycarbonyl-N-(3-methoxypropyl)piperidin-4-amine (0.50 g, 1.84 mmol) and 2,4-dichlorobenzaldehyde (0.64 g, 3.67 mmol) in dry tetrahydrofuran (10 ml) at room temperature was added sodium triacetoxyborohydride (0.97 g, 4.60 mmol). Aq. saturated sodium bicarbonate was added followed by dichloromethane (15 ml). After stirring for 5 min, the organic phase was isolated using a phase separator and evaporated to give a crude oil. Purified on a 40 g cartridge of silica using an ISCO combiflash by gradient elution with iso-hexane-ethyl acetate (10 to 40%) to give the required product 1-butoxycarbonyl-N-(3-methoxypropyl)-N-{[(2,4 dichloro)phenyl]methyl}piperidin-4-amine contaminated with 2,4-dichlorobenzyl alcohol as a colourless oil. Taken on to next step without further purification.

(ii) A solution of 1-butoxycarbonyl-N-(3-methoxypropyl)-N-{[(2,4-dichloro))phenyl]methyl}piperidin-4-amine (0.76 g) in dichloromethane (10 ml) was stirred at room temperature with trifluoroacetic acid (1.36 ml) overnight. The reaction mixture was evaporated and the resulting oil was dissolved in methanol and purified on SCX-2 column (5 g) eluting with methanol and methanolic ammonia to give a colourless oil (385 mg). The oil was dissolved in methanol and L-tartaric acid (174 mg) added, warmed to give a clear solution then allowed to stand over a vapour of diethyl ether. The crystals were filtered, washed with diethyl ether and dried in vacuo at 60° C. to give the title product as a colourless solid (508 mg). LCMS 3.37 min. [M+H] 331; ¹NMR (d⁶-DMSO): 7.60 (2H, d), 7.44 (1H, d), 3.88 (2H, s), 3.67 (2H, s), 3.34-3.23 (4H, m), 3.17 (3H, s), 2.88-2.68 (3H, m), 2.57-2.50 (2H, m), 1.88-1.77 (2H, m), 1.76-1.50 (4H, m).

Method 2

(i) To a stirred solution of 1-butoxycarbonyl-N-(3-methoxypropyl)piperidin-4-amine (8.16 g; 0.03 mol) and 2,4-dichlorobenzaldehyde (10.5 g, 0.06 mol) in dry THF (120 ml) was added in one portion at room temperature sodium triacetoxyborohydride (15.9 g; 0.075 mol). The reaction was stirred for 24 h. Dichloromethane and saturated aqueous sodium bicarbonate were then added and the product extracted several times with dichloromethane. The organic extracts were collected and washed with brine, dried over anhydrous magnesium sulphate. After filtration the solvent was removed in vacuo to leave a clear oil (18 g). This was purified in 2 batches using 2×120 g silica cartridges on an ISCO combiflash via gradient elution with iso-hexane-ethyl acetate (10-40%) to yield after removal of solvent the product 1-butoxycarbonyl-N-(3-methoxypropyl)-N-{[(2,4-dichloro)phenyl]methyl}piperidin-4-amine as a clear oil (9 g).

(ii) A solution of 1-butoxycarbonyl-N-(3-methoxypropyl)-N-{[(2,4-dichloro))phenyl]methyl}piperidin-4-amine (9 g; 0.025 mol)) in dichloromethane (100 ml) was stirred at room temperature with trifluoroacetic acid (16.5 ml) overnight under a nitrogen atmosphere. The solvent/TFA was removed in vacuo and the resulting oil dissolved in dichloromethane and aqueous sodium hydroxide added (2N; 30 ml). The product was extracted several times with dichloromethane and the combined extracts washed with water. After drying with magnesium sulphate and filtering, the solvent was removed to yield a clear oil (6.9 g). The oil was dissolved in methanol (40 ml) and warmed to 50° C. on a steam bath. L-tartaric acid (3.13 g; 0.0208 mol) was dissolved in methanol (15 ml) with heating and the solutions combined at 50° C. Diethyl ether (40 ml) was slowly added to the cooling solution. The crystals produced were filtered and washed with cold methanol-ether mixture and dried at 60° C. in vacuo to give the title product as a colourless solid (7.16 g).

### EXAMPLE 182

### N-(3-Methoxypropyl)-N-{[(4-fluoro-2-(trifluoromethyl))phenyl]methyl}piperidin-4-amine-L Tartrate

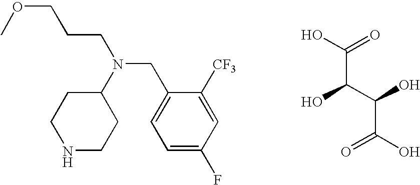

(i) To a stirred solution of 1-butoxycarbonyl-N-(3-methoxypropyl)piperidin-4-amine (0.50 g, 1.84 mmol) and 4-fluoro-2-trifluoromethylbenzaldehyde (0.70 g, 3.67 mmol) in dry tetrahydrofuran (10 ml) at room temperature was added sodium triacetoxyborohydride (0.97 g, 4.60 mmol). Aq. saturated sodium bicarbonate was added followed by dichloromethane (15 ml). After stirring for 5 min, the organic phase was isolated using a phase separator and evaporated to give a crude oil. Purified on a 40 g cartridge of silica using an ISCO combiflash by gradient elution with iso-hexane-ethyl acetate (10 to 40%) to give the required product 1-butoxycarbonyl-N-(3-methoxypropyl)-N-{[(4-fluoro-2-(trifluoromethyl))phenyl]methyl}piperidin-4-amine contaminated with 4-fluoro-2-trifluoromethylbenzylalcohol as a colourless oil. Taken on to next step without further purification.

(ii) A solution of 1-butoxycarbonyl-N-(3-methoxypropyl)-N-{[(4-fluoro-2-(trifluoromethyl))phenyl]methyl}piperidin-4-amine (0.18 g) in dichloromethane (10 ml) was stirred at room temperature with trifluoroacetic acid (0.31 ml) overnight. The reaction mixture was evaporated and the resulting oil was dissolved in methanol and purified on SCX-2 column (5 g) eluting with methanol and methanolic ammonia to give a colourless oil (95 mg). The oil was dissolved in methanol and L-tartaric acid (40 mg) added, warmed to give a clear solution then allowed to stand over a vapour of diethyl ether. The crystals were filtered, washed with diethyl ether and dried in vacuo at 60° C. to give the title product as a colourless solid (106 mg). LCMS Rt=4.26 min. [M+H] 349. ¹NMR (d⁶-DMSO): 7.90 (1H, t), 7.60-7.50 (2H, m), 3.88 (2H, s), 3.75 (2H, s), 3.34-3.23 (4H, m), 3.17 (3H, s), 2.88-2.70 (3H, m), 2.57-2.48 (2H, m), 1.88-1.77 (2H, m), 1.76-1.50 (4H, m).

Table 4 examples 183-193 were similarly prepared.

TABLE 4

| | | | LCMS (12 min |
|---|---|---|---|
| Example | | | or 6 min*) |
| No. | Structure | Name | M⁺ + 1 |
| 183 | 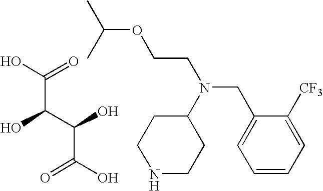 | N-{2-[(1-Methylethyl)oxy]ethyl})- N-{[(2-(trifluoromethyl))phenyl]methyl}piperidin-4-amine L-Tartrate | 4.79 min, 345 |
| 184 | 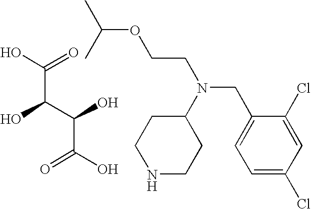 | N-{2-[(1-Methylethyl)oxy]ethyl})- N-{[(2,4-dichloro)phenyl]methyl}piperidin-4-amine L-Tartrate | 4.56 min, 345/347 |
| 185 | 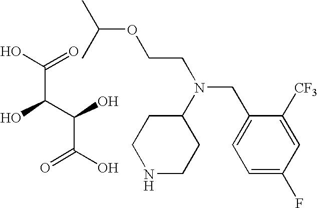 | N-{2-[(1-Methylethyl)oxy]ethyl})- N-{[(4-fluoro-2-(trifluoromethyl))phenyl]methyl}piperidin-4-amine L-Tartrate | 5.24 min, 363 |
| 186 | 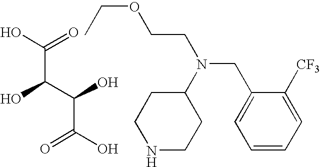 | N-[2-(Ethyloxy)ethyl]-N-{[(2- (trifluoromethyl))phenyl]methyl}piperidin-4-amine L-Tartrate | 4.47 min, 331 |
| 187 | 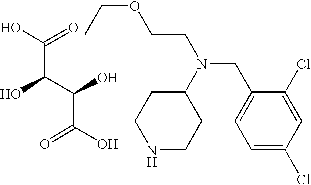 | N-[2-(Ethyloxy)ethyl]-N-{[2,4- dichlorophenyl]methyl}piperidin-4- amine L-Tartrate | 4.26 min, 331/333 |
| 188 | 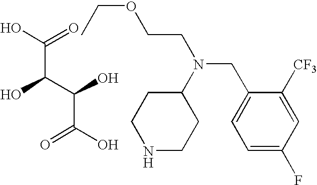 | N-[2-(Ethyloxy)ethyl]-N-{[(4- fluoro-2-(trifluoromethyl))phenyl]methyl}piperidin-4-amine L-Tartrate | 4.90 min, 349 |
| 189 | 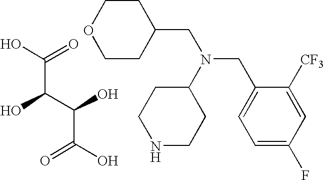 | N-{[(4-Fluoro-2-trifluoromethyl) phenyl]methyl}-N- (tetrahydro-2H-pyran-4ylmethyl)- piperidin-4-amine fumarate | 2.18 min*, 375.2 |
| 190 | 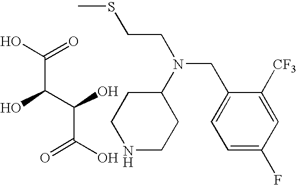 | N-[(2-(Methylthio)ethyl]-N-{[(4- fluoro-2-(trifluoromethyl))phenyl]methyl}piperidin-4-amine L-Tartrate | 5.19 min, 351 |
| 191 | 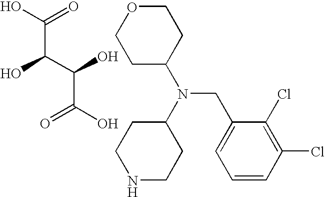 | N-{[(2,3-Dichloro)phenyl]methyl}- N-tetrahydro-2H-pyran-4-yl- piperidin-4-amine L-Tartrate | 4.63 min, 343.1/345.1 |
| 192 | 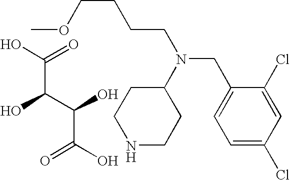 | N-{4-[(Methyl)oxy]butyl}-N-{[(2,4- dichloro)phenyl]methyl}piperidin-4- amine L-Tartrate | 3.50 (M⁺ +H) = 346 |
| 193 | 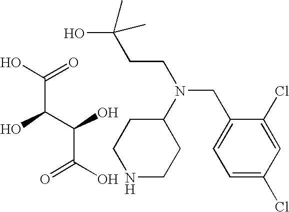 | N-(3-hydroxy-3-methylbutyl)-N- {[(2,4-dichlorophenyl)methyl}piperidin-4-amine L-Tartrate | 2.94 min, 346 |

### EXAMPLE 194

### N-(2-hydroxy-2-methylpropyl)-N-{[(2,4-dichlorophenyl)methyl}piperidin-4-amine L-Tartrate

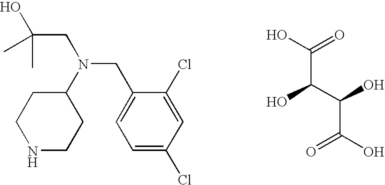

(i) To a stirred solution of isobutylene oxide (18.6 g, 0.257 mol) in acetonitrile (200 ml) cooled to 5° C. was added lithium perchlorate (27.7 g, 0.26 mol) portionwise. The suspension was stirred at room temperature for 0.5 h to give a solution. A solution of 1,1-dimethylethyl 4-({[2,4-dichlorophenyl]methyl}amino)piperidine-1-carboxylate (10.0 g, 27.83 mmol) in acetonitrile (200 ml) was then added dropwise over a period of approximately 30 min. The reaction mixture was heated at reflux for 24 h, cooled and concentrated under vacuum. The concentrate was taken up in dichloromethane (200 ml) and washed with water (150 ml) and brine. The organic phase was dried over anhydrous magnesium sulphate, filtered and evaporated to a pale yellow oil. The crude oil was purified using a combiflash on a redisep column (120 g) by gradient elution with iso-hexane-ethyl acetate (10-45%) over 35 min to give 1,1-dimethylethyl 4-({[2,4dichlorophenyl]methyl}{2-hydroxy-2-methylpropyl}amino)piperidine-1-carboxylate as a colourless oil (7.0 g).

(ii) To a stirred solution of 1,1-dimethylethyl 4-({[2,4-dichlorophenyl]methyl}{2-hydroxy-2-methylpropyl}amino)piperidine-1-carboxylate (7.0 g, 16.24 mmol) in dichloromethane (80 ml) at room temperature was added trifluoroacetic acid (18.5 g, 0.16 mol). The solution was stirred at room temperature for 17 h, concentrated to approximately half volume and with ice cooling made basic with aqueous sodium hydroxide (2M). The organic phase was separated and the aqueous phase extracted twice with dichloromethane. The combined organic phases was washed twice with water and then with brine, dried over anhydrous magnesium sulphate, filtered and evaporated to a pale yellow oil. The oil was dissolved in warm methanol and a solution of L-tartaric acid (1 eq) in methanol added. Diethyl ether was added and the resulting crystals filtered, washed with diethyl ether and dried under vacuum at 40-45° C. to give the title compound as a colourless solid 4.85 g. LCMS (12 min) Rt=4.54 min [M+H] 331.3. ¹H NMR (400 MHz, MeOH-D4) 7.51 (1H, d), 7.23 (1H, s), 7.14 (1H, d), 4.14 (1H, s), 3.75 (2H, s), 3.29-3.18 (2H, m), 2.75-2.56 (3H, m), 2.38 (2H, s), 1.93-1.82 (2H, m), 1.68-1.50 (2H, m) 0.92 (6H, s).

### EXAMPLE 195

### N-{2-[(Trifluoromethyl)oxy]ethyl}-N-{[(2,4-dichloro)phenyl]methyl}piperidin-4-amine L-Tartrate

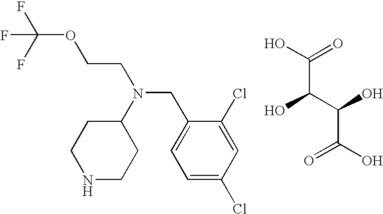

(i) Tetrabutylammonium sulfate (10.2 ml, 8.78 mmol) was added to diethyleneglycol (15 g, 141 mmol), followed by 50% aqueous NaOH solution (285 ml, ˜12.5 M). The reaction was stirred for 10 minutes, and then carbon disulfide (285 ml) was added dropwise over 20 minutes, followed by MeI (43.7 g, 308 mmol). The reaction was stirred at ambient temperature for 4 hours before water (50 ml) was added. The layers were separated and the aqueous layer was extracted with dichloromethane. The combined organic extracts were washed with aqueous saturated sodium chloride, dried over MgSO₄, filtered, and concentrated in vacuo. The crude material was purified on silica gel eluting with 70% dichloromethane/hexanes to yield (18.80 g, 47%) of dithiocarbonic acid S-methyl ester O-[2-(2-methylsulfanylthiocarboxyoxy-ethoxy)-ethyl] ester: ¹H NMR (400 MHz, CDCl₃): δ=4.6 (4H, m), 3.8 (4H, m), 2.5 (6H, s).

(iia) HF-pyridine complex (42 ml) followed by dithiocarbonic acid S-methyl ester O-[2-(2-methylsulfanylthiocarboxyoxy-ethoxy)-ethyl] ester (8.4 g, 29.3 mmol), was added to a cold (−78° C.) solution of 1,3-dibromo-5,5-dimethylhydantoin (51.18 g, 179 mmol) in dichloromethane (300 ml). The reaction was warmed to ambient temperature and stirred for 1.5 hours, then poured into cold aqueous saturated sodium chloride. The layers were separated, and the aqueous layer extracted with dichloromethane. The combined organic extracts were washed with cold 37% aqueous NaHSO₃ and cold aqueous saturated sodium chloride then dried over MgSO₄, filtered, and concentrated in vacuo at ambient temperature. The residue was purified by bulb to bulb distillation under mild vacuum at 120° C. and trapped at −78° C. to yield (5.81 g, 82%) of 1-trifluoromethoxy-2-(2-trifluoromethoxy-ethoxy)-ethane: ¹H NMR (400 MHz, CDCl₃): δ=4.0 (4H, m), 3.70 (4H, m).

(iib) A tube was charged with 1-trifluoromethoxy-2-(2-trifluoromethoxy-ethoxy)-ethane (5.81 g, 24 mmol). Trifluoroacetic acid (0.55 ml, 6.2 mmol) and trifluoroacetic anhydride (16 ml, 95.1 mmol) were added, the tube sealed well under N₂ atmosphere, and then immersed in a 60° C. heated oil bath and heated for 5 days. The tube was removed from the oil bath and cooled to room temperature. The reaction mixture was concentrated in vacuo with ambient temperature bath. The residue was partitioned between dichloromethane and water, the layers were separated, the organic layer was dried over MgSO₄, and concentrated in vacuo. The crude material was purified by bulb to bulb distillation with mild vacuum at 120° C. and trapped at −78° C. to yield (7.26 g, 58%) of trifluoro-methanesulfonic acid 2-trifluoromethoxy-ethyl ester: ¹H NMR (400 MHz, CDCl₃): δ=4.69 (2H, m), 4.27 (2H, m).

(iic) Trifluoro-methanesulfonic acid 2-trifluoromethoxy-ethyl ester (0.541 g, 2.06 mmol) was added to a solution of 4-(2,4-dichloro-benzylamino)-piperdine-1-carboxylic acid tert-butyl ester (0.76 g, 2.13 mmol) and K₂CO₃ (0.626 g, 4.53 mmol) in anhydrous acetonitrile (5 ml). The reaction mixture was stirred at ambient temperature overnight then water and dichloromethane were added. The layers were separated and the aqueous layer was extracted with dichloromethane. The combined organic extracts was dried over MgSO₄, filtered, and concentrated in vacuo. The crude material was purified on silica gel eluting with 15% EtOAc/hexanes to yield (0.59 g, 61%) 4-[(2,4-dichloromethyl-benzyl)-(2-trifluoromethoxy-ethyl)-amino]-piperdine-1-carboxylic acid tert-butyl ester: ¹H NMR (400 MHz, CDCl₃) δ=7.50 (1H, d), 7.35 (1H, d), 7.23 (1H, dd), 4.18 (2H, brs), 3.87-3.81 (2H, m), 3.78 (2H, s), 2.89-2.83 (2H, m), 2.67-2.56 (2H, m), 1.77 (2H, brd), 1.48-1.38 (12H, m).

(iii) 4-[(4-Fluoro-2-trifluoromethyl-benzyl)-(2-trifluoromethoxy-ethyl)-amino]-piperdine-1-carboxylic acid tert-butyl ester (0.59 g, 1.26 mmol) was added to a stirred solution of dichloromethane (30 ml) and anisole (0.684 ml, 6.29 mmol). The reaction was cooled to 0° C. Trifluoroacetic acid (10 ml, 130 mmol) was then added. The reaction was stirred for 30 minutes at 0° C. The reaction was loaded onto an SCX-2 (10 g) column and washed with methanol (200 ml). The product was then eluted with 2M ammonia in methanol (100 ml) and concentrated to yield (0.413 g, 88%) of (2,4-dichloro-benzyl)-piperdin-4-yl-(2-trifluoromethoxy-ethyl)-amine: mass spectrum (ion spray): m/z=371 (M⁺+1); ¹H NMR (400 MHz, CD₃OD): δ=7.60 (1H, d), 7.40 (1H, d), 7.29 (1H, dd), 3.92-3.88 (2H, m), 3.84 (2H, s), 3.10 (2H, brd), 2.93-2.89 (2H, m), 2.69-2.49 (3H, m), 1.83 (2H, brd), 1.57-1.45 (2H, m).

(iv) L-Tartaric acid (0.175 g, 1.17 mmol) was added to a solution of (2,4-dichloro-benzyl)-piperdin-4-yl-(2-trifluoromethoxy-ethyl)-amine (0.41 g, 1.11 mmol) in methanol (5 ml). The solution was sonicated for 30 minutes at ambient temperature and concentrated. Water (3 ml) was added to the residue, and the material was lyophilized to yield (0.56 g, 97%) of the title compound (ion spray): m/z=371 (M⁺+1); ¹H NMR (400 MHz, CD₃OD): δ=7.59 (1H, d), 7.43 (1H, d), 7.31 (1H, dd), 4.41 (2H, s), 3.95-3.90 (2H, m), 3.88 (2H, s), 3.45 (2H, brd), 3.00-2.82 (5H, m), 2.05 (2H, brd), 1.85-1.72 (2H, m).

### EXAMPLE 196

### N-{2-[(Trifluoromethyl)oxy]ethyl}-N-{[(4-fluoro-2-(trifluoromethyl))phenyl]methyl}piperidin-amine L-Tartrate

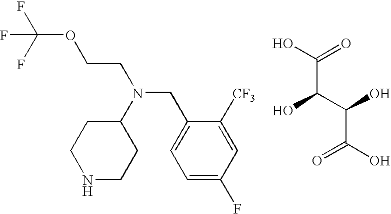

The title compound was prepared as example 195; mass spectrum (ion spray): m/z=388 (M); ¹H NMR (400 MHz, CD₃OD): δ=7.95 (1H, dd), 7.43 (1H, dd), 7.39-7.33 (1H, m), 4.44 (2H, s), 3.98-3.92 (4H, m), 3.45 (2H, brd), 3.00-2.81 (5H, m), 2.05 (2H, brd), 1.83-1.71 (2H, m).

### EXAMPLE 197

### N-{2-[(Trifluoromethyl)oxy]ethyl}-N-{[(2-(trifluoromethyl))phenyl]methyl}piperidin-4-amine L-Tartrate

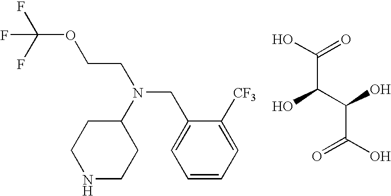

The title compound was prepared as example 195; mass spectrum (ion spray): m/z=371 (M+1); ¹H NMR (400 MHz, CD₃OD): δ=7.92 (1H, d), 7.66 (1H, d), 7.59 (1H, dd), 7.41 (1H, dd), 4.42 (2H, s), 4.00-3.92 (4H, m), 3.45 (2H, brd), 3.00-2.83 (5H, m), 2.05 (2H, brd), 1.85-1.72 (2H, m).

### EXAMPLE 198

### N-(Cyclopropylmethyl)-N-{[(4-fluoro-2-(trifluoromethyl))phenyl]methyl}piperidin-4-amine L-Tartrate

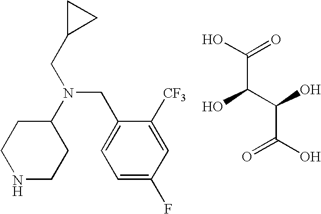

(i) In a 1 Li Parr bottle, N-boc-4-piperidone (80 g, 0.4 mol) is dissolved in 0.4 L THF. Under nitrogen stream, aminomethylcyclopropane (33 g, 0.464 mol, 1.16 equiv.) and 8 g of 10% Pd/C are added and the resulting suspension is hydrogenated under 40 psi H₂ for 1 hour. The catalyst is filtered over Celite and the solution is evaporated to dryness to afford 106 g (104%) of an oil which is used as it in the next step.

(ii) In a 3-L double jacketed-reactor with overhead stirring (anchor-type), secondary amine (104 g) is dissolved in THF (Roland—1.0 L; 0.050% w/v water). Powdered NaHB(OAc)₃ (104.0 g; 1.2 equiv) and 2-trifluoromethyl-4-fluoro-benzaldehyde (66.7 mL; 1.1 equiv) are added. Mass temperature rises from rt=22.5° C. to 27.3° C. within 40 min and then slowly decreases to rt=22.5° C.).

After 8 h, powdered NaHB(OAc)₃ (21.5 g; 0.25 equiv) and 2-trifluoromethyl-4-fluoro-benzaldehyde (11.1 mL; 0.2 equiv) are added. Mixture is allowed to stir overnight at rt=22.5° C. After 23 h, ¹H NMR ratio of starting material vs product is 1:5.4. NaHB(OAc)₃ (21.5 g; 0.25 equiv) and 2-trifluoromethyl-4-fluoro-benzaldehyde (11.1 mL; 0.2 equiv) are added. One hour later, ratio is 1:5.8. Reaction is allowed to stir at rt23° C. for another 24 h. ¹H NMR ratio of starting material vs product after a total of 48 h is 1:20. Reaction is left under minimum stirring for the weekend. The mixture is cooled down to 0° C. and water (400 mL) is added (ΔTm=12.5° C.). Once A™ max is reached, Tj is set to 20° C. Once Tm=20° C., MTBE (800 mL) is added. Layers are separated and aqueous layer, whose pH=5-6, is extracted by MTBE (400 mL). Organic layers are pooled and washed with NaOH 2N (2×400 mL), NaCl 10% (2×400 mL) and evaporated to dryness to yield tertiary amine containing 2-trifluoromethyl-4-fluororbenzylic alcohol (221.7 g—82.2% area).

(iii) In a 3-L double jacketed-reactor with overhead stirring (anchor-type), HCl 37% Merck p.a.—150 mL) is diluted in water (500 mL). The solution is heated up to 65° C. The neat tertiary amine (221.7 g) is added dropwise. However, after 10% of the addition, we observed that the tertiary amine is immiscible in the aqueous system and that no reaction occurs (no gas evolution). THF (150 mL) is added to the reactor in order to solubilise the tertiary amine. The reaction starts instantaneously. Addition of the remaining 90% of tertiary amine is restarted and completed within 20 min. After 30 min post-stirring at 65° C., HPLC shows that the reaction is completed. The mixture is cooled down to 20° C. within 1 h. Aqueous layer is washed with MTBE (3×400 mL). Tj is set to 10° C. and the mixture is basified by NaOH 15%. Tm rises from 18° C. to 28° C. Basic mixture (pH=14) is cooled to 23° C. and extracted with MTBE (2×800 mL). Organic layer is washed with NaCl 10% (400 mL) and then evaporated to dryness to yield (124 g—99.5% area). Yield from secondary amine: 92%.

(iv) In a 3-L double jacketed-reactor with overhead stirring (anchor-type), amine (115.5 g) is dissolved in i-PrOH. The solution is heated up to 70° C. Mixture is seeded with title product (1 g) after each quarter of addition of a solution of L-tartaric acid (52.5 g, 1.0 equiv) in water (40 mL), until crystallisation occurs. It effectively occurs during addition of the third quarter of L-tartaric acid solution. The remaining acid is added. The mixture is allowed to cool down to 20° C. slowly (75 min) and is then stirred at 20° C. during 1 h 30 min. Suspension is filtered on fritted glass (P3), filtration is quick: 5 min under 0.4 bar vacuum. Reactor is rinsed with mother liquors and the crystals are refiltered on the cake. Filtration is slower but no compression of the cake is noticed. The title product is washed with i-PrOH (750 mL) and dried under vaccuum at 40° C. Yield=(96%, 167.2 g)

### EXAMPLE 199

### 2-Methylpropan-2-ol1-[[(4-fluoro-2-(trifluoromethyl)phenyl)methyl]piperidin-4 amine]L-Tartrate

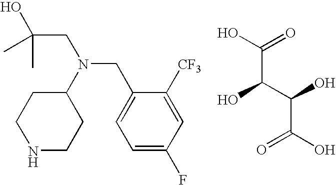

(i) Isobutylene oxide (2.5 mL, 27.74 mmol) was added to a solution of 4-(4-fluoro-2-trifluoromethyl-benzylamino)-piperdine-1-carboxylic acid tert-butyl ester (1.01 g, 26.83 mmol) in anhydrous methanol (11 mL). The reaction was stirred at ambient temperature for 3 days, then at reflux for 5 h. The reaction mixture was cooled to ambient temperature and LiClO₄ (0.42 g, 3.94 mmol) was added. The reaction was stirred at ambient temperature overnight, then at reflux for 5 days. The cooled reaction was poured into 100 mL of aqueous saturated NaHCO₃ and extracted with ethyl acetate (100 mL×3). The ethyl acetate was dried over sodium sulfate, filtered, and concentrated. The crude product was purified by flash chromatography on silica gel eluting with 30% EtOAc/hexanes to yield (0.85 g, 71%) of 4-[(4-fluoro-2-trifluoromethyl-benzyl)-(2-hydroxy-2-methyl-propyl)-amino]-piperdine-1-carboxylic acid tert-butyl ester: mass spectrum (ion spray): m/z=449 (M⁺+1); ¹H NMR (400 MHz, CDCl₃): δ=7.92-7.88 (1H, m), 7.34 (1H, dd), 7.29-7.23 (1H, m), 4.16 (2H, brs), 3.99 (2H, s), 2.60-2;45 (6H, m), 1.76 (2H, brd), 1.48-1.37 (11H, m), 1.16 (6H, s).

(ii) 4-[(4-Fluoro-2-trifluoromethyl-benzyl)-(2-hydroxy-2-methyl-propyl)-amino]-piperdine-1-carboxylic acid tert-butyl ester (0.845 g, 18.84 mmol) was added to a stirred solution of dichloromethane (5 mL) and anisole (9.0 mL, 82.8 mmol). The reaction was cooled to 0° C. Trifluoroacetic acid (6.0 mL, 72.9 mmol) was then added. The reaction was stirred for 5 minutes at 0° C. and then for 2 h at room temperature. The reaction was loaded onto an SCX-2 (10 g) column and washed with methanol (200 mL). The product was then eluted with 2M ammonia in methanol (100 mL) and concentrated to yield (0.603 g, 92%) of 1-(4-fluoro-2-trifluoromethyl-benzyl)-piperdin-4-yl-amino]-2-methyl-propan-2-ol: mass spectrum (ion spray): m/z=349 (M⁺+1); ¹H NMR (400 MHz, CDCl₃): δ=7.90 (1H, dd), 7.33 (1H, dd), 7.28-7.22 (1H, m), 3.99 (2H, s), 3.11 (2H, brd), 2.58 (2H, s), 2.50-2.38 (3H, m), 1.78 (2H, brd), 1.51-1.39 (2H, m), 1.15 (6H, s).

(v) L-Tartaric acid (0.25 g, 1.66 mmol) was added to a solution of 1-(4-fluoro-2-trifluoromethyl-benzyl)-piperdin-4-yl-amino]-2-methyl-propan-2-ol (0.58 g, 1.66 mmol) in methanol (15 mL). The solution was stirred for 1.5 h at ambient temperature and concentrated. The solid was dried in a vacuum oven at 45° C. overnight to yield (0.81 g, 99%) 1-(4-fluoro-2-trifluoromethyl-benzyl)-piperdin-4-yl-amino]-2-methyl-propan-2-ol tartrate: mass spectrum (ion spray): m/z=349 (M+1); ¹H NMR (400 MHz, CD₃OD): δ=8.17-8.10 (1H, m), 7.44-7.34 (2H, m), 4.41 (2H, s), 4.03 (2H, s), 3.44 (2H, brd), 2.91-2.73 (3H, m), 2.55 (2H, s), 2.02 (2H, brd), 1.83-1.69 (2H, brd), 1.19 (6H, s).

### EXAMPLE 200

### N-[1-(4-Fluoro-2-(trifluoromethyl)phenyl)ethyl]-N-(cyclopropylmethyl)piperidin-4-amine L-Tartrate

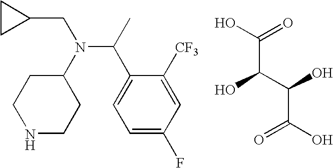

(i) To a solution of 4-oxo-piperidine-1-carboxylic acid tert-butyl ester (1.4 g, 7.03 mmol), cyclopropylmethylamine (500 mg, 7.03 mmol) and acetic acid (0.40 mL, 7.03 mmol) in 1,2-dichloroethane (69 mL) at 0° C. was added sodium triacetoxy-borohydride (2.08 g, 9.8 mmol). The reaction was warmed to ambient temperature and stirred for overnight under N₂. The reaction mixture was poured on to 2N NaOH (50 mL) and extracted with ethyl acetate (3×). The combined organic extracts were washed with aqueous saturated NaCl, dried (Na₂SO₄), filtered and concentrated. The crude product was purified by flash chromatography on silica gel eluting with 5% EtOH (10% NH₄OH)/chloroform to yield 1.32 g (74%) of 4-(Cyclopropylmethyl-amino)-piperidine-1-carboxylic acid tert-butyl ester: mass spectrum (ion spray): m/z=255.1 (4+1); ¹H NMR (400 MHz, CD₃OD): δ=4.11 (2H, m), 2.94-2.66 (3H, m), 2.52 (2H, d, J=7.0 Hz), 1.93 (2H, m), 1.50 (9H, s), 1.35-1.19 (2H, m), 1.05-0.91 (1H, m), 0.59-0.51 (2H, m), 0.24-0.18 (2H, m).

(ii) Benzotriazole (326 mg, 2.75 mmol) and 4-(cyclopropylmethyl-amino)-piperidine-1-carboxylic acid tert-butyl ester (700 mg, 2.75 mmol) were dissolved in dry benzene (30 mL). 4-fluoro-2-trifluoromethyl benzaldehyde (0.38 mL, 2.75 mmol) was then added and the reaction was heated under reflux for overnight with a Dean-Stark trap. The reaction mixture was concentrated and the crude was dissolved in dry THF (20 mL). The reaction was cooled to 0° C. and methylmagnesium bromide (3.0 M solution in Et₂O, 1.1 mL, 3.02 mmol) was added dropwise. The reaction was stirred at ambient temperature for 1 hour. The reaction was quenched with saturated ammonium chloride and extracted with ethyl acetate (2×). The combined organic extracts were washed with aqueous saturated sodium chloride, dried (Na₂SO₄), filtered and concentrated. The crude product was purified by flash chromatography on silica gel eluting with 25% EtOAc/hexane to yield (174 mg, 14%) of 4-{Cyclopropylmethyl-[1-(4-fluoro-2-trifluoromethyl-phenyl)-ethyl]-amino}-piperidine-1-carboxylic acid tert-butyl ester: mass spectrum (ion spray): m/z 445.1 (M⁺+1); ¹H NMR (400 MHz, CD₃OD): δ=8.13-8.05 (1H, m), 7.43-7.36 (2H, m), 4.43-4.34 (1H, m), 4.17-4.03 (2H, m), 2.76-2.46 (4H, m), 2.27 (1H, dd, J=14.9, 7.0 Hz), 1.81-1.71 (1H, m), 1.68-1.34 (16H, m), 0.88-0.77 (1H, m), 0.51-0.44 (2H, m), 0.13-0.04 (2H, m).

(iii) 4-{Cyclopropylmethyl-[1-(4-fluoro-2-trifluoromethyl-phenyl)-ethyl]-amino}-piperidine-1-carboxylic acid tert-butyl ester (0.142 mg, 0.32 mmol) was added to a stirred solution of dichloromethane (1.5 mL) and anisole (2.5 mL, 23 mmol). Trifluoroacetic acid (1.0 mL, 12.15 mmol) was then added. The reaction was stirred for 2 h at room temperature. The reaction was loaded onto an SCX-2 (10 g) column and washed with methanol (40 mL). The product was then eluted with 2M ammonia in methanol (25 mL) and concentrated to yield (0.104 g, 95%) of cyclopropylmethyl-[1-(4-fluoro-2-trifluoromethyl-phenyl)-ethyl]-piperidin-4-yl-amine: mass spectrum (ion spray): m/z=345.2(M+1); ¹H NMR (400 MHz, CD₃OD): δ=8.13-8.06 (1H, m), 7.43-7.35 (2H, m), 4.42-4.32 (1H, m), 3.16-3.01 (2H, m), 2.75 (1H, dd, J=15.2, 7.3 Hz), 2.67-2.57 (1H, m), 2.49-2.39 (2H, m), 2.28 (1H, dd, J=15.2, 7.3 Hz), 1.86-1.77 (1H, m), 1.61-1.46 (3H, m), 1.39 (d, 3H, J=6.6 Hz), 0.90-0.78 (1H, m), 0.50-0.44 (2H, m), 0.15-0.04 (2H, m).

(iv) L-Tartaric acid (39 mg, 0.26 mmol) was added to a solution of cyclopropylmethyl-[1-(4-fluoro-2-trifluoromethyl-phenyl)-ethyl]-piperidin-4-yl-amine (0.09 g, 0.26 mmol) in methanol (3 mL). The solution was stirred for 1.5 h at ambient temperature and concentrated. The solid was dried in a vacuum oven at 45° C. overnight to yield (0.125 g, 97%) of the title product: mass spectrum (ion spray): m/z=345.2 (M⁺+1); ¹H NMR (400 MHz, CD₃OD): δ=8.10-8.04 (1H, m), 7.45-7.38 (2H, m), 4.44-4.36 (3H, m), 3.50-3.36 (2H, m), 2.93-2.79 (3H, m), 2.75 (1H, dd, J=15.0, 5.3 Hz), 2.31 (1H, dd, J=15.0, 7.0 Hz), 2.06-1.97 (1H, m), 1.90-1.75 (3H, m), 1.41 (3H, d, J=6.6 Hz), 0.88-0.78 (1H, m), 0.52-0.45 (2H, m), 0.15-0.03 (2H, m).

HPLC Method: (100/0 to 5/95 0.2% formic acid in water/0.2% formic acid in acetonitrile) Xterra MS C₁₈ 2.1 mm×50 mm×3.5 micron, 6 minute run, 1.15 minutes retention time, 100% purity.

### EXAMPLE 201

### N-(3-Hydroxypropyl)-N-[[(2,4-dichlorophenyl)methyl](piperidin-4-amine L-Tartrate

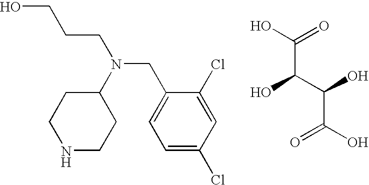

(i) To 10% Pd/C (1.0 g, 10% wt), under nitrogen, was added a solution of the N-(tert-butoxycarbonyl)-4-piperidone (10 g, 50 mmol) and propanolamine (3.76 g, 50 mmol) in ethanol (50 ml). This was hydrogenated for 1.5 hrs, at 65 psi hydrogen, using a PARR Hydrogenator. The catalyst was removed by filtration through Celite. Solvent was removed under vacuum to give the secondary amine as a colourless oil (12.8 g, 100%) with >98% purity. LCMS (6 mins gradient): Rt=1.93 (M⁺+1) 259.4.

(ii) Prepared as example 177 to give the title compound. LCMS (12 min) Rt=2.52, M⁺+1=317.1/319.1. ¹HNMR (MeOD): δ=7.58 (1H, d), 7.42 (1H, s), 7.33 (1H, d), 4.42 (2H, s), 3.80 (2H, s), 3.59-3.55 (2H, m), 3.46 (2H, brd), 3.32 (2H, s), 2.99-2.83 (3H, m), 2.73-2.68 (2H, m), 2.05-1.91 (2H, m), 1.90-1.87 (2H, m), 1.67-1.62 (2H, m).

### EXAMPLE 202

### N-(2-Hydroxyethyl)-N-[[(2,4-Dichlorophenyl)methyl](piperidin-4-amine] L-Tartrate

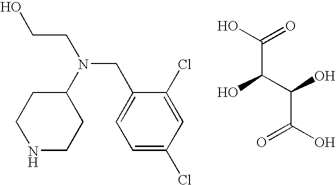

This compound was prepared using the same method as for example 177 replacing 4-fluoro-2-(trifluoromethyl)benzaldehyde with 2,4-dichlorobenzaldehyde. LCMS— (12 mins gradient): Rt=2.84 (M⁺+1) 303.1/305.1; ¹HNMR (MeOD): δ=7.51 (1H, d), 7.30 (1H, s), 7.19 (1H, d), 4.29 (2H, s), 3.7 (2H, s), 3.41-3.30 (4H, m), 3.20 (1H, s), 2.90-2.70 (3H, m), 2.65-2.54 (2H, m), 1.95-1.88 (2H, brd), 1.75-1.60 (2H, m).

### EXAMPLE 203

### 3-[[(4-Fluoro-2-(trifluoromethyl)phenyl)methyl](piperidin-4-yl)amino]propanenitrile L-Tartrate

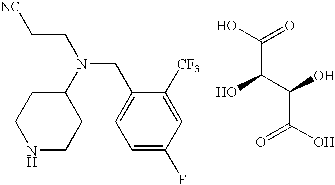

(i) To a solution of N-(tert-butoxycarbonyl)-4-piperidone (1.0 g, 5.0 mmol, 1 eq) and 3-aminopropionitrile (0.35 g, 5.0 mmol, 1 eq) in THF (20 ml) was added sodium triacetoxyborohydride (1.48 g, 7.0 mmol, 1.4 eq) and the mixture stirred for 16 hours. The mixture was diluted with water (100 ml) and 2N sodium hydroxide (10 ml), and then extracted with ethyl acetate (3×10 ml). The combined organic extracts were washed with brine (20 ml), dried (MgSO₄) and the solvent removed in vacuo to give 1,1-dimethylethyl 4-[(2-cyanoethyl)amino]piperidine-1-carboxylate (0.88 g, 70%) as a colourless oil. LCMS (6 min): Rt=1.97 min, (M⁺+23)=276.4.

(ii) Prepared using a method similar to that described for example 180(ii) with 1,1-dimethylethyl 4-[(2-cyanoethyl)amino]piperidine-1-carboxylate and 4-fluoro-2-(trifluoromethyl)benzaldehyde with additional purification using the Biotage Parallel Flex Purification System (UV-guided HPLC) followed by SCX-2 treatment prior to tartrate salt formation to give 3-[[(4-fluoro-2-(trifluoromethyl)phenyl)methyl](piperidin-4-yl)amino]propanenitrile L-Tartrate (117 mg, 38%) as a colourless solid. LCMS (12 min): Rt=4.53 min, (M⁺+1)=330.1; ¹H NMR (300 MHz, MeOD): δ=8.08-8.03 (1H, m, ArH), 7.47-7.38 (2H, m, ArH), 4.41 (2H, s, tartrate CH), 3.93 (2H, s, CH₂Ar), 3.49-3.45 (2H, m, NCH₂), 2.98-2.82 (5H, m, NCH), 2.58 (2H, t, J=6.3, CHCN), 2.06 (2H, br. d, J=13.6, CCH₂) and 1.87-1.76 (2H, m, CCH₂).

### EXAMPLE 204

### 3-[[(4-Fluoro-2-(trifluoromethyl)phenyl)methyl](piperidin-4-l)amino]butanenitrile L-Tartrate

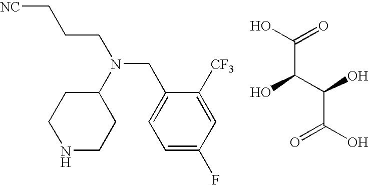

(i) Prepared using a method similar to that described for example (203 a) with 4-fluoro-2-(trifluoromethyl)benzylamine to give 1,1-dimethylethyl 4-({[4-fluoro-2-(trifluoromethyl)phenyl]methylamino)piperidine-1-carboxylate (8.98 g, 82%) as a colourless oil; LCMS (6 min): Rt=3.00 min, (M⁺+1)=377.1.

(ii) To a solution of 1,1-dimethylethyl 4-({[4-fluoro-2-(trifluoromethyl)phenyl]methyl}amino)piperidine-1-carboxylate (0.49 g, 1.3 mmol, 1 eq) in acetonitrile (10 ml) was added potassium carbonate (0.37 g, 2.6 mmol 2 eq), sodium iodide (0.19 g, 1.3 mmol, 1 eq) followed by 4-bromobutyronitrile (1.5 ml, 15 mmol, 12 eq). This was heated to reflux for 48 hr, whereby reaction was incomplete so a further portion of 4-bromobutyronitrile (0.37 g, 2.6 mmol, 12 eq) was added and heating to reflux was continued for 72 brs. The reaction mixture was cooled, filtered and evaporated; the residue was diluted with methanol (5 ml) and loaded onto SCX-2 ion exchange cartridge (5 g). The column was washed with methanol (15 ml) and the product eluted with 2M ammonia in methanol solution (15 ml) then the solvent removed in vacuo. This oil was purified by automated flash chromatography using an ISCO Combiflash system (40 g SiO₂) with a gradient of 0-40% ethyl acetate in iso-hexane over 40 minutes to give 1,1-dimethylethyl 4-({[4-fluoro-2-(trifluoromethyl)phenyl]methyl}(3-cyanopropyl)amino)piperidine-1-carboxylate (220 mg, 38%) as a colourless oil. LCMS (6 min): Rt=4.91 min, (M⁺+1)=444.4.

(iii) To a solution of 1,1-dimethylethyl 4-({[4-fluoro-2-(trifluoromethyl)phenyl]methyl}(3-cyanopropyl)amino)piperidine-1-carboxylate (220 mg, 0.4 mmol, 1 eq) in DCM (2 ml) was added trifluoroacetic acid (1 ml) and the mixture stirred at room temperature for 4 hours. The solvent was removed in vacuo, and then the residue was diluted with methanol (5 ml) and loaded onto SCX-2 ion exchange cartridge (5 g). The column was washed with methanol (15 ml), the product eluted with a solution of 2M ammonia in methanol (15 ml) and the solvent removed in vacuo. This was further purified on the Biotage Parallel Flex Purification System (UV-guided HPLC) followed by repeat SCX-2 treatment. The free base was taken up in hot methanol (1.0 ml) and added to L-tartaric acid (40 mg, 1 eq), diethyl ether was added slowly until crystallization occurred. After 16 hours the crystals were collected by filtration and dried in a vacuum oven at 40° C. for 8 hours to give 3-[[(4-Fluoro-2-(trifluoromethyl)phenyl)methyl](piperidin-4-yl)amino]butanenitrile L-Tartrate (122 mg, 62%) as a colourless solid. LCMS (12 min): Rt=4.40 min, (M⁺+1)=344.1 ¹H NMR (300 MHz, MeOD) δ=7.95-7.87 (1H, m, ArH), 7.45-7.41 (2H, m, ArH), 4.41 (2H, s, tartrate CH), 3.86 (2H, s, CH₂Ar), 3.49-3.45 (2H, m, NCH₂), 3.00-2.81 (3H, m, NCH), 2.70 (2H, t, J=6.5, NCH₂), 2.46 (2H, t, J=6.8, CHCN), 2.08-2.04 (2H, m, CCH₂) and 1.84-1.73 (4H, m, CCH₂), LCMS: 12 min, RT=4.40 min, (M⁺1)=344.1

### EXAMPLE 205

### N-(Cyclopropylmethyl)-N-{[(2,3-Dichloro)phenyl]methyl}piperidin-4-amine L-Tartrate

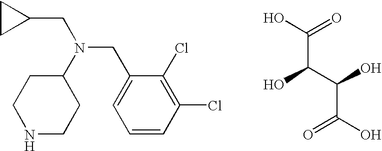

(i) Prepared using a method similar to that described for example (179b) staring with 1,1-dimethylethyl 4-{[(2,3-dichlorophenyl)methyl]amino}piperidine-1-carboxylate to give 1,1-dimethylethyl 4-{[(2,3-dichlorophenyl)methyl](cyclopropylmethyl)amino}-piperidine-1-carboxylate (5.27 g, 36%) as a yellow oil. LCMS: (6 min), Rt=3.40 min, M⁺+1)=413.4.

(ii) To a solution of 1,1-dimethylethyl 4-{[(2,3-dichlorophenyl)methyl](cyclopropylmethyl)amino}-piperidine-1-carboxylate (5.27 g, 12.5 mmol, 1 eq) in DCM (20 ml) was added trifluoroacetic acid (9.7 ml, 125 mmol, 10 eq) and the mixture stirred at room temperature for 4 h. Water (50 ml) and iso-hexane (50 ml) were added and the aqueous layer separated. This was basified with 2N aqueous sodium hydroxide (60 ml) and extracted with a 1:1 mixture of diethyl ether and iso-hexane (3×50 ml). The combined organic extracts were dried (MgSO₄) and the solvent removed in vacuo This oil was purified by automated flash chromatography using an ISCO Combiflash system (120 g SiO₂) with a gradient of 0-40% of a 5% ammonia in methanol solution in DCM for 30 mins to give an oil. The product was taken up in hot methanol (26 ml) and added to L-tartaric acid (1.25 mg, 1 eq), diethyl ether was added slowly until crystallization occurred. After 16 hours the crystals were collected by filtration and dried in a vacuum oven at 40° C. for 16 hours to give N-(Cyclopropylmethyl)-N-{[(2,3-dichloro)phenyl]methyl}piperidin-4-amine L-Tartrate (3.7 g, 64%) as a colourless solid. LCMS: (12 min): Rt=3.17 min, (M⁺1)=313.1. 1H NMR (300 MHz, MeOD): δ=7.61-7.57 (1H, m, ArH), 7.35-7.32 (1H, m, ArH), 7.23-7.17 (1H, m, ArH), 4.32 (2H, s, tartrate C₁H), 3.83 (2H, s, CH₂Ar), 3.40-3.36 (2H, m, NCH₂), 3.02-2.85 (3H, m, NCH), 2.42 (2H, d, J=6.6), 1.98 (2H, br d, J=13.4), 1.79-1.67 (2H, m), 0.82-0.69 (1H, m, CH), 0.40-0.34 (2H, m) and 0.03-0.00 (2H, m).

### EXAMPLE 206

### 3-2[(4-Fluoro-2-(trifluoromethyl)phenyl)methyl](piperidin-4-ylamino]2,2-dimethyl]propanenitrile L-Tartrate

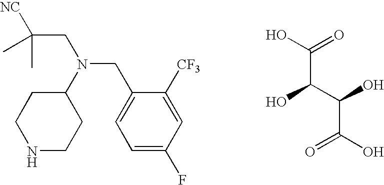

(i) To a solution of diisopropylamine (0.44 ml, 3.1 mmol, 2.5 eq) in THF (6 ml) at 0° C. was added dropwise a solution of 1.6M n-butyllithium in hexanes (1.9 ml, 3.1 mmol, 2.5 eq). This was stirred at 0° C. for 30 mins to form the lithium diisopropylamide. Half of the solution of this lithium diisopropylamide solution was added dropwise to a solution of 1,1-dimethylethyl 4-[(2-cyanoethyl)amino]piperidine-1-carboxylate (537 mg, 1.25 mmol, 1 eq) in TB (2 ml) at −78° C. After 30 min methyl iodide (0.08 ml, 1.25 mmol, 1 eq) was added and the mixture was allowed to slowly warm to room temperature over 30 mins. The solution was then cooled back to −78° C. and the second portion of lithium diisopropylamide was added dropwise followed by methyl iodide (0.08 ml, 1.25 mmol, 1 eq) 30 mins later. This was again allowed to warm slowly to room temperature over 30 mins then quenched with saturated aqueous ammonium chloride (20 ml). The aqueous layer was separated and extracted with ethyl acetate (3×20 ml), the combined organic layers were washed with brine, dried (MgSO₄) and the solvent removed in vacuo. To give an oil which was purified by automated flash chromatography using an ISCO Combiflash system (40 g SiO₂) with a gradient of 0-30% ethyl acetate in iso-hexane over 40 minutes to give 1,1-dimethylethyl 4-({[4-fluoro-2-(trifluoromethyl)phenyl]methyl}(2-cyano-2-methylpropyl)amino)piperidine-1-carboxylate (183 mg, 32%) as a yellow oil. LCMS: 6 min, Rt=5.74 min, (M⁺+1)=458.2.

(ii) Prepared using a method similar to that described for example (204 c) with 1,1-dimethylethyl 4-({[4-fluoro-2-(trifluoromethyl)phenyl]methyl}(2-cyano-2-methylpropyl)amino)piperidine-1-carboxylate to give 3-[[(4-Fluoro-2-(trifluoromethyl)phenyl)methyl](piperidin-4-yl)amino]2,2-dimethylpropanenitrile L-Tartrate (42 mg, 20%) as a colourless oil. LCMS: (12 min), Rt=5.14 min, (M⁺+1)=358.1. 1H NMR (300 MHz, MeOD): δ=8.05-7.96 (1H, m, ArH), 7.36-7.29 (2H, m, ArH), 4.29 (2H, s, tartrate CH), 4.00 (2H, s, CH₂Ar), 3.38-3.34 (2H, m, NCH₂), 2.83-2.62 (5H, m, NCH), 1.98 (2H, br. D, I=13.7, CCH₂), 1.75-1.67 (2H, m) and 1.23-1.22 (6H, m, 2×CH₃).

### EXAMPLE 207

### 4-[[(2,4-Dichlorophenyl)methyl]piperidin-4-yl)amino]-2,2-dimethylbutanenitrile L-Tartrate

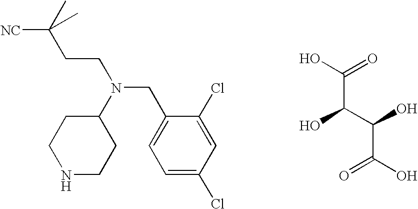

(i) To a solution of diisopropylamine (50.8 ml, 360 mmol, 1.2 eq) in THF (600 ml) at −78° C. was added dropwise over 40 mins a solution of 1.6M n-butyllithium in hexanes (206 ml, 330 mmol, 1.1 eq), maintaining the temperature below −68° C. After stirring for 1 hr isobutyronitrile (27.2 ml, 300 mmol, 1 eq) was added dropwise over 20 minutes, maintaining the temperature below −70° C. The reaction was stirred for 2 hrs before addition of 1,3-dimethyl-3,4,5,6-tetrahydro-2(1H)-pyrimidinone (DMFU) (54.4 ml, 450 mmol, 1.5 eq) in THE (40 ml) over 10 mins directly followed by bromoacetaldehyde diethyl acetal (45.2 ml, 300 mmol, 1 eq) over 10 minutes maintaining the temperature below −70° C. This was stirred at −78° C. for 45 mins and then allowed to warm to 0° C., where it was held for 30 min. The reaction mixture was poured onto saturated aqueous ammonium chloride (1 L), and the product extracted with diethyl ether (1 L). The combined organic layers were washed with brine (1 L), dried (MgSO₄) and the solvent removed in vacuo to give an oil which was purified by vacuum distillation (0.13 mBar) at 50° C. to afford 4,4-bis(ethyloxy)-2,2-dimethylbutanenitrile (55.8 g, 100%) as a colourless oil. ¹H NMR (300 MHz, CDCl₃): δ=4.73 (1H, t, J=5.6, CH), 3.74-3.50 (4H, m, 2×CH₂), 1.85 (2H, d, J=5.5, CH₂), 1.40 (6H, s, 2×CH₃) and 1.23 (6H, t, J=7.1, 2×CH₃).

(ii) To a 10% aqueous solution of oxalic acid (32.3 g, 358.7 mmol, 1.2 eq in 323 ml water) was added 4,4-bis(ethyloxy)-2,2-dimethylbutanenitrile (53.8 g, 290.4 mmol, 1 eq) and acetone (646 ml) and the reaction was heated to reflux for 140 mins. The solution was cooled and concentrated to half the volume in vacuo, and was then carefully decanted onto saturated sodium bicarbonate (1 L) and ice with stirring. The product was extracted in DCM (5×250 ml), the combined organic layers were dried (MgSO₄) and the solvent removed in vacuo to give 2,2-dimethyl-4-oxobutanenitrile (26.94 g, 84%) as a colourless oil. ¹H NMR (300 MHz, CDCl₃): δ=9.82 (1H, t, J=1.8, CHO), 2.66 (2H, d, J=1.9, CH₂) and 1.47 (6H, s, 2×CH₃).

(iii) To a solution of 1,1-dimethylethyl 4-{[(2,4-dichlorophenyl)methyl]amino}piperidine-1-carboxylate (7.91 g, 22.0 mmol, 1 eq) and 2,2-dimethyl-4-oxobutanenitrile (7.3 g, 66.0 mmol, 3 eq) in THF (100 ml) was added sodium triacetoxyborohydride (13.9 g, 66.0 mmol, 3 eq) was added and the mixture left to stir for 16 h. The reaction was quenched with water (200 ml), then 2N aqueous sodium hydroxide (200 ml), the aqueous layer was separated and extracted with ethyl acetate (2×100 ml). The combined organic layers were washed with brine (100 ml), dried (MgSO₄) and the solvent removed in vacuo. This was purified by automated flash chromatography using an ISCO Combiflash system (330 g SiO₂) with a gradient of 0-60% ethyl acetate in iso-hexane over 80 minutes to give 1,1-dimethylethyl 4-{[(2,4-dichlorophenyl)methyl](3-cyano-3-methylbutyl)amino}piperidine-1-carboxylate (12.19 g, 97%) as a colourless oil. LCMS (6 min): Rt=4.55 min, (M⁺+1)=454.4.

(iv) To a solution of 1,1-dimethylethyl 4-{[(2,4-dichlorophenyl)methyl](3-cyano-3-methylbutyl)amino}piperidine-1-carboxylate (93 mg, 0.2 mmol, 1 eq) in 1,4-dioxane (1 ml) was added dropwise a solution of 1M HCl in ether (1.0 ml, 6.2 mmol, 5 eq) and the mixture was stirred at room temperature overnight. The solvent was removed in vacuo, the residue was diluted with methanol (5 ml) and loaded onto SCX-2 ion exchange cartridge (2 g). The column was washed with methanol (5 ml), the product eluted with a solution of 2M ammonia in methanol (5 ml) and the solvent removed in vacuo. The free base was taken up in hot methanol (1 ml) and added to L-tartaric acid (27 mg, 1 eq), diethyl ether was added slowly until crystallization occurred. After 16 hours the crystals were collected by filtration and dried in a vacuum oven at 40° C. for 8 hours to give the title compound (75 mg, 73%) as a colourless solid. LCMS (6 min): Rt=3.06 min, (M⁺+1)=345.4; ¹H NMR (300 MHz, MeOD): δ=7.83 (1H, d, J=8.3, ArH), 7.66 (1H, d, J=2.1, ArH), 7.55 (1H, dd, J=2.1 and 8.3, ArH), 4.62 (2H, s, tartrate CH), 4.03 (2H, s, CH₂Ar), 3.69 (2H, br d, J=12.8, NCH₂), 3.24-3.10 (3H, m, NCH), 3.02-2.97 (2H, m, NCH), 2.30 (2H, br d, J=13.3), 2.12-2.00 (2H, m), 1.93-1.89 (2H, m) and 1.51 (6H, s, 2×CH₃).

Example 208 shown in Table 5 was prepared using a method similar to that described for examples (207b) and (207c) using 1,1-dimethylethyl 4-({[4-fluoro-2-(trifluoromethyl)phenyl]methyl}amino)piperidine-1-carboxylate and the appropriately substituted aldehyde

TABLE 5

| | | | LCMS |
|---|---|---|---|
| Example | | | (12 minute) |
| No. | Structure | Name | M⁺ + 1 |
| 208 | 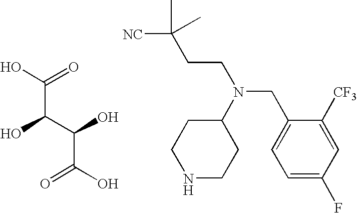 | 4-[[(4-Fluoro-2- (trifluoromethyl))phenyl]methyl](piperidin-4-yl)amino]-2,2- dimethylbutanenitrile L-Tartrate | 5.34 min, 372.1 |

### EXAMPLE 209

### 3-[[(2,4-Dichloro)phenyl]methyl}(piperidin-4-yl)amino]-butanenitrile L-Tartrate

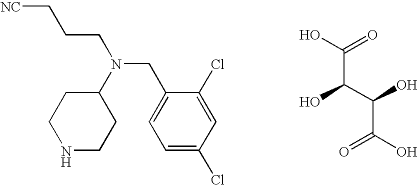

Prepared using a method similar to that described for examples (204b) and (207c) starting with 1,1-dimethylethyl 4-{[(2,4-dichlorophenyl)methyl]amiino}piperidine-1-carboxylate including additional purification using the Biotage Parallel Flex Purification System (UV-guided HPLC) followed by SCX-2 treatment prior to tartrate salt formation to give 3-[[(2,4-Dichloro)phenyl]methyl}(piperidin-4-yl)amino]-butanenitrile L-Tartrate (226 mg, 45%) as a colourless solid. LCMS (12 min): Rt=4.51 min, (M⁺+1)=326.0; ¹HNMR (300 MHz, MeOD): δ=7.35 (1H, d, J=8.3, ArH), 7.25 (1H, d, J=2.1, ArH), 7.14 (1H, dd, J=8.3 and 2.1, ArH), 4.21 (2H, s, tartrate CH), 3.60 (2H, s, CH₂Ar), 3.27 (2H, br d, J=12.6, NCH₂), 2.81-2.61 (3H, m, NCH), 2.50 (2H, t, J=6.5, NCH₂), 2.24 (2H, t, J=7.0, CHCN), 1.85 (2H, br d, J=13.4, CCH₂) and 1.71-1.49 (4H, m, CCH₂).

### EXAMPLE 210

### 3-[[(2(Trifluoromethyl)phenyl]methyl](piperidin-4-yl)amino]-butanenitrile L-Tartrate

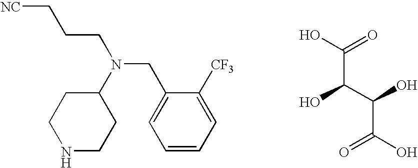

The title compound was prepared using a method similar to that described for example (204b) and (207c) starting with 1,1-dimethylethyl 4-({[2-(trifluoromethyl)phenyl]methyl}amino)piperidine-1-carboxylate including additional purification using the Biotage Parallel Flex Purification System (UV-guided HPLC) followed by SCX-2 treatment prior to tartrate salt formation to give 3-[[(2-(trifluoromethyl)phenyl]methyl](piperidin-4-yl)amino]-butanenitrile L-Tartrate (291 mg, 45%) as a colourless solid. LCMS (12 min): Rt=4.56 min, (M⁺+1) 326.1; ¹H NMR (300 MHz, MeOD): δ=7.79 (1H, d, J=7.7, ArH), 7.57-7.49 (2H, m, ArH), 7.347.29 (1H, m, ArH), 4.29 (2H, s, tartrate CH), 3.78 (2H, s, CH₂Ar), 3.35 (2H, br. d, J=12.8, NCH₂), 2.87-2.71 (3H, m, NCH), 2.59 (2H, t, I=6.5, NCH₂), 2.35 (2H, t, J=7.0, CHCN), 1.95 (2H, br. d, J=14.3, CCH₂) and 1.74-1.60 (4H, m, CCH₂).

### EXAMPLE 211

### N-(3-Methyl-3-hydroxybutyl)-N-{[4-fluoro-2-(trifluoromethyl)phenyl]methyl}piperidin-4-amine L-Tartrate

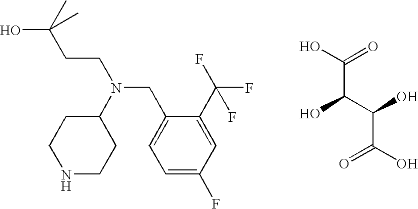

(i) A solution of N-butoxycarbonyl-4-piperidone (1.03 g, 5.18 mmol) and 4-fluoro-2-(trifluoromethyl)benzylamine (1.0 g, 5.18 mmol) in ethanol (15 ml) was stirred at room temperature under nitrogen overnight. The reaction mixture was cooled to 5° C. and sodium borohydride (0.39 g, 10.36 mmol) was added portionwise, stirred at room temperature for 1.5 h. The reaction mixture was concentrated in vacuo, diluted with water and extracted twice with diethyl ether. The extracts were washed with brine, dried over magnesium sulphate, filtered and evaporated to give an oil 2.31 g. The crude oil was purified using an ISCO combiflash on a redisep column (120 g) by gradient elution with iso-hexane-ethyl acetate (80-100%) over 20 min to obtain 1,1-dimethylethyl 4-({[4-fluoro-2-(trifluoromethyl)phenyl]methyl}amino)piperidine-1-carboxylate as a colourless oil (1.05 g).

(ii) A solution of 1,1-dimethylethyl 4-({[4-fluoro-2-(trifluoromethyl)phenyl]methyl}amino)piperidine-1-carboxylate (1.05 g, 2.79 mmol) and methylvinylketone (0.98 g, 13.95 mmol) in chloroform (20 ml) was heated with stirring at 60° C. for 3 days. The reaction solution was cooled and evaporated to give 1,1-dimethylethyl 4-({[4-fluoro-2-(trifluoromethyl)phenyl]methyl}{2-oxobutyl}amino)piperidine-1-carboxylate as an oil (1.24 g).

(iii) To a stirred solution of 4-({[4-fluoro-2-(trifluoromethyl)phenyl]methyl}(2-oxobutyl}amino)piperidine-1-carboxylate (1.24 g, 2.79 mmol) in dry diethyl ether (20 ml) cooled under nitrogen to 5° C. was added dropwise a solution of methylmagnesium chloride in tetrahydrofuran (3.0M, 2.8 ml). After addition, the reaction mixture was stirred at room temperature overnight. The reaction mixture was washed with aqueous saturated ammonium chloride, the organic phase dried over magnesium sulphate, filtered and evaporated to an oil. The oil was purified using a combiflash on a redisep column (40 g) by gradient elution with iso-hexane-ethyl acetate (50-80%) over 20 min to give 4-({[4-fluoro-2-(trifluoromethyl)phenyl]methyl}{2-hydroxy-2-methylbutyl}amino)piperidine-1-carboxylate as a colourless solid (1.19 g).

(iv) A solution of 4-({[4-fluoro-2-(trifluoromethyl)phenyl]methyl}{2-hydroxy-2-methylbutyl}amino)piperidine-1-carboxylate (1.19 g, 2.57 mmol) and trifluoroacetic acid (2.94 g, 25.70 mmol) in dichloromethane (10 ml) was stirred at room temperature overnight. The reaction solution was evaporated to an oil, the oil dissolved in methanol and converted to free base using and SCX-2 column (10 g) eluting with methanol and then methanol-ammonia. The latter was evaporated to give an oil, this was dissolved in methanol and L-tartaric acid (1 eq) added. The suspension was heated to give a clear solution and then allowed to stand over diethyl ether in a sealed container. The resulting crystals were filtered, dried under vacuum at 55° C. to give the title compound as a colourless solid, (0.84 g). LCMS 3.35 min [M+H] 363; ¹H NMR (400 MHz, DMSO-D6): δ=7.73 (1H, t), 7.63-7.56 (2H, m), 3.89 (2H, s), 3.71 (2H, s), 3.343.25 (2H, m), 2.90-2.73 (3H, m), 2.63-2.55 (2H, m), 1.90-1.62 (4H, m), 1.53-1.44 (2H, m), 1.00 (6H, s).

### EXAMPLE 212

### 1-{[(2,4-Dichloro)phenyl]methyl}(piperidin-4-yl)amino]cyclopentanol L-Tartrate

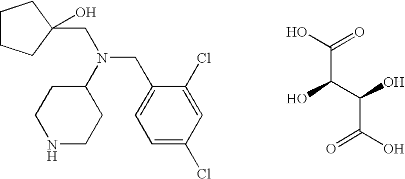

(i) Following the previous experimental procedure (i) using 2,4-dichlorobenzylamine in place of 4-fluoro-2-(trifluoromethyl)benzylamine, 1,1-dimethylethyl 4-({[2,4-dichlorophenyl]methyl}amino)piperidine-1-carboxylate was obtained as a pale yellow oil that crystallised on standing. Less pure fractions from the chromatography were evaporated and required product crystallised from ethyl acetate. Total yield (26.1 g).

(ii) To a stirred mixture of 1,1-dimethylethyl 4-({[2,4-dichlorophenyl]methyl}amino)piperidine-1-carboxylate (7.53 g, 20.96 mmol), anhydrous potassium carbonate (5.79 g, 41.90 mmol) and potassium iodide (3.48 g, 20.96 mmol) in dry acetonitrile (60 ml) was added methyl bromoacetate (8.02 g, 52.39 mmol). The mixture was heated at reflux under nitrogen for 20 h, cooled to room temperature and concentrated under vacuum. The concentrate was diluted with water and extracted twice with dichloromethane. The extracts were washed with brine, dried over magnesium sulphate, filtered and evaporated to a yellow oil. The crude oil was purified using a combiflash on a redisep column (120 g) by gradient elution with iso-hexane-ethyl acetate (0-35%) over 30 min to give 1,1-dimethyethyl 4-({[2,4-dichlorophenyl]methyl}{2-methoxy-2-oxoethyl}amino)piperidine-1-carboxylate as a yellow oil (6.71 g).

(iii) To a stirred solution of 1,1-dimethyethyl 4-({[2,4-dichlorophenyl]methyl}{2-methoxy-2-oxoethyl}amino)piperidine-1-carboxylate (472 mg, 1.09 mmol) in dry tetrahydrofuran (10 ml) cooled under nitrogen to 5° C. was added dropwise a solution of butyl-1,4-dimagnesium bromide in tetrahydrofuran (0.5M, 6.54 ml). The reaction mixture was then stirred at room temperature overnight, washed with a saturated solution of aqueous ammonium chloride and evaporated to an oil. The oil was dissolved in methanol and purified on a SCX-2 column (10 g) eluting with methanol and methanol-ammonia to give 1,1-dimethyethyl 4-({[2,4-dichlorophenyl]methyl}{[1-hydroxycyclopentyl]methyl}}amino)piperidine-1-carboxylate as a yellow oil (383 mg).

(iv) A solution of 1,1-dimethyethyl 4-({[2,4-dichlorophenyl]methyl} {[1-hydroxycyclopentyl]methyl}}amino)piperidine-1-carboxylate (376 mg, 0.82 mmol) and trifluoroacetic acid (937 mg, 8.20 mmol) in dichloromethane (9 ml) was stirred at room temperature for 24 h. The reaction solution was evaporated and the residue purified by SCX-2 chromatography eluting with methanol-ammonia to liberate the required product as the free base. The resulting oil was combined with L-tartaric acid in methanol and heated to give a clear solution. This was left to stand over diethyl ether in a sealed container, the resulting crystals were collected and dried under vacuum at 50° C. to give the title compound as a colourless solid (292 mg). LCMS 4.79 min [M+H] 357/9; ¹H NMR (400 MHz, MeOH-D4): δ=7.71 (1H, d), 7.45 (1H, d), 7.36 (1H, dd), 4.37 (1H, s), 3.97 (2H, s), 3.48 (2H, d), 2.91 (3H, m), 2.72 (2H, s), 2.15-2.08 (2H, m), 1.91-1.76 (4H, m), 1.63-1.55 (6H, m).

### EXAMPLE 213

### N-[1-Fluorocyclopropyl)methyl]-N-{[(2,4-dichloro)phenyl]methyl}piperidin-4-amine L-Tartrate

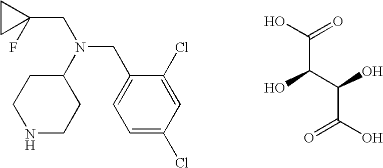

(i) To a solution of 1-fluoro-cyclopropanecarboxylic acid (200 mg, 1.93 mmol), 4-(2,4-Dichloro-benzylamino)-piperidine-1-carboxylic acid tert-butyl ester (694 mg, 1.93 mmol) and Diisoproplyethyl amine (0.68 mL, 3.86 mmol) in DMF (18 mL) was added TBTU (651 mg, 2.02 mmol). The reaction was stirred at ambient temperature for 3 hours under N₂. The reaction mixture was diluted with ethyl acetate and washed with H₂O, aqueous saturated NaHCO₃ and brine. The ethyl acetate was dried over sodium sulfate, filtered, and concentrated. The crude product was purified by flash chromatography on silica gel eluting with 25% EtOAc/hexanes to yield (0.455 g, 53%) of 4-[(2,4-Dichloro2-benzyl)-(2-fluoro-2-cyclopropanecarbonyl)-amino]-piperdine-1-carboxylic acid tert-butyl ester: mass spectrum (ion spray): m/z=445.1 (M⁺+1); ¹H NMR (400 MHz, CD₃OD): δ=7.49 (1H, s), 7.44-7.03 (2H, m), 4.72-4.44 (2H, m), 4.27-4.07 (3H, m), 2.95-2.68 (2H, m), 1.88-1.56 (4H, m), 1.48 (9H, s), 1.41-1.24 (4H, m).

(ii) 4-[(2,4-dichloro2-benzyl)-(2-fluoro-2-cyclopropanecarbonyl)-amino]-piperdine-1-carboxylic acid tert-butyl ester (0.24 g, 0.54 mmol) was added to a stirred solution of dichloromethane (2 mL) and anisole (3.0 mL, 27.6 mmol). Trifluoroacetic acid (11.0 mL, 12.15 mmol) was then added. The reaction was stirred for 2 h at room temperature. The reaction was loaded onto an SCX-2 (10 g) column and washed with methanol (40 mL). The product was then eluted with 2M ammonia in methanol (25 mL) and concentrated to yield (0.18 g, 95%) of 1-Fluoro-cyclopropanecarboxylic acid (2,4-dichloro-benzyl)-piperidin-4-yl-amide: mass spectrum (ion spray): m/z=345.1 (M+1); ¹H NMR (400 MHz, CD₃OD): δ=7.49 (1H, s), 7.43-7.27 (1H, m), 7.16-7.04 (1H, m), 4.72-4.43 (1H, m), 3.17-3.06 (2H, m), 2.75-2.62 (2H, m), 1.90-1.64 (4H, m), 1.46-1.29 (4H, m).

(iii) A solution of 1-fluoro-cyclopropanecarboxylic acid (2,4-dichloro-benzyl)-piperidin-4-yl-amide (166 mg, 0.48 mmol) in dry THF (5 mL) is heated to reflux and then was added borane-methyl sulfide complex (10 M solution in THF, 0.25 mL, 2.4 mmol). The resulting mixture was refluxed under nitrogen overnight. The reaction was cooled to room temperature and 5.0 N HCl (4 mL) was added dropwise. The reaction was heated to reflux and stir for 1 hour. The reaction was cooled to room temperature, diluted with ethyl acetate (50 mL). The aqueous layer was separated and the organic layer was washed with aqueous saturated NaHCO₃ and brine. The ethyl acetate was dried over sodium sulfate, filtered and concentrated. The crude product was purified by flash chromatography on silica gel eluting with 10% EtOH (1% NH₄OH)/chloroform to yield (0.144 g, 72%) of (2,4-Dichloro-benzyl)-(1-fluoro-cyclopropylmethyl)-piperdine-4-yl-amine. mass spectrum (ion spray): m/z=331.1 (M+1); ¹H NMR (400 MHz, CD₃OD): δ=7.74 (1H, d, J=8.3 Hz), 7.41 (1H, dd, J=1.7, 1.7 Hz), 7.34-7.29 (1H, m), 3.93 (2H, s), 3.17-3.1.0 (2H, m), 3.01 (2H, d, J=20.2 Hz), 2.84-2.73 (1H, m), 2.63-2.52 (2H, m), 1.88 (2H, d, J=11.4 Hz), 1.60-1.46 (2H, m), 0.98-0.85 (2H, m), 0.63-0.55 (2H, m).

(v) L-Tartaric acid (49 mg, 0.326 mmol) was added to a solution of (2,4-dichloro-benzyl)-(1-fluoro-cyclopropylmethyl)-piperdine-4-yl-amine (0.108 g, 0.326 mmol) in methanol (3 mL). The solution was stirred for 1.5 h at ambient temperature and concentrated. The solid was dried in a vacuum oven at 45° C. overnight to yield (0.15 g, 97%) of (2,4-dichloro-benzyl)-(1-fluoro-cyclopropylmethyl)-piperdine-4-yl-amine tartrate: mass spectrum (ion spray): m/z=331.2 (M⁺+1); ¹H NMR (400 MHz, CD₃OD): δ=7.73 (1H, d, J=8.3 Hz), 7.45-7.42 (1H, m), 7.34 (1H, dd, J=8.3, 1.8 Hz), 4.44 (2H, s), 3.97 (2H, s), 3.53-3.43 (2H, m), 3.09-2.92 (5H, m), 2.16-2.06 (2H, m), 1.90-1.75 (2H, m), 1.01-0.90 (2H, m), 0.64-0.55 (2H, m).

HPLC Method: (100/0 to 5/95 0.2% formic acid in water/0.2% formic acid in acetonitrile) Xterra MS C₁₈ 2.1 mm×50 mm×3.5 micron, 6 minute run, 2.15 minutes retention time, 100% purity.

The compounds of the present invention are inhibitors of the uptake of one or more monoamines selected from serotonin, norepinephrine and dopamine. They work by selectively inhibiting one or more of the biogenic amine (serotonin, norepinepbrine and dopamine) transporter proteins. Their selectivity profiles may be determined using the assays described below (see also J. Gobel, D. L. Saussy and A. Goetz, J. Pharmacol. Toxicolo. (1999), 42, 237-244). Compounds of Formula I and their pharmaceutically acceptable salts preferably exhibit a Kᵢ value less than 500 nM at one or more of these monoamine transporter proteins as determined using the scintillation proximity assay as described below. The compounds of Formula I exemplified above and their pharmaceutically acceptable salts exhibit a Kᵢ value less than 100 nM at one or more of these monoamine transporter proteins as determined using the assays described below. Preferred compounds of Formula I and their pharmaceutically acceptable salts exhibit a Kᵢ value less than 50 nM at one or more of these monoamine transporter proteins. Especially preferred compounds of Formula I and their pharmaceutically acceptable salts exhibit a Kᵢ value less than 20 nM at one or more of these monoamine transporter proteins. Preferably, compounds of the present invention which selectively inhibit one of the three biogenic amine transporters do so relative to the other two transporters by a factor of at least five, more preferably by a factor of at least ten. Preferably, compounds of the present invention which selectively inhibit two of the three biogenic amine transporters do so relative to the other transporter by a factor of at least five, more preferably by a factor of at least ten.

Biogenic amine transporters control the amount of neurotransmitters in the synaptic cleft. Inhibition of the respective transporter leads to a rise in that neurotransmitter. Inhibition of the individual transporters can be studied by a simple competitive binding assay using selective radioligands for the individual expressed human transporter site. Compounds may be compared for selectivity and potency on the human norepinephrine transporter (hNET), the h-serotonin transporter (hSERT) and the h-dopamine transporter (HDAT) using membranes prepared from HEK293 cells expressing the respective transporter site. Advantageously, the compounds of the present invention also have a reduced interaction (both as substrate and inhibitor) with the liver enzyme Cytochrome P450 (CYP2D6). That is to say, they preferably exhibit less than 75% metabolism via the CYP2D6 pathway according to the CYP2D6 substrate assay described below and they preferably exhibit an IC₅₀ of >61M according to the CYP2D6 inhibitor assay described below.

Generation of Stable Cell-Lines Expressing the Human Dopamine, Norepinephrine and Serotonin Transporters

Standard molecular cloning techniques were used to generate stable cell-lines expressing the human dopamine, norepinephrine and serotonin transporters. The polymerase chain reaction (PCR) was used in order to isolate and amplify each of the three full-length cDNAs from an appropriate cDNA library. Primers for PCR were designed using the following published sequence data:

Human dopamine transporter: GenBank M95167. Reference: Vandenbergh D J, Persico A M and Uhl G R. A human dopamine transporter cDNA predicts reduced glycosylation, displays a novel repetitive element and provides racially-dimorphic TaqI RFLPs. Molecular Brain Research (1992) volume 15, pages 161-166.

Human norepinephrine transporter: GenBank M65105. Reference: Pacholczyk T, Blakely, R D and Amara S G. Expression cloning of a cocaine- and antidepressant-sensitive human noradrenaline transporter. Nature (1991) volume 350, pages 350-354.

Human serotonin transporter: GenBank L05568. Reference: Ramamoorthy S, Bauman A L, Moore K R, Han H, Yang-Feng T, Chang A S, Ganaphthy V and Blakely R D. Antidepressant- and cocaine-sensitive human serotonin transporter: Molecular cloning, expression, and chromosomal localization. Proceedings of the National Academy of Sciences of the USA (1993) volume 90, pages 2542-2546.

The PCR products were cloned into a mammalian expression vector (eg pcDNA3.1 (Invitrogen)) using standard ligation techniques. The constructs were then used to stably transfect HEK293 cells using a commercially available lipofection reagent (Lipofectamine™—Invitrogen) following the manufacture's protocol.

Norepinephrine Binding Assay

The ability of compounds to compete with [³H]-Nisoxetine for its binding sites on cloned human norepinephrine membranes has been used as a measure of its ability to block norepinephrine uptake via its specific transporter.

Membrane Preparation:

Cell pastes from large scale production of HEK-293 cells expressing cloned human noradrenaline transporters were homogenised in 4 volumes 50 mM Tris.HCl containing 300 mM NaCl and 5 mM KCl, pH 7.4. The homogenate was centrifuged twice (40,000 g, 10 min, 4° C.) with pellet re-suspension in 4 volumes Tris.HCl buffer after the first spin and 8 volumes after the second spin. The suspended homogenate was centrifuged (100 g, 10 min, 4° C.) and the supernatant kept and re-centrifuged (40,000 g, 20 min, 4° C.). The pellet was resuspended in Tris.HCl buffer containing the above reagents along with 10% w/v sucrose and 0.1 mM phenylmethylsulfonyl fluoride (PMSF). The membrane preparation was stored in aliquots (1 ml) at −80° C. until required. The protein concentration of the membrane preparation was determined using a bicinchoninic acid (CA) protein assay reagent kit (available from Pierce).

[³H]-Nisoxetine Binding Assay:

Each well of a 96well microtitre plate was set up to contain the following:
50 μl 2 nM [N-methyl-3H]-Nisoxetine hydrochloride (70-87 Ci/mmol, from NEN Life Science Products)
75 μl Assay buffer (50 mM Tris.HCl pH 7.4 containing 300 mM NaCl and 5 mM KCl)
25 μl Test compound, assay buffer (total binding) or 10 μM Desipramine HCl (non-specific binding)
50 μl Wheatgerm agglutinin coated poly(vinyltoluene) (WGA PVT) SPA Beads (Amersham Biosciences RPNQ0001) (10 mg/ml)
50 μl Membrane (0.2 mg protein per ml.)

The microtitre plates were incubated at room temperature for 10 hours prior to reading in a Trilux scintillation counter. The results were analysed using an automatic spline fitting programme (Multicalc, Packard, Milton Keynes, UK) to provide Ki values for each of the test compounds.

Serotonin Binding Assay

The ability of a test compound to compete with [³H]-citalopram from its binding sites on cloned human serotonin membranes has been used as a measure of its ability to block serotonin uptake via its specific transporter (Ramamoorthy, S., Giovanetti, E., Qian, Y., Blakely, R., (1998) J. Biol. Chem. 273,2458).

Membrane Preparation:

The preparation of membrane is essentially similar to that for the norepinephrine transporter containing membrane described above. The membrane preparation was stored in aliquots (1 ml) at −70° C. until required. The protein concentration of the membrane preparation was determined using BCA protein assay reagent kit.

[³]-Citalopram Binding Assay:

Each well of a 96well microtitre plate was set up to contain the following:
50 μl 2 nM [³H]-Citalopram (60-86 Ci/mmol, Amersham Biosciences)
75 μl Assay buffer (50 mM Tris.HCl pH 7.4 containing 150 mM NaCl and 5 mM KCl)
25 μl Diluted compound, assay buffer (total binding) or 100 μM Fluoxetine (non-specific binding)
50 μl WGA PVT SPA Beads (40 mg/ml)
50 μl Membrane preparation (0.4 mg protein per ml)

The microtitre plates were incubated at room temperature for 10 hours prior to reading in a Trilux scintillation counter. The results were analysed using an automatic spline fitting programme (Multicalc, Packard, Milton Keynes, UK) to provide Ki (nM) values for each of the test compounds.

Dopamine Binding Assay

The ability to compete with [3H]-WIN35,428 for its binding sites on human cell membranes containing cloned human dopamine transporter has been used as a measure of its ability to block dopamine uptake via its specific transporter (Ramamoorthy et al 1998 supra).

Membrane Preparation:

Is essentially the same as for membranes containing cloned human serotonin transporter as described above.

[3H]-WIN35,428 Binding Assay:

Each well of a 96well microtitre plate was set up to contain the following:
50 μl 4 nM [3H]-WIN35,428428 (84-87 Ci/mmol, from NEN Life Science Products)
75 μl Assay buffer (50 mM Tris.HCl pH 7.4 containing 150 mM NaCl and 5 mM KCl)
25 μl Diluted compound, assay buffer (total binding) or 100 μM Nomifensine (non-specific binding)
50 μl WGA PVT SPA Beads (10 mg/ml) 50 μl Membrane preparation (0.2 mg protein per ml.)

The microtitre plates were incubated at room temperature for 120 minutes prior to reading in a Trilux scintillation counter. The results were analysed using an automatic spline fitting programme (Multicalc, Packard, Milton Keynes, UK) to provide Ki values for each of the test compounds.

CYP2D6 Assays

Cytochrome P450 2D6 (CYP2D6) is a mammalian enzyme which is commonly associated with the metabolism of around 30% pharmaceutical compounds. Moreover, this enzyme exhibits genetic polymorphism, resulting in the presence of both normal and poor metabolizers in the population. A low involvement of CYP2D6 in the metabolism of compounds (i.e. the compound being a poor substrate of CYP2D6) is desirable in order to reduce any variability from subject to subject in the pharmacokinetics of the compound. Also, compounds with a low inhibitor potential for CYP2D6 are desirable in order to avoid drug-drug interactions with co-administered drugs that are substrates of CYP2D6. Compounds may be tested both as substrates and as inhibitors of this enzyme by means of the following assays.

CYP2D6 Substrate Assay

Principle:

This assay determines the extent of the CYP2D6 enzyme involvement in the total oxidative metabolism of a compound in microsomes. Preferred compounds of the present invention exhibit less than 75% total metabolism via the CYP2D6 pathway.

For this in vitro assay, the extent of oxidative metabolism in human liver microsomes (HLM) is determined after a 30 minute incubation in the absence and presence of Quinidine, a specific chemical inhibitor of CYP2D6. The difference in the extent of metabolism in absence and presence of the inhibitor indicates the involvement of CYP2D6 in the metabolism of the compound.

Materials and Methods:

Human liver microsomes (mixture of 20 different donors, mixed gender) were acquired from Human Biologics (Scottsdale, Ariz., USA). Quinidine and β-NADPH (β-Nicotinamide Adenine Dinucleotide Phosphate, reduced form, tetrasodium salt) were purchased from Sigma (St Louis, Mo., USA). All the other reagents and solvents were of analytical grade. A stock solution of the new chemical entity (NCE) was prepared in a mixture of Acetonitrile/Water to reach a final concentration of acetonitrile in the incubation below 0.5%.

The microsomal incubation mixture (total volume 0.1 mL) contained the NCE (4 μM), β-NADPH (1 mM), microsomal proteins (0.5 mg/mL), and Quinidine (0 or 2 μM) in 100 mM sodium phosphate buffer pH 7.4. The mixture was incubated for 30 minutes at 37° C. in a shaking waterbath. The reaction was terminated by the addition of acetonitrile (75 μL). The samples were vortexed and the denaturated proteins were removed by centrifugation. The amount of NCE in the supernatant was analyzed by liquid chromatography/mass spectrometry (LC/MS) after addition of an internal standard. A sample was also taken at the start of the incubation (t=0), and analysed similarly.

Analysis of the NCE was performed by liquid chromatography/mass spectrometry. Ten μL of diluted samples (20 fold dilution in the mobile phase) were injected onto a Spherisorb CN Column, 5 μM and 2.1 mm×100 mm (Waters corp. Milford, Mass., USA). The mobile phase consisting of a mixture of Solvent A/Solvent B, 30/70 (v/v) was pumped (Alliance 2795, Waters corp. Milford, Mass., USA) through the column at a flow rate of 0.2 ml/minute. Solvent A and Solvent B were a mixture of ammonium formate 5.10⁻³ M pH 4.5/methanol in the proportions 95/5 (v/v) and 10/90 (v/v), for solvent A and solvent B, respectively. The NCE and the internal standard were quantified by monitoring their molecular ion using a mass spectrometer ZMD or ZQ (Waters-Micromass corp, Machester, UK) operated in a positive electrospray ionisation.

The extent of CYP2D6 involvement (% of CYP2D6 involvement) was calculated comparing the extent of metabolism in absence and in presence of quinidine in the incubation.

The extent of metabolism without inhibitor (%) was calculated as follows:

$$\frac{\begin{matrix}( NCE response in samples without inhibitor ) time 0 - \\ ( NCE response in samples without inhibitor ) time 30\end{matrix}}{( NCE response in samples without inhibitor ) time 0} \times 100$$

The extent of metabolism with inhibitor (%) was calculated as follows:

$$\frac{\begin{matrix}( NCE response in samples without inhibitor ) time 0 - \\ ( NCE response in samples with inhibitor ) time 30\end{matrix}}{( NCE response in samples without inhibitor ) time 0} \times 100$$

where the NCE response is the area of the NCE divided by the area of the internal standard in the LC/MS analysis chromatogram, time0 and time30 correspond to the 0 and 30 minutes incubation time.

The % of CYP2D6 involvement was calculated as follows:

$$\frac{\begin{matrix}( \% extent of metabolism without inhibitor ) - \\ ( \% extent of metabolism with inhibitor )\end{matrix}}{\% extent of metbolism without inhibitor} \times 100$$

CYP2D6 Inhibitor Assay

Principle:

The CYP2D6 inhibitor assay evaluates the potential for a compound to inhibit CYP2D6. This is performed by the measurement of the inhibition of the bufuralol 1′-hydroxylase activity by the compound compared to a control. The 1′-hydroxylation of bufuralol is a metabolic reaction specific to CYP2D6. Preferred compounds of the present invention exhibit an IC₅₀ higher than 6 μM for CYP2D6 activity, the IC₅₀ being the concentration of the compound that gives 50% of inhibition of the CYP2D6 activity.

Material and Methods:

Human liver microsomes (mixture of 20 different donors, mixed gender) were acquired from Human Biologics (Scottsdale, Ariz.). β-NADPH was purchased from Sigma (St Louis, Mo.). Bufuralol was purchased from Ultrafine (Manchester, UK). All the other reagents and solvents were of analytical grade.

Microsomal incubation mixture (total volume 0.1 mL) contained bufuralol 10 μM, β-NADPH (2 mM), microsomal proteins (0.5 mg/mL), and the new chemical entity (NCE) (0, 5, and 25 μM) in 100 mM sodium phosphate buffer pH 7.4. The mixture was incubated in a shaking waterbath at 37° C. for 5 minutes. The reaction was terminated by the addition of methanol (75 μL). The samples were vortexed and the denaturated proteins were removed by centrifugation. The supernatant was analyzed by liquid chromatography connected to a fluorescence detector. The formation of the 1′-hydroxybufuralol was monitored in control samples (0 μM NCE) and in the samples S incubated in presence of the NCE. The stock solution of NCE was prepared in a mixture of Acetonitrile/Water to reach a final concentration of acetonitrile in the incubation below 1.0%.

The determination of 1′hydroxybufuralol in the samples was performed by liquid chromatograhy with fluorimetric detection as described below. Twenty five μL samples were injected onto a Chromolith Performance RP-18e column (100 mm×4.6 mm) (Merck KGAa, Darmstadt, Germany). The mobile phase, consisting of a mixture of solvent A and solvent B whose the proportions changed according the following linear gradient, was pumped through the column at a flow rate of 1 ml/min:

| Time (minutes) | Solvent A (%) | Solvent B (%) |
|---|---|---|
| 0 | 65 | 35 |
| 2.0 | 65 | 35 |
| 2.5 | 0 | 100 |
| 5.5 | 0 | 100 |
| 6.0 | 65 | 35 |

Solvent A and Solvent B consisted of a mixture of 0.02 M potassium dihydrogenophosphate buffer pH3/methanol in the proportion 90/10 (v/v) for solvent A and 10/90 (v/v) for solvent B. The run time was 7.5 minutes. Formation of 1′-hydroxybufuralol was monitored by fluorimetric detection with extinction at λ 252 nm and emission at λ 302 nm.

The IC₅₀ of the NCE for CYP2D6 was calculated by the measurement of the percent of inhibition of the formation of the 1′-hydroxybufuralol in presence of the NCE compared to control samples (no NCE) at a known concentration of the NCE.

The percent of inhibition of the formation of the 1′-hydroxybufuralol is calculated as follows:

$$\frac{\begin{matrix}( {1}^{′} \text{-} hydroxybufuralol formed without inhibitor ) - \\ ( {1}^{′} hydroxybufuralol formed with inhibitor )\end{matrix}}{( {1}^{′} hydroxybufuralol area formed without inhibitor )} \times 100$$

The IC₅₀ is calculated from the percent inhibition of the formation of the 1′-hydroxybufuralol as follows (assuming competitive inhibition):

$$\frac{NCE Concentration \times ( 100 \text{-} Percent of inhibition )}{Percent of inhibition}$$

The IC₅₀ estimation is assumed valid if inhibition is between 20% and 80% (Moody G C, Griffin S J, Mather A N, McGinnity D F, Riley R J. 1999. Fully automated analysis of activities catalyzed by the major human liver cytochrome P450 (CYP) enzymes: assessment of human CYP inhibition potential. Xenobiotica, 29(1): 53-75).

1. A compound of formula I 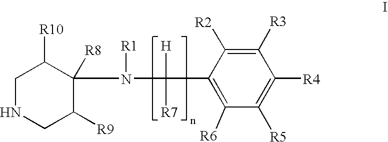
wherein
n is 1, 2 or 3;
R1 is C₂-C₁₀alkyl, C₂-C₁₀alkenyl, C₃-C₈cycloalkyl or C₄-C₁₀cycloalkylalkyl, wherein one C—C bond within any cycloalkyl moiety is optionally substituted by an O—C, S—C or C═C bond and wherein each group is optionally substituted with from 1 to 7 halogen substituents and/or with from 1 to 3 substituents each independently selected from hydroxy, cyano, C₁-C₄alkyl, C₁-C₄alkylthio (optionally substituted with from 1 to 3 halogen atoms) and C₁-C₄alkoxy (optionally substituted with from 1 to 3 halogen atoms);
R2 is H, C₁-C₄alkyl (optionally substituted with from 1 to 7 halogen atoms), C₁-C₄alkyl-S(O)ₓ— wherein x is 0, 1 or 2 (optionally substituted with from 1 to 7 halogen atoms), C₁-C₄alkoxy (optionally substituted with from 1 to 7 halogen atoms), cyano, halogen, phenyl (optionally substituted with from 1 to 3 substituents each independently selected from halogen, C₁-C₄alkyl and C₁-C₄alkoxy), phenoxy (optionally substituted with from 1 to 3 substituents each independently selected from halogen, C₁-C₄alkyl and C₁-C₄alkoxy) or —CO₂(C₁-C₄alkyl), or together with R3 forms a further benzene ring (optionally substituted with from 1 to 3 substituents each independently selected from halogen, C₁-C₄alkyl and C₁-C₄alkoxy);
R3 is H, C₁-C₄alkyl (optionally substituted with from 1 to 7 halogen atoms), C₁-C₄alkyl-S(O)ₓ— wherein x is 0, 1 or 2 (optionally substituted with from 1 to 7 halogen atoms), C₁-C₄alkoxy (optionally substituted with from 1 to 7 halogen atoms), cyano, halogen, phenyl (optionally substituted with from 1 to 3 substituents each independently selected from halogen, C₁-C₄alkyl and C₁-C₄alkoxy), phenoxy (optionally substituted with from 1 to 3 substituents each independently selected from halogen, C₁-C₄alkyl and C₁-C₄alkoxy) or —CO₂(C₁-C₄alkyl), or together with R2 or R4 forms a further benzene ring (optionally substituted with from 1 to 3 substituents each independently selected from halogen, C₁-C₄alkyl and C₁-C₄alkoxy);
R4 is H, C₁-C₄alkyl (optionally substituted with from 1 to 7 halogen atoms), C₁-C₄alkyl-S(O)ₓ— wherein x is 0, 1 or 2 (optionally substituted with from 1 to 7 halogen atoms), C₁-C₄alkoxy (optionally substituted with from 1 to 7 halogen atoms), cyano, halogen, phenyl (optionally substituted with from 1 to 3 substituents each independently selected from halogen, C₁-C₄alkyl and C₁-C₄alkoxy), phenoxy (optionally substituted with from 1 to 3 substituents each independently selected from halogen, C₁-C₄alkyl and C₁-C₄alkoxy) or —CO₂(C₁-C₄alkyl), or together with R3 forms a further benzene ring (optionally substituted with from 1 to 3 substituents each independently selected from halogen, C₁-C₄alkyl and C₁-C₄alkoxy);
R5 is H, C₁-C₄alkyl (optionally substituted with from 1 to 7 halogen atoms), C₁-C₄alkoxy (optionally substituted with from 1 to 7 halogen atoms) or halogen;
R6 is H, C₁-C₄alkyl (optionally substituted with from 1 to 7 halogen atoms), C₁-C₄alkoxy (optionally substituted with from 1 to 7 halogen atoms) or halogen;
R7 is H or C₁-C₄alkyl;
R8 is H or C₁-C₄alkyl;
R9 is H, halogen, hydroxy, cyano, C₁-C₄alkyl or C₁-C₄alkoxy; and
R10 is H, halogen, hydroxy, cyano, C₁-C₄alkyl or C₁-C₄alkoxy;
or a pharmaceutically acceptable salt thereof,
with the proviso that the compound N-ethyl-N-benzyl-4-piperidinamine is excluded.

2. A compound as claimed in claim 1 wherein n is 1 or 2.

3. A compound as claimed in any one of claims 1 or 2 wherein R7 is H or methyl.

4. A compound as claimed in any one of claims 1 or 2 wherein R8 is H.

5. A compound as claimed in any one of claims 1 or 2 wherein R9 is H or fluoro.

6. A compound as claimed in any one of claims 1 or 2 wherein R10 is H or fluoro.

7. A compound as claimed in any one of claims 1 or 2 wherein R1 is C₂-C₆alkyl, C₂-C₆alkenyl, C₃-C₆cycloalkyl or C₄-C₇cycloalkylalkyl, wherein one C—C bond within any cycloalkyl moiety is optionally substituted by an O—C bond and wherein each group is optionally substituted with from 1 to 3 halogen atoms or a hydroxy, cyano, C₁-C₄alkylthio (optionally substituted with from 1 to 3 halogen atoms) or C₁-C₄alkoxy (optionally substituted with from 1 to 3 halogen atoms) radical.

8. A compound as claimed in any one of claims 1 or 2 wherein R1 is C₂-C₆alkyl (optionally substituted with from 1 to 3 halogen atoms or a hydroxy, cyano, methylthio, methoxy, trifluoromethoxy, ethoxy, or isopropoxy radical), C₂-C₆alkenyl, C₃-C₆cycloalkyl or C₄-C₇cycloalkylalkyl (optionally substituted with a halogen atom or hydroxy radical), wherein one C—C bond within any cycloalkyl moiety is optionally substituted by an O—C bond.

9. A compound as claimed in any one of claims 1 or 2 wherein R1 is ethyl, 2-cyanoethyl, 2-hydroxyethyl, 2-methoxyethyl, 2-trifluoromethoxyethyl, 2-methylthioethyl, 2-ethoxyethyl, 2-isopropoxyethyl, 2,2,2-trifluoroethyl, n-propyl, isopropyl, 3-methoxypropyl, 3-hydroxypropyl, 3-cyanopropyl, 3,3,3-trifluoropropyl, n-butyl, isobutyl, 4-methoxybutyl, 4,4,4-trifluorobutyl, 2-methoxy-2-methylpropyl, 2-hydroxy-2-methylpropyl, 2-cyano-2-methylpropyl, n-pentyl, 3-methylbutyl, 3-cyano-3-methylbutyl, 3-hydroxy-3-methylbutyl, 1,2-dimethylpropyl, 2,2-dimethylpropyl, 2,2-dimethyl-3-hydroxypropyl, 1-ethylpropyl, 3,3-dimethylbutyl, 2-ethylbutyl, 2-methylpentyl, 2methyl-2-propenyl, cyclopentyl, tetrahydro-2H-pyran-4-yl, cycloheptylmethyl, cyclohexylmethyl, tetrahydro-2H-pyran-4-ylmethyl, cyclopentylmethyl, hydroxycyclopentylmethyl, cyclobutylmethyl, cyclopropylmethyl and fluorocyclopropylmethyl.

10. A compound as claimed in any one of claims 1 or 2 wherein R2 is H, methyl, trifluoromethyl, methylthio, tert-butylthio, trifluoromethylthio, methylsulfonyl, methoxy, ethoxy, difluoromethoxy, trifluoromethoxy, cyano, fluoro, chloro, bromo, phenyl or phenoxy, or together with R3 forms a further benzene ring.

11. A compound as claimed in any one of claims 1 or 2 wherein R3 is H, methyl, trifluoromethyl, trifluoromethylthio, methoxy, ethoxy, difluoromethoxy, trifluoromethoxy, cyano, fluoro, chloro, bromo, phenyl, phenoxy or CO₂CH₃, or together with R2 or R4 forms a further benzene ring.

12. A compound as claimed in any one of claims 1 or 2 wherein R4 is H, methyl, trifluoromethyl, methylthio, methoxy, trifluoromethoxy, cyano, fluoro, chloro, phenyl or CO₂CH₃, or together with R3 forms a further benzene ring.

13. A compound as claimed in any one of claims 1 or 2 wherein R5 is H, methyl, methoxy, fluoro or chloro.

14. A compound as claimed in any one of claims 1 or 2 wherein R6 is H, methyl, fluoro or chloro.

15. A compound as claimed in claim 1 wherein the group 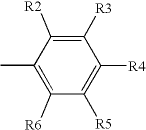
is phenyl, 2-methylphenyl, 2-(trifluoromethyl)phenyl, 2-(methylthio)phenyl, 2-(tertbutylthio)phenyl, 2-(trifluoromethylthio)phenyl, 2-(methylsulfonyl)phenyl, 2-methoxyphenyl, 2-ethoxyphenyl, 2-(difluoromethoxy)phenyl, 2-(trifluoromethoxy)phenyl, 2-cyanophenyl, 2-fluorophenyl, 2-chlorophenyl, 2-bromophenyl, 2-biphenyl, 2-phenoxyphenyl, 3-methylphenyl, 3-(trifluoromethyl)phenyl, 3-(trifluoromethylthio)phenyl, 3-methoxyphenyl, 3-ethoxyphenyl, 3-(difluoromethoxy)phenyl, 3-(trifluoromethoxy)phenyl, 3-cyanophenyl, 3-fluorophenyl, 3-chlorophenyl, 3-bromophenyl, 3-biphenyl, 3-phenoxyphenyl, 3-(methoxycarbonyl)phenyl, 4-methylphenyl, 4-(trifluoromethyl)phenyl, 4-(methylthio)phenyl, 4-methoxyphenyl, 4-(trifluoromethoxy)phenyl, 4-cyanophenyl, 4-fluorophenyl, 4-chlorophenyl, 4-biphenyl, 4-(methoxycarbonyl)phenyl, 2,3-dichlorophenyl, 2-chloro-3-methylphenyl, 2-chloro-3-(trifluoromethyl)phenyl, 2,4-dimethylphenyl, 2,4-bis(trifluoromethyl)phenyl, 2,4-dimethoxyphenyl, 2,4-difluorophenyl, 2,4-dichlorophenyl, 2-chloro-4-fluorophenyl, 2-fluoro-4-(trifluoromethyl)phenyl, 2-chloro-4-(methylsulfonyl)phenyl 2,5-dimethylphenyl, 2,5-dichlorophenyl, 2-chloro-5-(trifluoromethyl)phenyl, 2,6-dimethylphenyl, 2,6-dichlorophenyl, 2-chloro-6-fluorophenyl, 2-fluoro-6-(trifluoromethyl)phenyl, 3-chloro-2-methylphenyl, 3-chloro-2-fluorophenyl, 3-chloro-2-(trifluoromethyl)phenyl, 3,4-dichlorophenyl, 3-chloro-4-fluorophenyl, 3,5-dimethylphenyl, 3,5-dimethoxyphenyl, 3,5-difluorophenyl, 3,5-dichlorophenyl, 3-fluoro-5-(trifluoromethyl)phenyl, 5-fluoro-2-(trifluoromethylphenyl), 5-fluoro-2-methoxyphenyl, 4-fluoro-2(trifluoromethyl)phenyl, 4-chloro-3-(trifluoromethyl)phenyl, 2,3,6-trichlorophenyl, 2,3,5-trichlorophenyl, 3-chloro-2-fluoro-6-(trifluoromethyl)phenyl, 3-chloro-2-fluoro-5-(trifluoromethyl)phenyl, 2-chloro-6-fluoro-3-methylphenyl, 2-chloro-6-fluoro-5-methylphenyl, 1-naphthyl or 2-naphthyl.

16. A compound as claimed in claim 1 wherein R2, R3, R4, R5 and R6 are all H.

17. A compound as claimed in claim 1 wherein one of R2, R3, R4, R5 and R6 is not H and the others are H.

18. A compound as claimed claim 1 wherein two of R2, R3, R4, R5 and R6 are not H and the others are H.

19. A compound as claimed in claim 1 wherein three of R2, R3, R4, R5 and R6 are not H and the others are H.

20. A process for producing a compound as claimed in claim 1, which comprises deprotecting a compound of the formula 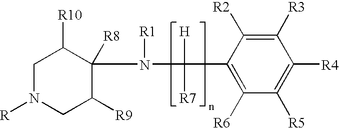
where R is an N-protecting group and n and R1 to R10 are as defined in claim 1, optionally followed-by the step of forming a pharmaceutically acceptable salt.

21. (canceled)

22. A method for inhibiting the uptake of one or more monoamines selected from serotonin, dopamine and norepinephrine in a mammal, comprising administering to a mammal in need of such inhibition an effective amount of a compound as claimed in claim 1 or a pharmaceutically acceptable salt thereof.

23. (canceled)

24. A pharmaceutical composition comprising a compound as claimed in claim 1 or a pharmaceutically acceptable salt thereof together with a pharmaceutically acceptable diluent or carrier.
